(19) United States
(12) Patent Application Publication
(10) Pub. No.: US 2012/0015299 A1
(43) Pub. Date: Jan. 19, 2012

(54) RESIST COMPOSITION, METHOD OF FORMING RESIST PATTERN, NOVEL COMPOUND, AND ACID GENERATOR

(75) Inventors: Yoshitaka Komuro, Kawasaki-shi (JP); Yoshiyuki Utsumi, Kawasaki-shi (JP)

(73) Assignee: TOKYO OHKA KOGYO CO., LTD., Kawasaki-shi (JP)

(21) Appl. No.: 13/179,864

(22) Filed: Jul. 11, 2011

(30) Foreign Application Priority Data
Jul. 15, 2010 (JP) 2010-160495
Mar. 18, 2011 (JP) 2011-061514

(51) Int. Cl. G03F 7/039; C07D 313/06; G03F 7/30

(52) U.S. Cl. 430/285.1; 430/270.1; 430/326; 549/268

(57) ABSTRACT

A resist composition including a base component (A) which exhibits changed solubility in an alkali developing solution under action of acid and an acid-generator component (B) which generates acid upon exposure, the acid-generator component (B) including an acid generator (B1) represented by general formula (b1-1) [in the formula, Y⁰ represents an alkylene group of 1 to 4 carbon atoms or a fluorinated alkylene group, R⁰ represents an alkyl group, an alkoxy group, a halogen atom, a halogenated alkyl group, a hydroxy group or an oxygen atom (═O); p represents 0 or 1; and Z⁺ represents an organic cation.

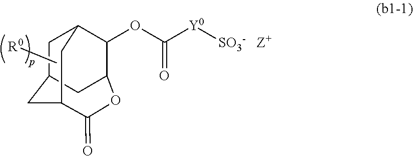

### TECHNICAL FIELD

The present invention relates to a resist composition, a method of forming a resist pattern using the same, a novel compound useful as an acid generator for a resist composition, and an acid generator.

Priority is claimed on Japanese Patent Application No. 2010-160495, filed Jul. 15, 2010, and Japanese Patent Application No. 2011-061514, filed Mar. 18, 2011, the contents of which are incorporated herein by reference.

### BACKGROUND ART

In lithography techniques, for example, a resist film composed of a resist material is formed on a substrate, and the resist film is subjected to selective exposure of radial rays such as light or electron beam through a mask having a predetermined pattern, followed by development, thereby forming a resist pattern having a predetermined shape on the resist film.

A resist material in which the exposed portions become soluble in a developing solution is called a positive-type, and a resist material in which the exposed portions become insoluble in a developing solution is called a negative-type.

In recent years, in the production of semiconductor elements and liquid crystal display elements, advances in lithography techniques have lead to rapid progress in the field of pattern miniaturization.

Typically, these miniaturization techniques involve shortening the wavelength (increasing the energy) of the exposure light source. Conventionally, ultraviolet radiation typified by g-line and i-line radiation has been used, but nowadays KrF excimer lasers and ArF excimer lasers are starting to be introduced in mass production. Furthermore, research is also being conducted into lithography techniques that use an exposure light source having a wavelength shorter (energy higher) than these excimer lasers, such as electron beam, extreme ultraviolet radiation (EUV), and X ray.

Resist materials for use with these types of exposure light sources require lithography properties such as a high resolution capable of reproducing patterns of minute dimensions, and a high level of sensitivity to these types of exposure light sources.

As a resist material that satisfies these conditions, a chemically amplified composition is used, which includes a base material component that exhibits a changed solubility in an alkali developing solution under the action of acid and an acid-generator component that generates acid upon exposure.

For example, a chemically amplified positive resist contains, as a base component (base resin), a resin which exhibits increased solubility in an alkali developing solution under action of acid, and an acid generator is typically used. If the resist film formed using the resist composition is selectively exposed during formation of a resist pattern, then within the exposed portions, acid is generated from the acid-generator component, and the action of this acid causes an increase in the solubility of the resin component in an alkali developing solution, making the exposed portions soluble in the alkali developing solution.

Currently, resins that contain structural units derived from (meth)acrylate esters within the main chain (acrylic resins) are now widely used as base resins for resist compositions that use ArF excimer laser lithography, as they exhibit excellent transparency in the vicinity of 193 nm (for example, see Patent Document 1).

On the other hand, as acid generators usable in a chemically amplified resist composition, various types have been proposed including, for example, onium salt acid generators such as iodonium salts and sulfonium salts; oxime sulfonate acid generators; diazomethane acid generators; nitrobenzylsulfonate acid generators; iminosulfonate acid generators; and disulfone acid generators.

Among these, as acid generators, onium salt acid generators having an onium ion such as triphenylsulfonium as the cation moiety are particularly used. As the anion moiety for onium salt acid generators, an alkylsulfonate ion or a fluorinated alkylsulfonate ion in which part or all of the hydrogen atoms within the aforementioned alkylsulfonate ion has been substituted with fluorine atoms is typically used.

Further, there have been proposed resist compositions containing a sulfonium compound or a iodonium compound as an acid generator which has a “lactone-containing cyclic group” having —C(═O)—O— in the ring structure in the anion moiety (for example, see Patent Documents 2 and 3). As an acid generator having a “lactone-containing cyclic group” in the anion moiety, for example, Patent Document 3 discloses on paragraph [0055] an acid generator having an adamantanelactone anion represented by chemical formula (B2-4) shown below as the anion moiety.

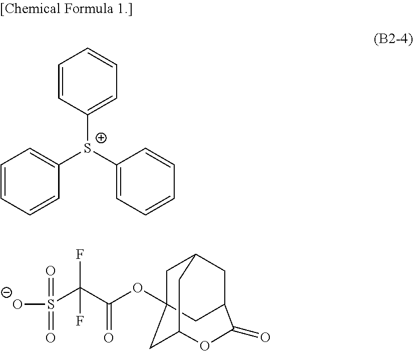

### DOCUMENTS OF RELATED ART

### Patent Document

[Patent Document 1] Japanese Unexamined Patent Application, First Publication No. 2003-241385
[Patent Document 2] Japanese Unexamined Patent Application, First Publication No. 2006-306856
[Patent Document 3] Japanese Unexamined Patent Application, First Publication No. 2010-39146

### SUMMARY OF THE INVENTION

Currently, among the aforementioned onium salt-based acid generators, onium salt-based acid generators having a perfluoroalkylsulfonic acid ion as the anion moiety are generally used.

In recent years, as miniaturization of resist patterns progress, further improvement in resist pattern shape and various lithography properties have been demanded for conventional chemically amplified resist compositions containing an onium salt-based acid generator having a perfluoroalkylsulfonic acid ion as the anion moiety.

However, Patent Documents 2 and 3 do not disclose a resist composition and an acid generator which satisfies the characteristics required in the formation of a resist pattern, such as roughness, mask reproducibility, exposure latitude and rectangularity of the resist pattern shape. Further, in the synthesis of the acid generator represented by the aforementioned chemical formula (B2-4), a tertiary alcohol is used as a starting material which exhibits a poor reactivity, and hence, the yield was deteriorated.

Therefore, there has been a demand for a compound more useful as an acid generator for a resist composition.

The present invention takes the above circumstances into consideration, with an object of providing a compound useful as an acid generator for a resist composition, an acid generator including the compound, a resist composition containing the acid generator, and a method of forming a resist pattern using the resist composition.

For solving the above-mentioned problems, the present invention employs the following aspects.

Specifically, a first aspect of the present invention is a resist composition including a base component (A) which exhibits changed solubility in an alkali developing solution under action of acid and an acid-generator component (B) which generates acid upon exposure, the acid-generator component (B) including an acid generator (B1) represented by general formula (b1-1) shown below.

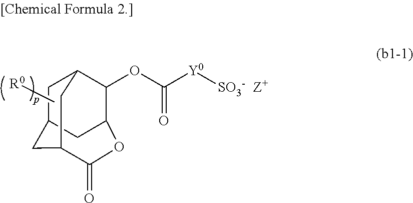

In the formula, Y⁰ represents an alkylene group of 1 to 4 carbon atoms which may have a substituent or a fluorinated alkylene group which may have a substituent; R⁰ represents an alkyl group, an alkoxy group, a halogen atom, a halogenated alkyl group, a hydroxy group or an oxygen atom (═O); p represents 0 or 1; and Z⁺ represents an organic cation.

A second aspect of the present invention is a method of forming a resist pattern, including forming a resist film on a substrate using a resist composition according to the first aspect, subjecting the resist film to exposure, and subjecting the resist film to alkali developing to form a resist pattern.

A third aspect of the present invention is a compound represented by general formula (b1-1) shown below.

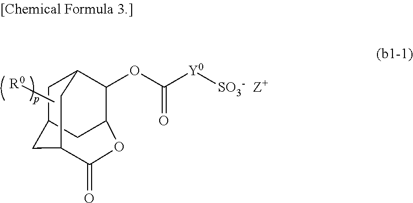

In the formula, Y⁰ represents an alkylene group of 1 to 4 carbon atoms which may have a substituent or a fluorinated alkylene group which may have a substituent; R⁰ represents an alkyl group, an alkoxy group, a halogen atom, a halogenated alkyl group, a hydroxy group or an oxygen atom (═O); p represents 0 or 1; and Z⁺ represents an organic cation.

A fourth aspect of the present invention is an acid generator including the compound of the third aspect.

In the present description and claims, an “alkyl group” includes linear, branched or cyclic, monovalent saturated hydrocarbon, unless otherwise specified.

The term “alkylene group” includes linear, branched or cyclic divalent saturated hydrocarbon, unless otherwise specified.

A “lower alkyl group” is an alkyl group of 1 to 5 carbon atoms.

A “halogenated alkyl group” is a group in which part or all of the hydrogen atoms of an alkyl group is substituted with a halogen atom. Examples of the halogen atom include a fluorine atom, a chlorine atom, a bromine atom and an iodine atom.

The term “aliphatic” is a relative concept used in relation to the term “aromatic”, and defines a group or compound that has no aromaticity.

The term “structural unit” refers to a monomer unit that contributes to the formation of a polymeric compound (polymer, copolymer).

The term “exposure” is used as a general concept that includes irradiation with any form of radiation.

The term “(meth)acrylic acid” is a generic term that includes either or both of acrylic acid having a hydrogen atom bonded to the α-position and methacrylic acid having a methyl group bonded to the α-position.

The term “(meth)acrylate ester” is a generic term that includes either or both of the acrylate ester having a hydrogen atom bonded to the α-position and the methacrylate ester having a methyl group bonded to the α-position.

The term “(meth)acrylate” is a generic term that includes either or both of the acrylate having a hydrogen atom bonded to the α-position and the methacrylate having a methyl group bonded to the α-position.

According to the present invention, there are provided a compound useful as an acid generator for a resist composition, an acid generator including the compound, a resist composition containing the acid generator, and a method of forming a resist pattern using the resist composition.

By using the resist composition and method of forming a resist pattern according to the present invention, excellent lithography properties such as roughness, mask reproducibility and exposure latitude can be achieved, and a resist pattern having an excellent shape with high rectangularity can be formed.

### DETAILED DESCRIPTION OF THE INVENTION

### Resist Composition

The resist composition according to the first aspect of the present invention includes a base component (A) which exhibits changed solubility in an alkali developing solution under action of acid (hereafter, referred to as “component (A)”) and an acid-generator component (B) which generates acid upon exposure (hereafter, referred to as “component (B)”).

With respect to a resist film formed using the resist composition, when a selective exposure is conducted during formation of a resist pattern, acid is generated from the component (B), and the generated acid acts on the component (A) to change the solubility of the component (A) in an alkali developing solution. As a result, the solubility of the exposed portions in an alkali developing solution is changed, whereas the solubility of the unexposed portions in an alkali developing solution remains unchanged. Therefore, the exposed portions are dissolved and removed by alkali developing in the case of a positive resist composition, whereas unexposed portions are dissolved and removed in the case of a negative resist composition, and hence, a resist pattern can be formed.

The resist composition of the present invention may be either a negative resist composition or a positive resist composition.

<Component (A)>

As the component (A), an organic compound typically used as a base component for a chemically amplified resist composition can be used alone, or two or more of such organic compounds can be mixed together.

Here, the term “base component” refers to an organic compound capable of forming a film, and is preferably an organic compound having a molecular weight of 500 or more. When the organic compound has a molecular weight of 500 or more, the film-forming ability is improved, and a resist pattern of nano level can be easily formed.

The “organic compound having a molecular weight of 500 or more” which can be used as a base component is broadly classified into non-polymers and polymers.

In general, as a non-polymer, any of those which have a molecular weight in the range of 500 to less than 4,000 is used. Hereafter, a non-polymer having a molecular weight in the range of 500 to less than 4,000 is referred to as a low molecular weight compound.

As a polymer, any of those which have a molecular weight of 1,000 or more is generally used. Hereafter, a polymer having a molecular weight of 1,000 or more is referred to as a polymeric compound. With respect to a polymeric compound, the “molecular weight” is the weight average molecular weight in terms of the polystyrene equivalent value determined by gel permeation chromatography (GPC). Hereafter, a polymeric compound is frequently referred to simply as a “resin”.

As the component (A), a resin component which exhibits changed solubility in an alkali developing solution under action of acid may be used. Alternatively, as the component (A), a low molecular weight material which exhibits changed solubility in an alkali developing solution under action of acid may be used.

When the resist composition of the present invention is a negative resist composition, for example, as the component (A), a base component that is soluble in an alkali developing solution is used, and a cross-linking agent is blended in the negative resist composition.

In the negative resist composition, when acid is generated from the component (B) upon exposure, the action of the generated acid causes cross-linking between the base component and the cross-linking agent, and the cross-linked portion becomes insoluble in an alkali developing solution. Therefore, in the formation of a resist pattern, by conducting selective exposure of a resist film formed by applying the negative resist composition onto a substrate, the exposed portions become insoluble in an alkali developing solution, whereas the unexposed portions remain soluble in an alkali developing solution, and hence, a resist pattern can be formed by alkali developing.

Generally, as the component (A) for a negative resist composition, a resin that is soluble in an alkali developing solution (hereafter, referred to as “alkali-soluble resin”) is used.

Examples of the alkali soluble resin include a resin having a structural unit derived from at least one of α-(hydroxyalkyl)acrylic acid and an alkyl ester of α-(hydroxyalkyl)acrylic acid (preferably an alkyl ester having 1 to 5 carbon atoms), as disclosed in Japanese Unexamined Patent Application, First Publication No. 2000-206694; a (meth)acrylic resin or polycycloolefin resin having a sulfoneamide group, as disclosed in U.S. Pat. No. 6,949,325; a resin having a fluorinated alcohol, as disclosed in U.S. Pat. No. 6,949,325, Japanese Unexamined Patent Application, First Publication No. 2005-336452 or Japanese Unexamined Patent Application, First Publication No. 2006-317803; and a polycyclolefin resin having a fluorinated alcohol, as disclosed in Japanese Unexamined Patent Application, First Publication No. 2006-259582. These resins are preferable in that a resist pattern can be formed with minimal swelling.

Here, the term “α-(hydroxyalkyl)acrylic acid” refers to one or both of acrylic acid in which a hydrogen atom is bonded to the carbon atom on the α-position having the carboxyl group bonded thereto, and α-hydroxyalkylacrylic acid in which a hydroxyalkyl group (preferably a hydroxyalkyl group of 1 to 5 carbon atoms) is bonded to the carbon atom on the α-position.

As the cross-linking agent, typically, an amino-based cross-linking agent such as a glycoluril having a methylol group or alkoxymethyl group, or a melamine-based cross-linking agent is preferable, as it enables formation of a resist pattern with minimal swelling. The amount of the cross-linker added is preferably within a range from 1 to 50 parts by weight, relative to 100 parts by weight of the alkali-soluble resin.

When the resist composition of the present invention is a positive resist composition, as the component (A), a base component which exhibits increased solubility in an alkali developing solution by action of acid (hereafter, referred to as “component (A0)”) is used.

More specifically, the component (A0) is substantially insoluble in an alkali developing solution prior to exposure, but when acid is generated from the component (B) upon exposure, the action of this acid causes an increase in the solubility of the base component in an alkali developing solution. Therefore, in the formation of a resist pattern, by conducting selective exposure of a resist film formed by applying the positive resist composition onto a substrate, the exposed portions changes from an insoluble state to a soluble state in an alkali developing solution, whereas the unexposed portions remain insoluble in an alkali developing solution, and hence, a resist pattern can be formed by alkali developing.

In the resist composition of the present invention, the component (A) is preferably a base component (A0) which exhibits increased solubility in an alkali developing solution under action of acid. That is, the resist composition of the present invention is preferably a positive resist composition.

The component (A0) may be a resin component (A1) that exhibits increased solubility in an alkali developing solution under the action of acid (hereafter, frequently referred to as “component (A1)”), a low molecular weight material (A2) that exhibits increased solubility in an alkali developing solution under the action of acid (hereafter, frequently referred to as “component (A2)”), or a mixture thereof.

[Component (A1)]

As the component (A1), a resin component (base resin) typically used as a base component for a chemically amplified resist composition can be used alone, or two or more of such resin components can be mixed together.

In the present invention, the component (A1) preferably has a structural unit derived from an acrylate ester or a structural unit derived from an acrylate ester having an atom other than hydrogen or a substituent bonded to the carbon atom on the α position.

In the present descriptions and the claims, the expression “structural unit derived from an acrylate ester” refers to a structural unit that is formed by the cleavage of the ethylenic double bond of an acrylate ester.

An “acrylate ester” refers to an acrylate ester having a hydrogen atom bonded to the carbon atom on the α position.

With respect to the “acrylate ester which has an atom other than hydrogen or a substituent bonded to the carbon atom on the α position”, examples of the atom other than hydrogen include halogen atoms, and examples of the substituent include an alkyl group of 1 to 5 carbon atoms and a halogenated alkyl group of 1 to 5 carbon atoms. Examples of the halogen atom include a fluorine atom, a chlorine atom, a bromine atom and an iodine atom.

With respect to the “structural unit derived from an acrylate ester”, the “α-position (the carbon atom on the α-position)” refers to the carbon atom having the carbonyl group bonded thereto, unless specified otherwise.

With respect to the acrylate ester, specific examples of the alkyl group of 1 to 5 carbon atoms for the substituent at the α-position include linear or branched alkyl groups such as a methyl group, an ethyl group, a propyl group, an isopropyl group, an n-butyl group, an isobutyl group, a tert-butyl group, a pentyl group, an isopentyl group, and a neopentyl group.

Specific examples of the halogenated alkyl group of 1 to 5 carbon atoms include groups in which part or all of the hydrogen atoms of the aforementioned “alkyl group of 1 to 5 carbon atoms for the substituent at the α-position” are substituted with halogen atoms. Examples of the halogen atom include a fluorine atom, a chlorine atom, a bromine atom and an iodine atom, and a fluorine atom is particularly desirable.

In the present invention, it is preferable that a hydrogen atom, an alkyl group of 1 to 5 carbon atoms or a halogenated alkyl group of 1 to 5 carbon atoms is bonded to the α-position of the acrylate ester, a hydrogen atom, an alkyl group of 1 to 5 carbon atoms or a fluorinated alkyl group of 1 to 5 carbon atoms is more preferable, and in terms of industrial availability, a hydrogen atom or a methyl group is the most desirable.

In the resist composition of the present invention, it is particularly desirable that the component (A1) has a structural unit (a1) derived from an acrylate ester which may have an atom other than hydrogen or a substituent bonded to the carbon atom on the α position and contains an acid dissociable, dissolution inhibiting group.

Further, in addition to the structural unit (a1), it is preferable that the component (A1) has a structural unit (a2) derived from an acrylate ester which may have an atom other than hydrogen or a substituent bonded to the carbon atom on the α position and contains a lactone-containing cyclic group.

Further, in addition to the structural unit (a1), it is preferable that the component (A1) has a structural unit (a3) derived from an acrylate ester which may have an atom other than hydrogen or a substituent bonded to the carbon atom on the α position and contains a polar group-containing aliphatic hydrocarbon group.

Furthermore, the component (A1) preferably has a structural unit (a0) derived from an acrylate ester which may have an atom other than hydrogen or a substituent bonded to the carbon atom on the α position and contains an —SO₂— group containing cyclic group.

Moreover, in the present invention, the component (A1) may also include a structural unit other than the aforementioned structural units (a1) to (a3) and (a0).

(Structural Unit (a1))

The structural unit (a1) is a structural unit derived from an acrylate ester which may have an atom other than hydrogen or a substituent bonded to the carbon atom on the α position and contains an acid dissociable, dissolution inhibiting group.

As the acid dissociable, dissolution inhibiting group in the structural unit (a1), any of the groups that have been proposed as acid dissociable, dissolution inhibiting groups for the base resins of chemically amplified resists can be used, provided the group has an alkali dissolution-inhibiting effect that renders the entire component (A1) insoluble in an alkali developing solution prior to dissociation, and then following dissociation by action of acid, increases the solubility of the entire component (A1) in the alkali developing solution. Generally, groups that form either a cyclic or chain-like tertiary alkyl ester with the carboxyl group of the (meth)acrylic acid, and acetal-type acid dissociable, dissolution inhibiting groups such as alkoxyalkyl groups are widely known.

Here, a tertiary alkyl ester describes a structure in which an ester is formed by substituting the hydrogen atom of a carboxyl group with a chain-like or cyclic tertiary alkyl group, and a tertiary carbon atom within the chain-like or cyclic tertiary alkyl group is bonded to the oxygen atom at the terminal of the carbonyloxy group (—C(═O)—O—). In this tertiary alkyl ester, the action of acid causes cleavage of the bond between the oxygen atom and the tertiary carbon atom.

The chain-like or cyclic alkyl group may have a substituent. Hereafter, for the sake of simplicity, groups that exhibit acid dissociability as a result of the formation of a tertiary alkyl ester with a carboxyl group are referred to as “tertiary alkyl ester-type acid dissociable, dissolution inhibiting groups”.

Examples of tertiary alkyl ester-type acid dissociable, dissolution inhibiting groups include aliphatic branched, acid dissociable, dissolution inhibiting groups and aliphatic cyclic group-containing acid dissociable, dissolution inhibiting groups.

In the present description and claims, the term “aliphatic branched” refers to a branched structure having no aromaticity.

The “aliphatic branched, acid dissociable, dissolution inhibiting group” is not limited to be constituted of only carbon atoms and hydrogen atoms (not limited to hydrocarbon groups), but is preferably a hydrocarbon group.

Further, the “hydrocarbon group” may be either saturated or unsaturated, but is preferably saturated.

Examples of aliphatic branched, acid dissociable, dissolution inhibiting groups include tertiary alkyl groups of 4 to 8 carbon atoms, and specific examples include a tert-butyl group, tert-pentyl group and tert-heptyl group.

The term “aliphatic cyclic group” refers to a monocyclic group or polycyclic group that has no aromaticity.

The “aliphatic cyclic group” within the structural unit (a1) may or may not have a substituent. Examples of substituents include lower alkyl groups of 1 to 5 carbon atoms, lower alkoxy groups of 1 to 5 carbon atoms, fluorine atom, fluorinated lower alkyl groups of 1 to 5 carbon atoms, and oxygen atom (═O).

The basic ring of the “aliphatic cyclic group” exclusive of substituents is not limited to be constituted from only carbon and hydrogen (not limited to hydrocarbon groups), but is preferably a hydrocarbon group.

Further, the “hydrocarbon group” may be either saturated or unsaturated, but is preferably saturated. Furthermore, the “aliphatic cyclic group” is preferably a polycyclic group.

As such aliphatic cyclic groups, groups in which one or more hydrogen atoms have been removed from a monocycloalkane or a polycycloalkane such as a bicycloalkane, tricycloalkane or tetracycloalkane which may or may not be substituted with a lower alkyl group, a fluorine atom or a fluorinated alkyl group, may be used. Specific examples include groups in which one or more hydrogen atoms have been removed from a monocycloalkane such as cyclopentane and cyclohexane; and groups in which one or more hydrogen atoms have been removed from a polycycloalkane such as adamantane, norbornane, isobornane, tricyclodecane or tetracyclododecane.

As the aliphatic cyclic group-containing acid dissociable, dissolution inhibiting group, for example, a group which has a tertiary carbon atom on the ring structure of the cycloalkyl group can be used. Specific examples include 2-methyl-2-adamantyl group and a 2-ethyl-2-adamantyl group. Further, groups having an aliphatic cyclic group such as an adamantyl group, cyclohexyl group, cyclopentyl group, norbornyl group, tricyclodecyl group or tetracyclododecyl group, and a branched alkylene group having a tertiary carbon atom bonded thereto, as the groups bonded to the oxygen atom of the carbonyl group (—C(O)—O—) within the structural units represented by general formulas (a1″-1) to (a1″-6) shown below, can be used.

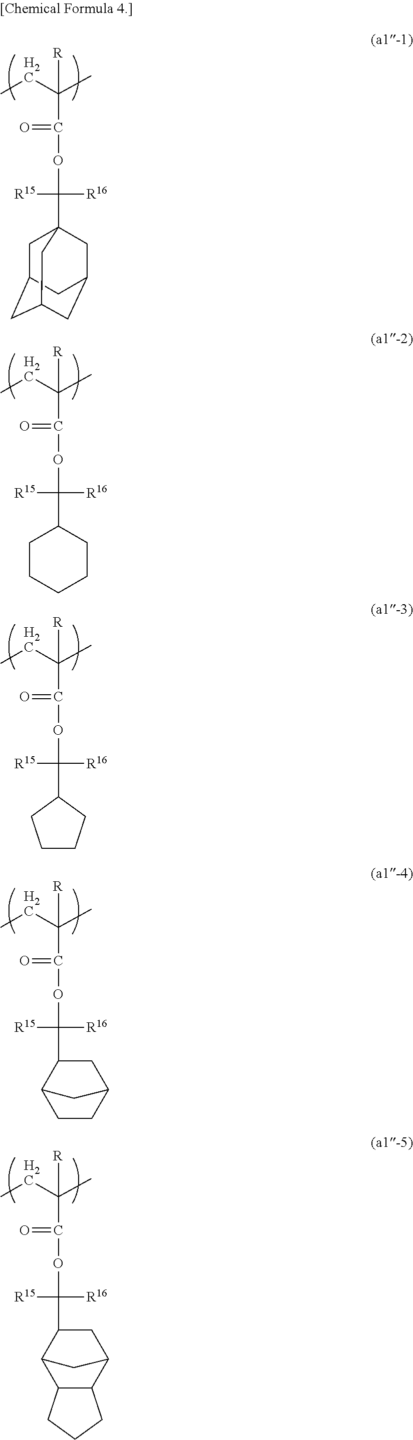

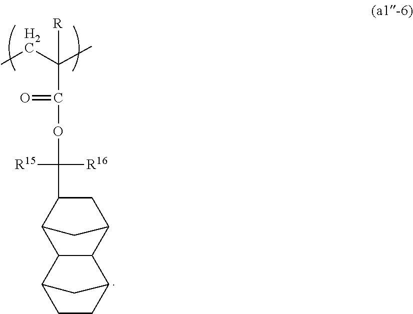

In the formulas, R represents a hydrogen atom, a lower alkyl group or a halogenated lower alkyl group; and R¹⁵ and R¹⁶ each independently represent an alkyl group (which may be linear or branched, and preferably has 1 to 5 carbon atoms).

In general formulas (a1″-1) to (a1″-6) above, the lower alkyl group or halogenated lower alkyl group for R are the same as the alkyl group of 1 to 5 carbon atoms or halogenated alkyl group of 1 to 5 carbon atoms which can be bonded to the α-position of the aforementioned acrylate ester.

An “acetal-type acid dissociable, dissolution inhibiting group” generally substitutes a hydrogen atom at the terminal of an alkali-soluble group such as a carboxy group or hydroxyl group, so as to be bonded with an oxygen atom. When acid is generated upon exposure, the generated acid acts to break the bond between the acetal-type acid dissociable, dissolution inhibiting group and the oxygen atom to which the acetal-type, acid dissociable, dissolution inhibiting group is bonded.

Examples of acetal-type acid dissociable, dissolution inhibiting groups include groups represented by general formula (p1) shown below.

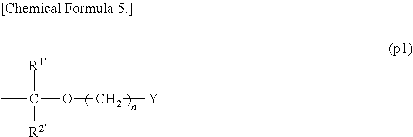

In the formula, R¹′ and R²′ each independently represent a hydrogen atom or a lower alkyl group; n represents an integer of 0 to 3; and Y represents a lower alkyl group or an aliphatic cyclic group.

In general formula (p1) above, n is preferably an integer of 0 to 2, more preferably 0 or 1, and most preferably 0.

As the lower alkyl group for R¹′ and R²′, the same lower alkyl groups as those described above for R can be used, although a methyl group or ethyl group is preferable, and a methyl group is particularly desirable.

In the present invention, it is preferable that at least one of R¹′ and R²′ be a hydrogen atom. That is, it is preferable that the acid dissociable, dissolution inhibiting group (p1) is a group represented by general formula (p1-1) shown below.

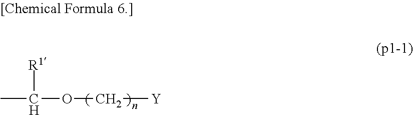

In the formula, R¹′, n and Y are the same as defined above.

As the lower alkyl group for Y, the same as the lower alkyl groups for R above can be used.

As the aliphatic cyclic group for Y, any of the aliphatic monocyclic/polycyclic groups which have been proposed for conventional ArF resists and the like can be appropriately selected for use. For example, the same groups described above in connection with the “aliphatic cyclic group” can be used.

Further, as the acetal-type, acid dissociable, dissolution inhibiting group, groups represented by general formula (p2) shown below can also be used.

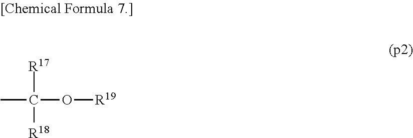

In the formula, R¹⁷ and R¹⁸ each independently represent a linear or branched alkyl group or a hydrogen atom; and R¹⁹ represents a linear, branched or cyclic alkyl group; or R¹⁷ and R¹⁹ each independently represents a linear or branched alkylene group, and the terminal of R¹⁷ is bonded to the terminal of R¹⁹ to form a ring.

The alkyl group for R¹⁷ and R¹⁸ preferably has 1 to 15 carbon atoms, and may be either linear or branched. As the alkyl group, an ethyl group or a methyl group is preferable, and a methyl group is most preferable.

It is particularly desirable that either one of R¹⁷ and R¹⁸ be a hydrogen atom, and the other be a methyl group.

R¹⁹ represents a linear, branched or cyclic alkyl group which preferably has 1 to 15 carbon atoms, and may be any of linear, branched or cyclic.

When R¹⁹ represents a linear or branched alkyl group, it is preferably an alkyl group of 1 to 5 carbon atoms, more preferably an ethyl group or methyl group, and most preferably an ethyl group.

When R¹⁹ represents a cycloalkyl group, it preferably has 4 to 15 carbon atoms, more preferably 4 to 12 carbon atoms, and most preferably 5 to 10 carbon atoms. As examples of the cycloalkyl group, groups in which one or more hydrogen atoms have been removed from a monocycloalkane or a polycycloalkane such as a bicycloalkane, tricycloalkane or tetracycloalkane, which may or may not be substituted with a fluorine atom or a fluorinated alkyl group, may be used. Examples of such groups include groups in which one or more hydrogen atoms have been removed from a monocycloalkane such as cyclopentane or cyclohexane; and groups in which one or more hydrogen atoms have been removed from a polycycloalkane such as adamantane, norbornane, isobornane, tricyclodecane or tetracyclododecane. Among these, a group in which one or more hydrogen atoms have been removed from adamantane is preferable.

In general formula (p2) above, R¹⁷ and R¹⁹ may each independently represent a linear or branched alkylene group (preferably an alkylene group of 1 to 5 carbon atoms), and the terminal of R¹⁹ may be bonded to the terminal of R¹⁷.

In such a case, a cyclic group is formed by R¹⁷, R¹⁹, the oxygen atom having R¹⁹ bonded thereto, and the carbon atom having the oxygen atom and R¹⁷ bonded thereto. Such a cyclic group is preferably a 4- to 7-membered ring, and more preferably a 4- to 6-membered ring. Specific examples of the cyclic group include tetrahydropyranyl group and tetrahydrofuranyl group.

As the structural unit (a1), it is preferable to use at least one member selected from the group consisting of structural units represented by formula (a1-0-1) shown below and structural units represented by formula (a1-0-2) shown below.

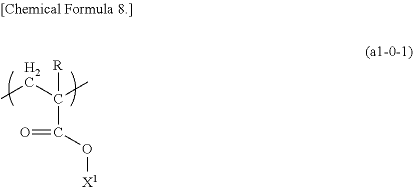

In the formula, R represents a hydrogen atom, a lower alkyl group or a halogenated lower alkyl group; and X¹ represents an acid dissociable, dissolution inhibiting group.

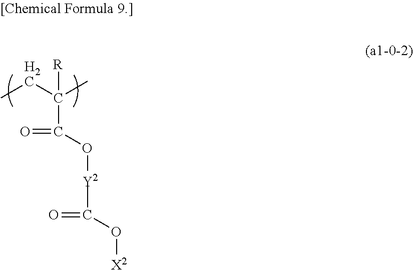

In the formula, R represents a hydrogen atom, a lower alkyl group or a halogenated lower alkyl group; X² represents an acid dissociable, dissolution inhibiting group; and Y² represents a divalent linking group.

In general formula (a1-0-1) above, the lower alkyl group or halogenated lower alkyl group for R are the same as the alkyl group of 1 to 5 carbon atoms or halogenated alkyl group of 1 to 5 carbon atoms which can be bonded to the α-position of the aforementioned acrylate ester.

X¹ is not particularly limited as long as it is an acid dissociable, dissolution inhibiting group. Examples thereof include the aforementioned tertiary alkyl ester-type acid dissociable, dissolution inhibiting groups and acetal-type acid dissociable, dissolution inhibiting groups, and tertiary alkyl ester-type acid dissociable, dissolution inhibiting groups are preferable.

In general formula (a1-0-2), R is the same as defined above.

X² is the same as defined for X¹ in general formula (a1-0-1).

As the divalent linking group for Y², an alkylene group, a divalent aliphatic cyclic group or a divalent linking group containing a hetero atom can be mentioned.

As the aliphatic cyclic group, the same as those used above in connection with the explanation of “aliphatic cyclic group” can be used, except that two hydrogen atoms have been removed therefrom.

When Y² represents an alkylene group, it preferably has 1 to 10 carbon atoms, more preferably 1 to 6, still more preferably 1 to 4, and most preferably 1 to 3.

When Y² represents a divalent aliphatic cyclic group, it is particularly desirable that the divalent aliphatic cyclic group be a group in which two or more hydrogen atoms have been removed from cyclopentane, cyclohexane, norbornane, isobornane, adamantane, tricyclodecane or tetracyclododecane.

When Y² represents a divalent linking group containing a hetero atom, examples thereof include —O—, —C(═O)—O—, —C(═O)—, —C(═O)—NH—, —NH— (H may be substituted with a substituent such as an alkyl group or an acyl group), —S—, —S(═O)₂—, —S(═O)₂—O—, and “-A-O—B— (wherein O is an oxygen atom, and each of A and B independently represents a divalent hydrocarbon group which may have a substituent)”.

When Y² represents a divalent linking group —NH— and the H in the formula is replaced with a substituent such as an alkyl group or an acyl group, the substituent preferably has 1 to 10 carbon atoms, more preferably 1 to 8 carbon atoms, and most preferably 1 to 5 carbon atoms.

When Y² is “A-O—B”, each of A and B independently represents a divalent hydrocarbon group which may have a substituent.

A hydrocarbon “has a substituent” means that part or all of the hydrogen atoms within the hydrocarbon group is substituted with groups or atoms other than hydrogen atom.

The hydrocarbon group for A may be either an aliphatic hydrocarbon group, or an aromatic hydrocarbon group. An “aliphatic hydrocarbon group” refers to a hydrocarbon group that has no aromaticity.

The aliphatic hydrocarbon group for A may be either saturated or unsaturated. In general, the aliphatic hydrocarbon group is preferably saturated.

As specific examples of the aliphatic hydrocarbon group for A, a linear or branched aliphatic hydrocarbon group, and an aliphatic hydrocarbon group having a ring in the structure thereof can be given.

The linear or branched aliphatic hydrocarbon group preferably has 1 to 10 carbon atoms, more preferably 1 to 8, still more preferably 2 to 5, and most preferably 2.

As a linear aliphatic hydrocarbon group, a linear alkylene group is preferable, and specific examples include a methylene group, an ethylene group [—(CH₂)₂-], a trimethylene group [—(CH₂)₃-], a tetramethylene group [—(CH₂)₄-] and a pentamethylene group [—(CH₂)₅-].

As the branched aliphatic hydrocarbon group, a branched alkylene group is preferable, and specific examples include alkylalkylene groups, e.g., alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)— and —C(CH₂CH₃)₂—; alkylethylene groups such as —CH(CH₃)CH₂—, —CH(CH₃)CH(CH₃)—, —C(CH₃)₂CH₂— and —CH(CH₂CH₃)CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group within the alkylalkylene group, a linear alkyl group of 1 to 5 carbon atoms is preferable.

The linear or branched aliphatic hydrocarbon group (chain-like aliphatic hydrocarbon group) may or may not have a substituent. Examples of the substituent include a fluorine atom, a fluorinated lower alkyl group of 1 to 5 carbon atoms, and an oxygen atom (═O).

As examples of the hydrocarbon group containing a ring, a cyclic aliphatic hydrocarbon group (a group in which two hydrogen atoms have been removed from an aliphatic hydrocarbon ring), and a group in which the cyclic aliphatic hydrocarbon group is bonded to the terminal of the aforementioned chain-like aliphatic hydrocarbon group or interposed within the aforementioned chain-like aliphatic hydrocarbon group, can be given.

The cyclic aliphatic hydrocarbon group preferably has 3 to 20 carbon atoms, and more preferably 3 to 12 carbon atoms.

The cyclic aliphatic hydrocarbon group may be either a polycyclic group or a monocyclic group. As the monocyclic group, a group in which two hydrogen atoms have been removed from a monocycloalkane of 3 to 6 carbon atoms is preferable. Examples of the monocycloalkane include cyclopentane and cyclohexane.

As the polycyclic group, a group in which two hydrogen atoms have been removed from a polycycloalkane of 7 to 12 carbon atoms is preferable. Examples of the polycycloalkane include adamantane, norbornane, isobornane, tricyclodecane and tetracyclododecane.

The cyclic aliphatic hydrocarbon group may or may not have a substituent. Examples of the substituent include a lower alkyl group of 1 to 5 carbon atoms, a fluorine atom, a fluorinated lower alkyl group of 1 to 5 carbon atoms, and an oxygen atom (═O).

As A, a linear aliphatic hydrocarbon group is preferable, more preferably a linear alkylene group, still more preferably a linear alkylene group of 2 to 5 carbon atoms, and most preferably an ethylene group.

Examples of the hydrocarbon group for A include a divalent aromatic hydrocarbon group in which one hydrogen atom has been removed from a benzene ring of a monovalent aromatic hydrocarbon group such as a phenyl group, a biphenyl group, a fluorenyl group, a naphthyl group, an anthryl group or a phenanthryl group; an aromatic hydrocarbon group in which part of the carbon atoms constituting the ring of the aforementioned divalent aromatic hydrocarbon group has been substituted with a hetero atom such as an oxygen atom, a sulfur atom or a nitrogen atom; and an aromatic hydrocarbon group in which one hydrogen atom has been removed from a benzene ring of an arylalkyl group such as a benzyl group, a phenethyl group, a 1-naphthylmethyl group, a 2-naphthylmethyl group, a 1-naphthylethyl group or a 2-naphthylethyl group.

The aromatic hydrocarbon group may or may not have a substituent. Examples of the substituent include an alkyl group of 1 to 5 carbon atoms, a fluorine atom, a fluorinated alkyl group of 1 to 5 carbon atoms, and an oxygen atom (═O).

As the hydrocarbon group for B, the same divalent hydrocarbon groups as those described above for A can be used.

As B, a linear or branched aliphatic hydrocarbon group is preferable, and a methylene group or an alkylmethylene group is particularly desirable.

The alkyl group within the alkyl methylene group is preferably a linear alkyl group of 1 to 5 carbon atoms, more preferably a linear alkyl group of 1 to 3 carbon atoms, and most preferably a methyl group.

Specific examples of the structural unit (a1) include structural units represented by general formulas (a1-1) to (a1-4) shown below.

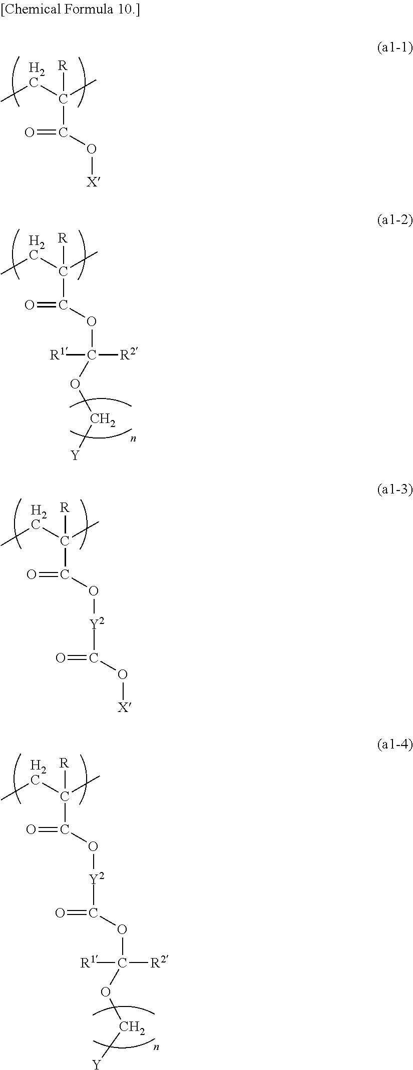

In the formulas, X′ represents a tertiary alkyl ester-type acid dissociable, dissolution inhibiting group; Y represents a lower alkyl group of 1 to 5 carbon atoms or an aliphatic cyclic group; n represents an integer of 0 to 3; Y² represents a divalent linking group; R is the same as defined above; and each of R¹′ and R²′ independently represents a hydrogen atom or a lower alkyl group of 1 to 5 carbon atoms.

Examples of the tertiary alkyl ester-type acid dissociable, dissolution inhibiting group for X′ include the same tertiary alkyl ester-type acid dissociable, dissolution inhibiting groups as those described above for X¹.

As R¹′, R²′, n and Y are respectively the same as defined for R¹′, R²′, n and Y in general formula (p1) described above in connection with the “acetal-type acid dissociable, dissolution inhibiting group”.

As examples of Y², the same groups as those described above for Y² in general formula (a1-0-2) can be given.

Specific examples of structural units represented by general formula (a1-1) to (a1-4) are shown below.

In the formulas shown below, R^{α} represents a hydrogen atom, a methyl group or a trifluoromethyl group.

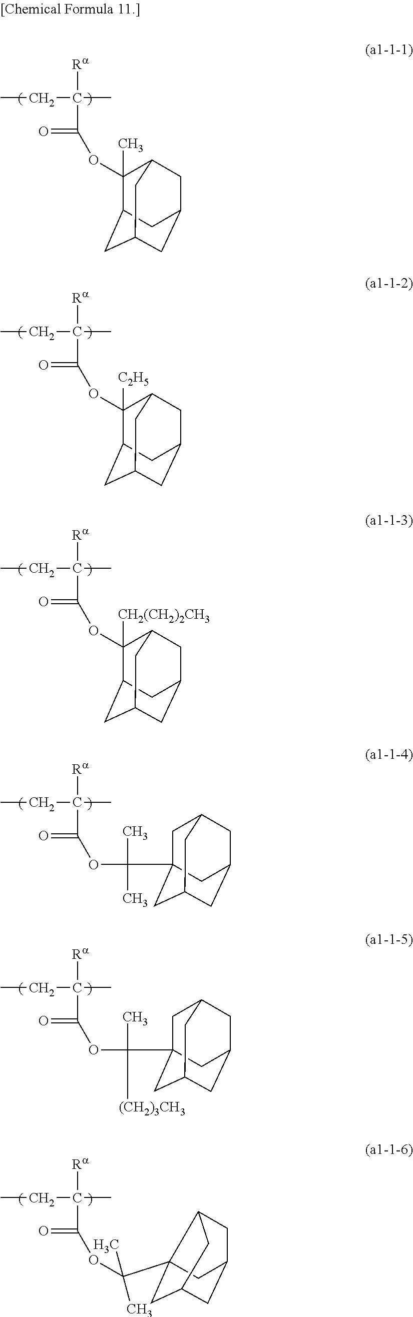

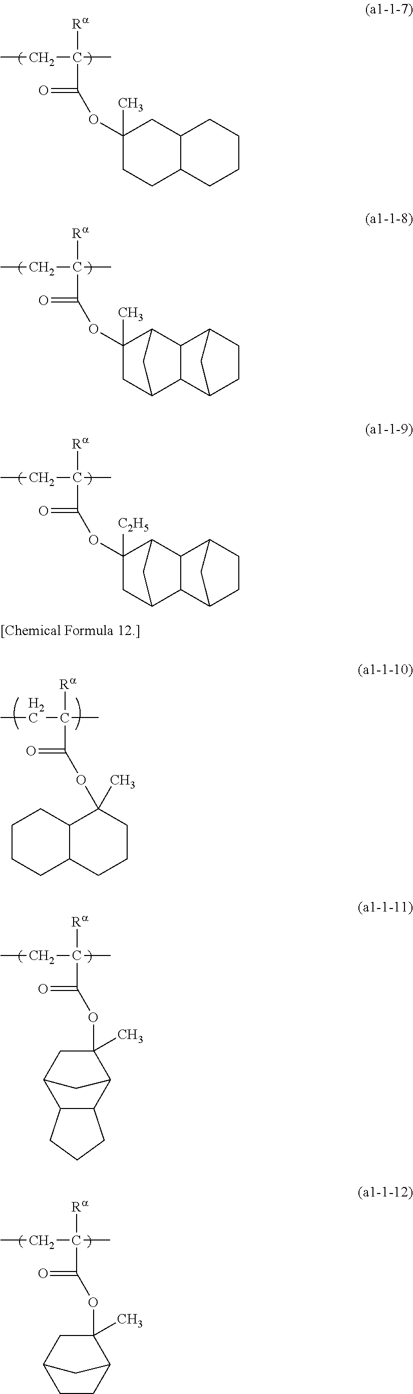

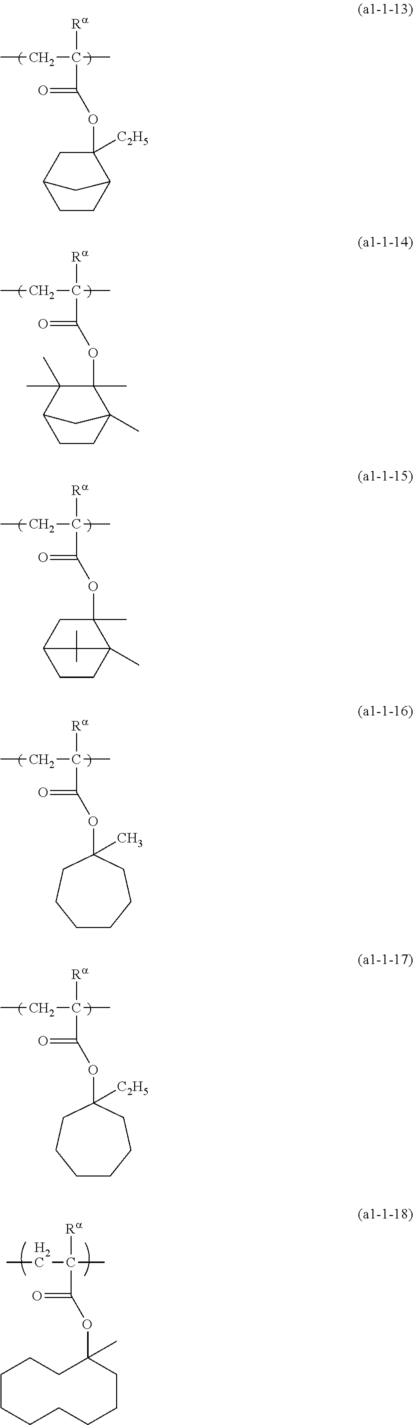

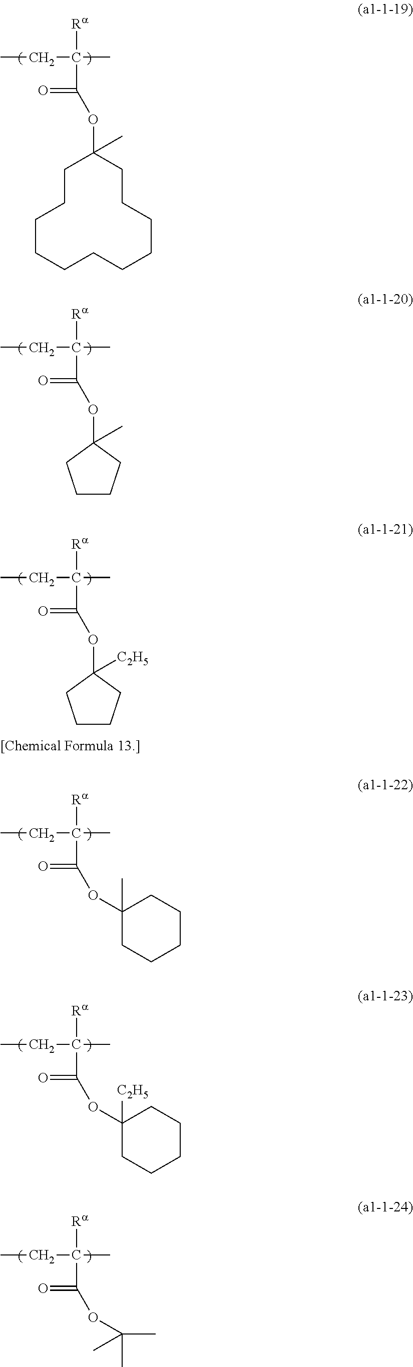

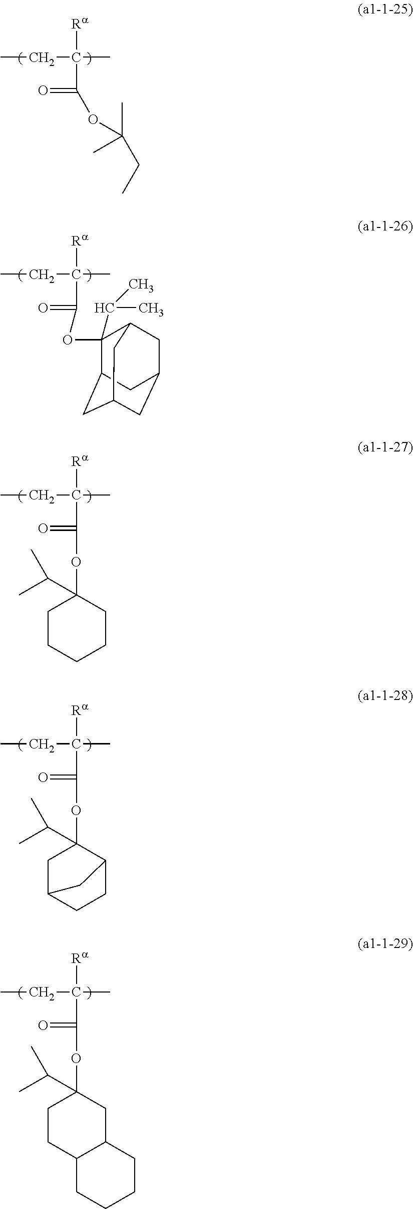

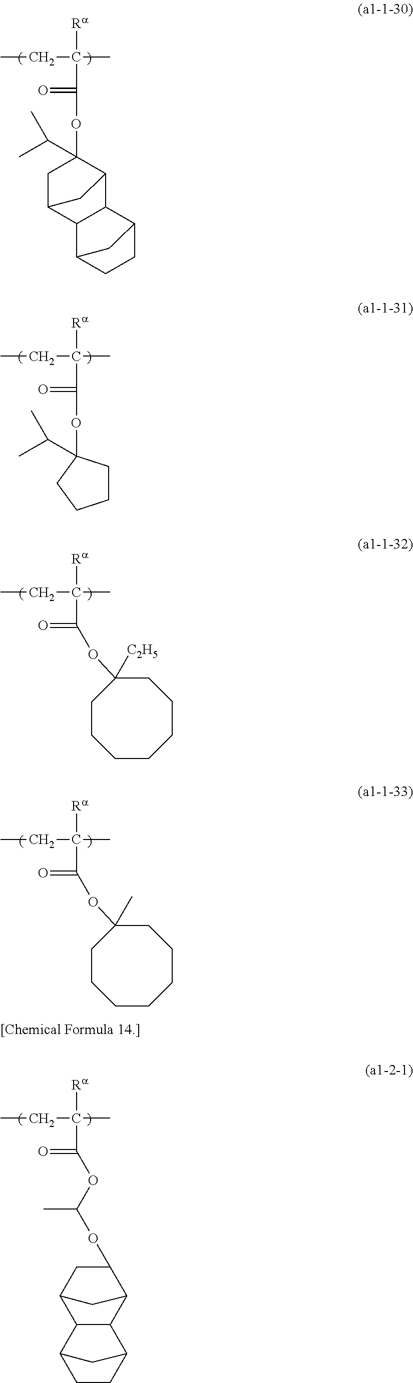

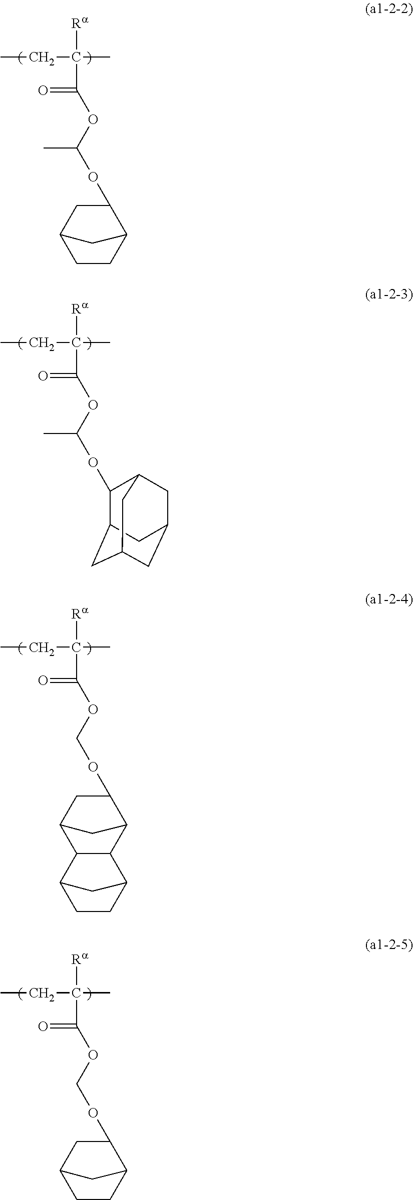

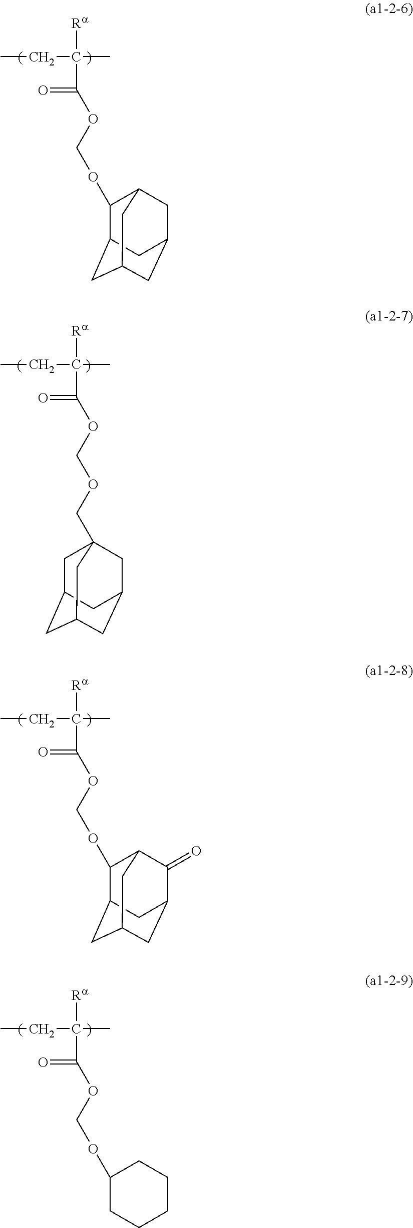

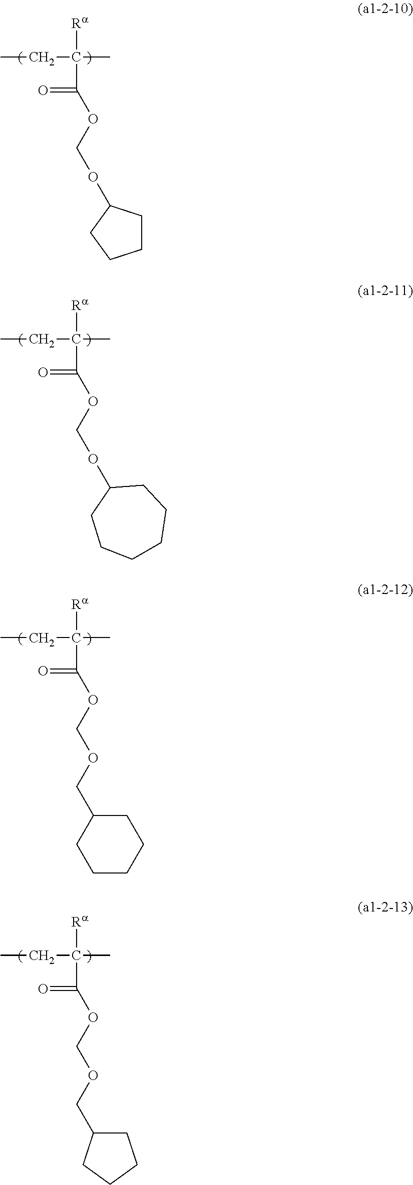

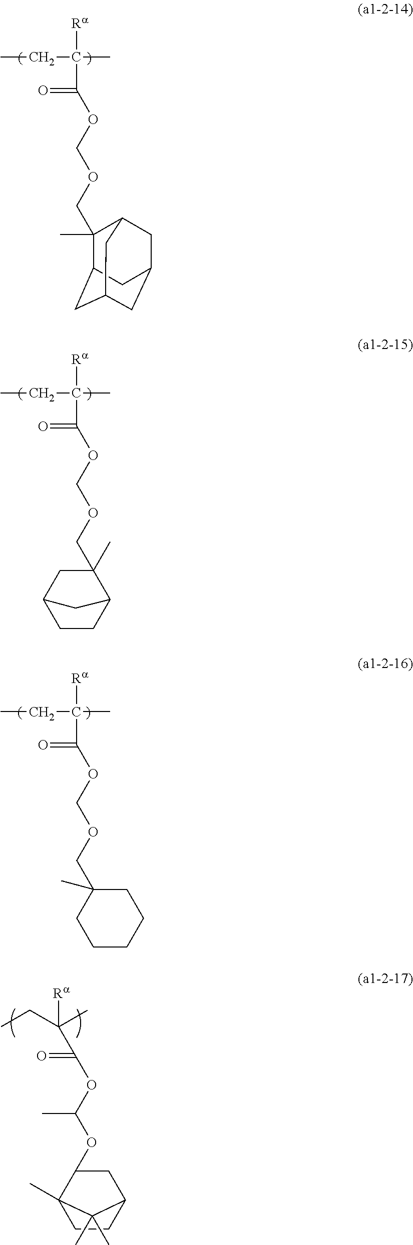

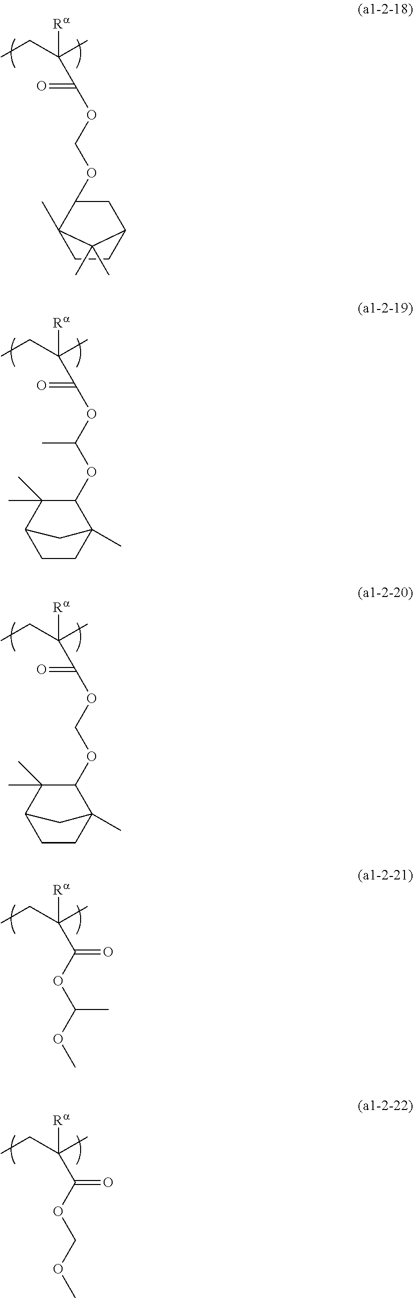

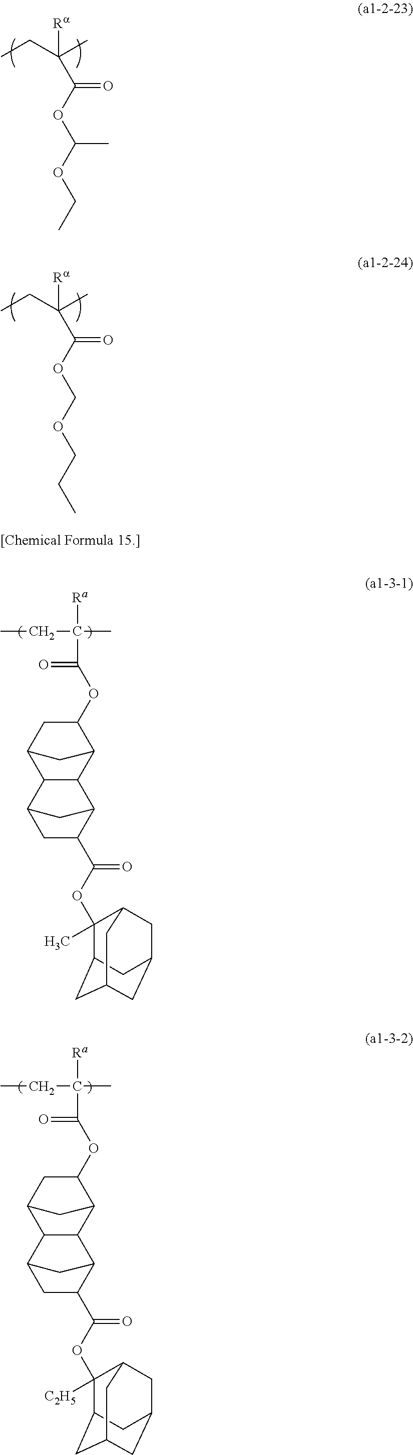

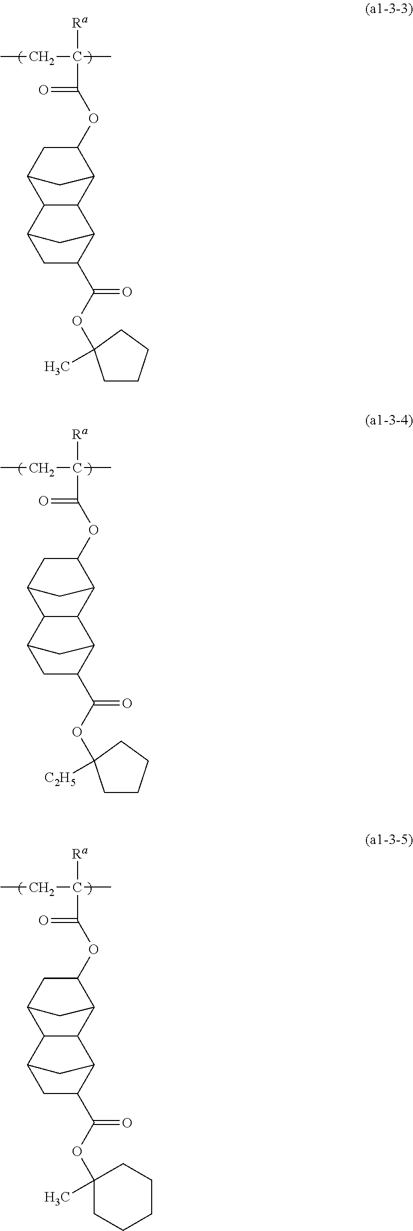

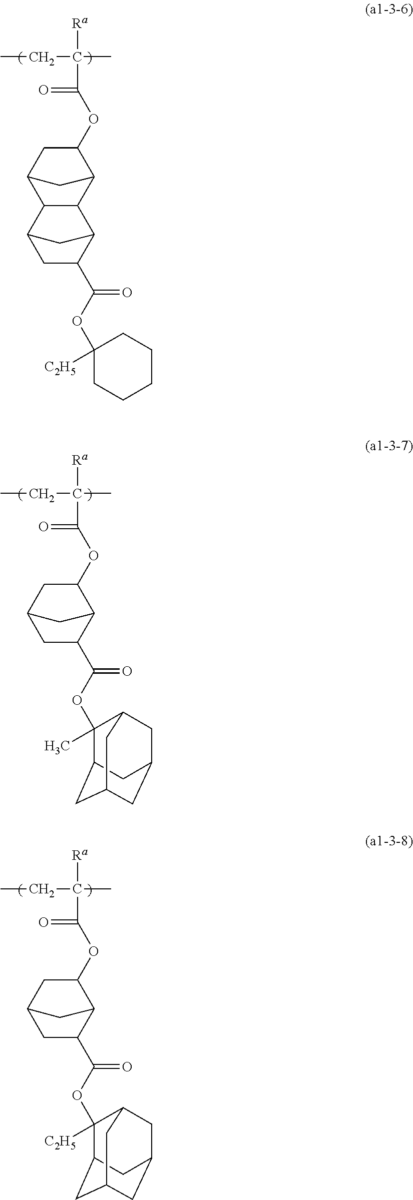

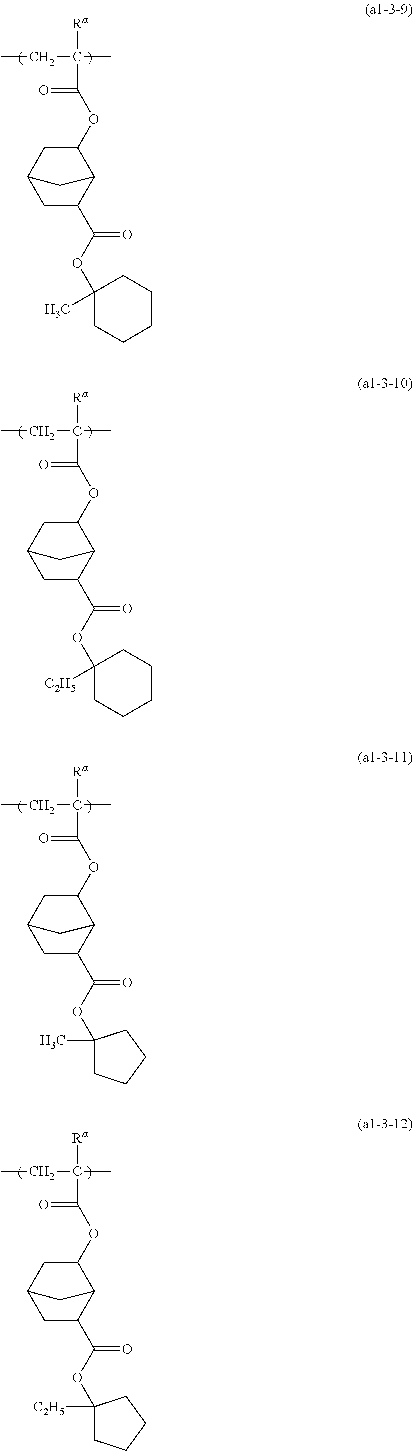

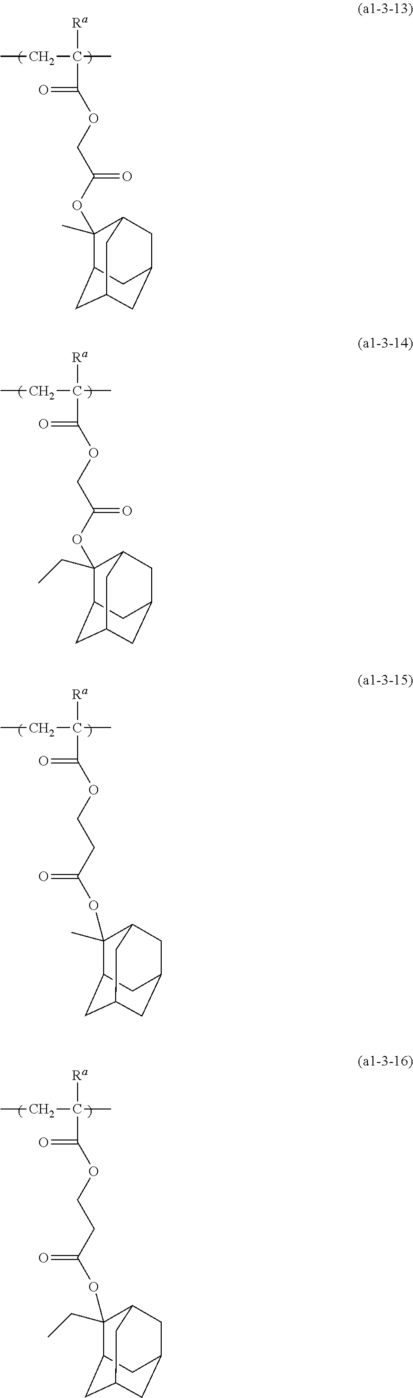

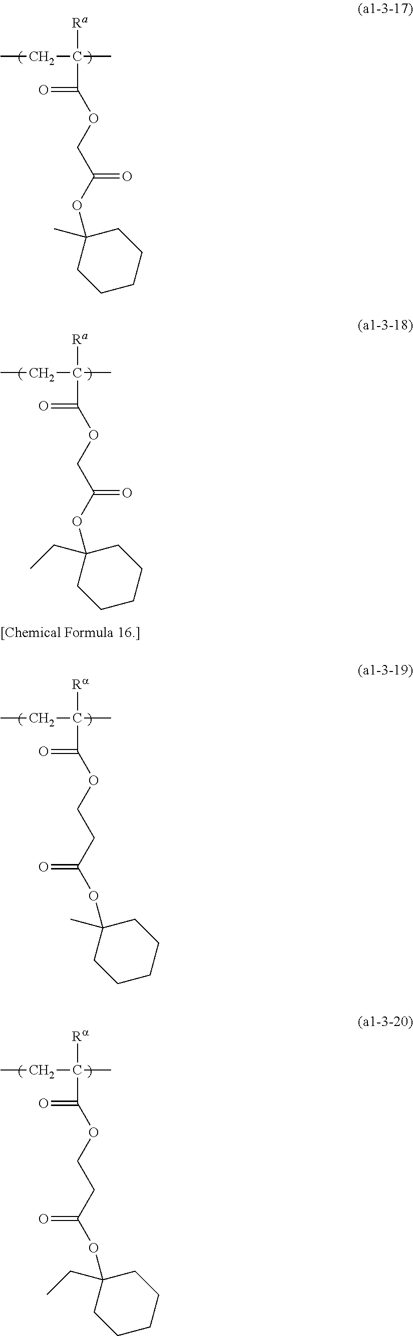

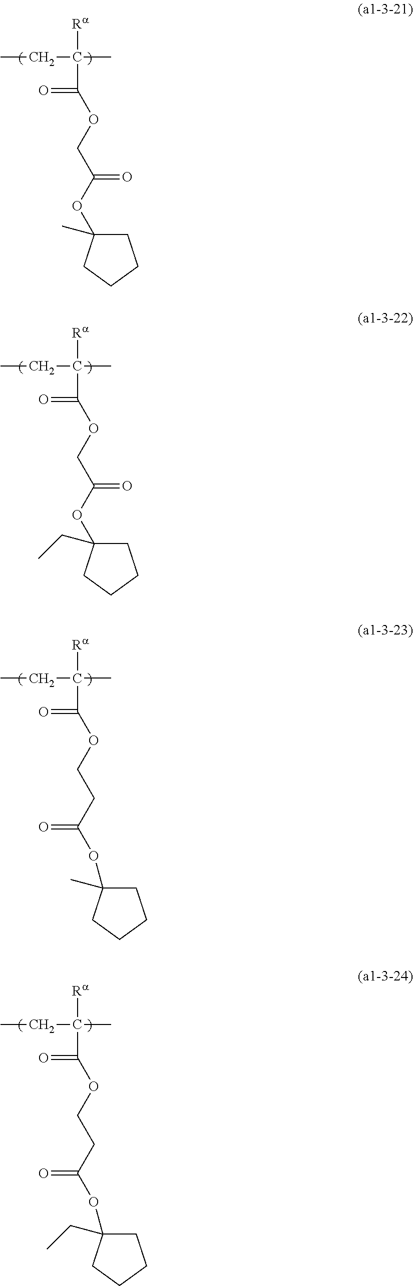

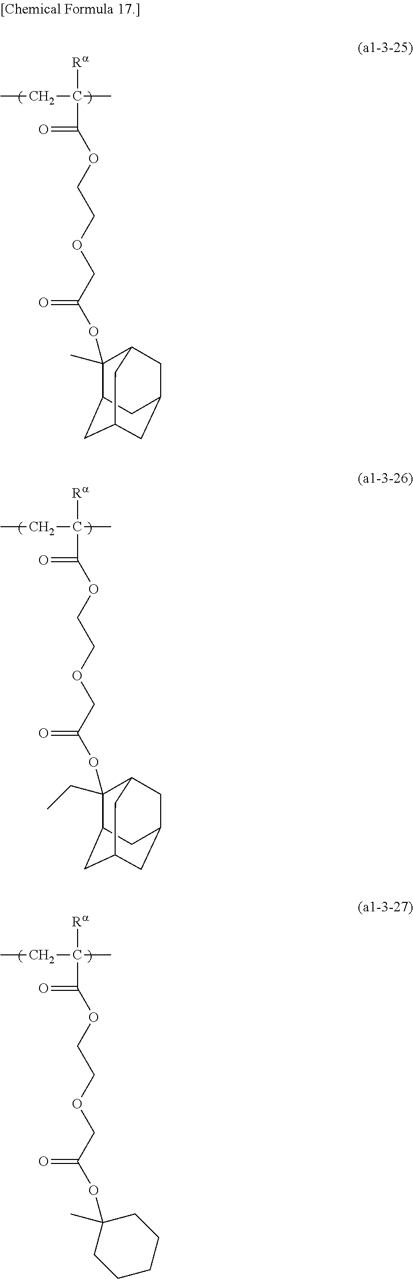

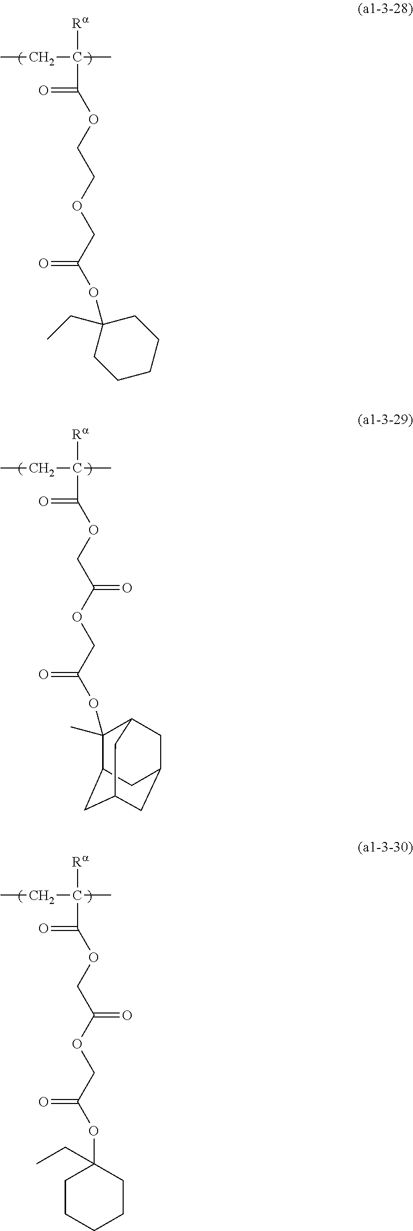

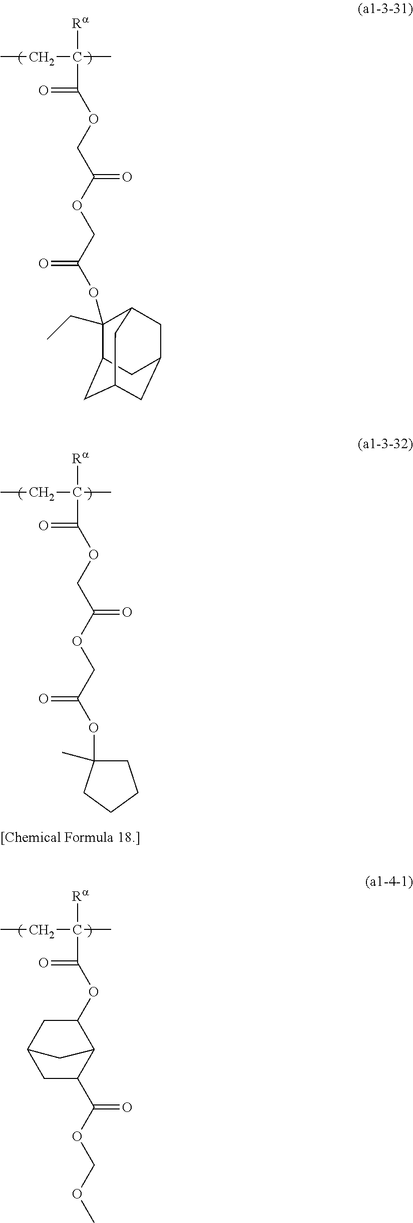

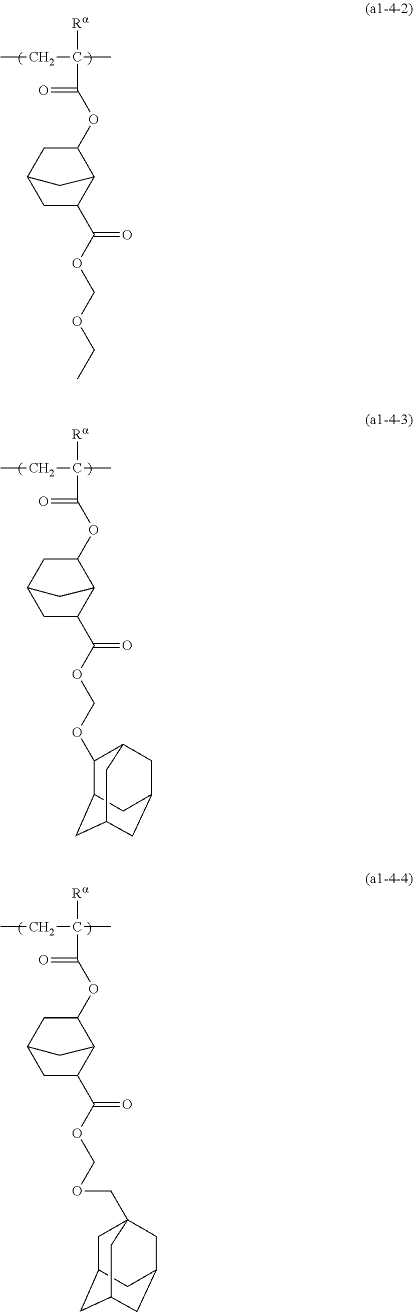

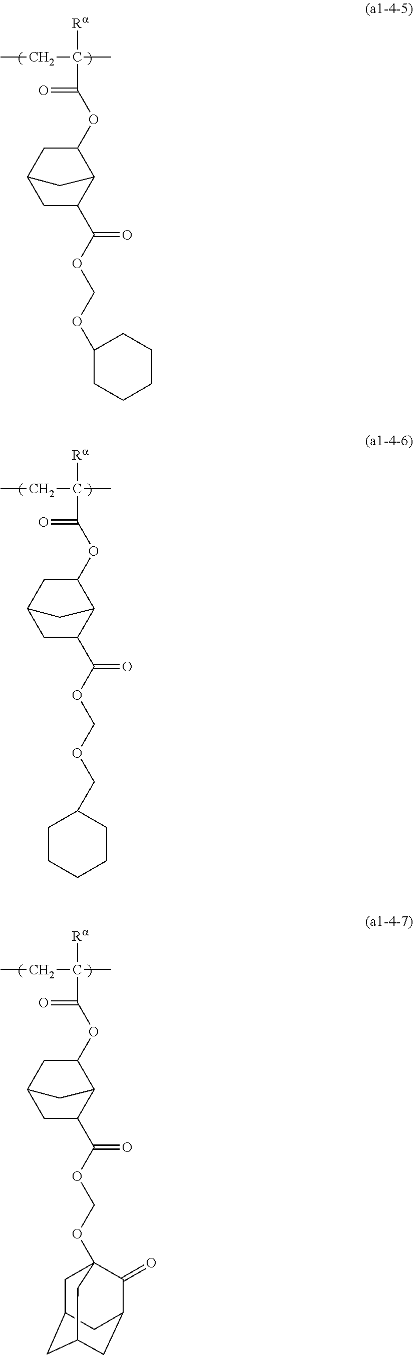

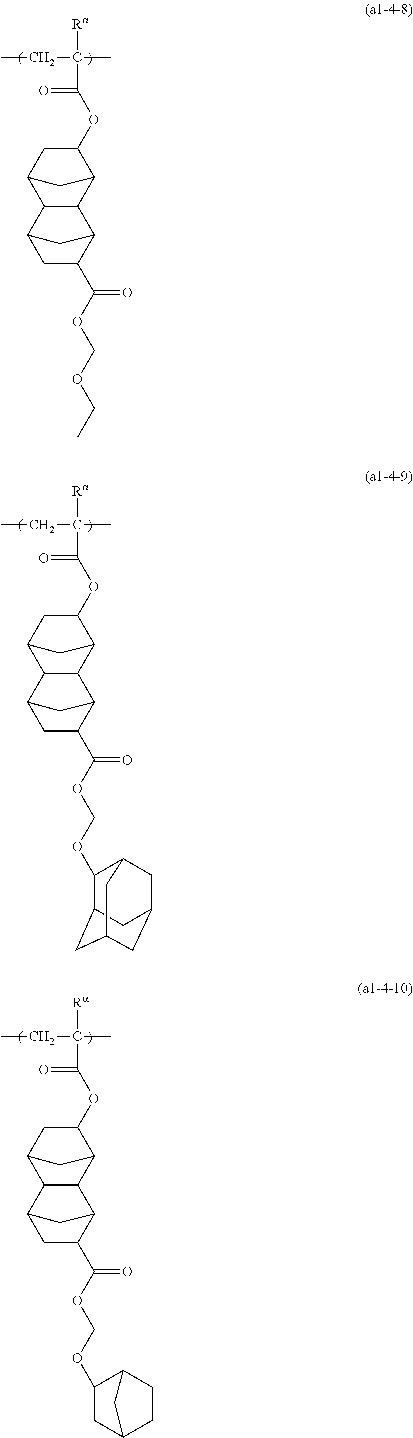

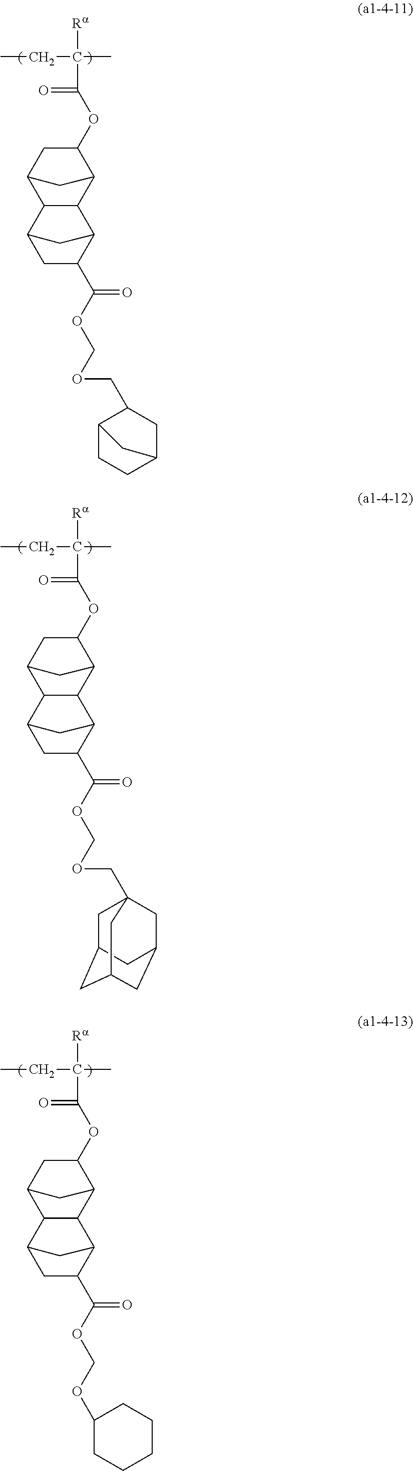

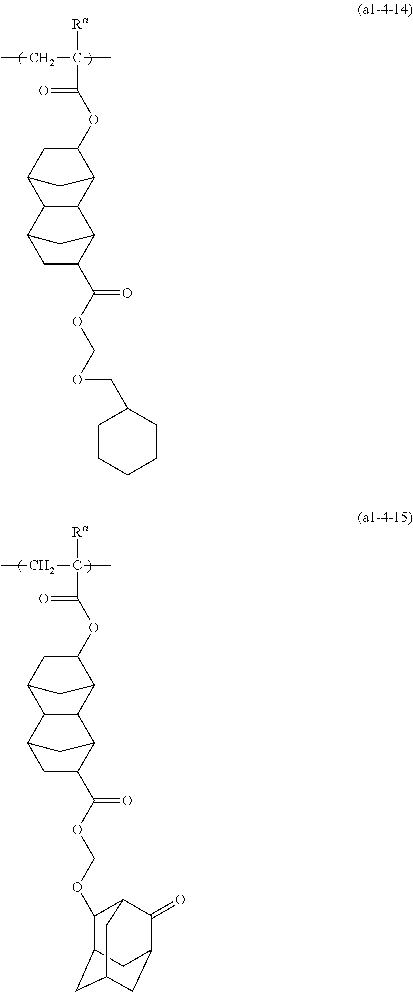

As the structural unit (a1), one type of structural unit may be used alone, or two or more types of structural units may be used in combination.

Among these, structural units represented by general formula (a1-1) or (a1-3) are preferable. More specifically, at least one structural unit selected from the group consisting of structural units represented by formulas (a1-1-1) to (a-1-1-4), (a1-1-16), (a1-1-17), (a1-1-20) to (a1-1-23), (a1-1-26), (a1-1-32), (a1-1-33) and (a1-3-25) to (a1-3-28) is more preferable.

Further, as the structural unit (a1), structural units represented by general formula (a1-1-01) shown below which includes the structural units represented by formulas (a1-1-1) to (a1-1-3) and (a1-1-26), structural units represented by general formula (a1-1-02) shown below which includes the structural units represented by formulas (a1-1-16), (a1-1-17), (a1-1-20) to (a1-1-23), (a1-1-32) and (a1-1-33), structural units represented by general formula (a1-3-01) shown below which include the structural units represented by formulas (a1-3-25) and (a1-3-26), structural units represented by general formula (a1-3-02) shown below which include the structural units represented by formulas (a1-3-27) and (a1-3-28), structural units represented by general formula (a1-3-03-1) shown below which include the structural units represented by formulas (a1-3-29) and (a1-3-31), and structural units represented by general formula (a1-3-03-2) shown below which include the structural units represented by formulas (a1-3-30) and (a1-3-32) are also preferable.

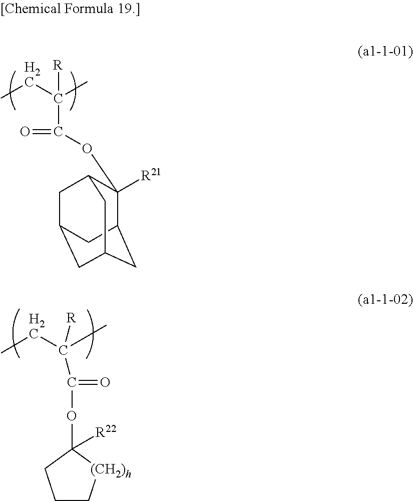

In the formula, R represents a hydrogen atom, a lower alkyl group or a halogenated lower alkyl group; R²¹ represents a lower alkyl group; R²² represents a lower alkyl group. h represents an integer of 1 to 6.

In general formula (a1-1-01), R is the same as defined above.

The lower alkyl group for R²¹ is the same as defined for the lower alkyl group for R above, a linear or branched alkyl group is preferable, and a methyl group, an ethyl group or an isopropyl group is particularly desirable.

In general formula (a1-1-02), R is the same as defined above.

The lower alkyl group for R²² is the same as defined for the lower alkyl group for R above, a linear or branched alkyl group is preferable, and a methyl group or an ethyl group is particularly desirable.

h is preferably 1 or 2.

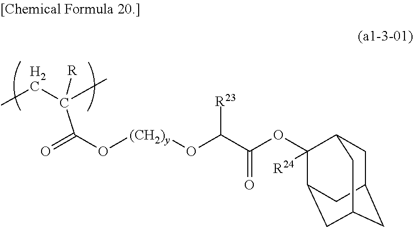

In the formula, R represents a hydrogen atom, a lower alkyl group or a halogenated lower alkyl group; R²⁴ represents a lower alkyl group; R²³ represents a hydrogen atom or a methyl group; and y represents an integer of 1 to 10.

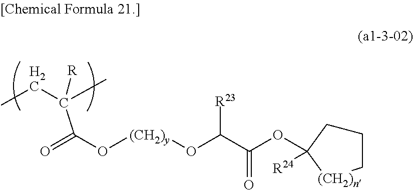

In the formula, R represents a hydrogen atom, a lower alkyl group or a halogenated lower alkyl group; R²⁴ represents a lower alkyl group; R²³ represents a hydrogen atom or a methyl group; y represents an integer of 1 to 10; and n′ represents an integer of 1 to 6.

In general formulas (a1-3-01) and (a1-3-02), R is the same as defined above.

R²³ is preferably a hydrogen atom.

The lower alkyl group for R²⁴ is the same as defined for the lower alkyl group for R, and is preferably a methyl group or an ethyl group.

y is preferably an integer of 1 to 8, more preferably an integer of 2 to 5, and most preferably 2.

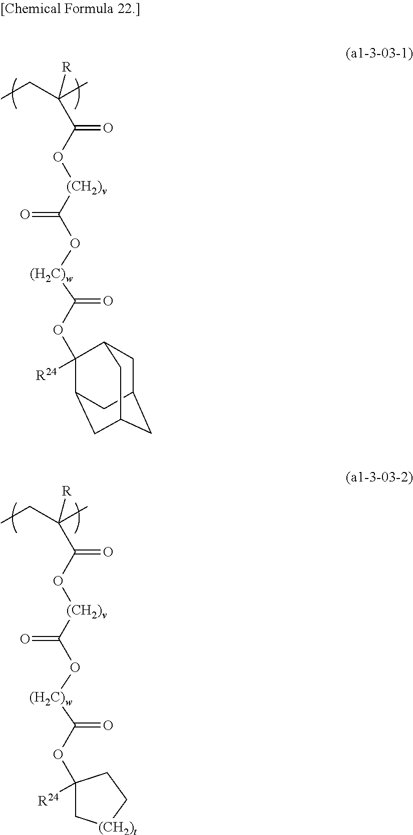

In the formulas, R and R²⁴ are the same as defined above; v represents an integer of 1 to 10; w represents an integer of 1 to 10; and t represents an integer of 0 to 3.

v is preferably an integer of 1 to 5, and most preferably 1 or 2.

w is preferably an integer of 1 to 5, and most preferably 1 or 2.

t is preferably an integer of 1 to 3, and most preferably 1 or 2.

In the component (A1), the amount of the structural unit (a1) based on the combined total of all structural units constituting the component (A1) is preferably 10 to 80 mol %, more preferably 20 to 70 mol %, and still more preferably 25 to 50 mol %. When the amount of the structural unit (a1) is at least as large as the lower limit of the above-mentioned range, a pattern can be easily formed using a resist composition prepared from the component (A1). On the other hand, when the amount of the structural unit (a1) is no more than the upper limit of the above-mentioned range, a good balance can be achieved with the other structural units.

(Structural Unit (a2))

The structural unit (a2) is a structural unit derived from an acrylate ester which may have an atom other than hydrogen or a substituent bonded to the carbon atom on the α position and contains a lactone-containing cyclic group.

The term “lactone-containing cyclic group” refers to a cyclic group including one ring containing a —O—C(O)— structure (lactone ring). The term “lactone ring” refers to a single ring containing a —O—C(O)— structure, and this ring is counted as the first ring. A lactone-containing cyclic group in which the only ring structure is the lactone ring is referred to as a monocyclic group, and groups containing other ring structures are described as polycyclic groups regardless of the structure of the other rings.

When the component (A1) is used for forming a resist film, the lactone-containing cyclic group of the structural unit (a2) is effective in improving the adhesion between the resist film and the substrate, and increasing the compatibility with the developing solution containing water.

As the structural unit (a2), there is no particular limitation, and an arbitrary structural unit may be used.

Specific examples of lactone-containing monocyclic groups include a group in which one hydrogen atom has been removed from a 4- to 6-membered lactone ring, such as a group in which one hydrogen atom has been removed from β-propionolatone, a group in which one hydrogen atom has been removed from γ-butyrolactone, and a group in which one hydrogen atom has been removed from δ-valerolactone. Further, specific examples of lactone-containing polycyclic groups include groups in which one hydrogen atom has been removed from a lactone ring-containing bicycloalkane, tricycloalkane or tetracycloalkane.

More specifically, examples of the structural unit (a2) include structural units represented by general formulas (a2-1) to (a2-5) shown below.

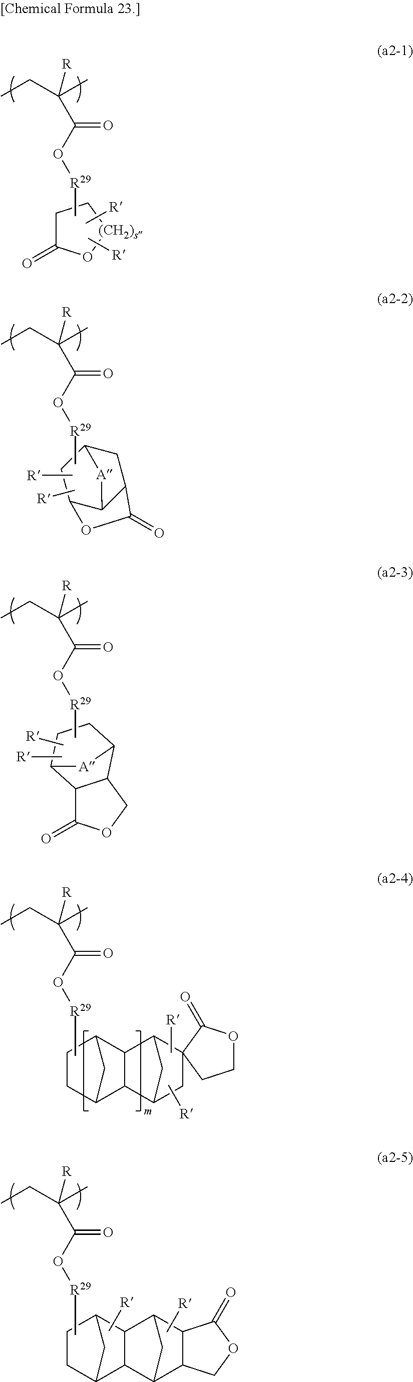

In the formulas, R represents a hydrogen atom, a lower alkyl group or a halogenated lower alkyl group; each R′ independently represents a hydrogen atom, an alkyl group of 1 to 5 carbon atoms, an alkoxy group of 1 to 5 carbon atoms or —COOR″, wherein R″ represents a hydrogen atom or an alkyl group; R²⁹ represents a single bond or a divalent linking group; s″ represents an integer of 0 to 2; A″ represents an oxygen atom, a sulfur atom or an alkylene group of 1 to 5 carbon atoms which may contain an oxygen atom or a sulfur atom; and m represents 0 or 1.

In general formulas (a2-1) to (a2-5), R is the same as defined for R in the structural unit (a1).

Examples of the alkyl group of 1 to 5 carbon atoms for R′ include a methyl group, an ethyl group, a propyl group, an n-butyl group and a tert-butyl group.

Examples of the alkoxy group of 1 to 5 carbon atoms for R′ include a methoxy group, an ethoxy group, an n-propoxy group, an iso-propoxy group, an n-butoxy group and a tert-butoxy group

In terms of industrial availability, R′ is preferably a hydrogen atom.

R″ preferably represents a hydrogen atom or a linear, branched or cyclic alkyl group of 1 to 15 carbon atoms.

When R″ is a linear or branched alkyl group, it preferably has 1 to 10 carbon atoms, more preferably 1 to 5 carbon atoms.

When R″ is a cyclic alkyl group (cycloalkyl group), it preferably has 3 to 15 carbon atoms, more preferably 4 to 12 carbon atoms, and most preferably 5 to 10 carbon atoms. As examples of the cycloalkyl group, groups in which one or more hydrogen atoms have been removed from a monocycloalkane or a polycycloalkane such as a bicycloalkane, tricycloalkane or tetracycloalkane, which may or may not be substituted with a fluorine atom or a fluorinated alkyl group, may be used. Examples of such groups include groups in which one or more hydrogen atoms have been removed from a monocycloalkane such as cyclopentane or cyclohexane; and groups in which one or more hydrogen atoms have been removed from a polycycloalkane such as adamantane, norbornane, isobornane, tricyclodecane or tetracyclododecane.

As A″, an alkylene group of 1 to 5 carbon atoms or —O— is preferable, more preferably an alkylene group of 1 to 5 carbon atoms, and most preferably a methylene group.

R²⁹ represents a single bond or a divalent linking group. Examples of divalent linking groups include the same divalent linking groups as those described above for Y² in general formula (a1-0-2). Among these, an alkylene group, an ester bond (—C(═O)—O—) or a combination thereof is preferable. The alkylene group as a divalent linking group for R²⁹ is preferably a linear or branched alkylene group. Specific examples include the same linear alkylene groups and branched alkylene groups as those described above for the aliphatic cyclic group A in Y².

s″ is preferably 1 or 2.

Specific examples of structural units represented by general formulas (a2-1) to (a2-5) are shown below.

In the formulas shown below, R^{α} represents a hydrogen atom, a methyl group or a trifluoromethyl group.

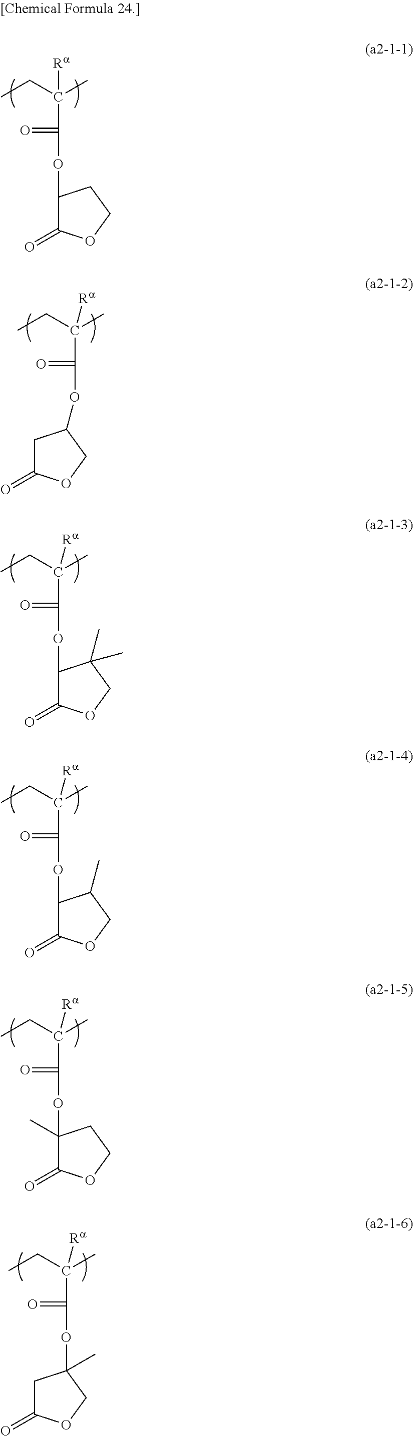

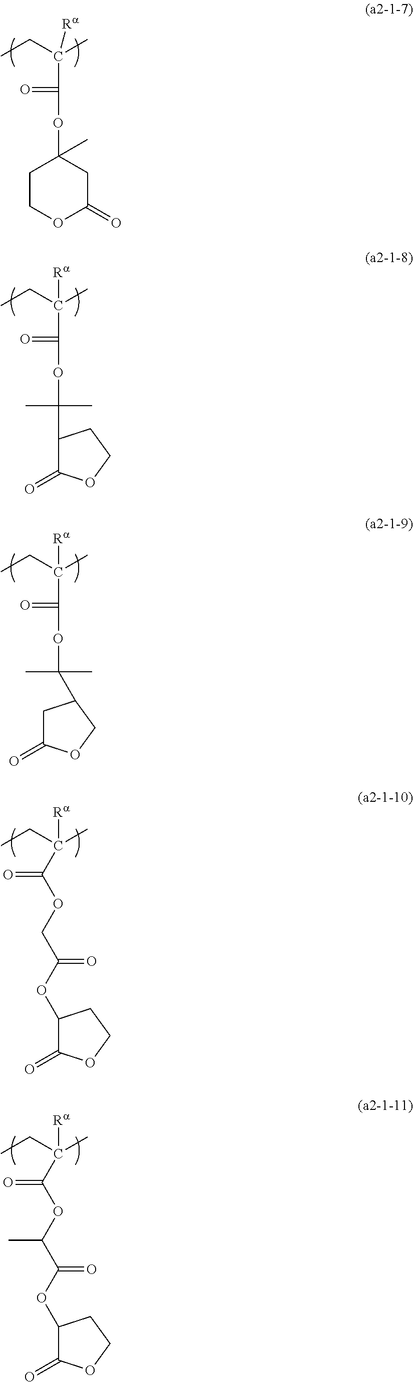

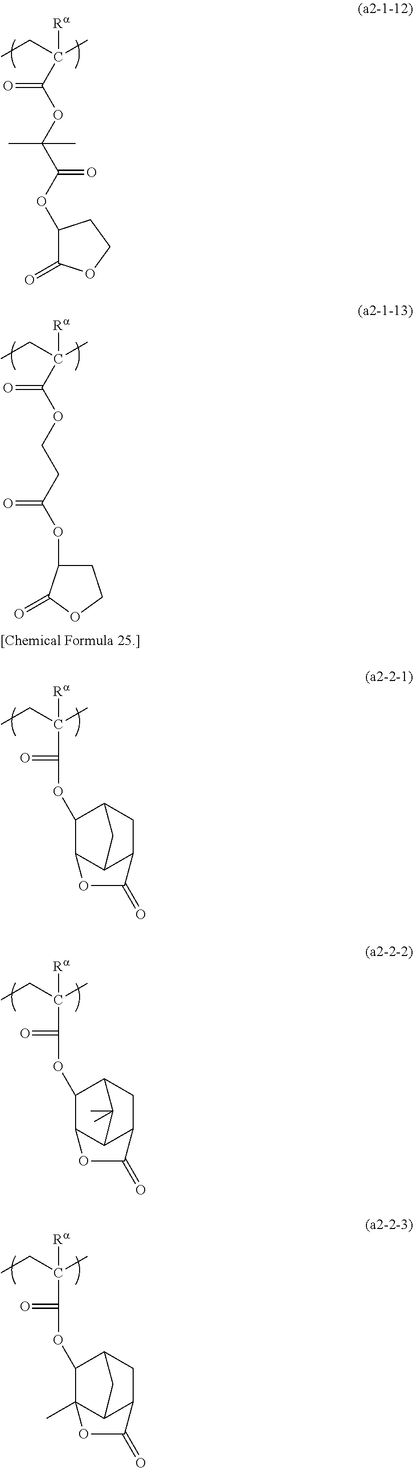

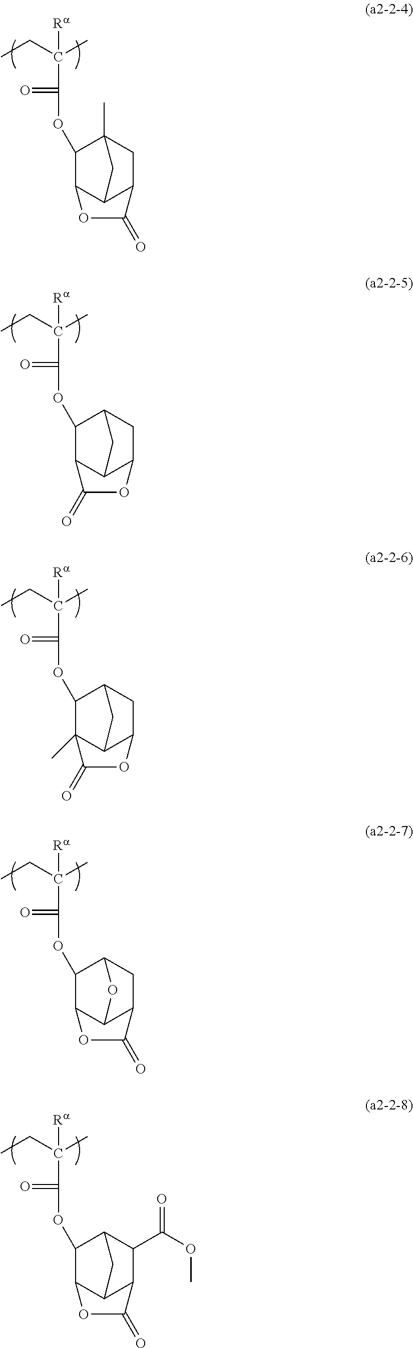

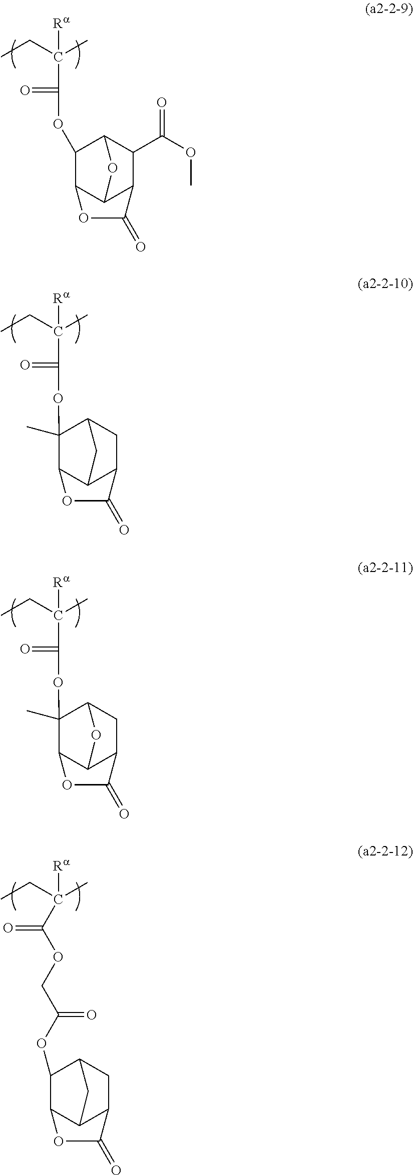

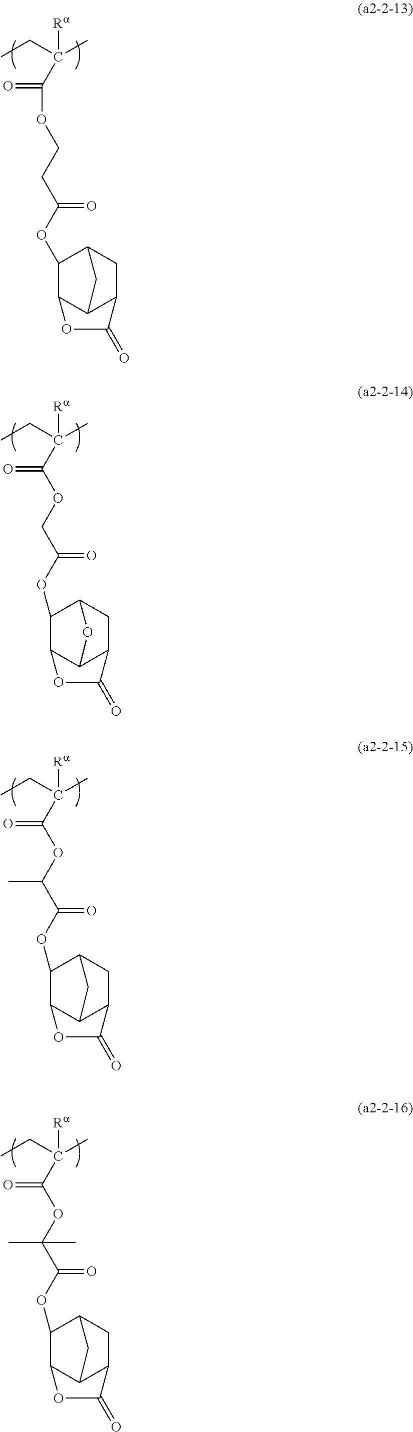

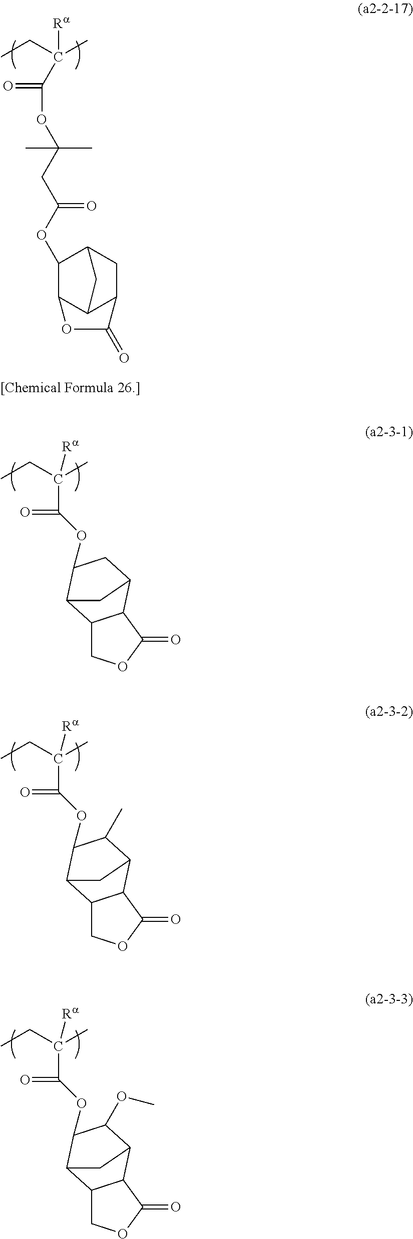

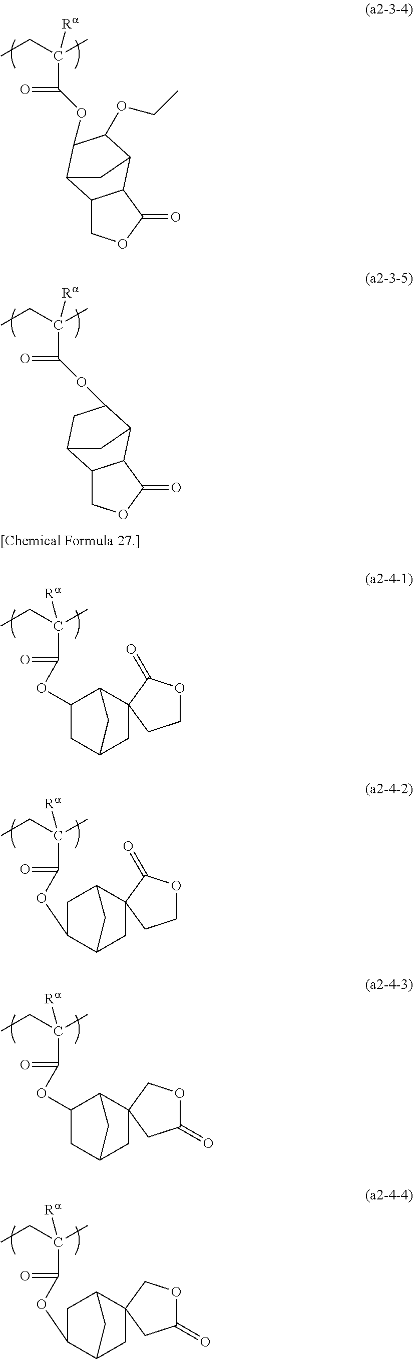

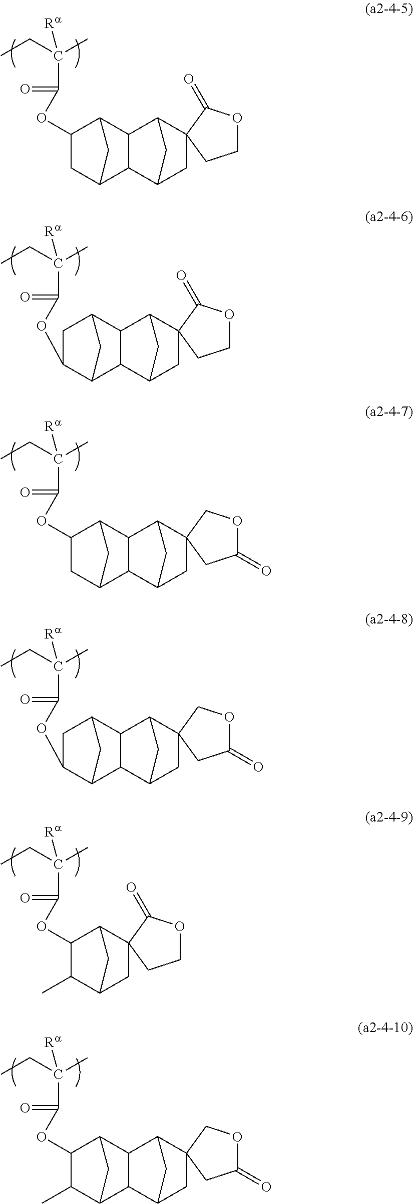

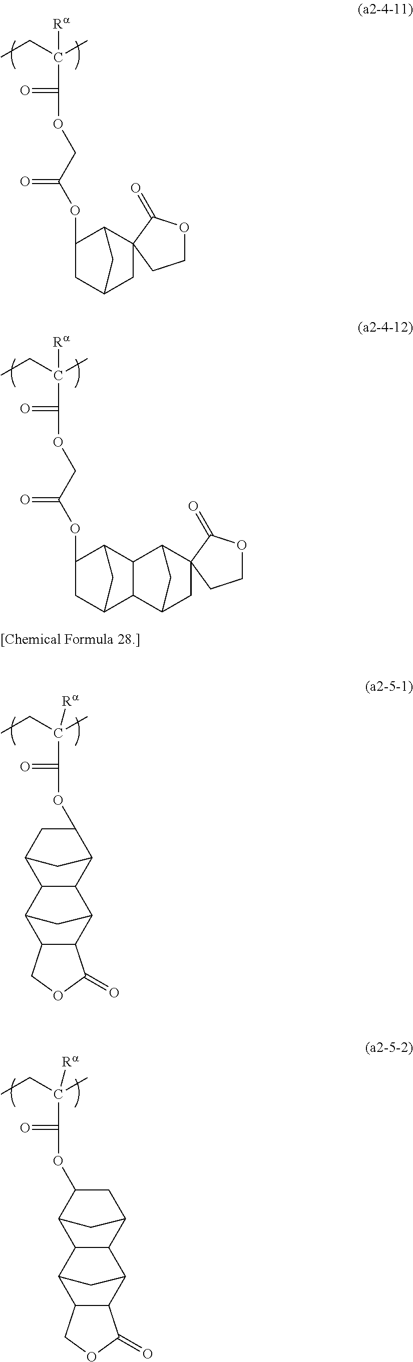

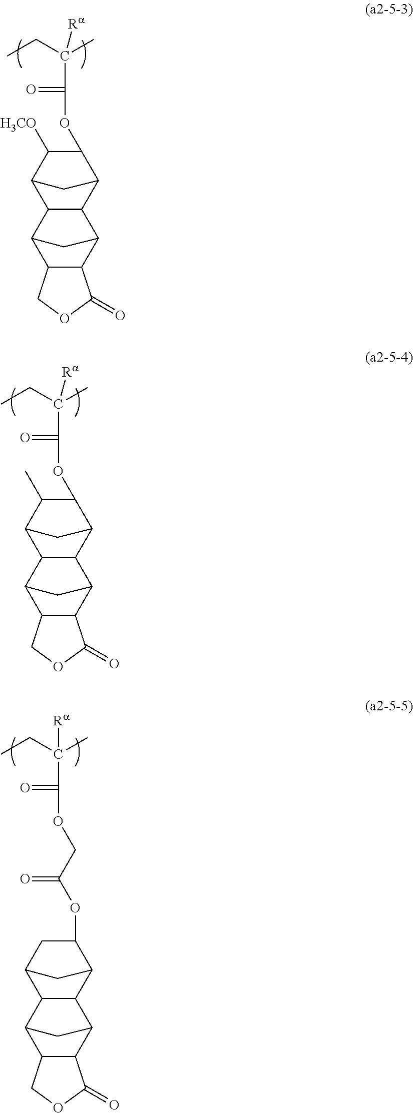

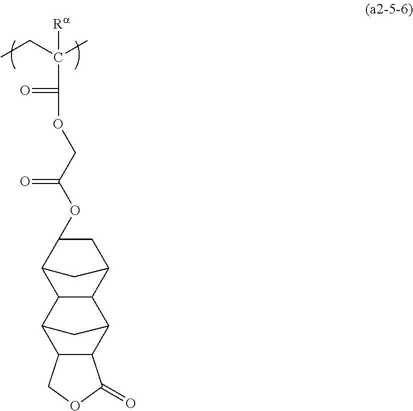

In the component (A1), as the structural unit (a2), one type of structural unit may be used, or two or more types may be used in combination.

As the structural unit (a2), at least one structural unit selected from the group consisting of formulas (a2-1) to (a2-5) is preferable, and at least one structural unit selected from the group consisting of formulas (a2-1) to (a2-3) is more preferable. Of these, it is preferable to use at least one structural unit selected from the group consisting of structural units represented by formulas (a2-1-1), (a2-1-2), (a2-2-1), (a2-2-7), (a2-3-1) and (a2-3-5).

In the component (A1), the amount of the structural unit (a2) based on the combined total of all structural units constituting the component (A1) is preferably 5 to 60 mol %, more preferably 10 to 50 mol %, and still more preferably 10 to 45 mol %. When the amount of the structural unit (a2) is at least as large as the lower limit of the above-mentioned range, the effect of using the structural unit (a2) can be satisfactorily achieved. On the other hand, when the amount of the structural unit (a2) is no more than the upper limit of the above-mentioned range, a good balance can be achieved with the other structural units.

(Structural Unit (a3))

The structural unit (a1) is a structural unit derived from an acrylate ester which may have an atom other than hydrogen or a substituent bonded to the carbon atom on the α position and contains a polar group-containing aliphatic hydrocarbon group.

When the component (A1) includes the structural unit (a3), the hydrophilicity of the component (A) is improved, and hence, the compatibility of the component (A) with the developing solution is improved. As a result, the alkali solubility of the exposed portions improves, which contributes to favorable improvements in the resolution.

Examples of the polar group include a hydroxyl group, cyano group, carboxyl group, or hydroxyalkyl group in which some of the hydrogen atoms of the alkyl group have been substituted with fluorine atoms, although a hydroxyl group is particularly desirable.

Examples of the aliphatic hydrocarbon group include linear or branched hydrocarbon groups (preferably alkylene groups) of 1 to 10 carbon atoms, and cyclic aliphatic hydrocarbon groups (cyclic groups). These cyclic groups can be selected appropriately from the multitude of groups that have been proposed for the resins of resist compositions designed for use with ArF excimer lasers. The cyclic group is preferably a polycyclic group, more preferably a polycyclic group of 7 to 30 carbon atoms.

Of the various possibilities, structural units derived from an acrylate ester that include an aliphatic polycyclic group that contains a hydroxyl group, cyano group, carboxyl group or a hydroxyalkyl group in which part of the hydrogen atoms of the alkyl group have been substituted with fluorine atoms are particularly desirable. Examples of the polycyclic group include groups in which two or more hydrogen atoms have been removed from a bicycloalkane, tricycloalkane, tetracycloalkane or the like. Specific examples include groups in which two or more hydrogen atoms have been removed from a polycycloalkane such as adamantane, norbornane, isobornane, tricyclodecane or tetracyclododecane. Of these polycyclic groups, groups in which two or more hydrogen atoms have been removed from adamantane, norbornane or tetracyclododecane are preferred industrially.

When the aliphatic hydrocarbon group within the polar group-containing aliphatic hydrocarbon group is a linear or branched hydrocarbon group of 1 to 10 carbon atoms, the structural unit (a3) is preferably a structural unit derived from a hydroxyethyl ester of acrylic acid. On the other hand, when the hydrocarbon group is a polycyclic group, structural units represented by formulas (a3-1), (a3-2) and (a3-3) shown below are preferable.

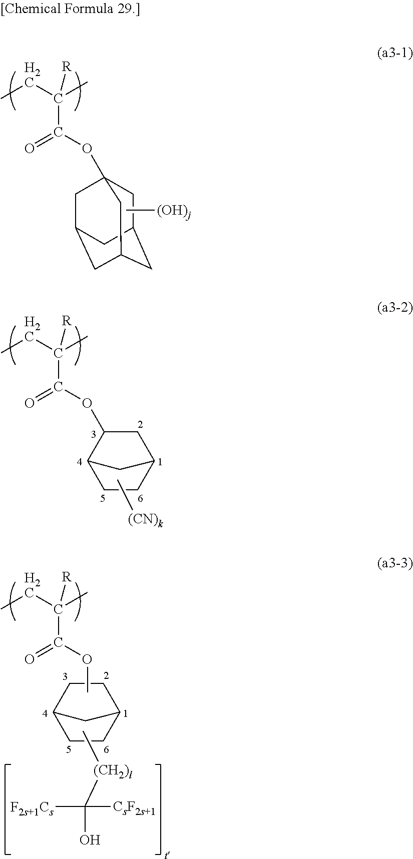

In the formulas, R is the same as defined above; j is an integer of 1 to 3; k is an integer of 1 to 3; t′ is an integer of 1 to 3; 1 is an integer of 1 to 5; and s is an integer of 1 to 3.

In formula (a3-1), j is preferably 1 or 2, and more preferably 1. When j is 2, it is preferable that the hydroxyl groups be bonded to the 3rd and 5th positions of the adamantyl group. When j is 1, it is preferable that the hydroxyl group be bonded to the 3rd position of the adamantyl group.

j is preferably 1, and it is particularly desirable that the hydroxyl group be bonded to the 3rd position of the adamantyl group.

In formula (a3-2), k is preferably 1. The cyano group is preferably bonded to the 5th or 6th position of the norbornyl group.

In formula (a3-3), t′ is preferably 1. l is preferably 1. s is preferably 1. Further, it is preferable that a 2-norbornyl group or 3-norbornyl group be bonded to the terminal of the carboxy group of the acrylic acid. The fluorinated alkyl alcohol is preferably bonded to the 5th or 6th position of the norbornyl group.

As the structural unit (a3), one type of structural unit may be used, or two or more types may be used in combination.

The amount of the structural unit (a3) within the component (A1) based on the combined total of all structural units constituting the component (A1) is preferably 5 to 50 mol %, more preferably 5 to 40 mol %, and still more preferably 5 to 25 mol %. When the amount of the structural unit (a3) is at least as large as the lower limit of the above-mentioned range, the effect of using the structural unit (a3) can be satisfactorily achieved. On the other hand, when the amount of the structural unit (a3) is no more than the upper limit of the above-mentioned range, a good balance can be achieved with the other structural units.

(Structural Unit (a0))

The structural unit (a0) is a structural unit derived from an acrylate ester which may have an atom other than hydrogen or a substituent bonded to the carbon atom on the α position and contains an —SO₂— group containing cyclic group.

By virtue of the structural unit (a0) containing a —SO₂— containing cyclic group, a resist composition containing the component (A1) including the structural unit (a0) is capable of improving the adhesion of a resist film to a substrate Further, the structural unit (a0) contributes to improvement in various lithography properties such as sensitivity, resolution, exposure latitude (EL margin), line width roughness (LWR), line edge roughness (LER) and mask reproducibility.

Here, an “—SO₂— containing cyclic group” refers to a cyclic group having a ring containing —SO₂— within the ring structure thereof, i.e., a cyclic group in which the sulfur atom (S) within —SO₂— forms part of the ring skeleton of the cyclic group.

In the —SO₂— containing cyclic group, the ring containing —SO₂— within the ring skeleton thereof is counted as the first ring. A cyclic group in which the only ring structure is the ring that contains —SO₂— in the ring skeleton thereof is referred to as a monocyclic group, and a group containing other ring structures is described as a polycyclic group regardless of the structure of the other rings.

The —SO₂— containing cyclic group may be either a monocyclic group or a polycyclic group.

As the —SO₂— containing cyclic group, a cyclic group containing —O—SO₂— within the ring skeleton thereof, i.e., a cyclic group containing a sultone ring in which —O—S—within the —O—SO₂— group forms part of the ring skeleton thereof is particularly desirable.

The —SO₂— containing cyclic group preferably has 3 to 30 carbon atoms, more preferably 4 to 20, still more preferably 4 to 15, and most preferably 4 to 12. Herein, the number of carbon atoms refers to the number of carbon atoms constituting the ring skeleton, excluding the number of carbon atoms within a substituent.

The —SO₂— containing cyclic group may be either a —SO₂— containing aliphatic cyclic group or a —SO₂— containing aromatic cyclic group. A —SO₂— containing aliphatic cyclic group is preferable.

Examples of the —SO₂— containing aliphatic cyclic group include aliphatic cyclic groups in which part of the carbon atoms constituting the ring skeleton has been substituted with a —SO₂— group or a —O—SO₂— group and has at least one hydrogen atom removed from the aliphatic hydrocarbon ring. Specific examples include an aliphatic hydrocarbon ring in which a —CH₂— group constituting the ring skeleton thereof has been substituted with a —SO₂— group and has at least one hydrogen atom removed therefrom; and an aliphatic hydrocarbon ring in which a —CH₂—CH₂— group constituting the ring skeleton has been substituted with a —O—SO₂— group and has at least one hydrogen atom removed therefrom.

The alicyclic hydrocarbon group preferably has 3 to 20 carbon atoms, and more preferably 3 to 12 carbon atoms.

The alicyclic hydrocarbon group may be either a monocyclic group or a polycyclic group. As the monocyclic group, a group in which two hydrogen atoms have been removed from a monocycloalkane of 3 to 6 carbon atoms is preferable. Examples of the monocycloalkane include cyclopentane and cyclohexane. As the polycyclic group, a group in which two hydrogen atoms have been removed from a polycycloalkane of 7 to 12 carbon atoms is preferable. Examples of the polycycloalkane include adamantane, norbornane, isobornane, tricyclodecane and tetracyclododecane.

The —SO₂— containing cyclic group may have a substituent. Examples of the substituent include an alkyl group, an alkoxy group, a halogen atom, a halogenated alkyl group, a hydroxy group, an oxygen atom (═O), —COOR″, —OC(═O)R″, a hydroxyalkyl group and a cyano group (wherein R″ represents a hydrogen atom or an alkyl group).

The alkyl group for the substituent is preferably an alkyl group of 1 to 6 carbon atoms. Further, the alkyl group is preferably a linear alkyl group or a branched alkyl group. Specific examples include a methyl group, an ethyl group, a propyl group, an isopropyl group, an n-butyl group, an isobutyl group, a tert-butyl group, a pentyl group, an isopentyl group, a neopentyl group and a hexyl group. Among these, a methyl group or ethyl group is preferable, and a methyl group is particularly desirable.

As the alkoxy group for the substituent, an alkoxy group of 1 to 6 carbon atoms is preferable. Further, the alkoxy group is preferably a linear alkoxy group or a branched alkyl group. Specific examples of the alkoxy group include the aforementioned alkyl groups for the substituent having an oxygen atom (—O—) bonded thereto.

Examples of the halogen atom for the substituent include a fluorine atom, a chlorine atom, a bromine atom and an iodine atom, and a fluorine atom is preferable.

Examples of the halogenated alkyl group for the substituent include groups in which part or all of the hydrogen atoms within the aforementioned alkyl groups has been substituted with the aforementioned halogen atoms.

As examples of the halogenated lower alkyl group for the substituent, groups in which part or all of the hydrogen atoms of the aforementioned alkyl groups for the substituent have been substituted with the aforementioned halogen atoms can be given. As the halogenated alkyl group, a fluorinated alkyl group is preferable, and a perfluoroalkyl group is particularly desirable.

In the —COOR″ group and the —OC(═O)R″ group, R″ preferably represents a hydrogen atom or a linear, branched or cyclic alkyl group of 1 to 15 carbon atoms.

When R″ represents a linear or branched alkyl group, it is preferably an alkyl group of 1 to 10 carbon atoms, more preferably an alkyl group of 1 to 5 carbon atoms, and most preferably a methyl group or an ethyl group.

When R″ is a cyclic alkyl group (cycloalkyl group), it preferably has 3 to 15 carbon atoms, more preferably 4 to 12 carbon atoms, and most preferably 5 to 10 carbon atoms. As examples of the cycloalkyl group, groups in which one or more hydrogen atoms have been removed from a monocycloalkane or a polycycloalkane such as a bicycloalkane, tricycloalkane or tetracycloalkane, which may or may not be substituted with a fluorine atom or a fluorinated alkyl group, may be used. Specific examples include groups in which one or more hydrogen atoms have been removed from a monocycloalkane such as cyclopentane and cyclohexane; and groups in which one or more hydrogen atoms have been removed from a polycycloalkane such as adamantane, norbornane, isobornane, tricyclodecane or tetracyclododecane.

The hydroxyalkyl group for the substituent preferably has 1 to 6 carbon atoms, and specific examples thereof include the aforementioned alkyl groups for the substituent in which at least one hydrogen atom has been substituted with a hydroxy group.

More specific examples of the —SO₂— containing cyclic group include groups represented by general formulas (3-1) to (3-4) shown below.

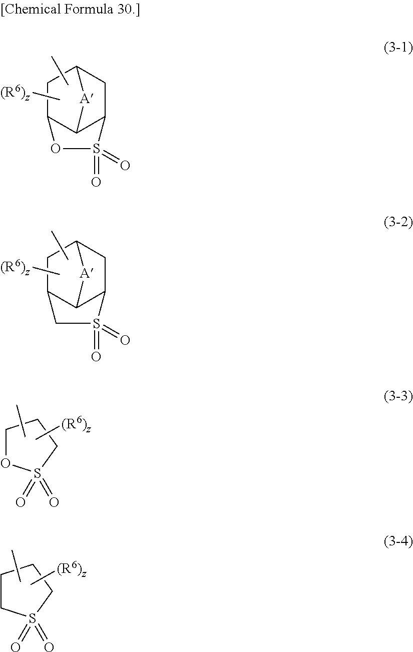

In the formulas, A′ represents an oxygen atom, a sulfur atom or an alkylene group of 1 to 5 carbon atoms which may contain an oxygen atom or a sulfur atom; z represents an integer of 0 to 2; and R⁶ represents an alkyl group, an alkoxy group, a halogenated alkyl group, a hydroxyl group, —COOR″, —OC(═O)R″, a hydroxyalkyl group or a cyano group, wherein R″ represents a hydrogen atom or an alkyl group.

In general formulas (3-1) to (3-4) above, A′ represents an oxygen atom (—O—), a sulfur atom (—S—) or an alkylene group of 1 to 5 carbon atoms which may contain an oxygen atom or a sulfur atom.

As the alkylene group of 1 to 5 carbon atoms represented by A′, a linear or branched alkylene group is preferable, and examples thereof include a methylene group, an ethylene group, an n-propylene group and an isopropylene group.

Examples of alkylene groups that contain an oxygen atom or a sulfur atom include the aforementioned alkylene groups in which —O— or —S— is bonded to the terminal of the alkylene group or present between the carbon atoms of the alkylene group. Specific examples of such alkylene groups include —O—CH₂—, —CH₂—O—CH₂—, —S—CH₂—, —CH₂—S—CH₂—.

As A′, an alkylene group of 1 to 5 carbon atoms or —O— is preferable, more preferably an alkylene group of 1 to 5 carbon atoms, and most preferably a methylene group.

z represents an integer of 0 to 2, and is most preferably 0.

When z is 2, the plurality of R⁶ may be the same or different from each other.

As the alkyl group, alkoxy group, halogenated alkyl group, —COOR″, —OC(═O)R″ and hydroxyalkyl group for R⁶, the same alkyl groups, alkoxy groups, halogenated alkyl groups, —COOR″, —OC(═O)R″ and hydroxyalkyl groups as those described above as the substituent for the —SO₂— containing cyclic group can be mentioned.

Specific examples of the cyclic groups represented by general formulas (3-1) to (3-4) are shown below. In the formulas shown below, “Ac” represents an acetyl group.

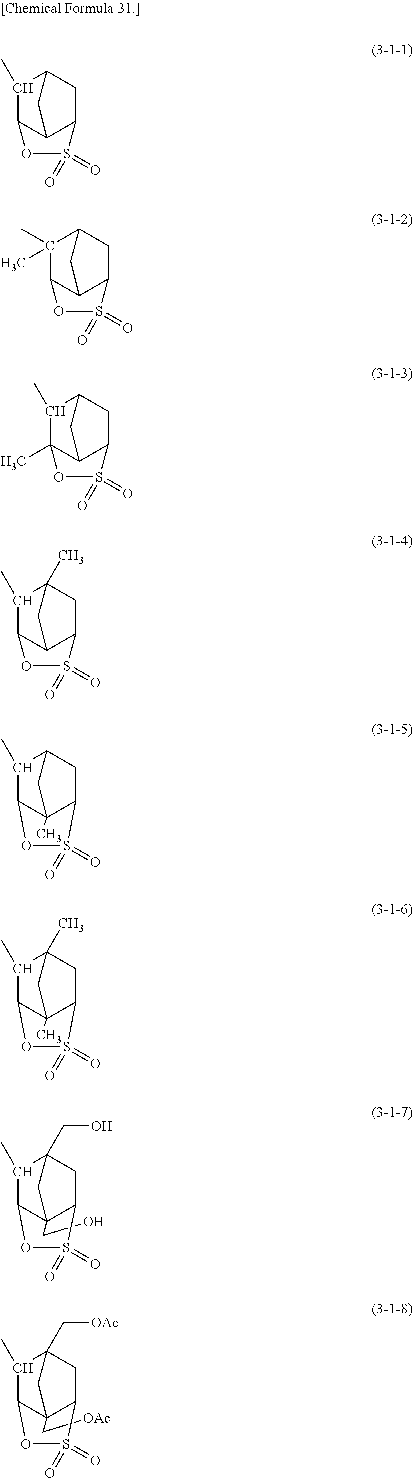

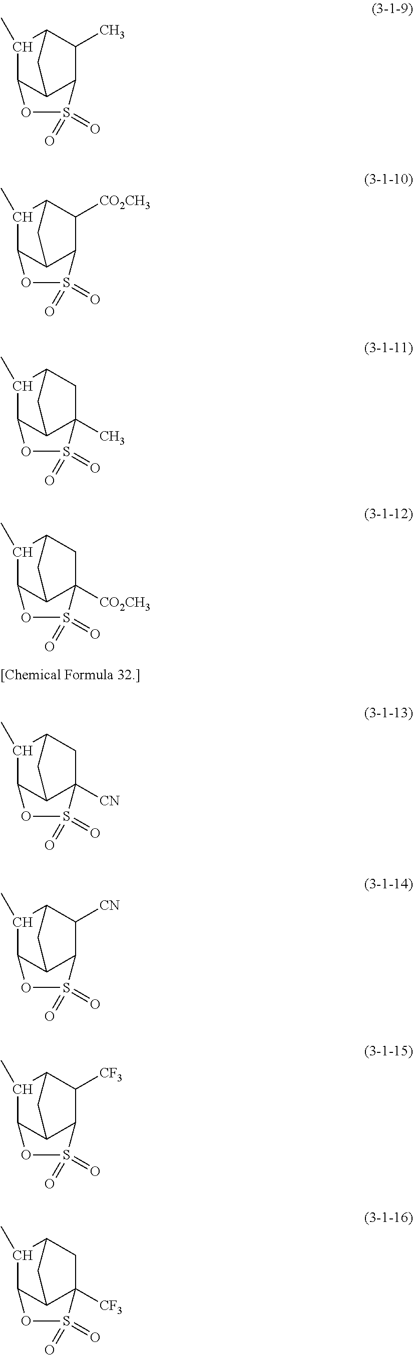

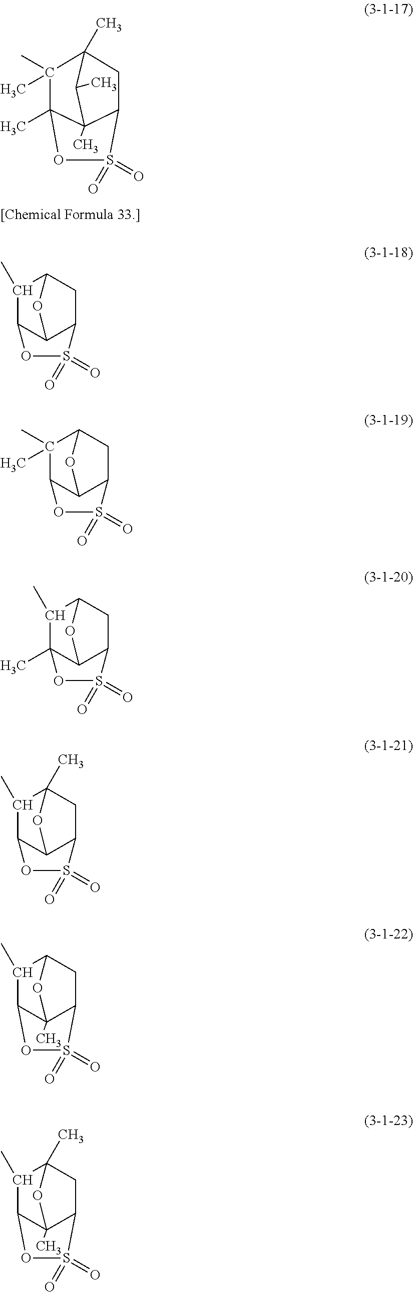

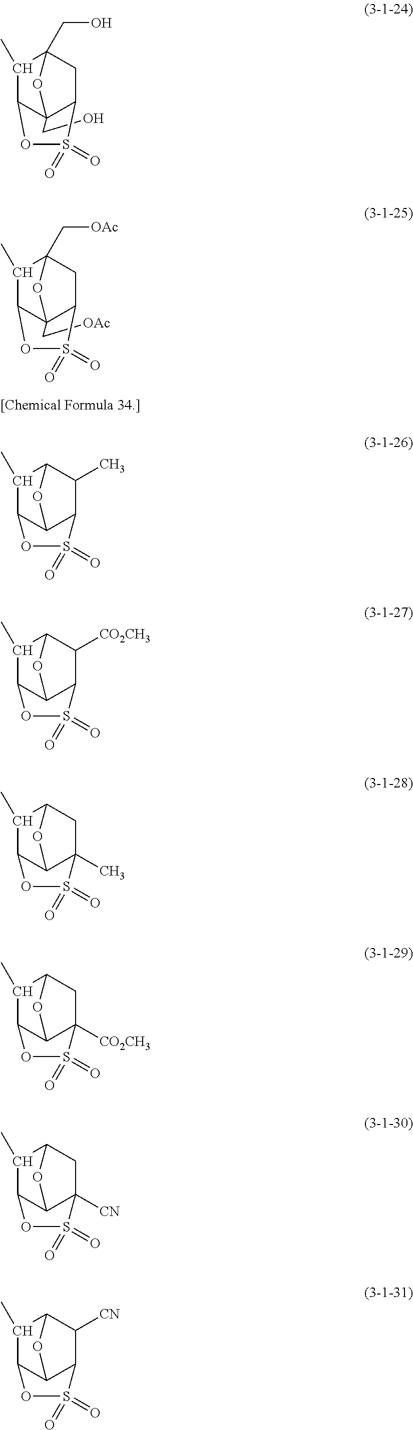

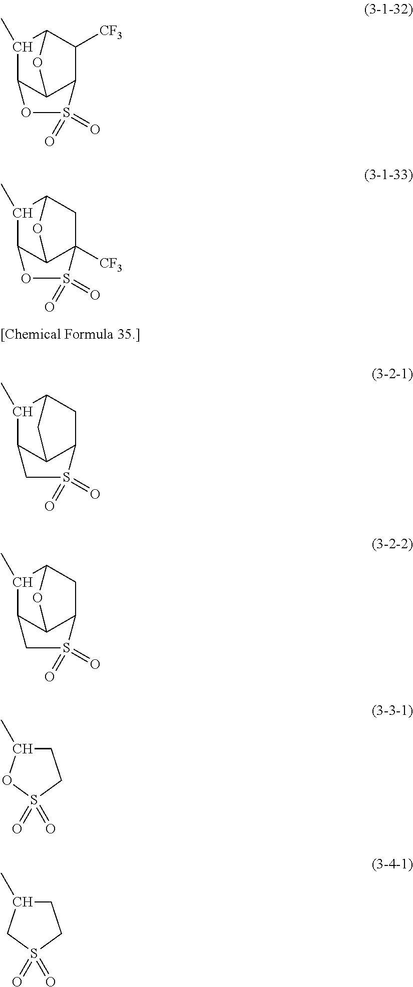

As the —SO₂— containing cyclic group, a group represented by the aforementioned general formula (3-1) is preferable, at least one member selected from the group consisting of groups represented by the aforementioned chemical formulas (3-1-1), (3-1-18), (3-3-1) and (3-4-1) is more preferable, and a group represented by chemical formula (3-1-1) is most preferable.

More specifically, examples of the structural unit (a0) include structural units represented by general formula (a0-0) shown below.

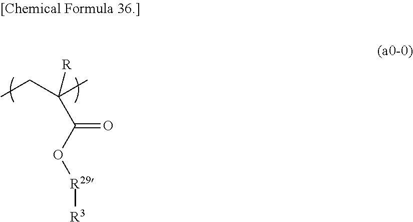

In the formula, R represents a hydrogen atom, an alkyl group of 1 to 5 carbon atoms or a halogenated alkyl group of 1 to 5 carbon atoms; R³ represents a —SO₂— containing cyclic group; and R²⁹′ represents a single bond or a divalent linking group.

In general formula (a0-0), R represents a hydrogen atom, an alkyl group of 1 to 5 carbon atoms or a halogenated alkyl group of 1 to 5 carbon atoms.

As the alkyl group for R, a linear or branched alkyl group of 1 to 5 carbon atoms is preferable, and specific examples thereof include a methyl group, an ethyl group, a propyl group, an isopropyl group, an n-butyl group, an isobutyl group, a tert-butyl group, a pentyl group, an isopentyl group and a neopentyl group.

The halogenated alkyl group for R is a group in which part or all of the hydrogen atoms of the aforementioned alkyl group has been substituted with halogen atoms. Examples of the halogen atom include a fluorine atom, a chlorine atom, a bromine atom and an iodine atom, and a fluorine atom is particularly desirable.

As R, a hydrogen atom, an alkyl group of 1 to 5 carbon atoms or a fluorinated alkyl group of 1 to 5 carbon atoms is preferable, and a hydrogen atom or a methyl group is particularly desirable in terms of industrial availability.

In formula (a0-0), R³ is the same as defined for the aforementioned —SO₂— containing group.

R²⁹′ may be a single bond or a divalent linking group. In terms of the effects of the present invention, a divalent linking group is preferable.

Examples of the divalent linking group for R²⁹′ include the same divalent linking groups as those described above for R²⁹ in the structural unit (a2).

As the divalent linking group for R²⁹′, an alkylene group, a divalent alicyclic hydrocarbon group or a divalent linking group containing a hetero atom is preferable. Among these, an alkylene group or a divalent linking group containing a hetero atom is more preferable, and a linear alkylene group is particularly desirable.

When R²⁹′ represents an alkylene group, it preferably has 1 to 10 carbon atoms, more preferably 1 to 6, still more preferably 1 to 4, and most preferably 1 to 3. Specific examples of alkylene groups include the aforementioned linear alkylene groups and branched alkylene groups.

When R²⁹′ represents a divalent alicyclic hydrocarbon group, as the alicyclic hydrocarbon group, the same alicyclic hydrocarbon groups as those described above for the “aliphatic hydrocarbon group containing a ring in the structure thereof” can be used.

As the alicyclic hydrocarbon group, a group in which two or more hydrogen atoms have been removed from cyclopentane, cyclohexane, norbornane, isobornane, adamantane, tricyclodecane or tetracyclododecane is particularly desirable.

When R²⁹′ represents a divalent linking group containing a hetero atom, preferable examples of the linking group include —O—, —C(═O)—O—, —C(═O)—, —O—C(═O)—O—, —C(═O)—NH—, —NH— (H may be substituted with a substituent such as an alkyl group or an acyl group), —S—, —S(═O)₂—, —S(═O)₂—O— and a group represented by general formula -A-O—B—, -[A-C(═O)—O]_{m′}—B— or -A-O—C(═O)—B-[in the formulas, each of A and B independently represents a divalent hydrocarbon group which may have a substituent, O represents an oxygen atom, and m′ represents an integer of 0 to 3].

When R²⁹′ represents —NH—, H may be substituted with a substituent such as an alkyl group, an aryl group (an aromatic group) or the like. The substituent (an alkyl group, an aryl group or the like) preferably has 1 to 10 carbon atoms, more preferably 1 to 8, and most preferably 1 to 5.

In the group represented by the formula -A-O—B—, -[A-C(═O)—O]_{m′}—B— or -A-O—C(═O)—B—, each of A and B independently represents a divalent hydrocarbon group which may have a substituent. As the divalent hydrocarbon group, the same groups as those described above for the “divalent hydrocarbon group which may have a substituent” for R²⁹′ can be mentioned.

As A, a linear aliphatic hydrocarbon group is preferable, more preferably a linear alkylene group, still more preferably a linear alkylene group of 1 to 5 carbon atoms, and a methylene group or an ethylene group is particularly desirable.

As B, a linear or branched aliphatic hydrocarbon group is preferable, and a methylene group, an ethylene group or an alkylmethylene group is more preferable. The alkyl group within the alkylmethylene group is preferably a linear alkyl group of 1 to 5 carbon atoms, more preferably a linear alkyl group of 1 to 3 carbon atoms, and most preferably a methyl group.

In the group represented by the formula -[A-C(═O)—O]_{m′}—B—, m represents an integer of 0 to 3, preferably an integer of 0 to 2, more preferably 0 or 1, and most preferably 1. Namely, it is particularly desirable that the group represented by the formula -[A-C(═O)—O]_{m′}—B— is a group represented by the formula -A-C(═O)—O—B—. Among these, a group represented by the formula —(CH₂)_{a′}—C(═O)—O—(CH₂)_{b′}— is preferable. In the formula, a′ is an integer of 1 to 10, preferably an integer of 1 to 8, more preferably an integer of 1 to 5, still more preferably 1 or 2, and most preferably 1. b′ is an integer of 1 to 10, preferably an integer of 1 to 8, more preferably an integer of 1 to 5, still more preferably 1 or 2, and most preferably 1.

Among these examples, as the divalent linking group containing a hetero atom, a linear group containing oxygen as the hetero atom (e.g., an ether bond or an ester bond) is preferable.

As the alkylene group, a linear or branched alkylene group is preferable. Specific examples include the same linear alkylene groups and branched alkylene groups as those described above for the aliphatic hydrocarbon group represented by R²⁹′.

As the divalent linking group containing an ester bond, a group represented by general formula: —R²—C(═O)—O— (in the formula, R² represents a divalent linking group) is particularly desirable. Namely, the structural unit (a0) is preferably a structural unit represented by general formula (a0-0-1) shown below.

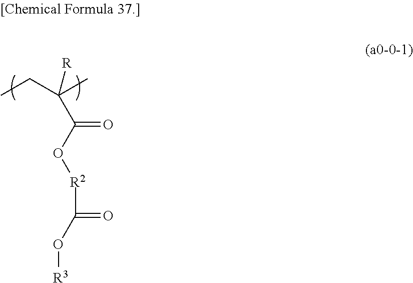

In the formula, R represents a hydrogen atom, an alkyl group of 1 to 5 carbon atoms or a halogenated alkyl group of 1 to 5 carbon atoms; R² represents a divalent linking group; and R³ represents an —SO₂— containing cyclic group.

R² is not particularly limited. For example, the same divalent linking groups as those described for R²⁹′ in general formula (a0-0) can be mentioned.

As the divalent linking group for R², an alkylene group, a divalent alicyclic hydrocarbon group or a divalent linking group containing a hetero atom is preferable.

As the linear or branched alkylene group, the divalent alicyclic hydrocarbon group and the divalent linking group containing a hetero atom, the same linear or branched alkylene group, divalent alicyclic hydrocarbon group and divalent linking group containing a hetero atom as those described above as preferable examples of R²⁹′ can be mentioned.

Among these, a linear or branched alkylene group, or a divalent linking group containing an oxygen atom as a hetero atom is more preferable.

As the linear alkylene group, a methylene group or an ethylene group is preferable, and a methylene group is particularly desirable.

As the branched alkylene group, an alkylmethylene group or an alkylethylene group is preferable, and —CH(CH₃)—, —C(CH₃)₂— or —C(CH₃)₂CH₂— is particularly desirable.

As the divalent linking group containing a hetero atom, a divalent linking group containing an ether bond or an ester bond is preferable, and a group represented by the aforementioned formula -A-O—B—, -[A-C(═O)—O]_{m′}—B— or -A-O—C(═O)—B— is more preferable. m′ represents an integer of 0 to 3.

Among these, a group represented by the formula -A-O—C(═O)—B— is preferable, and a group represented by the formula: —(CH₂)_{c}—O—C(═O)—(CH₂)_{d}— is particularly desirable. c represents an integer of 1 to 5, and preferably 1 or 2. d represents an integer of 1 to 5, and preferably 1 or 2.

In particular, as the structural unit (a0), a structural unit represented by general formula (a0-0-11) or (a0-0-12) shown below is preferable, and a structural unit represented by general formula (a0-0-12) is more preferable.

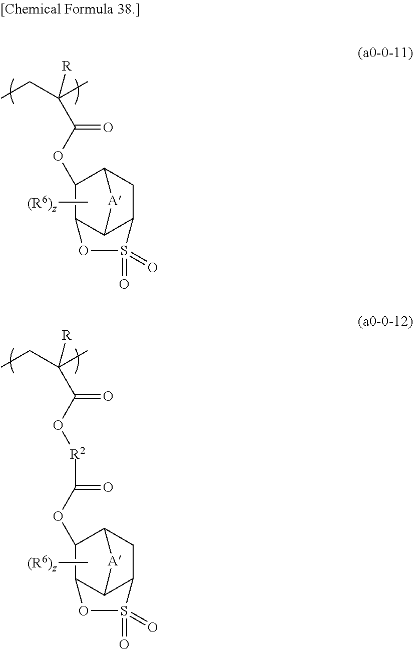

In the formulas, R, A′, R⁶, z and R² are the same as defined above.

In general formula (a0-0-11), A′ is preferably a methylene group, an ethylene group, an oxygen atom (—O—) or a sulfur atom (—S—).

As R², a linear or branched alkylene group or a divalent linking group containing an oxygen atom is preferable. As the linear or branched alkylene group and the divalent linking group containing an oxygen atom represented by R², the same linear or branched alkylene groups and the divalent linking groups containing an oxygen atom as those described above can be mentioned.

As the structural unit represented by general formula (a0-0-12), a structural unit represented by general formula (a0-0-12a) or (a0-0-12b) shown below is particularly desirable.

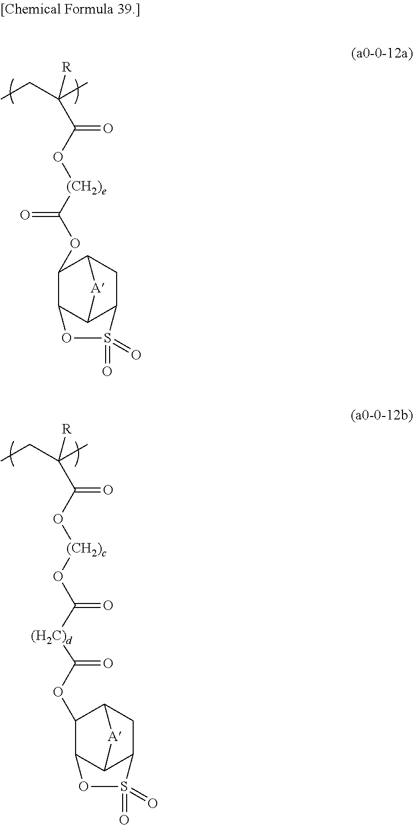

In the formulas, R and A′ are the same as defined above; and each of c to e independently represents an integer of 1 to 5.

As the structural unit (a0), one type of structural unit may be used alone, or two or more types of structural units may be used in combination.

In terms of achieving an excellent shape for a resist pattern formed using a positive resist composition containing the component (A1) and excellent lithography properties such as EL margin, LWR and mask reproducibility, the amount of the structural unit (a0) within the component (A1), based on the combined total of all structural units constituting the component (A1) is preferably 1 to 60 mol %, more preferably 5 to 55 mol %, still more preferably 10 to 50 mol %, and most preferably 15 to 45 mol %.

(Other Structural Units)

The component (A1) may also have a structural unit other than the above-mentioned structural units (a1) to (a3) and (a0), as long as the effects of the present invention are not impaired.

As such a structural unit, any other structural unit which cannot be classified as one of the above structural units (a1) to (a3) and (a0) can be used without any particular limitation, and any of the multitude of conventional structural units used within resist resins for ArF excimer lasers or KrF excimer lasers (and particularly for ArF excimer lasers) can be used.

As such a structural unit, for example, a structural unit (a4) derived from an acrylate ester containing a non-acid-dissociable aliphatic polycyclic group can be mentioned.

Structural Unit (a4)

The structural unit (a2) is a structural unit derived from an acylate ester which may have an atom other than hydrogen or a substituent bonded to the carbon atom on the α position and contains an acid non-dissociable aliphatic polycyclic group.

In the structural unit (a4), examples of this polycyclic group include the same groups as those described above in relation to the aforementioned structural unit (a1), and any of the multitude of conventional polycyclic groups used within the resin component of resist compositions for ArF excimer lasers or KrF excimer lasers (and particularly for ArF excimer lasers) can be used.

In consideration of industrial availability and the like, at least one polycyclic group selected from amongst a tricyclodecyl group, adamantyl group, tetracyclododecyl group, isobornyl group, and norbornyl group is particularly desirable. These polycyclic groups may be substituted with a linear or branched alkyl group of 1 to 5 carbon atoms. Specific examples of the structural unit (a4) include units with structures represented by general formulas (a4-1) to (a4-5) shown below.

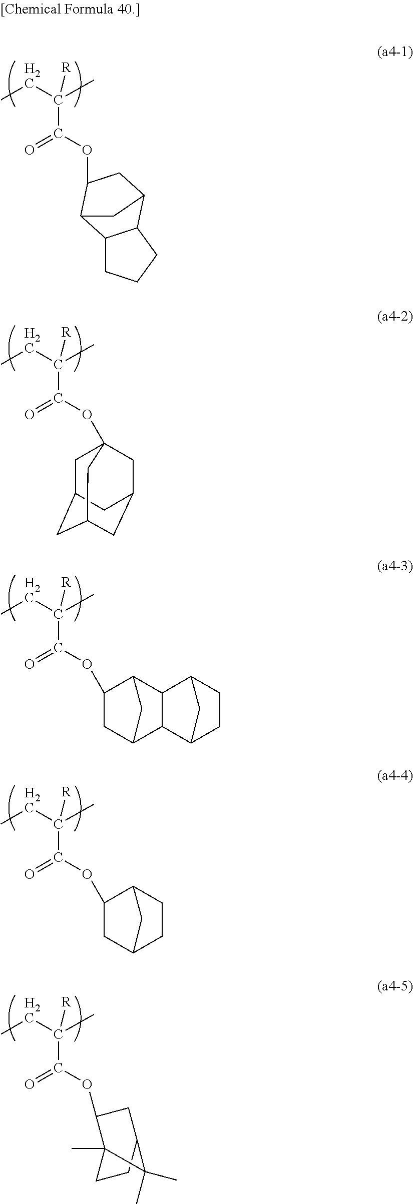

In the formulas, R is the same as defined above.

When the structural unit (a4) is included in the component (A1), the amount of the structural unit (a4) based on the combined total of all the structural units that constitute the component (A1) is preferably within the range from 1 to 30 mol %, and more preferably from 10 to 20 mol %.

In the resist composition of the present invention, the component (A1) is preferably a polymeric compound having a structural unit (a1). Further, the component (A1) is preferably a polymeric compound having the structural unit (a0) because the adhesion of the resist film to a substrate can be enhanced, and lithography properties can be improved.

Examples of the component (A1) include a copolymer consisting of the structural units (a1), (a2) and (a3), a copolymer consisting of the structural units (a1), (a2), (a3) and (a4), a copolymer consisting of the structural units (a1), (a2) and (a0), a copolymer consisting of the structural units (a1), (a3) and (a0), and a copolymer consisting of the structural units (a1), (a2), (a3) and (a0).

In the component (A), as the component (A1), one type may be used alone, or two or more types may be used in combination.

In the present invention, as the component (A1), a polymeric compound that includes a combination of structural units such as that shown below is particularly desirable.

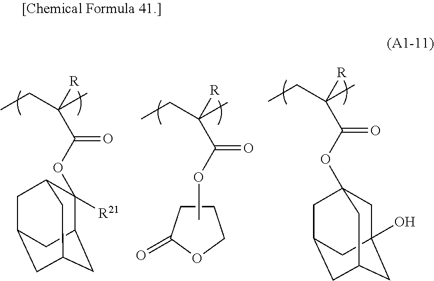

In the formula, R and R²¹ are the same as defined above, and the plurality of R may be the same or different from each other.

In formula (A1-11), the lower alkyl group for R²¹ is the same as the lower alkyl group for R above, preferably a methyl group or an ethyl group, and most preferably a methyl group.

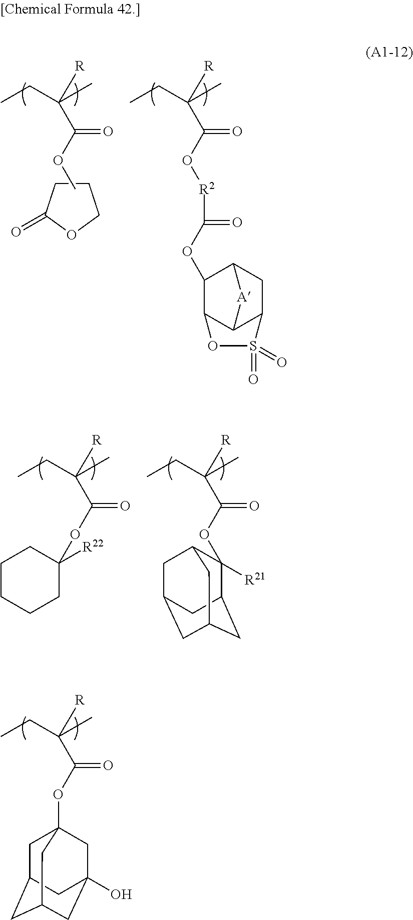

In the formula, R, R², A′, R²¹ and R²² are the same as defined above; and the plurality of R may be the same or different from each other.

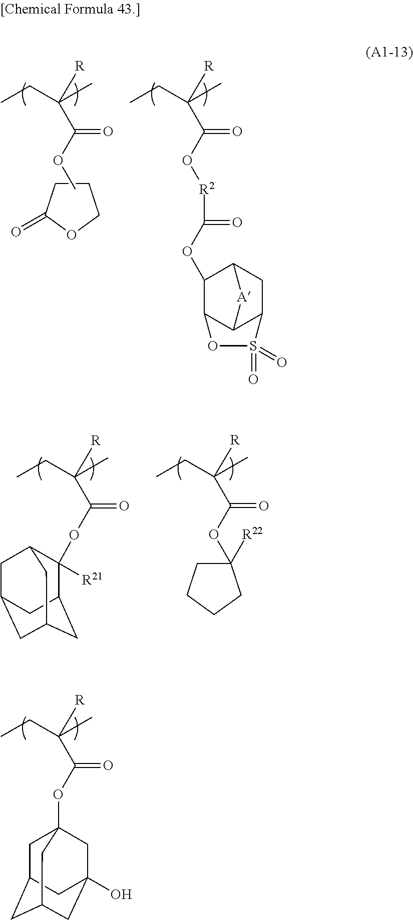

In the formula, R, R², A′, R²¹ and R²² are the same as defined above; and the plurality of R may be the same or different from each other.

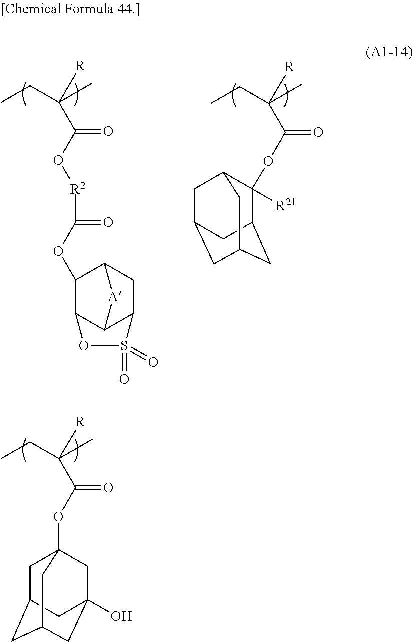

In the formulas, R, R², A′ and R²¹ are the same as defined above; and the plurality of R may be the same or different from each other.

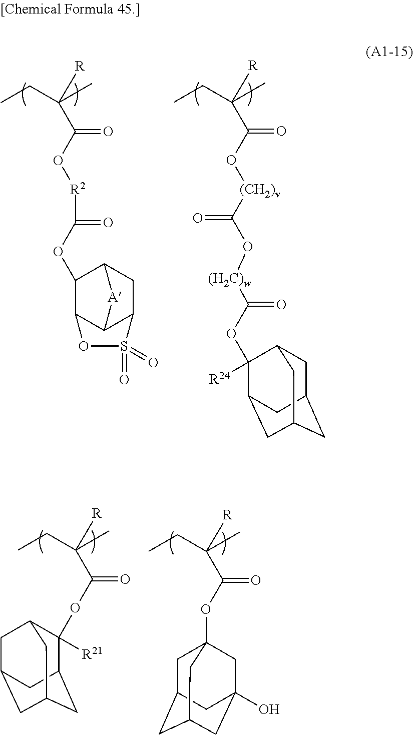

In the formula, R, R², A′, R²⁴, v, w and R²¹ are the same as defined above; and the plurality of R may be the same or different from each other.

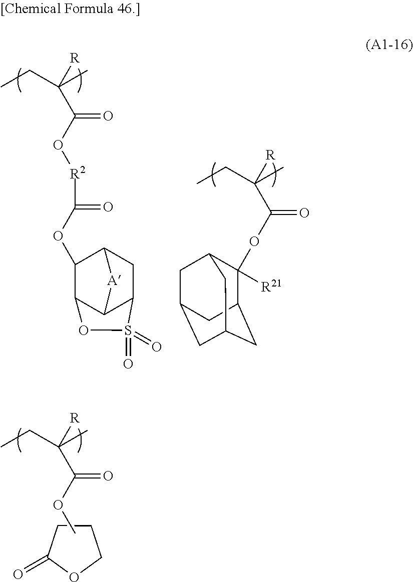

In the formulas, R, R², A′ and R²¹ are the same as defined above; and the plurality of R may be the same or different from each other.

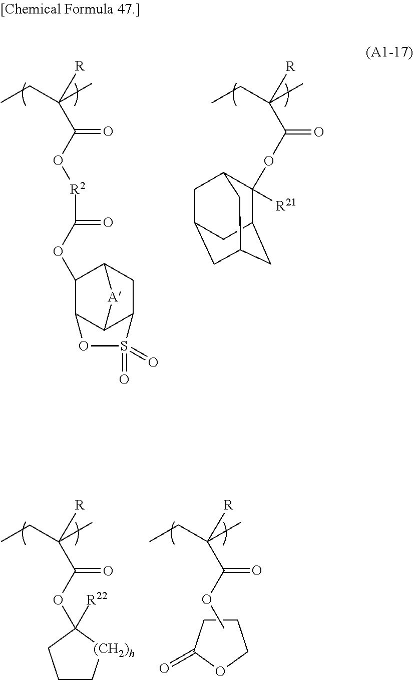

In the formula, R, R², A′, R²¹, R²² and h are the same as defined above; and the plurality of R may be the same or different from each other.

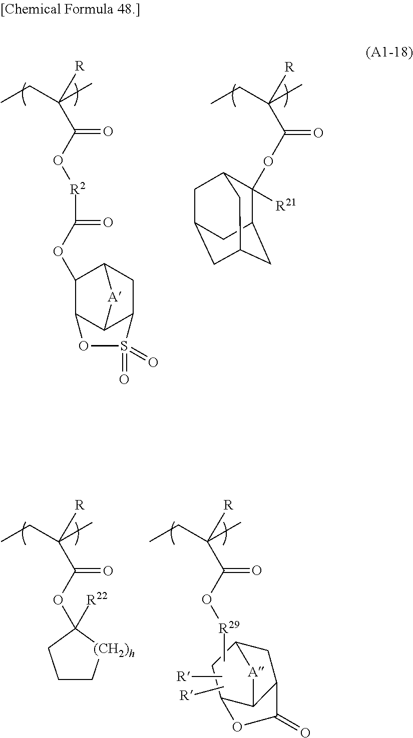

In the formula, R, R², A′, R²¹, R²², h, R²⁹, A″ and R′ are the same as defined above; wherein the plurality of R and R′ may be the same or different from each other.

The component (A1) can be obtained, for example, by a conventional radical polymerization or the like of the monomers corresponding with each of the structural units, using a radical polymerization initiator such as azobisisobutyronitrile (AIBN).

Furthermore, in the component (A1), by using a chain transfer agent such as HS—CH₂—CH₂—CH₂—C(CF₃)₂—OH, a —C(CF₃)₂—OH group can be introduced at the terminals of the component (A1). Such a copolymer having introduced a hydroxyalkyl group in which some of the hydrogen atoms of the alkyl group are substituted with fluorine atoms is effective in reducing developing defects and LER (line edge roughness:unevenness of the side walls of a line pattern).

The weight average molecular weight (Mw) (the polystyrene equivalent value determined by gel permeation chromatography) of the component (A1) is not particularly limited, but is preferably 1,000 to 50,000, more preferably 1,500 to 30,000, and most preferably 2,500 to 20,000. When the weight average molecular weight is no more than the upper limit of the above-mentioned range, the resist composition exhibits a satisfactory solubility in a resist solvent. On the other hand, when the weight average molecular weight is at least as large as the lower limit of the above-mentioned range, dry etching resistance and the cross-sectional shape of the resist pattern becomes satisfactory.

Further, the dispersity (Mw/Mn) of the component (A1) is preferably 1.0 to 5.0, more preferably 1.0 to 3.0, and most preferably 1.0 to 2.5. Here, Mn is the number average molecular weight.

In the resist composition of the present invention, the component (A) may contain “a base component which exhibits increased solubility in an alkali developing solution under action of acid” other than the component (A1).

Such base component other than the component (A1) is not particularly limited, and any of the multitude of conventional base components used within chemically amplified resist compositions (e.g., a novolak resin, a polyhydroxystyrene-based resin (PHS), a low molecular weight component (component (A2))) can be appropriately selected for use.

Examples of the component (A2) include low molecular weight compounds that have a molecular weight of at least 500 and less than 2,000, contains a hydrophilic group, and also contains an acid dissociable, dissolution inhibiting group described above in connection with the component (A1). Specific examples of the low molecular weight compound include compounds containing a plurality of phenol skeletons in which a part of the hydrogen atoms within hydroxyl groups have been substituted with the aforementioned acid dissociable, dissolution inhibiting groups.

In the resist composition of the present invention, as the component (A), one type may be used, or two or more types of compounds may be used in combination.

In the component (A), the amount of the component (A1) based on the total weight of the component (A) is preferably 25% by weight or more, more preferably 50% by weight or more, still more preferably 75% by weight or more, and may be even 100% by weight. When the amount of the component (A1) is 25% by weight or more, a resist pattern exhibiting a high resolution and a high rectangularity can be formed.

In the resist composition of the present invention, the amount of the component (A) can be appropriately adjusted depending on the thickness of the resist film to be formed, and the like.

<Component (B)>

In the resist composition of the present invention, the component (B) includes an acid generator (B1) containing a compound represented by general formula (b1) shown below (hereafter, this acid generator (B1) is referred to as “component (B1)”).

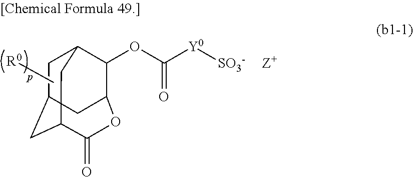

In the formula, Y⁰ represents an alkylene group of 1 to 4 carbon atoms which may have a substituent or a fluorinated alkylene group which may have a substituent; R⁰ represents an alkyl group, an alkoxy group, a halogen atom, a halogenated alkyl group, a hydroxy group or an oxygen atom (═O); p represents 0 or 1; and Z⁺ represents an organic cation.

Anion Moiety of Component (B1)

In formula (b1-1), Y⁰ represents an alkylene group of 1 to 4 carbon atoms which may have a substituent or a fluorinated alkylene group which may have a substituent.

The alkylene group for Y⁰ is preferably a linear or branched alkylene group, and preferably has 1 to 12 carbon atoms, more preferably 1 to 5, and most preferably 1 to 3.

As the fluorinated alkylene group for Y⁰, the aforementioned alkylene group for Y⁰ in which part or all of the hydrogen atoms has been substituted with fluorine atoms can be used.

Specific examples of Y⁰ include —CF₂—, —CF₂CF₂—, —CF₂CF₂CF₂—, —CF(CF₃)CF₂—, —CF(CF₂CF₃)—, —C(CF₃)₂—, —CF₂CF₂CF₂CF₂—, —CF(CF₃)CF₂CF₂—, —CF₂CF(CF₃)CF₂—, —CF(CF₃)CF(CF₃)—, —C(CF₃)₂CF₂—, —CF(CF₂CF₃)CF₂—, —CF(CF₂CF₂CF₃)—, —C(CF₃)(CF₂CF₃)—; —CHF—, —CH₂CF₂—, —CH₂CH₂CF₂—, —CH₂CF₂CF₂—, —CH(CF₃)CH₂—, —CH(CF₂CF₃)—, —C(CH₃)(CF₃)—, —CH₂CH₂CH₂CF₂—, —CH₂CH₂CF₂CF₂—, —CH(CF₃)CH₂CH₂—, —CH₂CH(CF₃)CH₂—, —CH(CF₃)CH(CF₃)—, —C(CF₃)₂CH₂—; —CH₂—, —CH₂CH₂—, —CH₂CH₂CH₂—, —CH(CH₃)CH₂—, —CH(CH₂CH₃)—, —C(CH₃)₂—, —CH₂CH₂CH₂CH₂—, —CH(CH₃)CH₂CH₂—, —CH₂CH(CH₃)CH₂—, —CH(CH₃)CH(CH₃)—, —C(CH₃)₂CH₂—, —CH(CH₂CH₃)CH₂—, —CH(CH₂CH₂CH₃)—, —C(CH₃)(CH₂CH₃)—, and CH(CH₃).

Y⁰ is preferably a fluorinated alkylene group, and particularly preferably a fluorinated alkylene group in which the carbon atom bonded to the adjacent sulfur atom is fluorinated. In such a case, an acid having a strong acid strength is generated from the component (B1) upon exposure. As a result, the resolution and the shape of a resist pattern formed can be improved. Further, the lithographic properties are further improved.

Examples of such fluorinated alkylene groups include —CF₂—, —CF₂CF₂—, —CF₂CF₂CF₂—, —CF(CF₃)CF₂—, —CF₂CF₂CF₂CF₂—, —CF(CF₃)CF₂CF₂—, —CF₂CF(CF₃)CF₂—, —CF(CF₃)CF(CF₃)—, —C(CF₃)₂CF₂—, —CF(CF₂CF₃)CF₂—; —CH₂CF₂—, —CH₂CH₂CF₂—, —CH₂CF₂CF₂—; —CH₂CH₂CH₂CF₂—, —CH₂CH₂CF₂CF₂—, and —CH₂CF₂CF₂CF₂—.

Of these, —CF₂—, —CF₂CF₂—, —CF₂CF₂CF₂— or CH₂CF₂CF₂— is preferable, —CF₂—, —CF₂CF₂— or —CF₂CF₂CF₂— is more preferable, and —CF₂— is particularly desirable in terms the effects of the present invention.

The alkylene group or fluorinated alkylene group may have a substituent.

The alkylene group or fluorinated alkylene group “has a substituent” means that part or all of the hydrogen atoms or fluorine atoms in the alkylene group or fluorinated alkylene group has been substituted with groups other than hydrogen atoms and fluorine atoms.

Examples of substituents for the alkylene group or fluorinated alkylene group include an alkoxy group of 1 to 4 carbon atoms, and a hydroxyl group.

In formula (b1-1), R⁰ represents an alkyl group, an alkoxy group, a halogen atom, a halogenated alkyl group, a hydroxy group or an oxygen atom (═O)

The alkyl group for R⁰ is preferably an alkyl group of 1 to 5 carbon atoms, and more preferably a methyl group, an ethyl group, a propyl group, an n-butyl group or a tert-butyl group.

The alkoxy group for R⁰ is preferably an alkoxy group having 1 to 5 carbon atoms, more preferably a methoxy group, an ethoxy group, an n-propoxy group, an iso-propoxy group, an n-butoxy group or a tert-butoxy group, and most preferably a methoxy group or an ethoxy group.

Examples of the halogen atom for R⁰ include a fluorine atom, chlorine atom, bromine atom and iodine atom, and a fluorine atom is preferable.

Examples of the halogenated alkyl group for R⁰ include groups in which part or all of the hydrogen atoms within the aforementioned alkyl groups for R⁰ has been substituted with the aforementioned halogen atoms.

In formula (b1-1), p represents 0 or 1, and is preferably 0.

Specific examples of the anion moiety of the component (B1) are shown below.

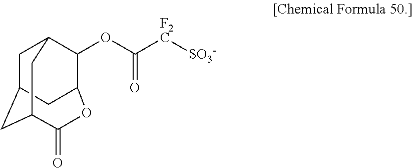

Cation Moiety of Component (B1)

In general formula (b1-1), as the organic cation for Z⁺, there is no particular limitation, and any of those conventionally known as cation moiety for an onium salt-based acid generator can be appropriately selected for use.

As the cation moiety, a sulfonium ion or an iodonium ion is preferable, and a sulfonium ion is particularly desirable.

As a preferable example of the organic cation for Z⁺, an organic cation represented by general formula (b1-c1) or (b1-c2) shown below can be given.

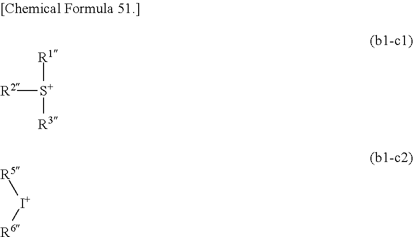

In the formulas, each of R¹″ to R³″, R⁵″ and R⁶″ independently represents an aryl group, alkyl group or alkenyl group which may have a substituent, provided that at least one of R¹″ to R³″ represents an aryl group, and at least one of R⁵″ and R⁶″ represents an aryl group. In formula (b1-c1), two of R¹″ to R³″ may be mutually bonded to form a ring with the sulfur atom.

In formula (b1-c1), each of R¹″ to R³″ independently represents an aryl group, alkyl group or alkenyl group which may have a substituent. Two of R¹″ to R³″ may be mutually bonded to form a ring with the sulfur atom.

Further, among R¹″ to R³″, at least one group represents an aryl group. Among R¹″ to R³″, two or more groups are preferably aryl groups, and it is particularly desirable that all of R¹″ to R³″ are aryl groups.

Examples of the aryl group for R¹″ to R³″ include an unsubstituted aryl group of 6 to 20 carbon atoms; a substituted aryl group in which part or all of the hydrogen atoms of the aforementioned unsubstituted aryl group has been substituted with an alkyl group, an alkoxy group, an alkoxyalkyloxy group, an alkoxycarbonylalkyloxy group, a halogen atom, a hydroxy group, an oxo group (═O), an aryl group, —C(═O)—O—R⁶′, —O—C(═O)—R⁷′ or —O—R⁸′.

Each of R⁶′, R⁷′ and R⁸′ independently represents a linear or branched saturated hydrocarbon group of 1 to 25 atoms, a cyclic saturated hydrocarbon group of 3 to 20 carbon atoms or a linear or branched, aliphatic unsaturated hydrocarbon group of 2 to 5 carbon atoms.

The linear or branched, saturated hydrocarbon group preferably has 1 to 25 carbon atoms, more preferably 1 to 15, and still more preferably 4 to 10.

Examples of the linear, saturated hydrocarbon group include a methyl group, an ethyl group, a propyl group, a butyl group, a pentyl group, a hexyl group, a heptyl group, an octyl group, a nonyl group and a decyl group.

Examples of the branched, saturated hydrocarbon group include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group and a 4-methylpentyl group, but excluding tertiary alkyl groups.

The linear or branched, saturated hydrocarbon group may have a substituent. Examples of the substituent include an alkoxy group, a halogen atom, a halogenated alkyl group, a hydroxyl group, an oxygen atom (═O), a cyano group and a carboxy group.

The alkoxy group as the substituent for the linear or branched saturated hydrocarbon group is preferably an alkoxy group having 1 to 5 carbon atoms, more preferably a methoxy group, an ethoxy group, an n-propoxy group, an iso-propoxy group, an n-butoxy group or a tert-butoxy group, and most preferably a methoxy group or an ethoxy group.

Examples of the halogen atom as the substituent for the linear or branched, saturated alkyl group include a fluorine atom, a chlorine atom, a bromine atom and an iodine atom, and a fluorine atom is preferable.

Example of the halogenated alkyl group as the substituent for the linear or branched, saturated hydrocarbon group includes a group in which part or all of the hydrogen atoms within the aforementioned linear or branched, saturated hydrocarbon group have been substituted with the aforementioned halogen atoms.

The cyclic saturated hydrocarbon group of 3 to 20 carbon atoms for R⁶′, R⁷′ and R⁸′ may be either a polycyclic group or a monocyclic group, and examples thereof include groups in which one hydrogen atom has been removed from a monocycloalkane, and groups in which one hydrogen atom has been removed from a polycycloalkane (e.g., a bicycloalkane, a tricycloalkane or a tetracycloalkane). More specific examples include groups in which one hydrogen atom has been removed from a monocycloalkane such as cyclopentane, cyclohexane, cycloheptane or cyclooctane; and groups in which one or more hydrogen atoms have been removed from a polycycloalkane such as adamantane, norbornane, isobornane, tricyclodecane or tetracyclododecane.

The cyclic, saturated hydrocarbon group may have a substituent. For example, part of the carbon atoms constituting the ring within the cyclic alkyl group may be substituted with a hetero atom, or a hydrogen atom bonded to the ring within the cyclic alkyl group may be substituted with a substituent.

In the former example, a heterocycloalkane in which part of the carbon atoms constituting the ring within the aforementioned monocycloalkane or polycycloalkane has been substituted with a hetero atom such as an oxygen atom, a sulfur atom or a nitrogen atom, and one hydrogen atom has been removed therefrom, can be used. Further, the ring may contain an ester bond (—C(═O)—O—). More specific examples include a lactone-containing monocyclic group, such as a group in which one hydrogen atom has been removed from γ-butyrolactone; and a lactone-containing polycyclic group, such as a group in which one hydrogen atom has been removed from a bicycloalkane, tricycloalkane or tetracycloalkane containing a lactone ring.

In the latter example, as the substituent, the same substituent groups as those for the aforementioned linear or branched alkyl group can be used.

Alternatively, R⁶′, R⁷′ and R⁸′ may be a combination of a linear or branched alkyl group and a cyclic group.

Examples of the combination of a linear or branched alkyl group with a cyclic alkyl group include groups in which a cyclic alkyl group as a substituent is bonded to a linear or branched alkyl group, and groups in which a linear or branched alkyl group as a substituent is bonded to a cyclic alkyl group.

Examples of the linear aliphatic unsaturated hydrocarbon group for R⁶′, R⁷′ and R⁸′ include a vinyl group, a propenyl group (an allyl group) and a butynyl group.

Examples of the branched aliphatic unsaturated hydrocarbon group for R⁶′, R⁷′ and R⁸′ include a 1-methylpropenyl group and a 2-methylpropenyl group.

The aforementioned linear or branched, aliphatic unsaturated hydrocarbon group may have a substituent. Examples of substituents include the same substituents as those which the aforementioned linear or branched alkyl group may have.

Among the aforementioned examples, as R⁷′ and R⁸′, in terms of improvement in lithography properties and shape of the resist pattern, a linear or branched, saturated hydrocarbon group of 1 to 15 carbon atoms or a cyclic saturated hydrocarbon group of 3 to 20 carbon atoms is preferable.

The unsubstituted aryl group for R¹″ to R³″ is preferably an aryl group having 6 to 10 carbon atoms because it can be synthesized at a low cost. Specific examples thereof include a phenyl group and a naphthyl group.

The alkyl group as the substituent for the substituted aryl group represented by R¹″ to R³″ is preferably an alkyl group having 1 to 5 carbon atoms, and a methyl group, an ethyl group, a propyl group, an n-butyl group, or a tert-butyl group is particularly desirable.

The alkoxy group as the substituent for the substituted aryl group is preferably an alkoxy group having 1 to 5 carbon atoms, and a methoxy group, an ethoxy group, an n-propoxy group, an iso-propoxy group, an n-butoxy group or a tert-butoxy group is particularly desirable.

The halogen atom as the substituent for the substituted aryl group is preferably a fluorine atom.

As the aryl group within the substituted aryl group, the same aryl groups as those described above for R¹″ to R³″ can be mentioned, and an aryl group of 6 to 20 carbon atoms is preferable, an aryl group of 6 to 10 carbon atoms is more preferable, and a phenyl group or a naphthyl group is still more preferable.

Examples of alkoxyalkyloxy groups as the substituent for the substituted aryl group include groups represented by a general formula shown below:

—O—C(R⁴⁷)(R⁴⁸)—O—R⁴⁹

In the formula, R⁴⁷ and R⁴⁸ each independently represents a hydrogen atom or a linear or branched alkyl group; and R⁴⁹ represents an alkyl group.

The alkyl group for R⁴⁷ and R⁴⁸ preferably has 1 to 5 carbon atoms, and may be either linear or branched. As the alkyl group, an ethyl group or a methyl group is preferable, and a methyl group is most preferable.

It is preferable that at least one of R⁴⁷ and R⁴⁸ be a hydrogen atom. It is particularly desirable that at least one of R⁴⁷ and R⁴⁸ be a hydrogen atom, and the other be a hydrogen atom or a methyl group.

The alkyl group for R⁴⁹ preferably has 1 to 15 carbon atoms, and may be linear, branched or cyclic.

The linear or branched alkyl group for R⁴⁹ preferably has 1 to 5 carbon atoms. Examples thereof include a methyl group, an ethyl group, a propyl group, an n-butyl group and a tert-butyl group.

The cyclic alkyl group for R⁴⁹ preferably has 4 to 15 carbon atoms, more preferably 4 to 12, and most preferably 5 to 10. Specific examples thereof include groups in which one or more hydrogen atoms have been removed from a monocycloalkane or a polycycloalkane such as a bicycloalkane, tricycloalkane or tetracycloalkane, and which may or may not be substituted with an alkyl group of 1 to 5 carbon atoms, a fluorine atom or a fluorinated alkyl group. Examples of the monocycloalkane include cyclopentane and cyclohexane. Examples of polycycloalkanes include adamantane, norbornane, isobornane, tricyclodecane and tetracyclododecane. Among these, a group in which one or more hydrogen atoms have been removed from adamantane is preferable.

Examples of the alkoxycarbonylalkyloxy group as the substituent for the substituted aryl group include groups represented by a general formula shown below:

—O—R⁵⁰—C(═O)—O—R⁵⁶

In the formula, R⁵⁰ represents a linear or branched alkylene group, and R⁵⁶ represents a tertiary alkyl group.

The linear or branched alkylene group for R⁵⁰ preferably has 1 to 5 carbon atoms, and examples thereof include a methylene group, an ethylene group, a trimethylene group, a tetramethylene group and a 1,1-dimethylethylene group.

Examples of the tertiary alkyl group for R⁵⁶ include a 2-methyl-2-adamantyl group, a 2-ethyl-2-alamantyl group, a 1-methyl-1-cyclopentyl group, a 1-ethyl-1-cyclopentyl group, a 1-methyl-1-cyclohexyl group, a 1-ethyl-1-cyclohexyl group, a 1-(1-alamantyl)-1-methylethyl group, a 1-(1-adamantyl)-1-methylpropyl group, a 1-(1-adamantyl)-1-methylbutyl group, a 1-(1-adamantyl)-1-methylpentyl group, a 1-(1-cyclopentyl)-1-methylethyl group, a 1-(1-cyclopentyl)-1-methylpropyl group, a 1-(1-cyclopentyl)-1-methylbutyl group, a 1-(1-cyclopentyl)-1-methylpentyl group, a 1-(1-cyclohexyl)-1-methylethyl group, a 1-(1-cyclohexyl)-1-methylpropyl group, a 1-(1-cyclohexyl)-1-methylbutyl group, a 1-(1-cyclohexyl)-1-methylpentyl group, a tert-butyl group, a tert-pentyl group and a tert-hexyl group.

Further, a group in which R⁵⁶ in the group represented by the aforementioned general formula: —O—R⁵⁰—C(═O)—O—R⁵⁶ has been substituted with R⁵⁶′ can also be mentioned. R⁵⁶′ represents a hydrogen atom, a fluorinated alkyl group or an aliphatic cyclic group which may contain a hetero atom.

Examples of the fluorinated alkyl group for R⁵⁶′ include groups in which part or all of the hydrogen atoms within the alkyl group for R⁴⁹ has been substituted with a fluorine atom.

Examples of the aliphatic cyclic group for R⁵⁶′ which may contain a hetero atom include an aliphatic cyclic group which does not contain a hetero atom, an alipahtic cyclic group containing a hetero atom in the ring structure, and an aliphatic cyclic group in which a hydrogen atom has been substituted with a hetero atom.

As an aliphatic cyclic group for R⁵⁶′ which does not contain a hetero atom, a group in which one or more hydrogen atoms have been removed from a monocycloalkane or a polycycloalkane such as a bicycloalkane, a tricycloalkane or a tetracycloalkane can be mentioned. Examples of the monocycloalkane include cyclopentane and cyclohexane. Examples of polycycloalkanes include adamantane, norbornane, isobornane, tricyclodecane and tetracyclododecane. Among these, a group in which one or more hydrogen atoms have been removed from adamantane is preferable.

Specific examples of the aliphatic cyclic group for R⁵⁶′ containing a hetero atom in the ring structure include groups represented by formulas (L1) to (L5) and (S1) to (S4) described later.

As the aliphatic cyclic group for R⁵⁶′ in which a hydrogen atom has been substituted with a hetero atom, an aliphatic cyclic group in which a hydrogen atom has been substituted with an oxygen atom (═O) can be mentioned.

The aryl group for each of R¹″ to R³″ is preferably a phenyl group or a naphthyl group.

Examples of the alkyl group for R¹″ to R³″ include linear, branched or cyclic alkyl groups of 1 to 10 carbon atoms. Among these, alkyl groups of 1 to 5 carbon atoms are preferable as the resolution becomes excellent. Specific examples thereof include a methyl group, an ethyl group, an n-propyl group, an isopropyl group, an n-butyl group, an isobutyl group, an n-pentyl group, a cyclopentyl group, a hexyl group, a cyclohexyl group, a nonyl group, and a decyl group, and a methyl group is most preferable because it is excellent in resolution and can be synthesized at a low cost.

The alkenyl group for R¹″ to R³″ preferably has 2 to 10 carbon atoms, more preferably 2 to 5, and still more preferably 2 to 4. Specific examples thereof include a vinyl group, a propenyl group (an allyl group), a butynyl group, a 1-methylpropenyl group and a 2-methylpropenyl group.

When two of R¹″ to R³″ are bonded to each other to form a ring with the sulfur atom, it is preferable that the two of R¹″ to R³″ form a 3 to 10-membered ring including the sulfur atom, and it is particularly desirable that the two of R¹″ to R³″ form a 5 to 7-membered ring including the sulfur atom.

When two of R¹″ to R³″ are bonded to each other to form a ring with the sulfur atom, the remaining one of R¹″ to R³″ is preferably an aryl group. As examples of the aryl group, the same as the above-mentioned aryl groups for R¹″ to R³″ can be given.

Specific examples of organic cation represented by general formula (b1-c1) include triphenylsulfonium, (3,5-dimethylphenyl)diphenylsulfonium, (4-(2-adamantoxymethyloxy)-3,5-dimethylphenyl)diphenylsulfonium, (4-(2-adamantoxymethyloxy)phenyl)diphenylsulfonium, (4-(tert-butoxycarbonylmethyloxy)phenyl)diphenylsulfonium, (4-(tert-butoxycarbonylmethyloxy)-3,5-dimethylphenyl)diphenylsulfonium, (4-(2-methyl-2-adamantyloxycarbonylmethyloxy)phenyl)diphenylsulfonium, (4-(2-methyl-2-adamantyloxycarbonylmethyloxy)-3,5-dimethylphenyl) diphenylsulfonium, tri(4-methylphenyl)sulfonium, dimethyl(4-hydroxynaphthyl)sulfonium, monophenyldimethylsulfonium, diphenylmonomethylsulfonium, (4-methylphenyl)diphenylsulfonium, (4-methoxyphenyl)diphenylsulfonium, tri(4-tert-butyl)phenylsulfonium, diphenyl(1-(4-methoxy)naphthyl)sulfonium, di(1-naphthyl)phenylsulfonium, 1-phenyltetrahydrothiophenium, 1-(4-methylphenyl)tetrahydrothiophenium, 1-(3,5-dimethyl-4-hydroxyphenyl)tetrahydrothiophenium, 1-(4-methoxynaphthalene-1-yl)tetrahydrothiophenium, 1-(4-ethoxynaphthalene-1-yl)tetrahydrothiophenium, 1-(4-n-butoxynaphthalene-1-yl)tetrahydrothiophenium, 1-phenyltetrahydrothiopyranium, 1-(4-hydroxyphenyl)tetrahydrothiopyranium, 1-(3,5-dimethyl-4-hydroxyphenyl)tetrahydrothiopyranium and 1-(4-methylphenyl)tetrahydrothiopyranium.

Preferable examples of the organic cation represented by formula (b1-c1) include the following.

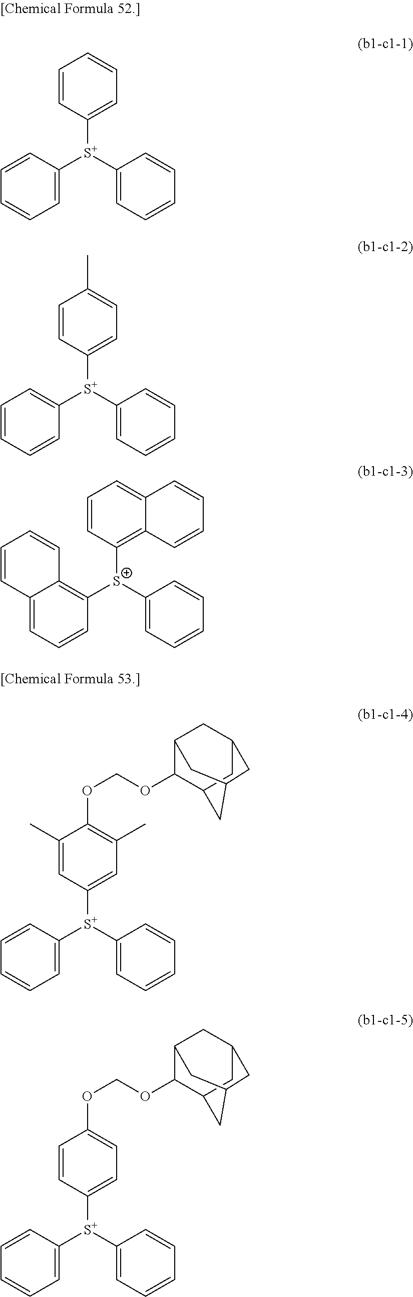

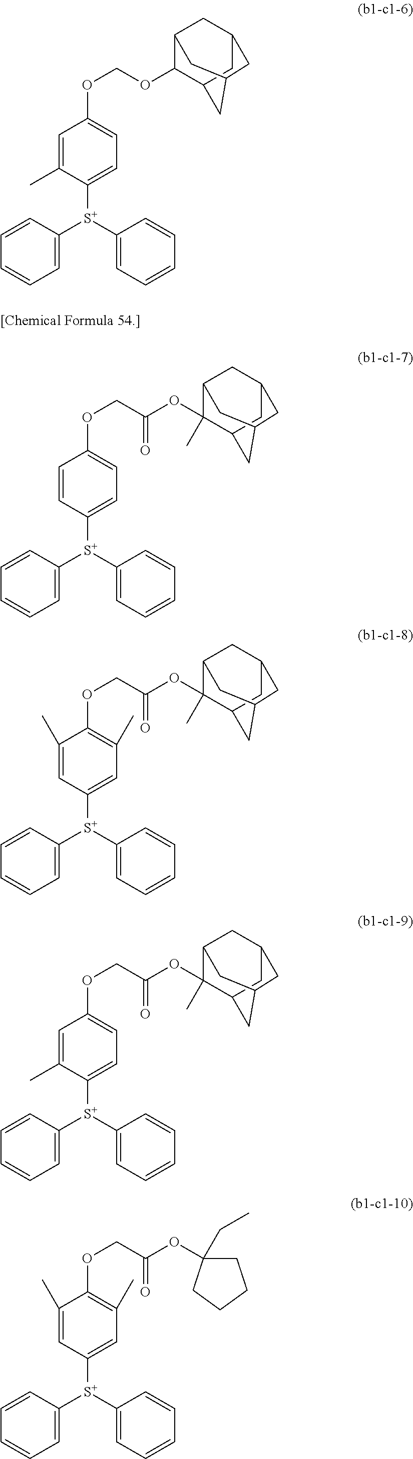

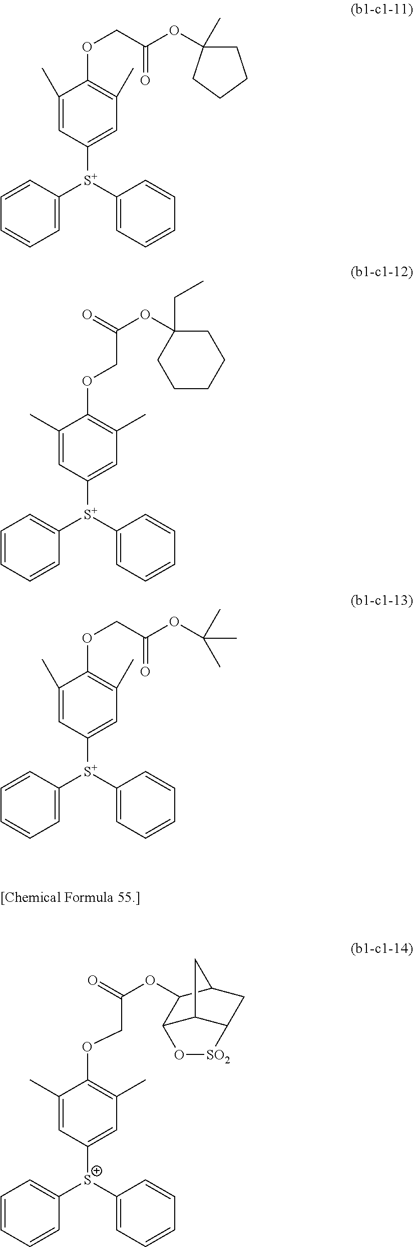

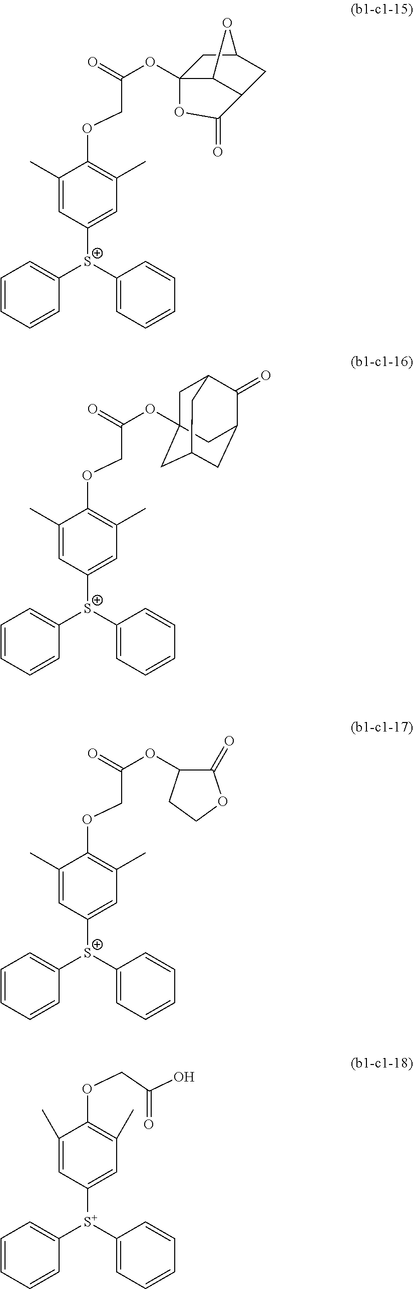

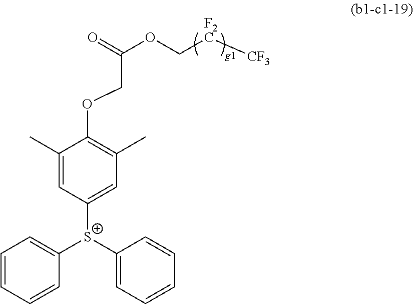

In the formula, g1 represents a recurring number, and is an integer of 1 to 5.

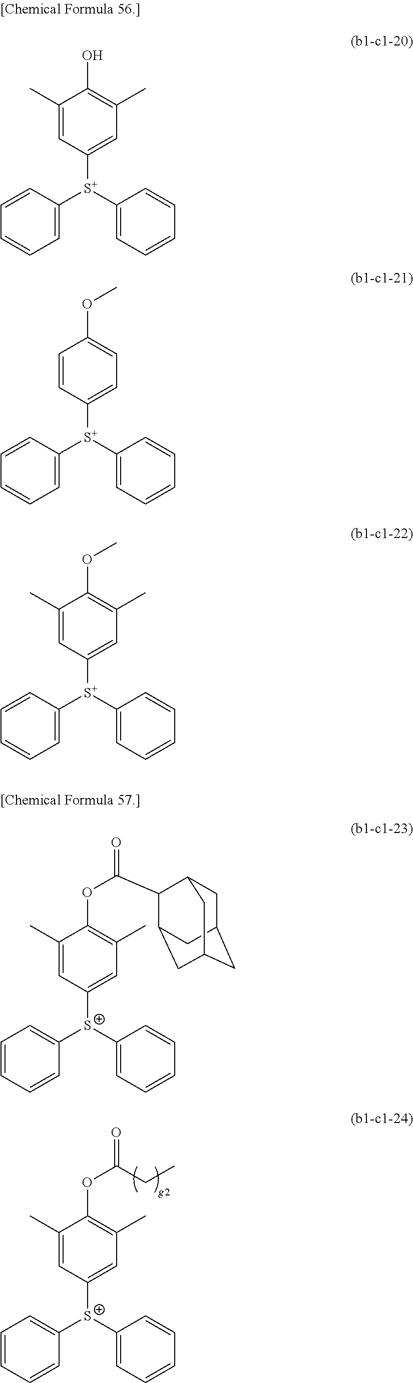

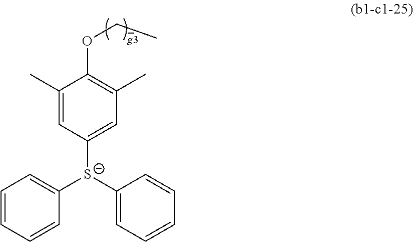

In the formula, g2 and g3 represent recurring numbers, wherein g2 is an integer of 0 to 20, and g3 is an integer of 0 to 20.

In formula (b1-c2), each of R⁵″ and R⁶″ independently represents an aryl group, alkyl group or alkenyl group which may have a substituent.

At least one of R⁵″ and R⁶″ represents an aryl group. It is preferable that both R⁵″ and R⁶″ represent an aryl group.

As the aryl group for R⁵″ and R⁶″, the same as the aryl groups for R¹″ to R³″ can be used.

As the alkyl group for R⁵″ and R⁶″, the same as the alkyl groups for R¹″ to R³″ can be used.

As the alkenyl group for R⁵″ and R⁶″, the same as the alkenyl groups for R¹″ to R³″ can be used.

It is particularly desirable that both of R⁵″ and R⁶″ represents a phenyl group. Specific examples of the cation moiety represented by general formula (b1-c2) include diphenyliodonium and bis(4-tert-butylphenyl)iodonium.

Furthermore, as a preferable example of the monovalent organic cation for Z⁺, a cation represented by general formula (I-1) or (I-2) shown below can be mentioned.

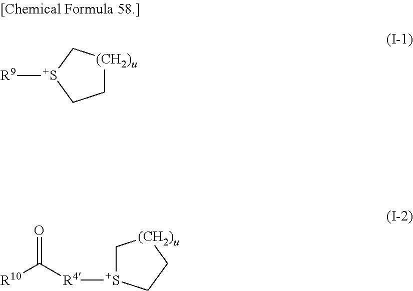

In formulas (I-1) and (I-2), each of R⁹ and R¹⁰ independently represents a phenyl group or naphthyl group which may have a substituent, an alkyl group of 1 to 5 carbon atoms, an alkoxy group or a hydroxy group. Examples of the substituent are the same as the substituents described above in relation to the substituted aryl group for R¹″ to R³″ (i.e., an alkyl group, an alkoxy group, an alkoxyalkyloxy group, an alkoxycarbonylalkyloxy group, a halogen atom, a hydroxy group, an oxo group (═O), an aryl group, —C(═O)—O—R⁶′, —O—C(═O)—R⁷′, —O—R⁸′, a group in which R⁵⁶′ in the aforementioned general formula —O—R⁵⁰—C(═O)—O—R⁵⁶ has been substituted with R⁵⁶′).

R⁴′ represents an alkylene group of 1 to 5 carbon atoms.

u is an integer of 1 to 3, and most preferably 1 or 2.

Preferable examples of the organic cation represented by formula (I-1) or (I-2) are shown below.

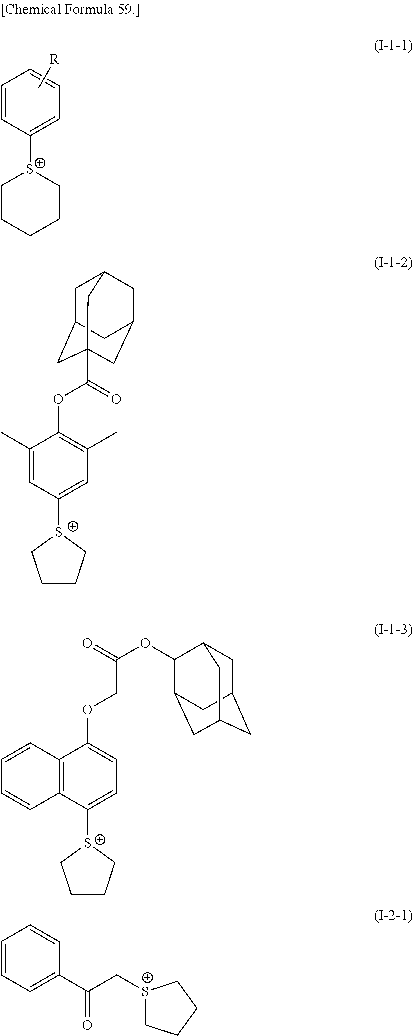

Furthermore, as a preferable example of the monovalent organic cation for Z⁺, a cation represented by general formula (I-5) or (I-6) shown below can be mentioned.

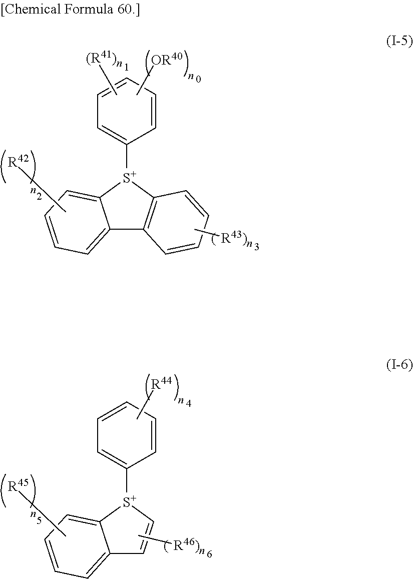

In the formulas, R⁴⁰ represents a hydrogen atom or an alkyl group; R⁴¹ represents an alkyl group, an acetyl group, a carboxy group or a hydroxyalkyl group; each of R⁴² to R⁴⁶ independently represents an alkyl group, an acetyl group, an alkoxy group, a carboxy group, or a hydroxyalkyl group; each of n₀ to n₅ independently represents an integer of 0 to 3, provided that n₀+n₁ is 5 or less; and n₆ represents an integer of 0 to 2.

In general formula (I-5), the alkyl group for R⁴⁰ is preferably an alkyl group of 1 to 15 carbon atoms, more preferably an alkyl group of 1 to 10 carbon atoms, and still more preferably an alkyl group of 1 to 4 carbon atoms. Among these, a linear or branched alkyl group is preferable.

In general formulas (I-5) and (I-6), with respect to R⁴¹ to R⁴⁶, the alkyl group is preferably an alkyl group of 1 to 5 carbon atoms, more preferably a linear or branched alkyl group, and most preferably a methyl group, an ethyl group, a propyl group, an isopropyl group, an n-butyl group or a tert butyl group.

The alkoxy group is preferably an alkoxy group of 1 to 5 carbon atoms, more preferably a linear or branched alkoxy group, and most preferably a methoxy group or ethoxy group.

The hydroxyalkyl group is preferably the aforementioned alkyl group in which one or more hydrogen atoms have been substituted with hydroxy groups, and examples thereof include a hydroxymethyl group, a hydroxyethyl group and a hydroxypropyl group.

If there are two or more of the OR⁴⁰ group, as indicated by the value of n₀, then the two or more of the OR⁴⁰ group may be the same or different from each other.

If there are two or more of an individual R⁴¹ to R⁴⁶ group, as indicated by the corresponding value of n₁ to n₆, then the two or more of the individual R⁴¹ to R⁴⁶ group may be the same or different from each other.

n₀ is preferably 0 or 1.

n₁ is preferably 0 to 2.

It is preferable that n₂ and n₃ each independently represent 0 or 1, and more preferably 0.

n₄ is preferably 0 to 2, and more preferably 0 or 1.

n₅ is preferably 0 or 1, and more preferably 0.

n₆ is preferably 0 or 1.

Preferable examples of the organic cation represented by formula (I-5) or (I-6) are shown below.

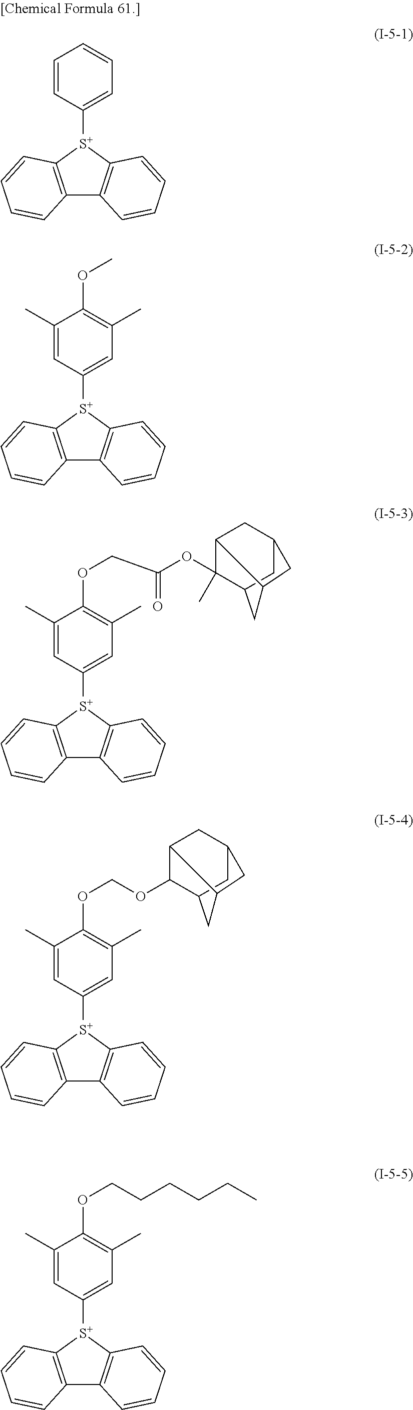

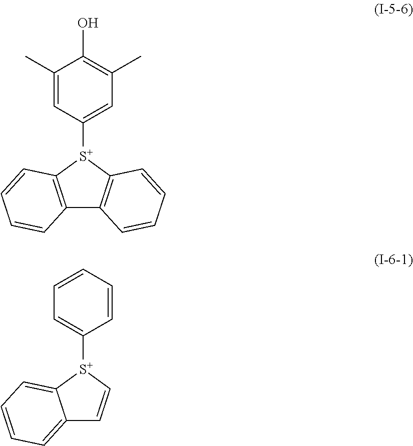

Among the aforementioned examples, as the component (B1), in terms of improvement in the lithography properties and the shape of the resist pattern, a compound represented by general formula (b1-1-0) shown below is particularly desirable.

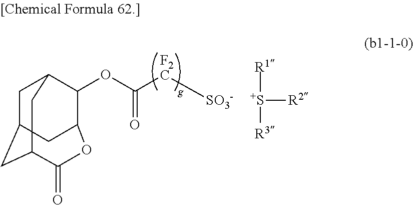

In the formula, g represents an integer of 1 to 4; and R¹″ to R³″ are the same as defined above.

As the component (B1), one type of compound may be used, or two or more types of compounds may be used in combination.

In the resist composition of the present invention, the amount of the component (B1), relative to 100 parts by weight of the component (A) is preferably within the range of 0.1 to 50 parts by weight, more preferably within the range of 0.1 to 30 parts by weight, and most preferably within the range of 1 to 20 parts by weight.

When the amount of the component (B1) is at least as large as the lower limit of the above-mentioned range, various lithography properties of the resist composition are improved, such as roughness, mask reproducibility and exposure latitude. Further, a resist pattern having an excellent shape with high rectangularity can be reliably obtained. On the other hand, when the amount is no more than the upper limit of the above-mentioned range, a uniform solution can be obtained and the storage stability becomes satisfactory.

In the component (B), the amount of the component (B1) based on the total weight of the component (B) is preferably 20% by weight or more, more preferably 40% by weight, and may be even 100% by weight. The amount of the component (B1) is most preferably 100% by weight. When the amount of the component (B1) is at least as large as the lower limit of the above-mentioned range, various lithography properties of the resist composition are improved. Further, a resist pattern having an excellent shape can be obtained.

[Component (B2)]

In the resist composition of the present invention, if desired, the component (B) may further contain an acid generator other than the component (B1) (hereafter, referred to as “component (B2)”).

The component (B2) is not particularly limited as long as it is an acid generator that does not fall under the category of the component (B1). Examples of such an acid generator are numerous, and include onium salt acid generators such as iodonium salts and sulfonium salts; oxime sulfonate acid generators; diazomethane acid generators such as bisalkyl or bisaryl sulfonyl diazomethanes and poly(bis-sulfonyl)diazomethanes; nitrobenzylsulfonate acid generators; iminosulfonate acid generators; and disulfone acid generators.

As the component (B2), an onium salt acid generator represented by general formula (b-1) or (b-2) shown below can be preferably used.

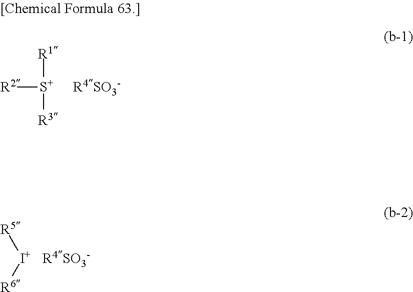

In formula (b-1), each of R¹″ to R³″ independently represents an aryl group, alkyl group or alkenyl group which may have a substituent, provided that at least one of R¹″ to R³″ represents an aryl group, and two of R¹″ to R³″ may be bonded to each other to form a ring with the sulfur atom. In formula (b-2), R⁵″ and R⁶″ each independently represent an aryl group, alkyl group or alkenyl group which may have a substituent, provided that and at least one of R⁵″ and R⁶″ represents an aryl group. R⁴″ represents a halogenated alkyl group, an aryl group or an alkenyl group which may have a substituent.

In general formula (b-1), R¹″ to R³″ are respectively the same as defined for R¹″ to R³″ in general formula (b1-c1).

In general formula (b-2), R⁵″ and R⁶″ are respectively the same as defined for R⁵″ to R⁶″ in general formula (b1-c2).

In formulas (b-1) and (b-2), R⁴″ represents a halogenated alkyl group, an aryl group or an alkenyl group which may have a substituent.

As an example of the halogenated alkyl group for R⁴″, a group in which part of or all of the hydrogen atoms of a linear, branched or cyclic alkyl group have been substituted with halogen atoms can be given. Examples of the aforementioned halogen atom include a fluorine atom, a chlorine atom, a bromine atom and an iodine atom, and a fluorine atom is preferable.

When the alkyl group within the halogenated alkyl group is a linear or branched alkyl group, it preferably has 1 to 10 carbon atoms, more preferably 1 to 8 carbon atoms, and most preferably 1 to 4 carbon atoms. On the other hand, when the alkyl group within the halogenated alkyl group is a cyclic alkyl group, it preferably has 4 to 15 carbon atoms, more preferably 4 to 10 carbon atoms, and most preferably 6 to 10 carbon atoms.

In the halogenated alkyl group, the percentage of the number of halogen atoms based on the total number of halogen atoms and hydrogen atoms (halogenation ratio (%)) is preferably 10 to 100%, more preferably 50 to 100%, and most preferably 100%. Higher halogenation ratios are preferable, as they result in increased acid strength.

The aryl group for R⁴″ is preferably an aryl group of 6 to 20 carbon atoms.

The alkenyl group for R⁴″ is preferably an alkenyl group of 2 to 10 carbon atoms.

With respect to R⁴″, the expression “may have a substituent” means that part of or all of the hydrogen atoms within the aforementioned linear, branched or cyclic alkyl group, halogenated alkyl group, aryl group or alkenyl group may be substituted with substituents (atoms other than hydrogen atoms, or groups).

R⁴″ may have one substituent, or two or more substituents.

Examples of the substituent include a halogen atom, a hetero atom, an alkyl group, and a group represented by the formula X-Q²- (in the formula, Q² represents a divalent linking group containing an oxygen atom; and X represents a hydrocarbon group of 3 to 30 carbon atoms which may have a substituent).

Examples of halogen atoms and alkyl groups as substituents for R⁴″ include the same halogen atoms and alkyl groups as those described above with respect to the halogenated alkyl group for R⁴″.

Examples of hetero atoms include an oxygen atom, a nitrogen atom, and a sulfur atom.

In the group represented by formula X-Q²-, Q² represents a divalent linking group containing an oxygen atom.

Q² may contain an atom other than an oxygen atom. Examples of atoms other than oxygen include a carbon atom, a hydrogen atom, a sulfur atom and a nitrogen atom.

Examples of divalent linking groups containing an oxygen atom include non-hydrocarbon, oxygen atom-containing linking groups such as an oxygen atom (an ether bond; —O—), an ester bond (—C(═O)—O—), an amido bond (—C(═O)—NH—), a carbonyl group (—C(═O)—) and a carbonate bond (—O—C(═O)—O—); and combinations of the aforementioned non-hydrocarbon, hetero atom-containing linking groups with an alkylene group.

Specific examples of the combinations of the aforementioned non-hydrocarbon, hetero atom-containing linking groups and an alkylene group include —R⁹¹—O—, —R⁹²—O—C(═O)—, —C(═O)—O—R⁹³—O—C(═O)— (in the formulas, each of R⁹¹ to R⁹³ are the same as defined above, and independently represents an alkylene group).

The alkylene group for R⁹¹ to R⁹³ is preferably a linear or branched alkylene group, and preferably has 1 to 12 carbon atoms, more preferably 1 to 5, and most preferably 1 to 3.

Specific examples of alkylene groups include a methylene group [—CH₂—]; alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)— and —C(CH₂CH₃)₂—; an ethylene group [—CH₂CH₂—]; alkylethylene groups such as —CH(CH₃)CH₂—, —CH(CH₃)CH(CH₃)—, —C(CH₃)₂CH₂— and —CH(CH₂CH₃)CH₂—; a trimethylene group (n-propylene group) [—CH₂CH₂CH₂—]; alkyltrimethylene groups such as —CH(CH₃)CH₂CH₂— and —CH₂CH(CH₃)CH₂—; a tetramethylene group [—CH₂CH₂CH₂CH₂—]; alkyltetramethylene groups such as —CH(CH₃)CH₂CH₂CH₂— and —CH₂CH(CH₃)CH₂CH₂—; and a pentamethylene group [—CH₂CH₂CH₂CH₂CH₂—].

As Q², a divalent linking group containing an ester bond or an ether bond is preferable, and —R⁹¹—O—, —R⁹²—O—C(═O)— or —C(═O)—O—R⁹³—O—C(═O)— is more preferable.

In the group represented by the formula X-Q²-, the hydrocarbon group for X may be either an aromatic hydrocarbon group or an aliphatic hydrocarbon group.

The aromatic hydrocarbon group is a hydrocarbon group having an aromatic ring. The aromatic hydrocarbon ring preferably has 3 to 30 carbon atoms, more preferably 5 to 30, still more preferably 5 to 20, still more preferably 6 to 15, and most preferably 6 to 12. Here, the number of carbon atoms within a substituent(s) is not included in the number of carbon atoms of the aromatic hydrocarbon group.

Specific examples of aromatic hydrocarbon groups include an aryl group which is an aromatic hydrocarbon ring having one hydrogen atom removed therefrom, such as a phenyl group, a biphenyl group, a fluorenyl group, a naphthyl group, an anthryl group or a phenanthryl group; and an alkylaryl group such as a benzyl group, a phenethyl group, a 1-naphthylmethyl group, a 2-naphthylmethyl group, a 1-naphthylethyl group, or a 2-naphthylethyl group. The alkyl chain within the arylalkyl group preferably has 1 to 4 carbon atom, more preferably 1 or 2, and most preferably 1.

The aromatic hydrocarbon group may have a substituent. For example, part of the carbon atoms constituting the aromatic ring within the aromatic hydrocarbon group may be substituted with a hetero atom, or a hydrogen atom bonded to the aromatic ring within the aromatic hydrocarbon group may be substituted with a substituent.

In the former example, a heteroaryl group in which part of the carbon atoms constituting the ring within the aforementioned aryl group has been substituted with a hetero atom such as an oxygen atom, a sulfur atom or a nitrogen atom, and a heteroarylalkyl group in which part of the carbon atoms constituting the aromatic hydrocarbon ring within the aforementioned arylalkyl group has been substituted with the aforementioned heteroatom can be used.

In the latter example, as the substituent for the aromatic hydrocarbon group, an alkyl group, an alkoxy group, a halogen atom, a halogenated alkyl group, a hydroxyl group, an oxygen atom (═O) or the like can be used.

The alkyl group as the substituent for the aromatic hydrocarbon group is preferably an alkyl group of 1 to 5 carbon atoms, and a methyl group, an ethyl group, a propyl group, an n-butyl group or a tert-butyl group is particularly desirable.

The alkoxy group as the substituent for the aromatic hydrocarbon group is preferably an alkoxy group having 1 to 5 carbon atoms, more preferably a methoxy group, ethoxy group, n-propoxy group, iso-propoxy group, n-butoxy group or tert-butoxy group, and most preferably a methoxy group or an ethoxy group.

Examples of the halogen atom as the substituent for the aromatic hydrocarbon group include a fluorine atom, a chlorine atom, a bromine atom and an iodine atom, and a fluorine atom is preferable.

Example of the halogenated alkyl group as the substituent for the aromatic hydrocarbon group includes a group in which part or all of the hydrogen atoms within the aforementioned alkyl group have been substituted with the aforementioned halogen atoms.

The aliphatic hydrocarbon group for X may be either a saturated aliphatic hydrocarbon group, or an unsaturated aliphatic hydrocarbon group. Further, the aliphatic hydrocarbon group may be linear, branched or cyclic.

In the aliphatic hydrocarbon group for X, part of the carbon atoms constituting the aliphatic hydrocarbon group may be substituted with a substituent group containing a hetero atom, or part or all of the hydrogen atoms constituting the aliphatic hydrocarbon group may be substituted with a substituent group containing a hetero atom.

As the “hetero atom” for X, there is no particular limitation as long as it is an atom other than carbon and hydrogen. Examples of hetero atoms include a halogen atom, an oxygen atom, a sulfur atom and a nitrogen atom. Examples of the halogen atom include a fluorine atom, a chlorine atom, an iodine atom and a bromine atom.

The substituent group containing a hetero atom may consist of a hetero atom, or may be a group containing a group or atom other than a hetero atom.

Specific examples of the substituent group for substituting part of the carbon atoms include —O—, —C(═O)—O—, —C(═O)—, —O—C(═O)—O—, —C(═O)—NH—, —NH— (the H may be replaced with a substituent such as an alkyl group or an acyl group), —S—, —S(═O)₂— and —S(═O)₂—O—. When the aliphatic hydrocarbon group is cyclic, the aliphatic hydrocarbon group may contain any of these substituent groups in the ring structure.

Examples of the substituent group for substituting part or all of the hydrogen atoms include an alkoxy group, a halogen atom, a halogenated alkyl group, a hydroxyl group, an oxygen atom (═O) and a cyano group.

The aforementioned alkoxy group is preferably an alkoxy group having 1 to 5 carbon atoms, more preferably a methoxy group, ethoxy group, n-propoxy group, iso-propoxy group, n-butoxy group or tert-butoxy group, and most preferably a methoxy group or an ethoxy group.

Examples of the aforementioned halogen atom include a fluorine atom, a chlorine atom, a bromine atom and an iodine atom, and a fluorine atom is preferable.

Example of the aforementioned halogenated alkyl group includes a group in which part or all of the hydrogen atoms within an alkyl group of 1 to 5 carbon atoms (e.g., a methyl group, an ethyl group, a propyl group, an n-butyl group or a tert-butyl group) have been substituted with the aforementioned halogen atoms.

As the aliphatic hydrocarbon group, a linear or branched saturated hydrocarbon group, a linear or branched monovalent unsaturated hydrocarbon group, or a cyclic aliphatic hydrocarbon group (aliphatic cyclic group) is preferable.

The linear saturated hydrocarbon group (alkyl group) preferably has 1 to 20 carbon atoms, more preferably 1 to 15, and most preferably 1 to 10. Specific examples include a methyl group, an ethyl group, a propyl group, a butyl group, a pentyl group, a hexyl group, a heptyl group, an octyl group, a nonyl group, a decanyl group, an undecyl group, a dodecyl group, a tridecyl group, an isotridecyl group, a tetradecyl group, a pentadecyl group, a hexadecyl group, an isohexadecyl group, a heptadecyl group, an octadecyl group, a nonadecyl group, an icosyl group, a henicosyl group and a docosyl group.

The branched saturated hydrocarbon group (alkyl group) preferably has 3 to 20 carbon atoms, more preferably 3 to 15, and most preferably 3 to 10. Specific examples include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group and a 4-methylpentyl group.

The unsaturated hydrocarbon group preferably has 2 to 10 carbon atoms, more preferably 2 to 5, still more preferably 2 to 4, and most preferably 3. Examples of linear monovalent unsaturated hydrocarbon groups include a vinyl group, a propenyl group (an allyl group) and a butynyl group. Examples of branched monovalent unsaturated hydrocarbon groups include a 1-methylpropenyl group and a 2-methylpropenyl group.

Among the above-mentioned examples, as the unsaturated hydrocarbon group, a propenyl group is particularly desirable.

The aliphatic cyclic group may be either a monocyclic group or a polycyclic group. The aliphatic cyclic group preferably has 3 to 30 carbon atoms, more preferably 5 to 30, still more preferably 5 to 20, still more preferably 6 to 15, and most preferably 6 to 12.

As the aliphatic cyclic group, a group in which one or more hydrogen atoms have been removed from a monocycloalkane or a polycycloalkane such as a bicycloalkane, tricycloalkane or tetracycloalkane can be used. Specific examples include groups in which one or more hydrogen atoms have been removed from a monocycloalkane such as cyclopentane or cyclohexane; and groups in which one or more hydrogen atoms have been removed from a polycycloalkane such as adamantane, norbornane, isobornane, tricyclodecane or tetracyclododecane.

When the aliphatic cyclic group does not contain a hetero atom-containing substituent group in the ring structure thereof, the aliphatic cyclic group is preferably a polycyclic group, more preferably a group in which one or more hydrogen atoms have been removed from a polycycloalkane, and a group in which one or more hydrogen atoms have been removed from adamantane is particularly desirable.

When the aliphatic cyclic group contains a hetero atom-containing substituent group in the ring structure thereof, the hetero atom-containing substituent group is preferably —O—, —C(═O)—O—, —S—, —S(═O)₂— or —S(═O)₂—O—. Specific examples of such aliphatic cyclic groups include groups represented by formulas (L1) to (L5) and (S1) to (S4) shown below.

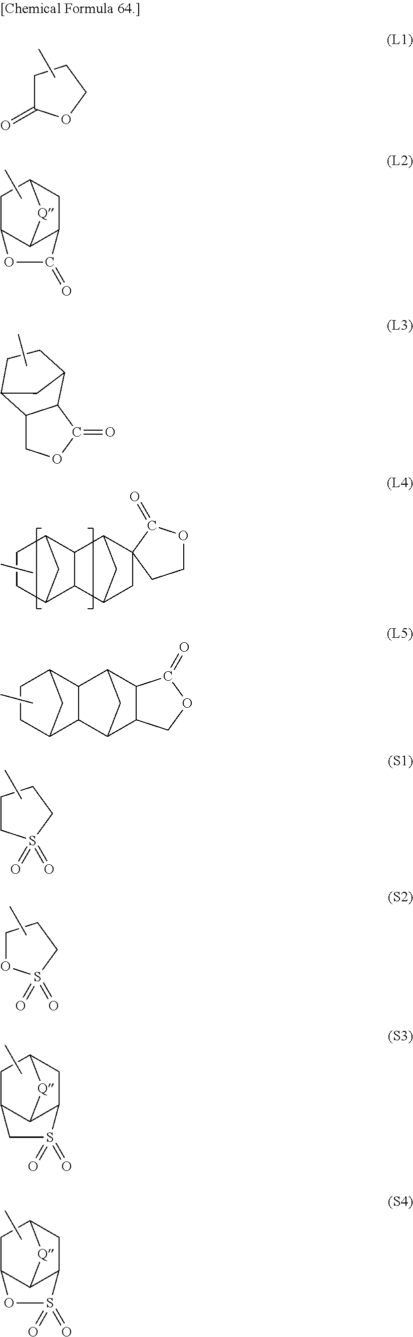

In the formula, Q″ represents an alkylene group of 1 to 5 carbon atoms, —O—, —S—, —O—R⁹⁴— or —S—R⁹⁵— (wherein each of R⁹⁴ and R⁹⁵ independently represents an alkylene group of 1 to 5 carbon atoms); and m represents 0 or 1.

As the alkylene group for Q″, R⁹⁴ and R⁹⁵, the same alkylene groups as those described above for R⁹¹ to R⁹³ can be used.

In these aliphatic cyclic groups, part of the hydrogen atoms bonded to the carbon atoms constituting the ring structure may be substituted with a substituent. Examples of substituents include an alkyl group, an alkoxy group, a halogen atom, a halogenated alkyl group, a hydroxyl group and an oxygen atom (═O).

As the alkyl group, an alkyl group of 1 to 5 carbon atoms is preferable, and a methyl group, an ethyl group, a propyl group, an n-butyl group or a tert-butyl group is particularly desirable.

As the alkoxy group and the halogen atom, the same groups as the substituent groups for substituting part or all of the hydrogen atoms can be used.

Among the examples described above, as X, a cyclic group which may have a substituent is preferable. The cyclic group may be either an aromatic hydrocarbon group which may have a substituent, or an aliphatic cyclic group which may have a substituent, and an aliphatic cyclic group which may have a substituent is preferable.

As the aromatic hydrocarbon group, a naphthyl group which may have a substituent, or a phenyl group which may have a substituent is preferable.

As the aliphatic cyclic group which may have a substituent, an aliphatic polycyclic group which may have a substituent is preferable. As the aliphatic polycyclic group, the aforementioned group in which one or more hydrogen atoms have been removed from a polycycloalkane, and groups represented by the aforementioned formulas (L2) to (L5), (S3) and (S4) are preferable.

Further, in the present invention, it is particularly desirable that X have a polar moiety, because it results in improved lithographic properties and resist pattern shape.

Specific examples of X having a polar moiety include those in which a part of the carbon atoms constituting the aliphatic hydrocarbon group for X is substituted with a substituent group containing a hetero atom such as —O—, —C(═O)—O—, —C(═O)—, —O—C(═O)—O—, —C(═O)—NH—, —NH— (wherein H may be substituted with a substituent such as an alkyl group or an acyl group), —S—, —S(═O)₂— and —S(═O)₂—O—.

In the present invention, R⁴″ preferably has X-Q²- as a substituent. In this case, R⁴″ is preferably a group represented by formula X-Q²-Y³— [wherein Q² and X are the same as defined above; and Y³ represents an alkylene group of 1 to 4 carbon atoms which may have a substituent, or a fluorinated alkylene group of 1 to 4 carbon atoms which may have a substituent].

In the group represented by the formula X-Q²-Y³—, as the alkylene group for Y³, the same alkylene group as those described above for Q² in which the number of carbon atoms is 1 to 4 can be used.

As the fluorinated alkylene group for Y³, the aforementioned alkylene group in which part or all of the hydrogen atoms has been substituted with fluorine atoms can be used.

Specific examples of Y³ include —CF₂—, —CF₂CF₂—, —CF₂CF₂CF₂—, —CF(CF₃)CF₂—, —CF(CF₂CF₃)—, —C(CF₃)₂—, —CF₂CF₂CF₂CF₂—, —CF(CF₃)CF₂CF₂—, —CF₂CF(CF₃)CF₂—, —CF(CF₃)CF(CF₃)—, —C(CF₃)₂CF₂—, —CF(CF₂CF₃)CF₂—, —CF(CF₂CF₂CF₃)—, —C(CF₃)(CF₂CF₃)—; —CHF—, —CH₂CF₂—, —CH₂CH₂CF₂—, —CH₂CF₂CF₂—, —CH(CF₃)CH₂—, —CH(CF₂CF₃)—, —C(CH₃)(CF₃)—, —CH₂CH₂CH₂CF₂—, —CH₂CH₂CF₂CF₂—, —CH(CF₃)CH₂CH₂—, —CH₂CH(CF₃)CH₂—, —CH(CF₃)CH(CF₃)—, —C(CF₃)₂CH₂—; —CH₂—, —CH₂CH₂—, —CH₂CH₂CH₂—, —CH(CH₃)CH₂—, —CH(CH₂CH₃)—, —C(CH₃)₂—, —CH₂CH₂CH₂CH₂—, —CH(CH₃)CH₂CH₂—, —CH₂CH(CH₃)CH₂—, —CH(CH₃)CH(CH₃)—, —C(CH₃)₂CH₂—, —CH(CH₂CH₃)CH₂—, —CH(CH₂CH₂CH₃)—, and —C(CH₃)(CH₂CH₃)—.

Y³ is preferably a fluorinated alkylene group, and particularly preferably a fluorinated alkylene group in which the carbon atom bonded to the adjacent sulfur atom is fluorinated. Examples of such fluorinated alkylene groups include —CF₂—, —CF₂CF₂—, —CF₂CF₂CF₂—, —CF(CF₃)CF₂—, —CF₂CF₂CF₂CF₂—, —CF(CF₃)CF₂CF₂—, —CF₂CF(CF₃)CF₂—, —CF(CF₃)CF(CF₃)—, —C(CF₃)₂CF₂—, —CF(CF₂CF₃)CF₂—; —CH₂CF₂—, —CH₂CH₂CF₂—, —CH₂CF₂CF₂—; —CH₂CH₂CH₂CF₂—, —CH₂CH₂CF₂CF₂—, and —CH₂CF₂CF₂CF₂—.

Of these, —CF₂—, —CF₂CF₂—, —CF₂CF₂CF₂— or CH₂CF₂CF₂— is preferable, —CF₂—, —CF₂CF₂— or —CF₂CF₂CF₂— is more preferable, and —CF₂— is particularly desirable.

The alkylene group or fluorinated alkylene group may have a substituent. The alkylene group or fluorinated alkylene group “has a substituent” means that part or all of the hydrogen atoms or fluorine atoms in the alkylene group or fluorinated alkylene group has been substituted with groups other than hydrogen atoms and fluorine atoms.

Examples of substituents which the alkylene group or fluorinated alkylene group may have include an alkyl group of 1 to 4 carbon atoms, an alkoxy group of 1 to 4 carbon atoms, and a hydroxyl group.

Specific examples of suitable onium salt acid generators represented by formula (b-1) or (b-2) include diphenyliodonium trifluoromethanesulfonate or nonafluorobutanesulfonate; bis(4-tert-butylphenyl)iodonium trifluoromethanesulfonate or nonafluorobutanesulfonate; triphenylsulfonium trifluoromethanesulfonate, heptafluoropropanesulfonate or nonafluorobutanesulfonate; tri(4-methylphenyl)sulfonium trifluoromethanesulfonate, heptafluoropropanesulfonate or nonafluorobutanesulfonate; dimethyl(4-hydroxynaphthyl)sulfonium trifluoromethanesulfonate, heptafluoropropanesulfonate or nonafluorobutanesulfonate; monophenyldimethylsulfonium trifluoromethanesulfonate, heptafluoropropanesulfonate or nonafluorobutanesulfonate; diphenylmonomethylsulfonium trifluoromethanesulfonate, heptafluoropropanesulfonate or nonafluorobutanesulfonate; (4-methylphenyl)diphenylsulfonium trifluoromethanesulfonate, heptafluoropropanesulfonate or nonafluorobutanesulfonate; (4-methoxyphenyl)diphenylsulfonium trifluoromethanesulfonate, heptafluoropropanesulfonate or nonafluorobutanesulfonate; tri(4-tert-butyl)phenylsulfonium trifluoromethanesulfonate, heptafluoropropanesulfonate or nonafluorobutanesulfonate; diphenyl(1-(4-methoxy)naphthyl)sulfonium trifluoromethanesulfonate, heptafluoropropanesulfonate or nonafluorobutanesulfonate; di(1-naphthyl)phenylsulfonium trifluoromethanesulfonate, heptafluoropropanesulfonate or nonafluorobutanesulfonate; 1-phenyltetrahydrothiophenium trifluoromethanesulfonate, heptafluoropropanesulfonate or nonafluorobutanesulfonate; 1-(4-methylphenyl)tetrahydrothiophenium trifluoromethanesulfonate, heptafluoropropanesulfonate or nonafluorobutanesulfonate; 1-(3,5-dimethyl-4-hydroxyphenyl)tetrahydrothiophenium trifluoromethanesulfonate, heptafluoropropanesulfonate or nonafluorobutanesulfonate; 1-(4-methoxynaphthalene-1-yl)tetrahydrothiophenium trifluoromethanesulfonate, heptafluoropropanesulfonate or nonafluorobutanesulfonate; 1-(4-ethoxynaphthalene-1-yl)tetrahydrothiophenium trifluoromethanesulfonate, heptafluoropropanesulfonate or nonafluorobutanesulfonate; 1-(4-n-butoxynaphthalene-1-yl)tetrahydrothiophenium trifluoromethanesulfonate, heptafluoropropanesulfonate or nonafluorobutanesulfonate; 1-phenyltetrahydrothiopyranium trifluoromethanesulfonate, heptafluoropropanesulfonate or nonafluorobutanesulfonate; 1-(4-hydroxyphenyl)tetrahydrothiopyranium trifluoromethanesulfonate, heptafluoropropanesulfonate or nonafluorobutanesulfonate; 1-(3,5-dimethyl-4-hydroxyphenyl)tetrahydrothiopyranium trifluoromethanesulfonate, heptafluoropropanesulfonate or nonafluorobutanesulfonate; and 1-(4-methylphenyl)tetrahydrothiopyranium trifluoromethanesulfonate, heptafluoropropanesulfonate or nonafluorobutanesulfonate.

By using any of these onium salt acid generators in combination with the component (B1), in the formation of a resist pattern, the critical resolution, sensitivity, exposure latitude (EL margin), mask error factor (MEF), line width roughness (LWR), line edge roughness (LER), circularity, critical dimension uniformity (CDU) or pattern shape can be improved.

Further, as the component (B2), among those represented by the aforementioned general formula (b-1) or (b-2), an onium salt acid generator having an anion moiety represented by any one of formulas (b1) to (b8) shown below is particularly desirable.

By using such an onium salt acid generator in combination with the component (B1), in the formation of a resist pattern, critical resolution, sensitivity, EL margin, MEF, LWR, LER, circularity, CDU or pattern shape can be particularly improved.

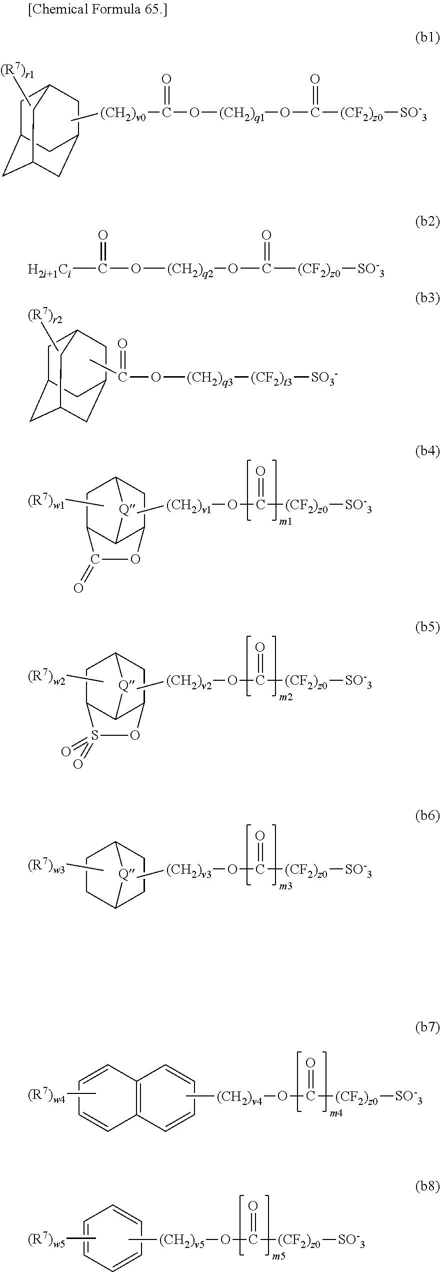

In the formulas, z0 represents an integer of 1 to 3; each of q1 and q2 independently represents an integer of 1 to 5; q3 represents an integer of 1 to 12; t3 represents an integer of 1 to 3; each of r1 and r2 independently represents an integer of 0 to 3; i represents an integer of 1 to 20; R⁷ represents a substituent; each of m1 to m5 independently represents 0 or 1; each of v0 to v5 independently represents an integer of 0 to 3; each of w1 to w5 independently represents an integer of 0 to 3; and Q″ is the same as defined above.

As the substituent for R⁷, the same groups as those which the aforementioned aliphatic hydrocarbon group or aromatic hydrocarbon group for X may have as a substituent can be used.

If there are two or more of the R⁷ group, as indicated by the values r1, r2, and w1 to w5, then the two or more of the R⁷ groups may be the same or different from each other.

Further, as the component (B2), an onium salt acid generator in which the anion moiety in general formula (b-1) or (b-2) is replaced by an anion represented by general formula (b-3) or (b-4) shown below can also be preferably used. By using such an onium salt acid generator in combination with the component (B1), in the formation of a resist pattern, critical resolution, sensitivity, EL margin, MEF, LWR, LER, circularity, CDU or pattern shape can be further improved.

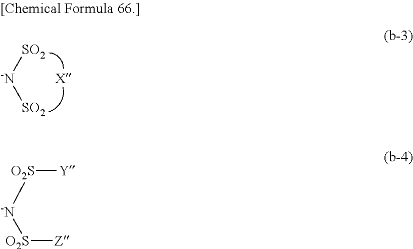

In the formulas, X″ represents an alkylene group of 2 to 6 carbon atoms in which at least one hydrogen atom has been substituted with a fluorine atom; and each of Y″ and Z″ independently represents an alkyl group of 1 to 10 carbon atoms in which at least one hydrogen atom has been substituted with a fluorine atom.

X″ represents a linear or branched alkylene group in which at least one hydrogen atom has been substituted with a fluorine atom, and the alkylene group has 2 to 6 carbon atoms, preferably 3 to 5 carbon atoms, and most preferably 3 carbon atoms.

Each of Y″ and Z″ independently represents a linear or branched alkyl group in which at least one hydrogen atom has been substituted with a fluorine atom, and the alkyl group has 1 to 10 carbon atoms, preferably 1 to 7 carbon atoms, and most preferably 1 to 3 carbon atoms.

The smaller the number of carbon atoms of the alkylene group for X″ or those of the alkyl group for Y″ and Z″ within the above-mentioned range of the number of carbon atoms, the more the solubility in a resist solvent is improved.

Further, in the alkylene group for X″ or the alkyl group for Y″ and Z″, it is preferable that the number of hydrogen atoms substituted with fluorine atoms is as large as possible because the acid strength increases and the transparency to high energy radiation of 200 nm or less or electron beam is improved.

The fluorination ratio of the alkylene group or alkyl group is preferably from 70 to 100%, more preferably from 90 to 100%, and it is particularly desirable that the alkylene group or alkyl group be a perfluoroalkylene group or perfluoroalkyl group in which all hydrogen atoms are substituted with fluorine atoms.

Further, as the component (B2), an onium salt acid generator in which the anion moiety in general formula (b-1) or (b-2) is replaced by an anion represented by general formula (b-5) shown below can also be preferably used.

By using such an onium salt acid generator in combination with the component (B1), in the formation of a resist pattern, critical resolution, sensitivity, EL margin, MEF, LWR, LER, circularity, CDU or pattern shape can be particularly improved.

[Chemical Formula 67.]

R^{0′}—SO₃⁻Z⁺ (b-5)

In the formula, R⁰′ represents a hydrocarbon group of 1 to 12 carbon atoms which may have a substituent, with the provision that the carbon atom adjacent to the sulfur atom within the —SO₃⁻ group has no fluorine atom bonded thereto; and Z⁺ represents an organic cation.

In general formula (b-5), the hydrocarbon group for R⁰′ may or may not have a substituent, provided that the carbon atom adjacent to the sulfur atom within the —SO₃⁻ group has no fluorine atom bonded thereto. Therefore, upon exposure, the acid-generator component represented by formula (b-5) generates a sulfonic acid exhibiting a weaker acid strength than the acid generated from an acid generator in which a fluorine atom is bonded to the carbon atom adjacent to the sulfur atom within —SO₃⁻. As a result, in the present invention, the shape of a resist pattern formed can be improved. Further, the lithography properties are also improved.

The substituent preferably contains no fluorine atom, and examples thereof include a lower alkyl group of 1 to 5 carbon atoms and an oxygen atom (═O).

The hydrocarbon group of 1 to 12 carbon atoms represented by R⁰′ may be either an aliphatic hydrocarbon group or an aromatic hydrocarbon group. By virtue of using a hydrocarbon group of 1 to 12 carbon atoms, the rectangularity of the resist pattern is improved.

When the hydrocarbon group for R⁰′ is an aliphatic hydrocarbon group, the aliphatic hydrocarbon group may be either saturated or unsaturated, but in general, the aliphatic hydrocarbon group is preferably saturated.

Further, the aliphatic hydrocarbon group may be either a chain-like (linear or branched) hydrocarbon group, or a cyclic hydrocarbon group.

As the chain-like hydrocarbon group, a linear or branched alkyl group is preferable. The alkyl group preferably has 1 to 10 carbon atoms, more preferably 1 to 8, and still more preferably 3 to 8.

Specific examples of linear or branched alkyl groups include a methyl group, an ethyl group, an n-propyl group, an isopropyl group, an n-butyl group, an isobutyl group, a tert-butyl group, a pentyl group, an isopentyl group, a neopentyl group, an n-hexyl group, an n-heptyl group and an n-octyl group. Among these, a methyl group, an n-propyl group and an n-octyl group are preferable, and an n-octyl group is particularly desirable.

Specific examples of the component (B1) having a sulfonate ion as the anion moiety in which R⁰′ represents a linear or branched alkyl group include onium salts having a cation represented by general formula (b1-c1), (b1-c2), (I-1), (I-2), (I-5) or (I-6) above as the cation moiety, and a sulfonate ion represented by general formula (b-5-1) shown below as the anion moiety.

[Chemical Formula 68.]

CₐH₂ₐ₊₁SO₃⁻ (b-5-1)

In the formula, a represents an integer of 1 to 10.

In general formula (b-5-1), a represents an integer of 1 to 10, and preferably 1 to 8.

Specific examples of sulfonate ions represented by general formula (b-5-1) include a methanesulfonate (MS) ion, an ethanesulfonate ion, an n-propanesulfonate ion, an n-butanesulfonate ion and an n-octanesulfonate ion.

Examples of cyclic hydrocarbon groups as the hydrocarbon group for R⁰′ include an aliphatic cyclic group and a group in which at least one hydrogen atom within a chain-like hydrocarbon group have been substituted with an aliphatic cyclic group (aliphatic cyclic group-containing group).

As the “aliphatic cyclic group”, the same aliphatic cyclic groups as those described above in connection with the acid dissociable, dissolution inhibiting group for the component (A) can be used. The aliphatic cyclic group preferably has 3 to 12 carbon atoms, and more preferably 4 to 10.

The aliphatic cyclic group may be either a polycyclic group or a monocyclic group.

As the monocyclic group, a group in which one hydrogen atom has been removed from a monocycloalkane of 3 to 6 carbon atoms is preferable, and specific examples thereof include a cyclopentyl group and a cyclohexyl group.

The polycyclic group preferably has 7 to 12 carbon atoms, and specific examples thereof include an adamantyl group, a norbornyl group, an isobornyl group, a tricyclodecanyl group and a tetracyclododecanyl group.

Among the aforementioned examples, a polycyclic group is preferable, and an adamantyl group, a norbornyl group or a tetracyclododecanyl group is preferable from an industrial viewpoint. As described above, these aliphatic cyclic groups may or may not have a substituent.

As the aliphatic cyclic group within the “aliphatic cyclic group-containing group”, the same groups as those described above can be used. As the chain-like hydrocarbon group to which the aliphatic cyclic group is bonded to form the “aliphatic cyclic group-containing group”, a linear or branched alkyl group is preferable, and a lower alkyl group of 1 to 5 carbon atoms is more preferable. Specific examples thereof include a methyl group, an ethyl group, an n-propyl group, an isopropyl group, an n-butyl group, an isobutyl group, a tert-butyl group, a pentyl group, an isopentyl group and a neopentyl group. Among these, a linear alkyl group is preferable, and from industrial viewpoint, a methyl group or an ethyl group is more preferable.

Specific examples of sulfonate ions in which R⁰′ is a cyclic hydrocarbon group include sulfonate ions represented by formulas (b-5-21) to (b-5-26) shown below.

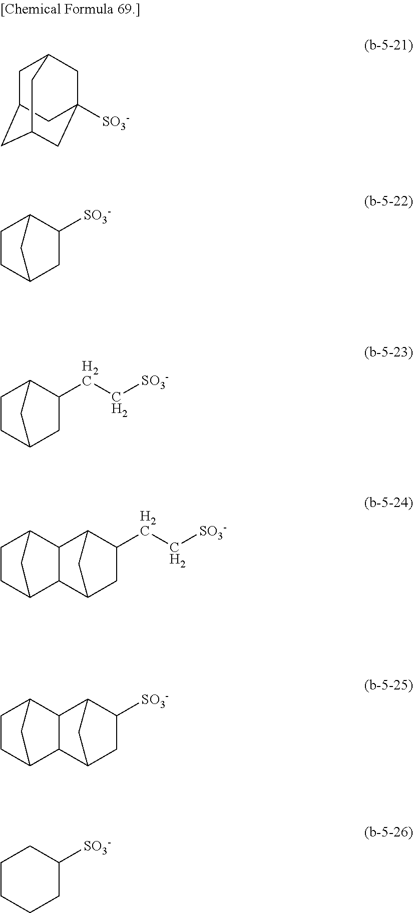

Further, as a sulfonate ion in which R⁰′ represents a cyclic hydrocarbon group, an ion represented by general formula (b-5-3) shown below is also preferable.

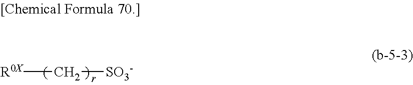

In the formula, R^{0X} represents a cyclic alkyl group of 4 to 12 carbon atoms that has an oxygen atom (═O) as a substituent; and r represents 0 or 1.

In general formula (b-5-3), R^{0X} represents a cyclic alkyl group of 4 to 12 carbon atoms that has an oxygen atom (═O) as a substituent

The expression “has an oxygen atom as a substituent” means that two hydrogen atoms bonded to a carbon atom constituting the cyclic alkyl group of 4 to 12 carbon atoms are substituted with an oxygen atom (═O).

The cyclic alkyl group represented by R^{0X} is not particularly limited as long as it has 4 to 12 carbon atoms, and may be either polycyclic or monocyclic. Examples thereof include a group in which one hydrogen atom has been removed from a monocycloalkane or a polycycloalkane such as a bicycloalkane, tricycloalkane or tetracycloalkane. As the monocyclic group, a group in which one hydrogen atom has been removed from a monocycloalkane of 3 to 8 carbon atoms is preferable, and specific examples thereof include a cyclopentyl group, a cyclohexyl group, a cycloheptyl group and a cyclooctyl group. The polycyclic group preferably has 7 to 12 carbon atoms, and specific examples thereof include an adamantyl group, a norbornyl group, an isobornyl group, a tricyclodecanyl group and a tetracyclododecanyl group.

As R^{0X}, a polycyclic alkyl group of 4 to 12 carbon atoms that has an oxygen atom (═O) as a substituent is preferable. From an industrial viewpoint, a group in which two hydrogen atoms bonded to a carbon atom constituting an adamantyl group, a norbornyl group or a tetracyclododecyl group are substituted with an oxygen atom (═O) is preferable, and a norbornyl group having an oxygen atom (═O) as a substituent is particularly desirable.

R^{0X} may have a substituent other than an oxygen atom. As an example of such a substituent, a lower alkyl group of 1 to 5 carbon atoms can be given.

In general formula (b-5-3), r represents 0 or 1, and preferably 1.

Specific examples of preferable anions represented by formula (b-5-3) include anions represented by formulas (b-5-31) and (b-5-32) shown below.

Of these, in terms of the effects of using in combination with the component (B1), a camphorsulfonate ion represented by formula (b-5-31) shown below is preferable.

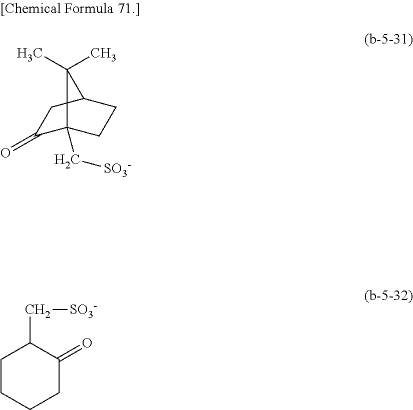

Examples of the aromatic hydrocarbon group as the hydrocarbon group for R⁰′ in formula (b-5) include a phenyl group, a tolyl group, a xylyl group, a mesityl group, a phenethyl group and a naphthyl group. As described above, the aromatic hydrocarbon group may or may not have a substituent.

Specific examples of aromatic hydrocarbon groups for R⁰′ include groups represented by general formula (b-5-41) or (b-5-42) shown below.

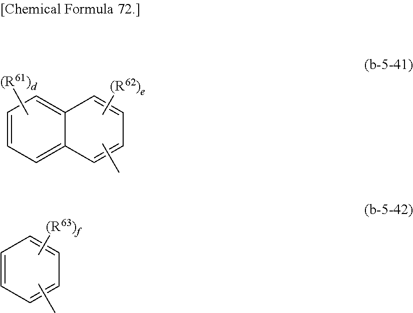

In formula (b-5-41), each of R⁶¹ and R⁶² independently represents an alkyl group of 1 to 5 carbon atoms, an alkoxy group of 1 to 5 carbon atoms or a halogen atom.

Examples of the alkyl group for R⁶¹ and R⁶² include a methyl group, an ethyl group, an n-propyl group, an isopropyl group, an n-butyl group, an isobutyl group, a tert-butyl group, a pentyl group, an isopentyl group and a neopentyl group. Among these, a methyl group is particularly desirable.

Examples of the alkoxy group for R⁶¹ and R⁶² include a methoxy group, an ethoxy group, an n-propoxy group, an isopropoxy group, an n-butoxy group and a tert-butoxy group. Among these, a methoxy group or an ethoxy group is particularly desirable.

Each of d and e independently represents an integer of 0 to 4, preferably 0 to 2, and most preferably 0.

If there are two or more of the R⁶¹ group and/or R⁶² group, as indicated by the value d and/or e, then the two or more of the R⁶¹ group and/or the R⁶² group may be the same or different from each other.

In formula (b-5-42), R⁶³ represents an alkyl group of 1 to 5 carbon atoms, an alkoxy group of 1 to 5 carbon atoms or a halogen atom.

Examples of the alkyl group for R⁶³ include a methyl group, an ethyl group, an n-propyl group, an isopropyl group, an n-butyl group, an isobutyl group, a tert-butyl group, a pentyl group, an isopentyl group and a neopentyl group. Among these, a methyl group is particularly desirable.

Examples of the alkoxy group for R⁶³ include a methoxy group, an ethoxy group, an n-propoxy group, an isopropoxy group, an n-butoxy group and a tert-butoxy group. Among these, a methoxy group or an ethoxy group is particularly desirable.

f represents an integer of 0 to 3, preferably 1 or 2, and most preferably 1.

If there are two ore more of the R⁶³ group, as indicated by the value f, the two or more of the R⁶³ group may be the same or different from each other.

Examples of sulfonates having an aromatic hydrocarbon group represented by formula (b-5-42) include benzene sulfonate, perfluorobenzenesulfonate and p-toluenesulfonate.

Further, an onium salt-based acid generator in which the anion moiety (R⁴″SO₃⁻) in general formula (b-1) or (b-2) has been replaced with R^{a}—COO⁻ (in the formula, R^{a} represents an alkyl group or a fluorinated alkyl group) can also be used as the component (B2) (the cation moiety is the same as that in general formula (b-1) or (b-2)).

In the formula above, as R^{a}, the same groups as those described above for R⁴″ can be used.

Specific examples of the group represented by the formula “R^{a}—COO⁻” include a trifluoroacetic acid ion, an acetic acid ion, and a 1-adamantanecarboxylic acid ion.

Further, onium salts having a cation moiety represented by general formula (I-1), (I-2), (I-5) or (I-6) above, and having a fluorinated alkylsulfonate ion (e.g., the anion moiety (R⁴″SO₃⁻) in general formula (b-1) or (b-2) above) or an anion moiety represented by general formula (b1) to (b8), (b-3), (b-4) or (b-5) above as the anion moiety, can be used.

In the present description, an oximesulfonate acid generator is a compound having at least one group represented by general formula (B-1) shown below, and has a feature of generating acid by irradiation. Such an oximesulfonate acid generator can also be preferably used as the component (B2). By using such an oximesulfonate acid generator in combination with the component (B1), in the formation of a resist pattern, critical resolution, sensitivity, EL margin, MEF, LWR, LER, circularity, CDU or pattern shape can be further improved.

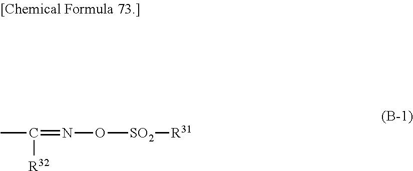

In the formula, each of R³¹ and R³² independently represents an organic group.

The organic group for R³¹ and R³² refers to a group containing a carbon atom, and may include atoms other than carbon atoms (e.g., a hydrogen atom, an oxygen atom, a nitrogen atom, a sulfur atom, a halogen atom (such as a fluorine atom and a chlorine atom) and the like).

As the organic group for R³¹, a linear, branched, or cyclic alkyl group or aryl group is preferable. The alkyl group or the aryl group may have a substituent. The substituent is not particularly limited, and examples thereof include a fluorine atom and a linear, branched, or cyclic alkyl group having 1 to 6 carbon atoms. The alkyl group or the aryl group “has a substituent” means that part or all of the hydrogen atoms of the alkyl group or the aryl group is substituted with a substituent.

The alkyl group preferably has 1 to 20 carbon atoms, more preferably 1 to 10 carbon atoms, still more preferably 1 to 8 carbon atoms, still more preferably 1 to 6 carbon atoms, and most preferably 1 to 4 carbon atoms. As the alkyl group, a partially or completely halogenated alkyl group (hereinafter, sometimes referred to as a “halogenated alkyl group”) is particularly desirable. The “partially halogenated alkyl group” refers to an alkyl group in which part of the hydrogen atoms are substituted with halogen atoms and the “completely halogenated alkyl group” refers to an alkyl group in which all of the hydrogen atoms are substituted with halogen atoms. Examples of halogen atoms include fluorine atoms, chlorine atoms, bromine atoms and iodine atoms, and fluorine atoms are particularly desirable. In other words, the halogenated alkyl group is preferably a fluorinated alkyl group.

The aryl group preferably has 4 to 20 carbon atoms, more preferably 4 to 10 carbon atoms, and most preferably 6 to 10 carbon atoms. As the aryl group, partially or completely halogenated aryl group is particularly desirable. The “partially halogenated aryl group” refers to an aryl group in which some of the hydrogen atoms are substituted with halogen atoms and the “completely halogenated aryl group” refers to an aryl group in which all of hydrogen atoms are substituted with halogen atoms.

As R³¹, an alkyl group of 1 to 4 carbon atoms which has no substituent or a fluorinated alkyl group of 1 to 4 carbon atoms is particularly desirable.

As the organic group for R³², a linear, branched, or cyclic alkyl group, aryl group, or cyano group is preferable. Examples of the alkyl group and the aryl group for R³² include the same alkyl groups and aryl groups as those described above for R³¹.

As R³², a cyano group, an alkyl group of 1 to 8 carbon atoms having no substituent or a fluorinated alkyl group of 1 to 8 carbon atoms is particularly desirable.

Preferred examples of the oxime sulfonate acid generator include compounds represented by general formula (B-2) or (B-3) shown below.

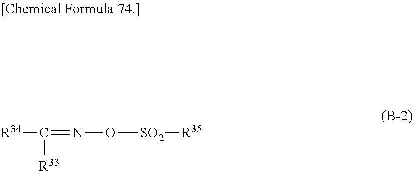

In the formula, R³³ represents a cyano group, an alkyl group having no substituent or a halogenated alkyl group; R³⁴ represents an aryl group; and R³⁵ represents an alkyl group having no substituent or a halogenated alkyl group.

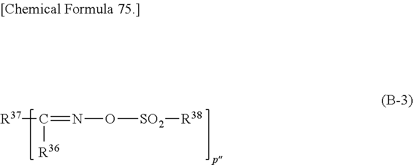

In the formula, R³⁶ represents a cyano group, an alkyl group having no substituent or a halogenated alkyl group; R³⁷ represents a divalent or trivalent aromatic hydrocarbon group; R³⁸ represents an alkyl group having no substituent or a halogenated alkyl group; and p″ represents 2 or 3.

In general formula (B-2), the alkyl group having no substituent or the halogenated alkyl group for R³³ preferably has 1 to 10 carbon atoms, more preferably 1 to 8 carbon atoms, and most preferably 1 to 6 carbon atoms.

As R³³, a halogenated alkyl group is preferable, and a fluorinated alkyl group is more preferable.

The fluorinated alkyl group for R³³ preferably has 50% or more of the hydrogen atoms thereof fluorinated, more preferably 70% or more, and most preferably 90% or more.

Examples of the aryl group for R³⁴ include groups in which one hydrogen atom has been removed from an aromatic hydrocarbon ring, such as a phenyl group, a biphenyl group, a fluorenyl group, a naphthyl group, an anthryl group, and a phenanthryl group, and heteroaryl groups in which some of the carbon atoms constituting the ring(s) of these groups are substituted with hetero atoms such as an oxygen atom, a sulfur atom, and a nitrogen atom. Of these, a fluorenyl group is preferable.

The aryl group for R³⁴ may have a substituent such as an alkyl group of 1 to 10 carbon atoms, a halogenated alkyl group, or an alkoxy group. The alkyl group and halogenated alkyl group as the substituent preferably has 1 to 8 carbon atoms, and more preferably 1 to 4 carbon atoms. Further, the halogenated alkyl group is preferably a fluorinated alkyl group.

The alkyl group having no substituent or the halogenated alkyl group for R³⁵ preferably has 1 to 10 carbon atoms, more preferably 1 to 8 carbon atoms, and most preferably 1 to 6 carbon atoms.

As R³⁵, a halogenated alkyl group is preferable, and a fluorinated alkyl group is more preferable.

In terms of enhancing the strength of the acid generated, the fluorinated alkyl group for R³⁵ preferably has 50% or more of the hydrogen atoms fluorinated, more preferably 70% or more, still more preferably 90% or more. A completely fluorinated alkyl group in which 100% of the hydrogen atoms are substituted with fluorine atoms is particularly desirable.

In general formula (B-3), as the alkyl group having no substituent and the halogenated alkyl group for R³⁶, the same alkyl group having no substituent and the halogenated alkyl group described above for R³³ can be used.

Examples of the divalent or trivalent aromatic hydrocarbon group for R³⁷ include groups in which one or two hydrogen atoms have been removed from the aryl group for R³⁴.

As the alkyl group having no substituent or the halogenated alkyl group for R³⁸, the same one as the alkyl group having no substituent or the halogenated alkyl group for R³⁵ can be used.

p″ is preferably 2.

Specific examples of suitable oxime sulfonate acid generators include α-(p-toluenesulfonyloxyimino)-benzyl cyanide, α-(p-chlorobenzenesulfonyloxyimino)-benzyl cyanide, α-(4-nitrobenzenesulfonyloxyimino)-benzyl cyanide, α-(4-nitro-2-trifluoromethylbenzenesulfonyloxyimino)-benzyl cyanide, α-(benzenesulfonyloxyimino)-4-chlorobenzyl cyanide, α-(benzenesulfonyloxyimino)-2,4-dichlorobenzyl cyanide, α-(benzenesulfonyloxyimino)-2,6-dichlorobenzyl cyanide, α-(benzenesulfonyloxyimino)-4-methoxybenzyl cyanide, α-(2-chlorobenzenesulfonyloxyimino)-4-methoxybenzyl cyanide, α-(benzenesulfonyloxyimino)-thien-2-yl acetonitrile, α-(4-dodecylbenzenesulfonyloxyimino)benzyl cyanide, α-[(p-toluenesulfonyloxyimino)-4-methoxyphenyl]acetonitrile, α-[(dodecylbenzenesulfonyloxyimino)-4-methoxyphenyl]acetonitrile, α-(tosyloxyimino)-4-thienyl cyanide, α-(methylsulfonyloxyimino)-1-cyclopentenyl acetonitrile, α-(methylsulfonyloxyimino)-1-cyclohexenyl acetonitrile, α-(methylsulfonyloxyimino)-1-cycloheptenyl acetonitrile, α-(methylsulfonyloxyimino)-1-cyclooctenyl acetonitrile, α-(trifluoromethylsulfonyloxyimino)-1-cyclopentenyl acetonitrile, α-(trifluoromethylsulfonyloxyimino)-cyclohexyl acetonitrile, α-(ethylsulfonyloxyimino)-ethyl acetonitrile, α-(propylsulfonyloxyimino)-propyl acetonitrile, α-(cyclohexylsulfonyloxyimino)-cyclopentyl acetonitrile, α-(cyclohexylsulfonyloxyimino)-cyclohexyl acetonitrile, α-(cyclohexylsulfonyloxyimino)-1-cyclopentenyl acetonitrile, α-(ethylsulfonyloxyimino)-1-cyclopentenyl acetonitrile, α-(isopropylsulfonyloxyimino)-1-cyclopentenyl acetonitrile, α-(n-butylsulfonyloxyimino)-1-cyclopentenyl acetonitrile, α-(ethylsulfonyloxyimino)-1-cyclohexenyl acetonitrile, α-(isopropylsulfonyloxyimino)-1-cyclohexenyl acetonitrile, α-(n-butylsulfonyloxyimino)-1-cyclohexenyl acetonitrile, α-(methylsulfonyloxyimino)-phenyl acetonitrile, α-(methylsulfonyloxyimino)-p-methoxyphenyl acetonitrile, α-(trifluoromethylsulfonyloxyimino)-phenyl acetonitrile, α-(trifluoromethylsulfonyloxyimino)-p-methoxyphenyl acetonitrile, α-(ethylsulfonyloxyimino)-p-methoxyphenyl acetonitrile, α-(propylsulfonyloxyimino)-p-methylphenyl acetonitrile, and α-(methylsulfonyloxyimino)-p-bromophenyl acetonitrile.

Further, oxime sulfonate acid generators disclosed in Japanese Unexamined Patent Application, First Publication No. Hei 9-208554 (Chemical Formulas 18 and 19 shown in paragraphs [0012] to [0014]) and oxime sulfonate acid generators disclosed in WO 2004/074242A2 (Examples 1 to 40 described at pages 65 to 85) may be preferably used.

Furthermore, as preferable examples, the following can be used.

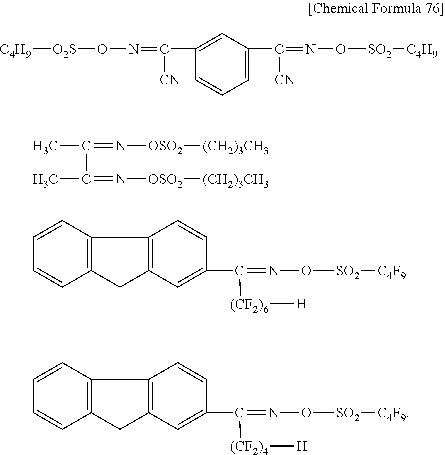

Of the aforementioned diazomethane acid generators, specific examples of suitable bisalkyl or bisaryl sulfonyl diazomethanes include bis(isopropylsulfonyl)diazomethane, bis(p-toluenesulfonyl)diazomethane, bis(1,1-dimethylethylsulfonyl)diazomethane, bis(cyclohexylsulfonyl)diazomethane, and bis(2,4-dimethylphenylsulfonyl)diazomethane.

Further, diazomethane acid generators disclosed in Japanese Unexamined Patent Application, First Publication No. Hei 11-035551, Japanese Unexamined Patent Application, First Publication No. Hei 11-035552 and Japanese Unexamined Patent Application, First Publication No. Hei 11-035573 may be preferably used.

Furthermore, as examples of poly(bis-sulfonyl)diazomethanes, those disclosed in Japanese Unexamined Patent Application, First Publication No. Hei 11-322707, including 1,3-bis(phenylsulfonyldiazomethylsulfonyl)propane, 1,4-bis(phenylsulfonyldiazomethylsulfonyl)butane, 1,6-bis(phenylsulfonyldiazomethylsulfonyl)hexane, 1,10-bis(phenylsulfonyldiazomethylsulfonyl)decane, 1,2-bis(cyclohexylsulfonyldiazomethylsulfonyl)ethane, 1,3-bis(cyclohexylsulfonyldiazomethylsulfonyl)propane, 1,6-bis(cyclohexylsulfonyldiazomethylsulfonyl)hexane, and 1,10-bis(cyclohexylsulfonyldiazomethylsulfonyl)decane, may be given.

As the component (B2), one type of acid generator may be used, or two or more types may be used in combination.

In the positive resist composition of the present invention, the total amount of the component (B) relative to 100 parts by weight of the component (A) is preferably 0.5 to 50 parts by weight, and more preferably 1 to 40 parts by weight. When the amount of the component (B) is within the above-mentioned range, formation of a resist pattern can be satisfactorily performed. Further, by virtue of the above-mentioned range, a uniform solution can be obtained and the storage stability becomes satisfactory.

<Optional Components>

[Component (D)]

It is preferable that the resist composition of the present invention further includes a nitrogen-containing organic compound (D) (hereafter referred to as the component (D)) as an optional component.

As the component (D), there is no particular limitation as long as it functions as an acid diffusion control agent, i.e., a quencher which traps the acid generated from the component (B) upon exposure. A multitude of these components (D) have already been proposed, and any of these known compounds may be used. Among these, an aliphatic amine, particularly a secondary aliphatic amine or tertiary aliphatic amine, and an aromatic amine is preferable.

An aliphatic amine is an amine having one or more aliphatic groups, and the aliphatic groups preferably have 1 to 12 carbon atoms.

Examples of these aliphatic amines include amines in which at least one hydrogen atom of ammonia (NH₃) has been substituted with an alkyl group or hydroxyalkyl group of no more than 12 carbon atoms (i.e., alkylamines or alkylalcoholamines), and cyclic amines.

Specific examples of alkylamines and alkylalcoholamines include monoalkylamines such as n-hexylamine, n-heptylamine, n-octylamine, n-nonylamine, and n-decylamine; dialkylamines such as diethylamine, di-n-propylamine, di-n-heptylamine, di-n-octylamine, and dicyclohexylamine; trialkylamines such as trimethylamine, triethylamine, tri-n-propylamine, tri-n-butylamine, tri-n-hexylamine, tri-n-pentylamine, tri-n-heptylamine, tri-n-octylamine, tri-n-nonylamine, tri-n-decylamine, and tri-n-dodecylamine; and alkyl alcohol amines such as diethanolamine, triethanolamine, diisopropanolamine, triisopropanolamine, di-n-octanolamine, and tri-n-octanolamine. Among these, trialkylamines of 5 to 10 carbon atoms are preferable, and tri-n-pentylamine and tri-n-octylamine are particularly desirable.

Examples of the cyclic amine include heterocyclic compounds containing a nitrogen atom as a hetero atom. The heterocyclic compound may be a monocyclic compound (aliphatic monocyclic amine), or a polycyclic compound (aliphatic polycyclic amine).

Specific examples of the aliphatic monocyclic amine include piperidine, and piperazine.

The aliphatic polycyclic amine preferably has 6 to 10 carbon atoms, and specific examples thereof include 1,5-diazabicyclo[4.3.0]-5-nonene, 1,8-diazabicyclo[5.4.0]-7-undecene, hexamethylenetetramine, and 1,4-diazabicyclo[2.2.2]octane.

Further, aliphatic amines other than those described above can be used. Examples of other aliphatic amines include tris(2-methoxymethoxyethyl)amine, tris{2-(2-methoxyethoxy)ethyl}amine, tris{2-(2-methoxyethoxymethoxy)ethyl}amine, tris{2-(1-methoxyethoxy)ethyl}amine, tris{2-(1-ethoxyethoxy)ethyl}amine, tris{2-(1-ethoxypropoxy)ethyl}amine and tris[2-{2-(2-hydroxyethoxy)ethoxy}ethyl]amine.

Examples of aromatic amines include aniline compounds such as aniline, N,N-n-butyl-aniline, 2,6-diisopropylaniline, N-isopropylaniline, 3-isopropoxyaniline and N-ethylaniline, pyridine, 4-dimethylaminopyridine, pyrrole, indole, pyrazole, imidazole and derivatives thereof, as well as diphenylamine, triphenylamine and tribenzylamine.

As the component (D), one type of compound may be used alone, or two or more types may be used in combination.

The component (D) is typically used in an amount within a range from 0.01 to 5.0 parts by weight, relative to 100 parts by weight of the component (A). When the amount of the component (D) is within the above-mentioned range, the shape of the resist pattern and the post exposure stability of the latent image formed by the pattern-wise exposure of the resist layer are improved.

[Component (E)]

Furthermore, in the resist composition of the present invention, for preventing any deterioration in sensitivity, and improving the resist pattern shape and the post exposure stability of the latent image formed by the pattern-wise exposure of the resist layer, at least one compound (E) (hereafter referred to as the component (E)) selected from the group consisting of an organic carboxylic acid, or a phosphorus oxo acid or derivative thereof can be added.

Examples of suitable organic carboxylic acids include acetic acid, malonic acid, citric acid, malic acid, succinic acid, benzoic acid, and salicylic acid.

Examples of phosphorus oxo acids include phosphoric acid, phosphonic acid and phosphinic acid. Among these, phosphonic acid is particularly desirable.

Examples of oxo acid derivatives include esters in which a hydrogen atom within the above-mentioned oxo acids is substituted with a hydrocarbon group. Examples of the hydrocarbon group include an alkyl group of 1 to 5 carbon atoms and an aryl group of 6 to 15 carbon atoms.

Examples of phosphoric acid derivatives include phosphoric acid esters such as di-n-butyl phosphate and diphenyl phosphate.

Examples of phosphonic acid derivatives include phosphonic acid esters such as dimethyl phosphonate, di-n-butyl phosphonate, phenylphosphonic acid, diphenyl phosphonate and dibenzyl phosphonate.

Examples of phosphinic acid derivatives include phosphinic acid esters such as phenylphosphinic acid.

As the component (E), one type may be used alone, or two or more types may be used in combination.

As the component (E), an organic carboxylic acid is preferred, and salicylic acid is particularly desirable.

The component (E) is typically used in an amount within a range from 0.01 to 5.0 parts by weight, relative to 100 parts by weight of the component (A).

If desired, other miscible additives can also be added to the resist composition of the present invention. Examples of such miscible additives include additive resins for improving the performance of the resist film, surfactants for improving the applicability, dissolution inhibitors, plasticizers, stabilizers, colorants, halation prevention agents, and dyes.

[Component (S)]

The resist composition of the present invention can be produced by dissolving the materials for the resist composition in an organic solvent (hereafter, referred to as “component (S)”).

The component (S) may be any organic solvent which can dissolve the respective components to give a uniform solution, and one or more kinds of any organic solvent can be appropriately selected from those which have been conventionally known as solvents for a chemically amplified resist.

Examples of the component (S) include lactones such as γ-butyrolactone; ketones such as acetone, methyl ethyl ketone, cyclohexanone (CH), methyl-n-pentyl ketone, methyl isopentyl ketone, and 2-heptanone; polyhydric alcohols, such as ethylene glycol, diethylene glycol, propylene glycol and dipropylene glycol; compounds having an ester bond, such as ethylene glycol monoacetate, diethylene glycol monoacetate, propylene glycol monoacetate, and dipropylene glycol monoacetate; polyhydric alcohol derivatives including compounds having an ether bond, such as a monoalkylether (e.g., monomethylether, monoethylether, monopropylether or monobutylether) or monophenylether of any of these polyhydric alcohols or compounds having an ester bond (among these, propylene glycol monomethyl ether acetate (PGMEA) and propylene glycol monomethyl ether (PGME) are preferable); cyclic ethers such as dioxane; esters such as methyl lactate, ethyl lactate (EL), methyl acetate, ethyl acetate, butyl acetate, methyl pyruvate, ethyl pyruvate, methyl methoxypropionate, and ethyl ethoxypropionate; and aromatic organic solvents such as anisole, ethylbenzylether, cresylmethylether, diphenylether, dibenzylether, phenetole, butylphenylether, ethylbenzene, diethylbenzene, pentylbenzene, isopropylbenzene, toluene, xylene, cymene and mesitylene.

The component (S) can be used individually, or in combination as a mixed solvent.

Among these, cyclohexanone (CH), γ-butyrolactone, propylene glycol monomethyl ether acetate (PGMEA), propylene glycol monomethyl ether (PGME) and ethyl lactate (EL) are preferable, and γ-butyrolactone, PGMEA and PGME are particularly desirable.

Further, among the mixed solvents, a mixed solvent obtained by mixing PGMEA with a polar solvent is preferable. The mixing ratio (weight ratio) of the mixed solvent can be appropriately determined, taking into consideration the compatibility of the PGMEA with the polar solvent, but is preferably in the range of 1:9 to 9:1, more preferably from 2:8 to 8:2.

Specifically, when EL is mixed as the polar solvent, the PGMEA:EL weight ratio is preferably from 1:9 to 9:1, and more preferably from 2:8 to 8:2. Alternatively, when PGME is mixed as the polar solvent, the PGMEA:PGME weight ratio is preferably from 1:9 to 9:1, more preferably from 2:8 to 8:2, and still more preferably 3:7 to 7:3. Alternatively, when cyclohexanone (CH) is mixed as the polar solvent, the PGMEA:CH weight ratio is preferably from 1:9 to 9:1, and more preferably from 2:8 to 9:1.

Further, as the component (S), a mixed solvent of at least one of PGMEA and EL with γ-butyrolactone is also preferable. The mixing ratio (former:latter) of such a mixed solvent is preferably from 70:30 to 95:5.

The amount of the component (S) is not particularly limited, and is adjusted appropriately to a concentration that enables application of a coating solution to a substrate in accordance with the thickness of the coating film. In general, the component (S) is used in an amount that yields a solid content for the resist composition that is preferably within a range from 0.5 to 20% by weight, and more preferably from 1 to 15% by weight.

Dissolving of the components for a resist composition in the component (S) can be conducted by simply mixing and stirring each of the above components together using conventional methods, and where required, the composition may also be mixed and dispersed using a dispersion device such as a dissolver, a homogenizer, or a triple roll mill. Furthermore, following mixing, the composition may also be filtered using a mesh, or a membrane filter or the like.

As described above, according to the resist composition of the present invention, excellent lithography properties such as roughness, mask reproducibility and exposure latitude can be achieved, and a resist pattern having an excellent shape with high rectangularity can be formed. The reasons why these effects can be achieved has not been elucidated yet, but are presumed as follows.

The resist composition of the present invention includes an acid generator (B1) containing a compound represented by general formula (b1-1).

The component (B1) has an adamantanelactone group in the anion moiety, and “—O—C(═O)—Y⁰—SO₃⁻” is bonded to a specific position of the adamantanelactone group.

By virtue of having an adamantanelactone group in the anion moiety, the component (B1) has a bulky skeleton and a polar unit. As a result, the interaction between the component (B1) and the base component (A) can be enhanced, thereby achieving excellent lithography properties and enabling formation of a resist pattern having an excellent shape.

Further, by virtue of “—O—C(═O)—Y⁰—SO₃⁻” being bonded to a specific position of the adamantanelactone group, various merits in terms of synthesis (improvement in yield, reactivity and purity) can be obtained, as compared to other bonding positions.

In the resist composition of the present invention, by using the acid generator (B1) in combination with the component (A) containing a polymeric compound having the structural unit (a0), an interaction occurs between the component (B1) and the component (A), which results in improved adhesion of the resist film to the substrate, and improved lithography properties.

<<Method of Forming a Resist Pattern>>

The method of forming a resist pattern according to the second aspect of the present invention includes: using a resist composition according to the first aspect of the present invention to form a resist film on a substrate; conducting exposure of the resist film; and alkali-developing the resist film to form a resist pattern.

The method for forming a resist pattern according to the present invention can be performed, for example, as follows.

Firstly, a resist composition of the present invention is applied onto a substrate using a spinner or the like, and a prebake (post applied bake (PAB)) is conducted under temperature conditions of 80 to 150° C. for 40 to 120 seconds, preferably 60 to 90 seconds to form a resist film. Then, for example, using an electron lithography system or the like, the resist film is selectively exposed to an electron beam (EB) through a desired mask pattern, followed by post exposure bake (PEB) under temperature conditions of 80 to 150° C. for 40 to 120 seconds, preferably 60 to 90 seconds. Subsequently, alkali developing is conducted using an alkali developing solution such as a 0.1 to 10% by weight aqueous solution of tetramethylammonium hydroxide (TMAH), preferably followed by rinsing with pure water, and drying. If desired, bake treatment (post bake) can be conducted following the alkali developing. In this manner, a resist pattern that is faithful to the mask pattern can be obtained.

The substrate is not specifically limited and a conventionally known substrate can be used. For example, substrates for electronic components, and such substrates having wiring patterns formed thereon can be used. Specific examples of the material of the substrate include metals such as silicon wafer, copper, chromium, iron and aluminum; and glass. Suitable materials for the wiring pattern include copper, aluminum, nickel, and gold.

Further, as the substrate, any one of the above-mentioned substrates provided with an inorganic and/or organic film on the surface thereof may be used. As the inorganic film, an inorganic antireflection film (inorganic BARC) can be used. As the organic film, an organic antireflection film (organic BARC) can be used.

The wavelength to be used for exposure is not particularly limited and the exposure can be conducted using radiation such as ArF excimer laser, KrF excimer laser, F₂ excimer laser, extreme ultraviolet rays (EUV), vacuum ultraviolet rays (VUV), electron beam (EB), X-rays, and soft X-rays.

The resist composition of the present invention is effective to KrF excimer laser, ArF excimer laser, EB and EUV, and particularly effective to EB or EUV.

The exposure of the resist film can be either a general exposure (dry exposure) conducted in air or an inert gas such as nitrogen, or immersion exposure (immersion lithography).

In immersion lithography, exposure (immersion exposure) is conducted in a state where the region between the lens and the resist layer formed on a wafer (which was conventionally filled with air or an inert gas such as nitrogen) is filled with a solvent (a immersion medium) that has a larger refractive index than the refractive index of air.

More specifically, in immersion lithography, the region between the resist film formed in the above-described manner and lens at the lowermost portion of the exposure apparatus is filled with a solvent (a immersion medium) that has a larger refractive index than the refractive index of air, and in this state, the resist film is subjected to exposure (immersion exposure) through a desired mask pattern.

The immersion medium preferably exhibits a refractive index larger than the refractive index of air but smaller than the refractive index of the resist film to be subjected to immersion exposure. The refractive index of the immersion medium is not particularly limited as long at it satisfies the above-mentioned requirements.

Examples of this immersion medium which exhibits a refractive index that is larger than the refractive index of air but smaller than the refractive index of the resist film include water, fluorine-based inert liquids, silicon-based solvents and hydrocarbon-based solvents.

Specific examples of the fluorine-based inert liquids include liquids containing a fluorine-based compound such as C₃HCl₂F₅, C₄F₉OCH₃, C₄F₉OC₂H₅ or C₅H₃F₇ as the main component, which have a boiling point within a range from 70 to 180° C. and preferably from 80 to 160° C. A fluorine-based inert liquid having a boiling point within the above-mentioned range is advantageous in that the removal of the immersion medium after the exposure can be conducted by a simple method.

As a fluorine-based inert liquid, a perfluoroalkyl compound in which all of the hydrogen atoms of the alkyl group are substituted with fluorine atoms is particularly desirable. Examples of these perfluoroalkyl compounds include perfluoroalkylether compounds and perfluoroalkylamine compounds.

Specifically, one example of a suitable perfluoroalkylether compound is perfluoro(2-butyl-tetrahydrofuran) (boiling point 102° C.), and an example of a suitable perfluoroalkylamine compound is perfluorotributylamine (boiling point 174° C.).

The method of forming a resist pattern according to the present invention is also applicable to a double exposure method or a double patterning method.

<<Compound>>

The compound according to the third aspect of the present invention is a compound represented by general formula (b1-1) shown below, and is the same as the component (B1) contained in the component (B) of the resist composition according to the first aspect.

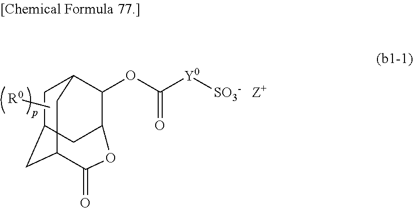

In the formula, Y⁰ represents an alkylene group of 1 to 4 carbon atoms which may have a substituent or a fluorinated alkylene group which may have a substituent; R⁰ represents an alkyl group, an alkoxy group, a halogen atom, a halogenated alkyl group, a hydroxy group or an oxygen atom (═O); p represents 0 or 1; and Z⁺ represents an organic cation.

The explanation of the compound of the present invention is the same as the explanation of the aforementioned component (B1).

(Production Method of Compound)

The compound of the present invention (compound (b1-1) represented by general formula (b1-1)) can be produced, for example, by reacting a compound (1′) represented by general formula (1′) shown below with a compound (2′) represented by general formula (2′) shown below to obtain a compound (3′), and then reacting the compound (3′) with a compound (4′) represented by general formula (4′) shown below.

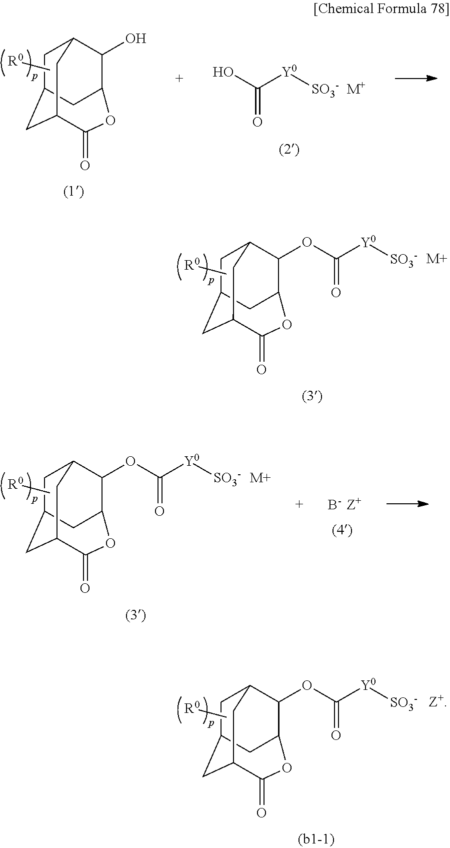

In the formulas, Y⁰, R⁰, p and Z⁺ are respectively the same as defined for Y⁰, R⁰, p and Z⁺ in the aforementioned formula (b1-1); M⁺ represents an alkali metal ion or an ammonium ion which may have a substituent; and B⁻ represents a non-nucleophilic ion.

M⁺ represents an alkali metal ion, or an ammonium ion which may have a substituent.

Examples of alkali metal ions include a sodium ion, a lithium ion and a potassium ion, and a sodium ion or a lithium ion is preferable.

As an example of the ammonium ion which may have a substituent, a group represented by general formula (b1-2-2) shown below can be given.

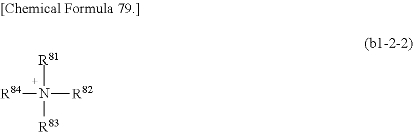

In the formula, each of R⁸¹ to R⁸⁴ independently represents a hydrogen atom or a hydrocarbon group which may have a substituent, provided that at least one of R⁸¹ to R⁸⁴ represents a hydrocarbon group; and at least two of R⁸¹ to R⁸⁴ may be mutually bonded to form a ring.

In formula (b1-2-2), each of R⁸¹ to R⁸⁴ independently represents a hydrogen atom or a hydrocarbon group which may have a substituent, provided that at least one of R⁸¹ to R⁸⁴ represents a hydrocarbon group.

As the hydrocarbon group for R⁸¹ to R⁸⁴, the same groups as those described above for X can be mentioned.

The hydrocarbon group may be either an aliphatic hydrocarbon group or an aromatic hydrocarbon group. When the hydrocarbon group is an aliphatic hydrocarbon group, it is particularly desirable that the hydrocarbon group is an alkyl group of 1 to 12 carbon atoms which may have a substituent.

At least one of R⁸¹ to R⁸⁴ is a hydrocarbon group, and it is preferable that two or three groups are hydrocarbon groups.

At least two of R⁸¹ to R⁸⁴ may be mutually bonded to form a ring. For example, two of R⁸¹ to R⁸⁴ may be bonded to form a ring, three of R⁸¹ to R⁸⁴ may be bonded to form a ring, or two of R⁸¹ to R⁸⁴ may be bonded to form a ring, and the remaining two may be bonded to form another ring.

The ring which is formed by at least two of R⁸¹ to R⁸⁴ bonded together with the nitrogen atom (i.e., the hetero ring containing nitrogen as a hetero atom) may be either an aliphatic hetero ring, or an aromatic hetero ring. Further, the hetero ring may be either a monocyclic group or a polycyclic group.

Specific examples of the ammonium ion represented by general formula (b1-2-2) include ammonium ions derived from an amine.

Here, an “ammonium ion derived from an amine” refers to an amine having a hydrogen atom bonded to the nitrogen atom to become a cation, and a tertiary ammonium ion in which a substituent has been bonded to the nitrogen atom of an amine.

The amine from which the ammonium ion is derived may be either an aliphatic amine or an aromatic amine.

As the aliphatic amine, an amine in which at least one hydrogen atom of ammonia (NH₃) has been substituted with an alkyl group or hydroxyalkyl group of no more than 12 carbon atoms (i.e., alkylamines or alkylalcoholamines), or a cyclic amine is particularly desirable.

Specific examples of alkylamines and alkylalcoholamines include monoalkylamines such as n-hexylamine, n-heptylamine, n-octylamine, n-nonylamine, and n-decylamine; dialkylamines such as diethylamine, di-n-propylamine, di-n-heptylamine, di-n-octylamine, and dicyclohexylamine; trialkylamines such as trimethylamine, triethylamine, tri-n-propylamine, tri-n-butylamine, tri-n-hexylamine, tri-n-pentylamine, tri-n-heptylamine, tri-n-octylamine, tri-n-nonylamine, tri-n-decylamine, and tri-n-dodecylamine; and alkyl alcohol amines such as diethanolamine, triethanolamine, diisopropanolamine, triisopropanolamine, di-n-octanolamine, and tri-n-octanolamine.

Examples of the cyclic amine include heterocyclic compounds containing a nitrogen atom as a hetero atom. The heterocyclic compound may be a monocyclic compound (aliphatic monocyclic amine), or a polycyclic compound (aliphatic polycyclic amine).

Specific examples of the aliphatic monocyclic amine include piperidine, and piperazine.

The aliphatic polycyclic amine preferably has 6 to 10 carbon atoms, and specific examples thereof include 1,5-diazabicyclo[4.3.0]-5-nonene, 1,8-diazabicyclo[5.4.0]-7-undecene, hexamethylenetetramine, and 1,4-diazabicyclo[2.2.2]octane.

Examples of aromatic amines include aniline, pyridine, 4-dimethylaminopyridine (DMAP), pyrrole, indole, pyrazole, and imidazole.

Examples of the tertiary ammonium ion include a tetramethylammonium ion, a tetraethylammonium ion and a tetrabutylammonium ion.

As the ammonium ion represented by general formula (b1-2-2), a group in which at least one of R⁸¹ to R⁸⁴ is an alkyl group and at least one is a hydrogen atom is particularly desirable.

Especially, a group in which three of R⁸¹ to R⁸⁴ are alkyl groups, and the remaining one is a hydrogen atom (i.e., a trialkylammonium ion), or a group in which two of R⁸¹ to R⁸⁴ are alkyl groups, and the remaining two are hydrogen atoms (i.e., dialkylammonium ion) is preferable.

It is preferable that each of the alkyl groups within the trialkylammonium ion or the dialkylammonium ion independently has 1 to 10 carbon atoms, more preferably 1 to 8, and most preferably 1 to 5. Specific examples include a methyl group, an ethyl group, a propyl group, a butyl group, a pentyl group, a hexyl group, a heptyl group, an octyl group, a nonyl group and a decyl group. Among these, an ethyl group is particularly desirable.

In formula (4′), B⁻ represents a non-nucleophilic ion. Examples of non-nucleophilic ions include a halogen ion such as a bromine ion or a chlorine ion; an ion capable of forming an acid exhibiting a lower acidity than the compound (3′); BF₄⁻, AsF₆⁻, SbF₆⁻, PF₆⁻ and ClO₄⁻.

Examples of ions for B⁻ which are capable of forming an acid exhibiting a lower acidity than the compound (3′) include sulfonic acid ions such as a p-toluenesulfonate ion, a methanesulfonate ion and a benzenesulfonate ion.

Reaction Between Compound (1′) and Compound (2′)

The compound (3′) can be produced, for example, by dissolving the compound (1′) and the compound (2′) in an aprotic organic solvent such as dichloroethane, benzene, toluene, ethylbenzene, chlorobenzene, acetonitrile or N,N-dimethylformamide, followed by stirring in the presence of an acidic catalyst, and performing a dehydration/condensation reaction.

In the dehydration/condensation reaction, in terms of improving the yield, purity and the like of the obtained compound (3′), it is particularly desirable to use an aromatic organic solvent (e.g., toluene, xylene or chlorobenzene) as the organic solvent.

The reaction temperature of the dehydration/condensation reaction is preferably about 20 to 200° C., and more preferably 50 to 150° C. The reaction time depends on the reactivity of the compounds (1′) and (2′), the reaction temperature or the like. However, in general, the reaction time is preferably 1 to 30 hours, and more preferably 3 to 30 hours.

The amount of the compound (2′) used in the dehydration/condensation reaction is not particularly limited, but in general, the amount of the compound (2′) is preferably about 0.2 to 3 moles, more preferably about 0.5 to 2 moles, and most preferably about 0.75 to 1.5 mole, per 1 mole of the compound (1′).

Examples of the acidic catalyst include an organic acid such as p-toluenesulfonic acid, and an organic acid such as sulfuric acid or hydrochloric acid. These acidic catalysts may be used individually or in a combination of two or more.

In the dehydration/condensation reaction, the acidic catalyst may be used in a catalyst amount. In general, the amount of the acidic catalyst used is about 0.001 to 5 moles, per 1 mole of the compound (1′).

The dehydration/condensation reaction may be conducted while removing water by using a Dean-Stark apparatus. In this manner, the reaction time can be shortened.

Further, in the dehydration/condensation reaction, a dehydrating agent such as 1,1′-carbonyldiimidazole or N,N′-dicyclohexylcarbodiimide may also be used.

When a dehydrating agent is used, in general, the amount of the dehydrating agent is preferably 0.2 to 5 moles, more preferably 0.5 to 3 moles, per 1 mole of the compound (1′).

Reaction Between Compound (3′) and Compound (4′)

The reaction between the compound (3′) and the compound (4′) can be effected by dissolving the compounds in a solvent such as water, dichloromethane, acetonitrile, methanol, chloroform or methylene chloride, followed by stirring.

The reaction temperature is preferably 0 to 150° C., and more preferably 0 to 100° C. The reaction time depends on the reactivity of the compounds (3′) and (4′), the reaction temperature or the like. However, in general, the reaction time is preferably 0.5 to 10 hours, and more preferably 1 to 5 hours.

In general, the amount of the compound (4′) used in the reaction is preferably 0.5 to 2 moles, per 1 mole of the compound (3′).

After the reaction, the compound (b1-1) within the reaction mixture may be separated and purified. The separation and purification can be conducted by a conventional method. For example, any one of concentration, solvent extraction, distillation, crystallization, recrystallization and chromatography can be used alone, or two or more of these methods may be used in combination.

The structure of the compound of the present invention obtained in the manner described above can be confirmed by a general organic analysis method such as ¹H-nuclear magnetic resonance (NMR) spectrometry, ¹³C-NMR spectrometry, ¹⁹F-NMR spectrometry, infrared absorption (IR) spectrometry, mass spectrometry, elementary analysis and X-ray diffraction analysis.

The compound of the present invention described above is a novel compound useful as an acid generator for a resist composition, and can be blended in a resist composition as an acid generator.

<<Acid Generator>>

The acid generator according to a fourth aspect of the present invention is an acid generator including the compound (b1-1).

The acid generator is useful for a chemically amplified resist composition, for example, the acid-generator component (B) of the resist composition according to the first aspect of the present invention.

### EXAMPLES

As follows is a description of examples of the present invention, although the scope of the present invention is by no way limited by these examples.

In the following examples, a compound represented by a chemical formula (1) is designated as “compound (1)”, and the same applies for compounds represented by other formulas.

### Synthesis of Novel Compound

### Examples 1 to 45

The novel compound of the present invention was synthesized in accordance with the examples shown below.

In the NMR analysis, the internal standard for ¹H-NMR is tetramethylsilane (TMS), and the internal standard for ¹⁹F-NMR is hexafluorobenzene (the peak of hexafluorobenzene was regarded as −160 ppm).

### Example 1

### Synthesis of Compound (B1-1-1)

i) Synthesis of Compound (3)

A mixture containing 5 g of a compound (1), 6.5 g of a compound (2), 0.05 g of p-toluenesulfonic acid monohydrate and 50 g of toluene was prepared, and reflux was conducted under normal pressure for 22 hours. Then, the mixture was cooled to room temperature to obtain a slurry, and the slurry was filtered, followed by disperse washing with 50 g of t-butylmethylether 3 times, thereby obtaining a compound (3).

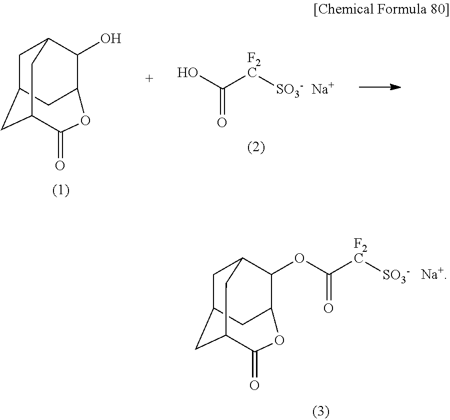

The obtained compound (3) was analyzed by ¹H-NMR and ¹⁹F-NMR, and the structure thereof was identified by the following results.

¹H-NMR (DMSO, 400 MHz): δ(ppm)=5.02 (s, 1H, CH), 4.21 (s, 1H, CH), 2.93 (s, 1H, CH), 1.41-2.29 (m, 10H, Adamantanelactone).

¹⁹F-NMR (DMSO, 376 MHz): δ(ppm)=−107.3.

From the results of the NMR analyses, it was confirmed that the compound (3) had a structure as shown above.

ii) Synthesis of Compound (B1-1-1)

A mixture containing 3 g of 4-methylphenyldiphenylsulfonium bromide, 3.34 g of the compound (3), 30 g of dichloromethane and 30 g of pure water was stirred for 1 hour. Then, the organic solvent phase was collected by a separation operation, followed by washing with 30 g of a 1% by weight hydrochloric acid, and washing with 30 g of pure water 4 times. Thereafter, the organic solvent phase was concentrated and dried, thereby obtaining a compound (B1-1-1).

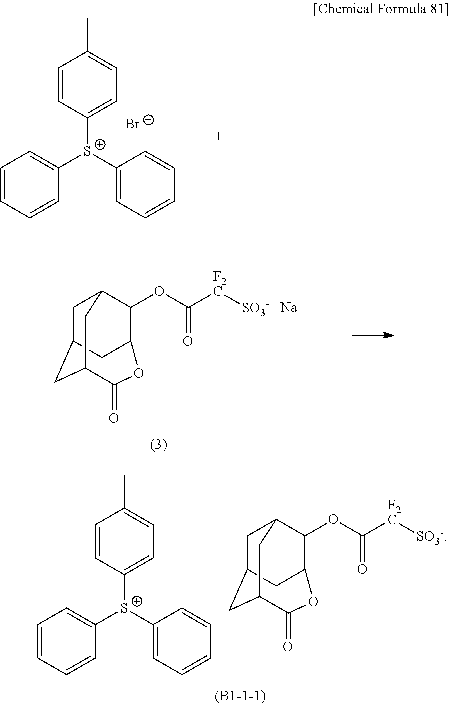

The obtained compound (B1-1-1) was analyzed by ¹H-NMR and ¹⁹F-NMR, and the structure thereof was identified by the following results.

¹H-NMR (DMSO, 400 MHz): δ(ppm)=7.53-7.95 (m, 14H, Ph), 5.02 (s, 1H, CH), 4.21 (s, 1H, CH), 2.93 (s, 1H, CH), 2.52 (s, 3H, CH), 1.41-2.29 (m, 10H, Adamantanelactone).

¹⁹F-NMR (DMSO, 376 MHz): δ(ppm)=−107.3.

From the results of the NMR analyses, it was confirmed that the compound (B1-1-1) had a structure as shown above.

### Example 2

### Synthesis of Compound (B1-1-2)

A compound (4) and a compound (3) were reacted in the same manner as in the synthesis example described in ii) of Example 1, thereby obtaining a compound (B1-1-2).

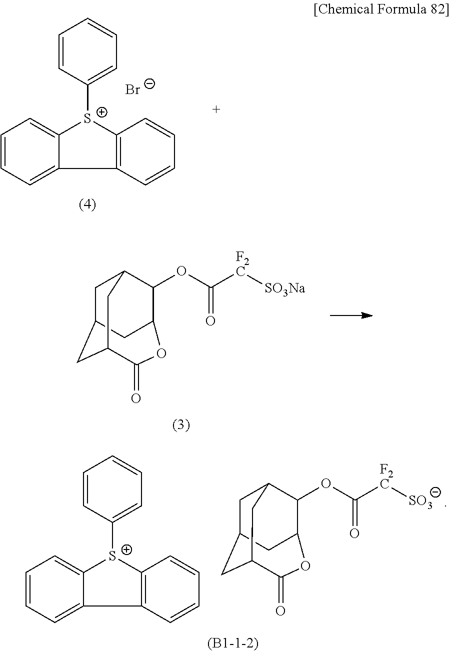

The obtained compound (B1-1-2) was analyzed by ¹H-NMR and ¹⁹F-NMR, and the structure thereof was identified by the following results.

¹H-NMR (DMSO, 400 MHz): δ(ppm)=8.50 (d, 2H, ArH), 8.37 (d, 2H, ArH), 7.93 (t, 2H, ArH), 7.55-7.75 (m, 7H, ArH), 5.02 (s, 1H, CH), 4.21 (s, 1H, CH), 2.93 (s, 1H, CH), 1.41-2.29 (m, 10H, Adamantanelactone).

¹⁹F-NMR (DMSO, 376 MHz): δ(ppm)=−107.3.

From the results of the NMR analyses, it was confirmed that the compound (B1-1-2) had a structure as shown above.

### Example 3

### Synthesis of Compound (B1-1-3)

A compound (5) and a compound (3) were reacted in the same manner as in the synthesis example described in ii) of Example 1, thereby obtaining a compound (B1-1-3). In the reaction formula below, “MS” represents methanesulfonate (the same applies below).

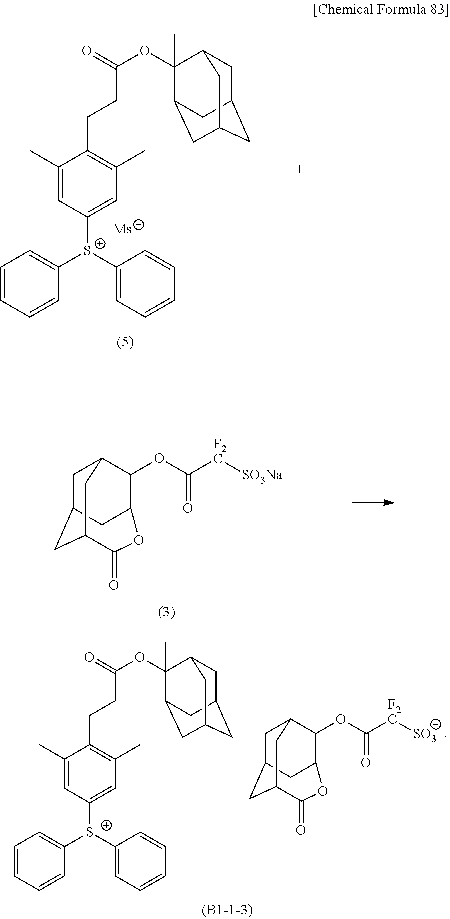

The obtained compound (B1-1-3) was analyzed by ¹H-NMR and ¹⁹F-NMR, and the structure thereof was identified by the following results.

¹H-NMR (DMSO, 400 MHz): δ(ppm)=7.75-7.86 (m, 10H, ArH), 7.61 (s, 2H, ArH), 5.02 (s, 1H, CH), 4.62 (s, 2H, CH₂), 4.21 (s, 1H, CH), 2.93 (s, 1H, CH), 1.41-2.31 (m, 33H, CH₃+Adamantane+Adamantanelactone).

¹⁹F-NMR (DMSO, 376 MHz): δ(ppm)=−107.3.

From the results of the NMR analyses, it was confirmed that the compound (B1-1-3) had a structure as shown above.

### Example 4

### Synthesis of Compound (B1-1-4)

A compound (6) and a compound (3) were reacted in the same manner as in the synthesis example described in ii) of Example 1, thereby obtaining a compound (B1-1-4).

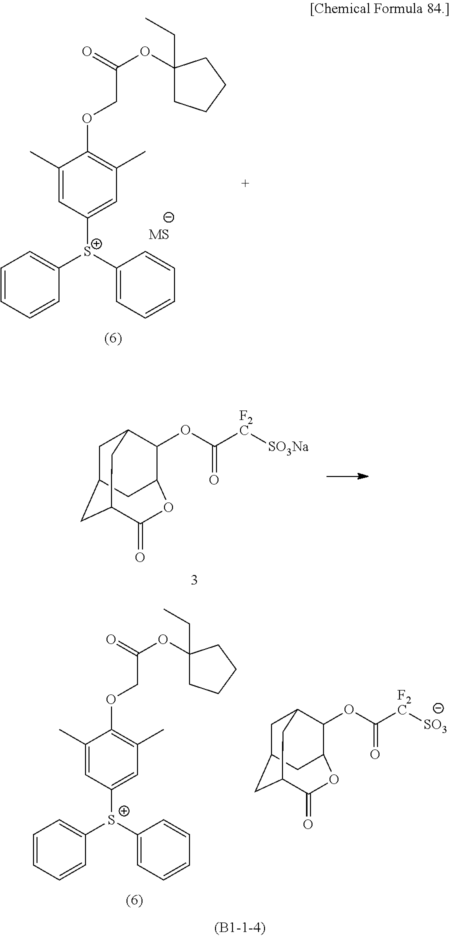

The obtained compound (B1-1-4) was analyzed by ¹H-NMR and ¹⁹F-NMR, and the structure thereof was identified by the following results.

¹H-NMR (DMSO, 400 MHz): δ(ppm)=7.76-7.82 (m, 10H, ArH), 7.59 (s, 2H, ArH), 5.02 (s, 1H, CH), 4.55 (s, 2H, CH₂), 4.21 (s, 1H, CH), 2.93 (s, 1H, CH), 1.41-2.29 (m, 26H, CH₃+cyclopentyl+Adamantanelactone), 0.77-0.81 (t, 3H, CH₃).

¹⁹F-NMR (DMSO, 376 MHz): δ(ppm)=−107.3.

From the results of the NMR analyses, it was confirmed that the compound (B1-1-4) had a structure as shown above.

### Example 5

### Synthesis of Compound (B1-1-5)

A compound (7) and a compound (3) were reacted in the same manner as in the synthesis example described in ii) of Example 1, thereby obtaining a compound (B1-1-5).

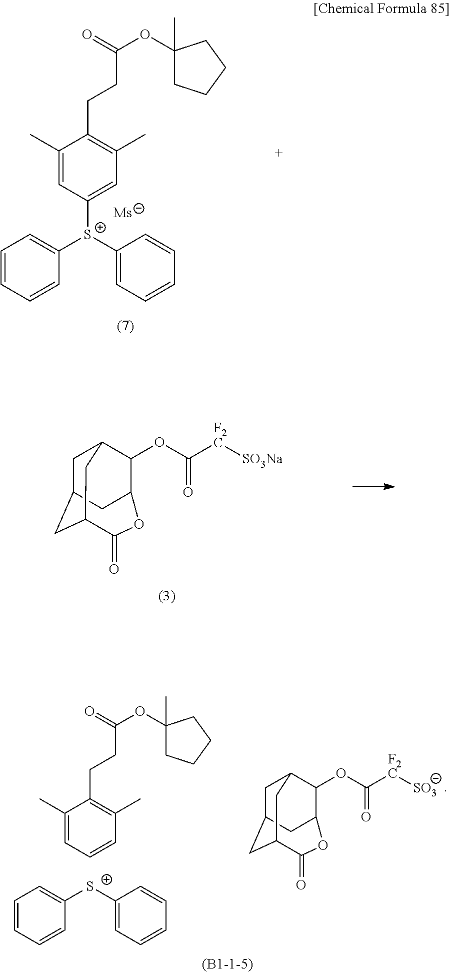

The obtained compound (B1-1-5) was analyzed by ¹H-NMR and ¹⁹F-NMR, and the structure thereof was identified by the following results.

¹H-NMR (DMSO, 400 MHz): δ(ppm)=7.76-7.82 (m, 10H, ArH), 7.59 (s, 2H, ArH), 5.02 (s, 1H, CH), 4.55 (s, 2H, CH₂), 4.21 (s, 1H, CH), 2.93 (s, 1H, CH), 1.43-2.29 (m, 27H, CH₃+cyclopentyl+Adamantanelactone).

¹⁹F-NMR (DMSO, 376 MHz): δ(ppm)=−107.3.

From the results of the NMR analyses, it was confirmed that the compound (B1-1-5) had a structure as shown above.

### Example 6

### Synthesis of Compound (B1-1-6)

A compound (8) and a compound (3) were reacted in the same manner as in the synthesis example described in ii) of Example 1, thereby obtaining a compound (B1-1-6).

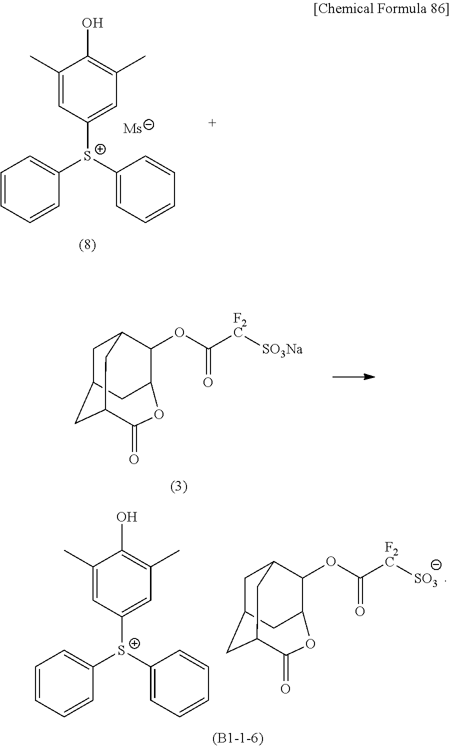

The obtained compound (B1-1-6) was analyzed by ¹H-NMR and ¹⁹F-NMR, and the structure thereof was identified by the following results.

¹H-NMR (DMSO, 400 MHz): δ(ppm)=10.05 (s, 1H₂OH), 7.64-7.87 (m, 10H, ArH), 7.56 (s, 2H, ArH), 5.02 (s, 1H, CH), 4.21 (s, 1H, CH), 2.93 (s, 1H, CH), 1.41-2.29 (m, 16H, CH₃+Adamantanelactone).

¹⁹F-NMR (DMSO, 376 MHz): δ(ppm)=−107.3.

From the results of the NMR analyses, it was confirmed that the compound (B1-1-6) had a structure as shown above.

### Example 7

### Synthesis of Compound (B1-1-7)

A compound (9) and a compound (3) were reacted in the same manner as in the synthesis example described in ii) of Example 1, thereby obtaining a compound (B1-1-7).

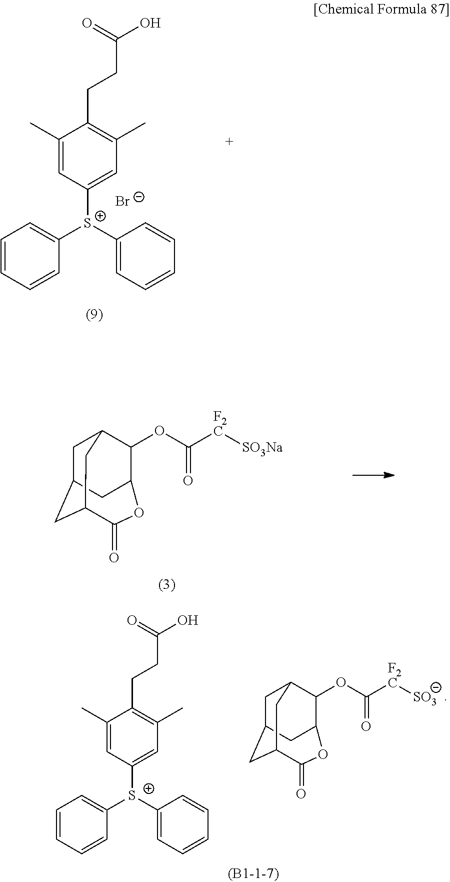

The obtained compound (B1-1-7) was analyzed by ¹H-NMR and ¹⁹F-NMR, and the structure thereof was identified by the following results.

¹H-NMR (DMSO, 400 MHz): δ(ppm)=7.71-7.89 (m, 10H, ArH), 7.59 (s, 2H, ArH), 5.02 (s, 1H, CH), 4.53 (s, 2H, CH₂), 4.21 (s, 1H, CH), 2.93 (s, 1H, CH), 1.41-2.30 (d, 16H, CH₃+Adamantanelactone).

¹⁹F-NMR (DMSO, 376 MHz): δ(ppm)=−107.3.

From the results of the NMR analyses, it was confirmed that the compound (B1-1-7) had a structure as shown above.

### Example 8

### Synthesis of Compound (B1-1-8)

A compound (10) and a compound (3) were reacted in the same manner as in the synthesis example described in ii) of Example 1, thereby obtaining a compound (B1-1-8).

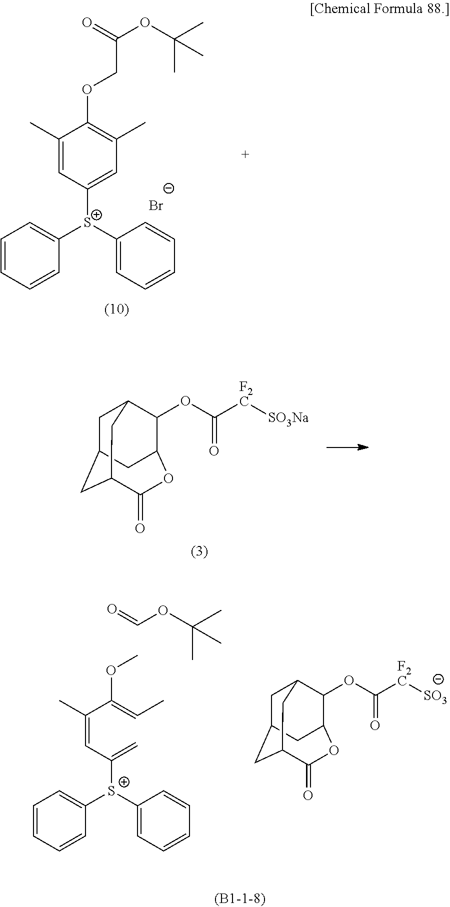

The obtained compound (B1-1-8) was analyzed by ¹H-NMR and ¹⁹F-NMR, and the structure thereof was identified by the following results.

¹H-NMR (DMSO, 400 MHz): δ(ppm)=7.75-7.86 (m, 10H, ArH), 7.63 (s, 2H, ArH), 5.02 (s, 1H, CH), 4.55 (s, 2H, CO—CH₂), 4.21 (s, 1H, CH), 2.93 (s, 1H, CH), 1.41-2.30 (s, 25H, ArCH₃+t-Butyl+Adamantanelatone).

¹⁹F-NMR (DMSO, 376 MHz): δ(ppm)=−107.3

From the results of the NMR analyses, it was confirmed that the compound (B1-1-8) had a structure as shown above.

### Example 9

### Synthesis of Compound (B1-1-9)

A compound (11) and a compound (3) were reacted in the same manner as in the synthesis example described in ii) of Example 1, thereby obtaining a compound (B1-1-9).

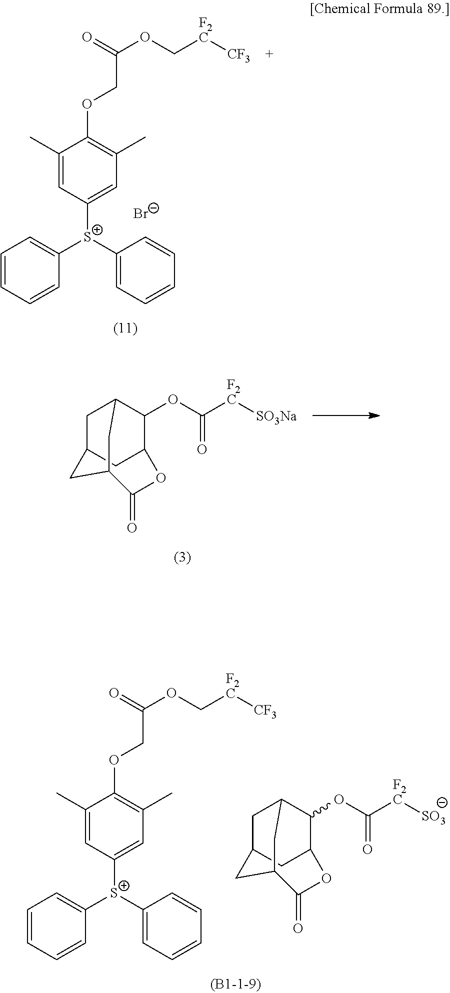

The obtained compound (B1-1-9) was analyzed by ¹H-NMR and ¹⁹F-NMR, and the structure thereof was identified by the following results.

¹H-NMR (DMSO, 400 MHz): δ(ppm)=7.75-7.87 (m, 10H, ArH), 7.63 (s, 2H, ArH), 5.02 (s, 1H, CH), 4.94 (t, 2H₂OCH₂CF₂), 4.84 (s, 2H₂OCH₂), 4.21 (s, 1H, CH), 2.93 (s, 1H, CH), 2.37 (s, 6H, CH₃), 1.41-2.29 (m, 10H, Adamantanelactone).

¹⁹F-NMR (DMSO, 376 MHz): δ(ppm)=−107.3.

From the results of the NMR analyses, it was confirmed that the compound (B1-1-9) had a structure as shown above.

### Example 10

### Synthesis of Compound (B1-1-10)

A compound (12) and a compound (3) were reacted in the same manner as in the synthesis example described in ii) of Example 1, thereby obtaining a compound (B1-1-10).

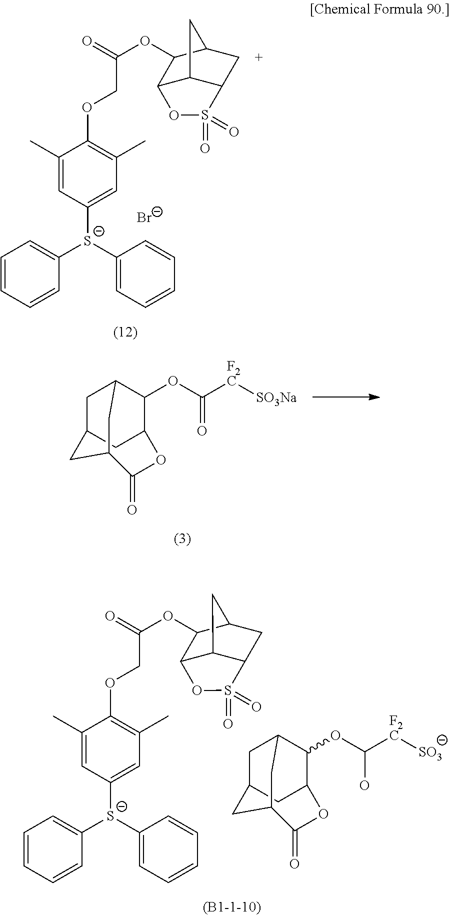

The obtained compound (B1-1-10) was analyzed by ¹H-NMR and ¹⁹F-NMR, and the structure thereof was identified by the following results.

¹H-NMR (DMSO, 400 MHz): δ(ppm)=7.72-7.83 (m, 10H, ArH), 7.59 (s, 2H, ArH), 5.02 (s, 1H, CH), 4.90 (m, 1H, sultone), 4.62-4.68 (m, 3H, CH₂O+sultone), 4.21 (s, 1H, CH), 3.83-3.89 (m, 1H, sultone), 3.43 (m, 1H, sultone), 2.93 (s, 1H, CH), 1.41-2.49 (m, 21H, sultone+Ar—CH₃+Adamantanelactone).

¹⁹F-NMR (DMSO, 376 MHz): δ(ppm)=−107.3.

From the results of the NMR analyses, it was confirmed that the compound (B1-1-10) had a structure as shown above.

### Example 11

### Synthesis of Compound (B1-1-11)

A compound (13) and a compound (3) were reacted in the same manner as in the synthesis example described in ii) of Example 1, thereby obtaining a compound (B1-1-11).

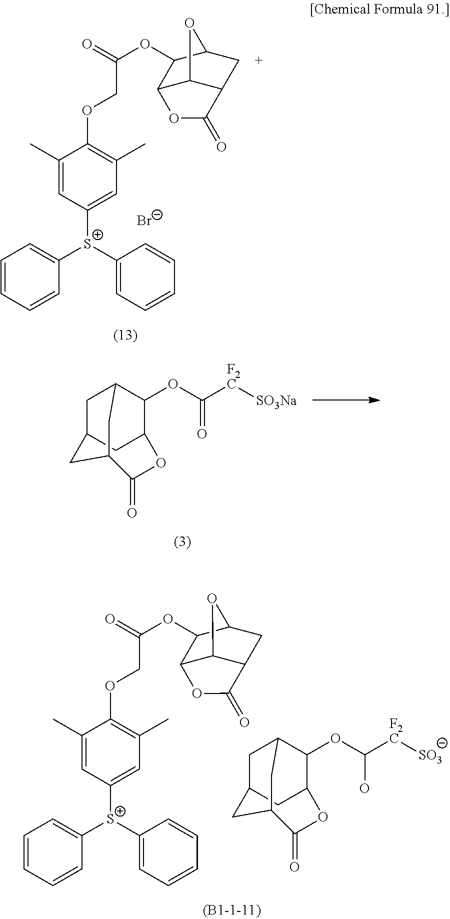

The obtained compound (B1-1-11) was analyzed by ¹H-NMR and ¹⁹F-NMR, and the structure thereof was identified by the following results.

¹H-NMR (DMSO, 400 MHz): δ(ppm)=7.74-7.84 (m, 10H, ArH), 7.61 (s, 2H, ArH), 5.42 (t, 1H, oxo-norbornane), 5.02 (s, 1H, CH), 4.97 (s, 1H, oxo-norbornane), 4.67-4.71 (m, 4H, CH₂+oxo-norbornane), 4.21 (s, 1H, CH), 2.93 (s, 1H, CH), 2.69-2.73 (m, 1H, oxo-norbornane), 2.32 (s, 6H, Ar—CH₃), 1.41-2.16 (m, 12H, oxo-norbornane+Adamantanelactone).

¹⁹F-NMR (DMSO, 376 MHz): δ(ppm)=−107.3

From the results of the NMR analyses, it was confirmed that the compound (B1-1-11) had a structure as shown above.

### Example 12

### Synthesis of Compound (B1-1-12)

A compound (14) and a compound (3) were reacted in the same manner as in the synthesis example described in ii) of Example 1, thereby obtaining a compound (B1-1-12).

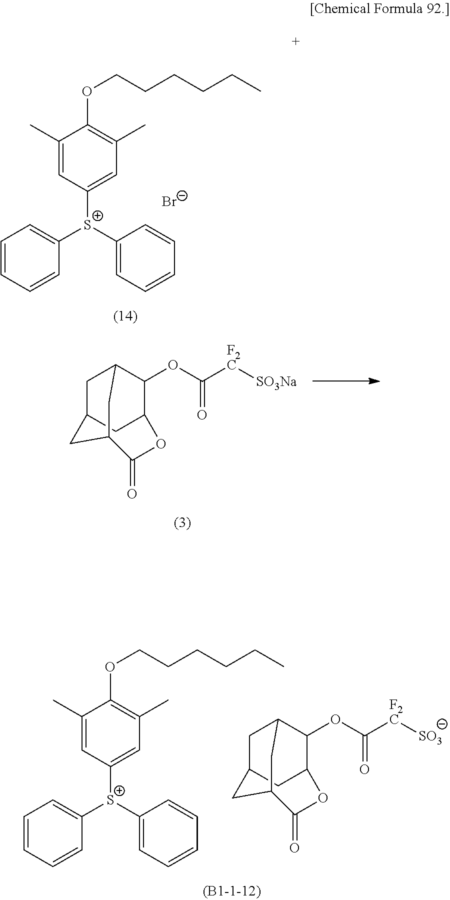

The obtained compound (B1-1-12) was analyzed by ¹H-NMR and ¹⁹F-NMR, and the structure thereof was identified by the following results.

¹H-NMR (DMSO, 400 MHz): δ(ppm)=7.73-7.85 (m, 10H, ArH), 7.59 (s, 2H, ArH), 5.02 (s, 1H, CH), 4.21 (s, 1H, CH), 3.83 (t, 2H₂OCH₂), 2.93 (s, 1H, CH), 1.41-2.33 (m, 20H, CH₃+CH₂+Adamantanelactone), 1.45 (m, 4H, CH₂), 1.29 (m, 4H, CH₂), 0.87 (t, 3H, CH₃).

¹⁹F-NMR (DMSO, 376 MHz): δ(ppm)=−107.3.

From the results of the NMR analyses, it was confirmed that the compound (B1-1-12) had a structure as shown above.

### Example 13

### Synthesis of Compound (B1-1-13)

A compound (15) and a compound (3) were reacted in the same manner as in the synthesis example described in ii) of Example 1, thereby obtaining a compound (B1-1-13).

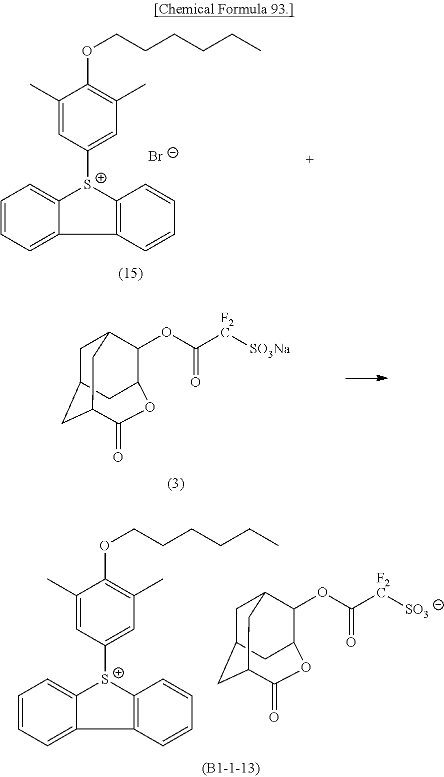

The obtained compound (B1-1-13) was analyzed by ¹H-NMR and ¹⁹F-NMR, and the structure thereof was identified by the following results.

¹H-NMR (DMSO, 400 MHz): δ(ppm)=8.53 (d, 2H, ArH), 8.27 (d, 2H, ArH), 7.95 (t, 2H, ArH), 7.74 (t, 2H, ArH), 7.20 (s, 1H, ArH), 6.38 (s, 1H, ArH), 5.02 (s, 1H, CH), 4.21 (s, 1H, CH), 4.05 (t, 2H, cation-OCH₂), 2.93 (s, 1H, CH), 2.86 (s, 3H, ArCH₃), 1.41-2.29 (m, 15H, Adamantanelactone+ArCH₃+CH₂), 1.37 (quin, 2H, CH₂), 1.24-1.26 (m, 4H, CH₂), 0.82 (t, 3H, CH₃).

¹⁹F-NMR (DMSO, 376 MHz): δ(ppm)=−107.3.

From the results of the NMR analyses, it was confirmed that the compound (B1-1-13) had a structure as shown above.

### Example 14

### Synthesis of Compound (B1-1-14)

A compound (16) and a compound (3) were reacted in the same manner as in the synthesis example described in ii) of Example 1, thereby obtaining a compound (B1-1-14).

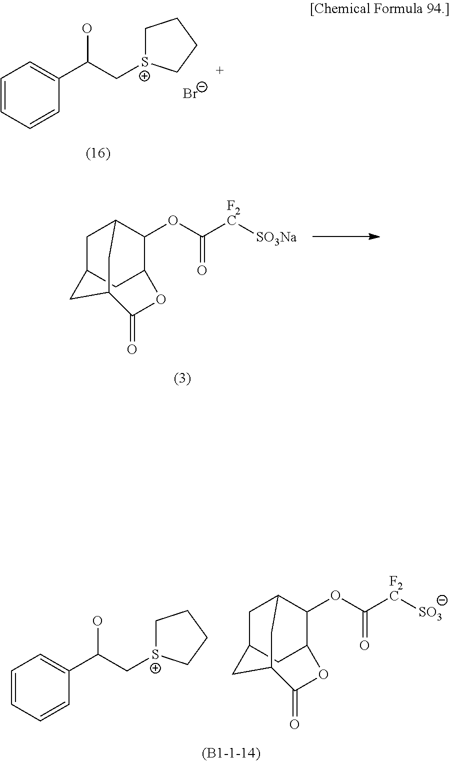

The obtained compound (B1-1-14) was analyzed by ¹H-NMR and ¹⁹F-NMR, and the structure thereof was identified by the following results.

¹H-NMR (DMSO, 400 MHz): δ(ppm)=7.99-8.01 (d, 2H, Ar), 7.73-7.76 (t, 1H, Ar), 7.58-7.61 (t, 2H, Ar), 5.31 (s, 2H, SCH₂C═O), 5.02 (s, 1H, CH), 4.21 (s, 1H, CH), 3.49-3.62 (m, 4H, CH₂), 2.93 (s, 1H, CH), 1.41-2.49 (m, 14H, CH₂S+Adamantanelactone).

¹⁹F-NMR (DMSO, 376 MHz): δ(ppm)=−107.3.

From the results of the NMR analyses, it was confirmed that the compound (B1-1-14) had a structure as shown above.

### Example 15

### Synthesis of Compound (B1-1-15)

A compound (17) and a compound (3) were reacted in the same manner as in the synthesis example described in ii) of Example 1, thereby obtaining a compound (B1-1-15).

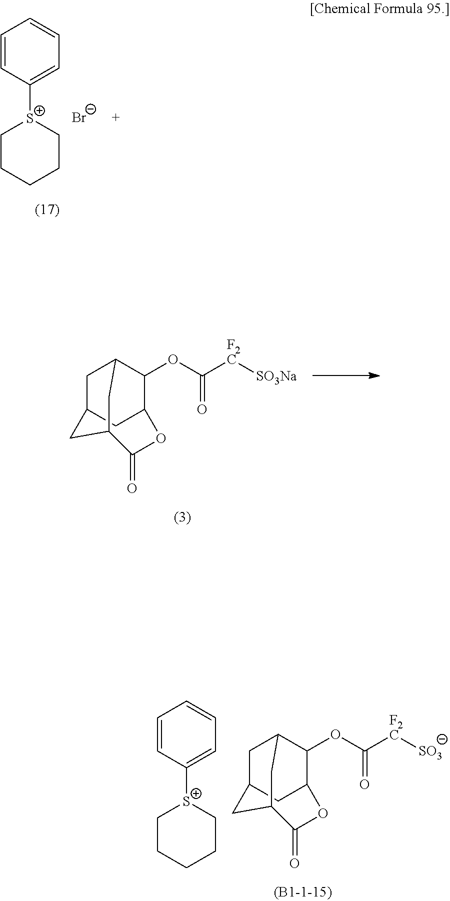

The obtained compound (B1-1-15) was analyzed by ¹H-NMR and ¹⁹F-NMR, and the structure thereof was identified by the following results.

¹H-NMR (DMSO, 400 MHz): δ(ppm)=8.02-8.05 (m, 2H, Phenyl), 7.61-7.73 (m, 3H, Phenyl), 5.02 (s, 1H, CH), 4.21 (s, 1H, CH), 3.76-3.86 (m, 4H, SCH₂), 2.93 (s, 1H, CH), 1.41-2.29 (m, 16H, CH₂+Adamantanelactone).

¹⁹F-NMR (DMSO, 376 MHz): δ(ppm)=−107.3.

From the results of the NMR analyses, it was confirmed that the compound (B1-1-15) had a structure as shown above.

### Example 16

### Synthesis of Compound (B1-1-16)

A compound (18) and a compound (3) were reacted in the same manner as in the synthesis example described in ii) of Example 1, thereby obtaining a compound (B1-1-16).

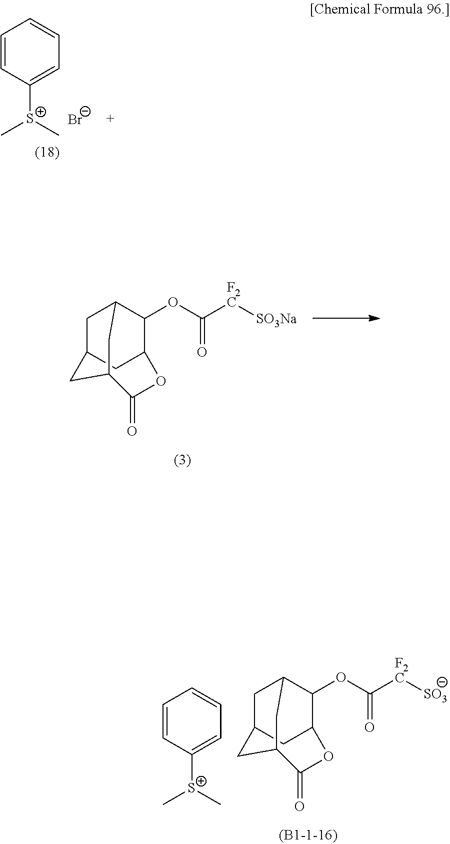

The obtained compound (B1-1-16) was analyzed by ¹H-NMR and ¹⁹F-NMR, and the structure thereof was identified by the following results.

¹H-NMR (DMSO, 400 MHz): δ(ppm)=8.04-8.09 (m, 2H, Phenyl), 7.69-7.79 (m, 3H, Phenyl), 5.02 (s, 1H, CH), 4.21 (s, 1H, CH), 3.29 (s, 6H, CH₃), 2.93 (s, 1H, CH), 1.41-2.29 (m, 10H, Adamantanelactone).

¹⁹F-NMR (DMSO, 376 MHz): δ(ppm)=−107.3.

From the results of the NMR analyses, it was confirmed that the compound (B1-1-16) had a structure as shown above.

### Example 17

### Synthesis of Compound (B1-1-17)

A compound (19) and a compound (3) were reacted in the same manner as in the synthesis example described in ii) of Example 1, thereby obtaining a compound (B1-1-17).

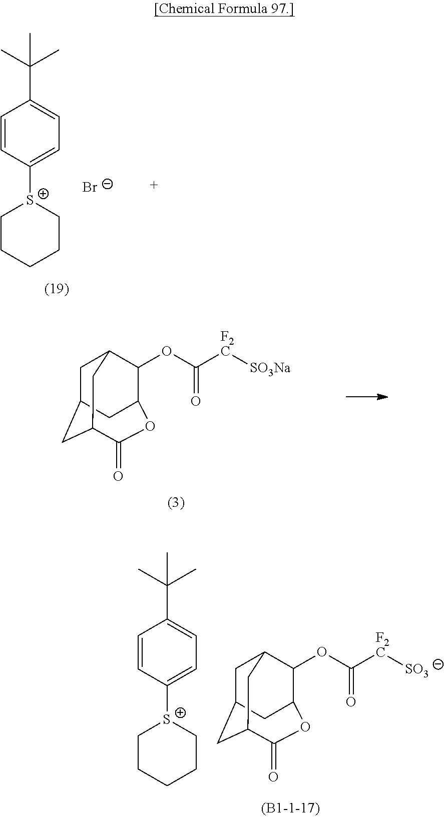

The obtained compound (B1-1-17) was analyzed by ¹H-NMR and ¹⁹F-NMR, and the structure thereof was identified by the following results.

¹H-NMR (DMSO, 400 MHz): δ(ppm)=8.07 (d, 2H, Phenyl), 7.81 (d, 2H, Phenyl), 5.02 (s, 1H, CH), 4.21 (s, 1H, CH), 4.10 (t, 2H, CH₂), 3.59 (d, 2H, CH₂), 2.93 (s, 1H, CH), 1.41-2.29 (m, 16H, CH₂+CH₂+Adamantanelactone), 1.71-2.19 (m, 4H, CH₂), 1.23 (s, 9H, t-Bu).

¹⁹F-NMR (DMSO, 376 MHz): δ(ppm)=−107.3.

From the results of the NMR analyses, it was confirmed that the compound (B1-1-17) had a structure as shown above.

### Example 18

### Synthesis of Compound (B1-1-18)

A compound (20) and a compound (3) were reacted in the same manner as in the synthesis example described in ii) of Example 1, thereby obtaining a compound (B1-1-18).

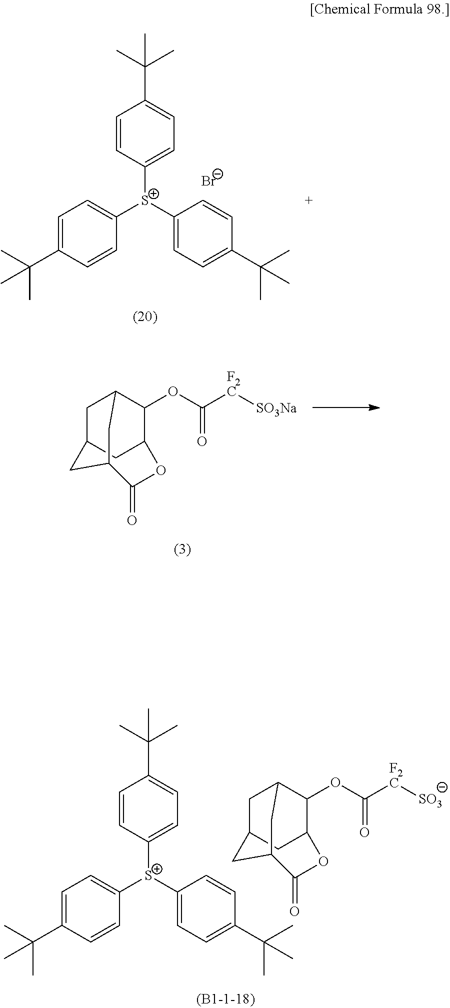

The obtained compound (B1-1-18) was analyzed by ¹H-NMR and ¹⁹F-NMR, and the structure thereof was identified by the following results.

¹H-NMR (DMSO, 400 MHz): δ(ppm)=7.84 (d, 6H, ArH), 7.78 (d, 6H, ArH), 5.02 (s, 1H, CH), 4.21 (s, 1H, CH), 2.93 (s, 1H, CH), 1.41-2.29 (m, 10H, Adamantanelactone), 1.33 (s, 27H, tBu-CH₃).

¹⁹F-NMR (DMSO, 376 MHz): δ(ppm)=−107.3.

From the results of the NMR analyses, it was confirmed that the compound (B1-1-18) had a structure as shown above.

### Example 19

### Synthesis of Compound (B1-1-19)

A compound (21) and a compound (3) were reacted in the same manner as in the synthesis example described in ii) of Example 1, thereby obtaining a compound (B1-1-19).

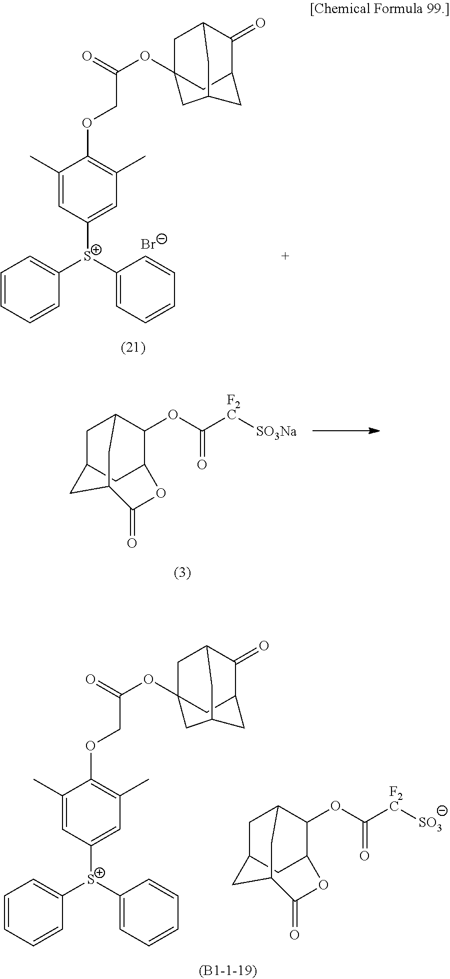

The obtained compound (B1-1-19) was analyzed by ¹H-NMR and ¹⁹F-NMR, and the structure thereof was identified by the following results.

¹H-NMR (DMSO, 400 MHz): δ(ppm)=7.72-7.84 (m, 10H, ArH), 7.59 (s, 2H, ArH), 5.02 (s, 1H, CH), 4.56 (s, 2H, CH₂), 4.21 (s, 1H, CH), 2.93 (s, 1H, CH), 2.49 (m, 2H, Adamantane), 1.41-2.34 (m, 27H, Adamantan+Adamantanelactone).

¹⁹F-NMR (DMSO, 376 MHz): δ(ppm)=−107.3.

From the results of the NMR analyses, it was confirmed that the compound (B1-1-19) had a structure as shown above.

### Example 20

### Synthesis of Compound (B1-1-20)

A compound (22) and a compound (3) were reacted in the same manner as in the synthesis example described in ii) of Example 1, thereby obtaining a compound (B1-1-20).

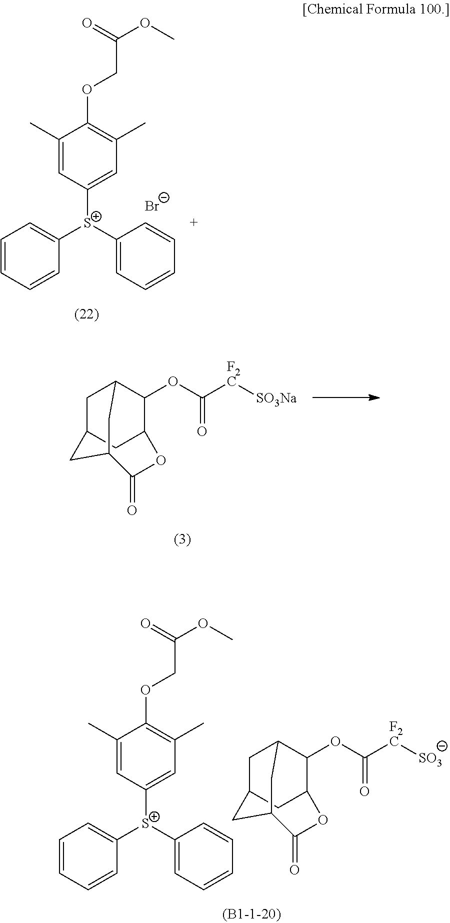

The obtained compound (B1-1-20) was analyzed by ¹H-NMR and ¹⁹F-NMR, and the structure thereof was identified by the following results.

¹H-NMR (DMSO, 400 MHz): δ(ppm)=7.72-7.84 (m, 10H, ArH), 7.59 (s, 2H, ArH), 5.02 (s, 1H, CH), 4.64 (s, 2H, CH₂), 4.21 (s, 1H, CH), 3.70 (s, 3H₂OCH₃), 2.93 (s, 1H, CH), 1.41-2.29 (m, 16H, CH₃+Adamantanelactone).

¹⁹F-NMR (DMSO, 376 MHz): δ(ppm)=−107.3

From the results of the NMR analyses, it was confirmed that the compound (B1-1-20) had a structure as shown above.

### Example 21

### Synthesis of Compound (B1-1-21)

A compound (23) and a compound (3) were reacted in the same manner as in the synthesis example described in ii) of Example 1, thereby obtaining a compound (B1-1-21).

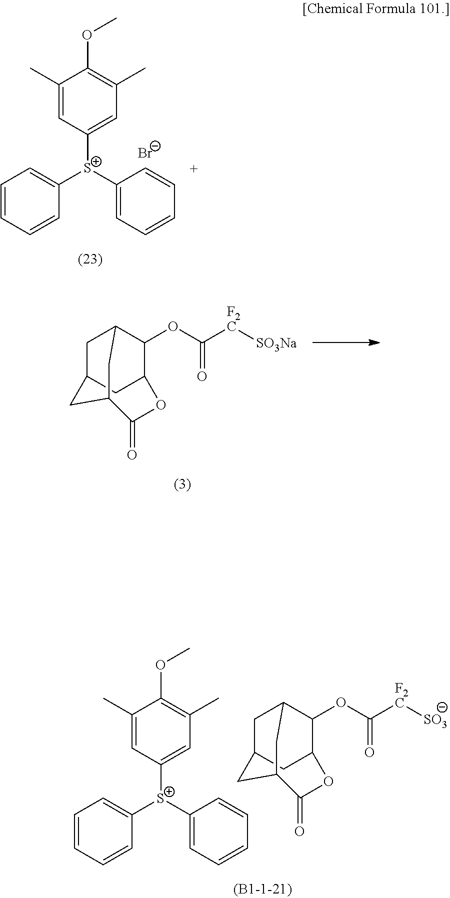

The obtained compound (B1-1-21) was analyzed by ¹H-NMR and ¹⁹F-NMR, and the structure thereof was identified by the following results.

¹H-NMR (DMSO, 400 MHz): δ(ppm)=7.78-7.89 (m, 10H, ArH), 7.64 (s, 2H, ArH), 5.02 (s, 1H, CH), 4.21 (s, 1H, CH), 3.79 (s, 3H₂OCH₃), 2.93 (s, 1H, CH), 2.32 (s, 6H, CH₃), 1.41-2.29 (m, 10H, Adamantanelactone).

¹⁹F-NMR (DMSO, 376 MHz): δ(ppm)=−107.3.

From the results of the NMR analyses, it was confirmed that the compound (B1-1-21) had a structure as shown above.

### Example 22

### Synthesis of Compound (B1-1-22)

A compound (24) and a compound (3) were reacted in the same manner as in the synthesis example described in ii) of Example 1, thereby obtaining a compound (B1-1-22).

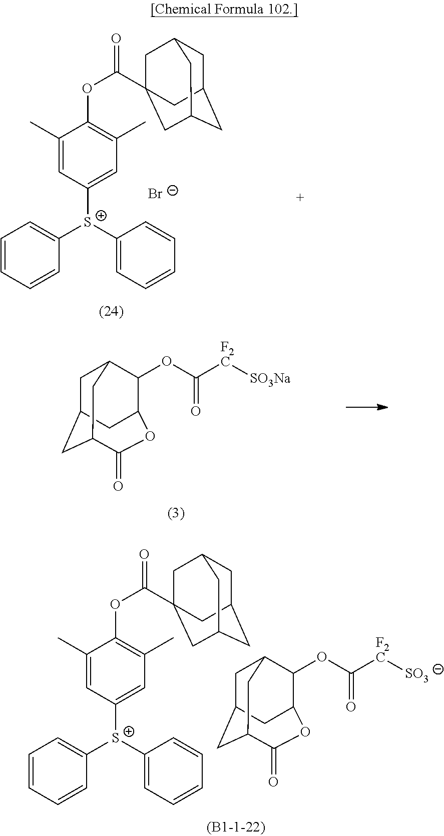

The obtained compound (B1-1-22) was analyzed by ¹H-NMR and ¹⁹F-NMR, and the structure thereof was identified by the following results.

¹H-NMR (DMSO, 400 MHz): δ(ppm)=7.76-7.87 (m, 10H, ArH), 7.69 (s, 2H, ArH), 5.02 (s, 1H, CH), 4.21 (s, 1H, CH), 2.93 (s, 1H, CH), 1.41-2.29 (m, 31H, CH₃+Adamantane+Adamantanelactone).

¹⁹F-NMR (DMSO, 376 MHz): δ(ppm)=−107.3.

From the results of the NMR analyses, it was confirmed that the compound (B1-1-22) had a structure as shown above.

### Example 23

### Synthesis of Compound (B1-1-23)

A compound (25) and a compound (3) were reacted in the same manner as in the synthesis example described in ii) of Example 1, thereby obtaining a compound (B1-1-23).

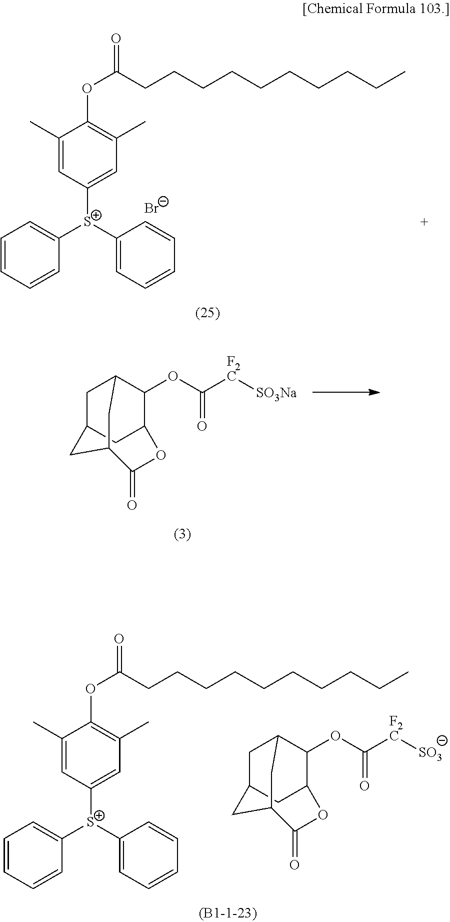

The obtained compound (B1-1-23) was analyzed by ¹H-NMR and ¹⁹F-NMR, and the structure thereof was identified by the following results.

¹H-NMR (DMSO, 400 MHz): δ(ppm)=7.79-7.93 (m, 12H, ArH), 5.02 (s, 1H, CH), 4.21 (s, 1H, CH), 2.93 (s, 1H, CH), 2.73 (t, 2H, CO—CH₂), 1.41-2.29 (m, 18H, ArCH₃+CH₂+Adamantanelactone), 1.25-1.38 (m, 14H, CH₂), 0.85 (t, 3H, CH₃).

¹⁹F-NMR (DMSO, 376 MHz): δ(ppm)=−107.3.

From the results of the NMR analyses, it was confirmed that the compound (B1-1-23) had a structure as shown above.

### Example 24

### Synthesis of Compound (B1-1-24)

A compound (26) and a compound (3) were reacted in the same manner as in the synthesis example described in ii) of Example 1, thereby obtaining a compound (B1-1-24).

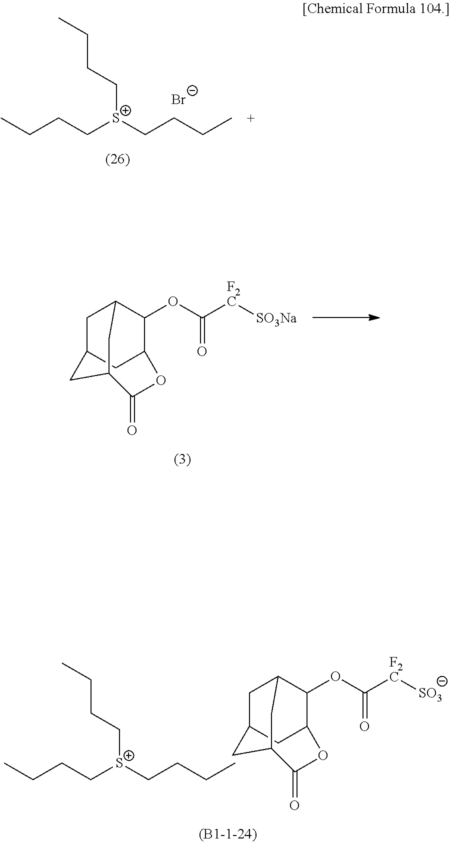

The obtained compound (B1-1-24) was analyzed by ¹H-NMR and ¹⁹F-NMR, and the structure thereof was identified by the following results.

¹H-NMR (DMSO, 400 MHz): δ(ppm)=5.02 (s, 1H, CH), 4.21 (s, 1H, CH), 3.36 (t, 6H, CH₂), 2.93 (s, 1H, CH), 1.35-2.29 (m, 22H, CH₂+CH₂+Adamantanelactone), 0.81-0.93 (m, 9H, CH₃).

¹⁹F-NMR (DMSO, 376 MHz): δ(ppm)=−107.3.

From the results of the NMR analyses, it was confirmed that the compound (B1-1-24) had a structure as shown above.

### Example 25

### Synthesis of Compound (B1-1-25)

A compound (27) and a compound (3) were reacted in the same manner as in the synthesis example described in ii) of Example 1, thereby obtaining a compound (B1-1-25).

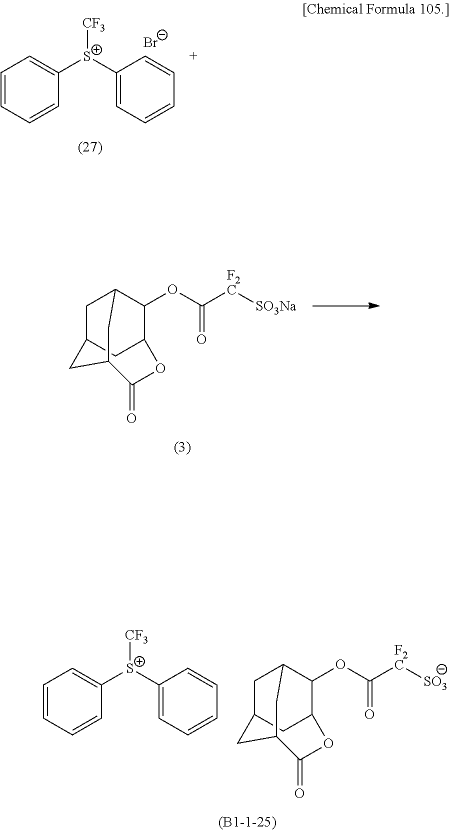

The obtained compound (B1-1-25) was analyzed by ¹H-NMR and ¹⁹F-NMR, and the structure thereof was identified by the following results.

¹H-NMR (DMSO, 400 MHz): δ(ppm)=8.29 (d, 4H, ArH), 7.93-8.09 (m, 6H, ArH), 5.02 (s, 1H, CH), 4.21 (s, 1H, CH), 2.93 (s, 1H, CH), 1.41-2.29 (m, 10H, Adamantanelactone).

¹⁹F-NMR (DMSO, 376 MHz): δ(ppm)=−47.9, −107.3.

From the results of the NMR analyses, it was confirmed that the compound (B1-1-25) had a structure as shown above.

### Example 26

### Synthesis of Compound (B1-1-26)

A compound (28) and a compound (3) were reacted in the same manner as in the synthesis example described in ii) of Example 1, thereby obtaining a compound (B1-1-26).

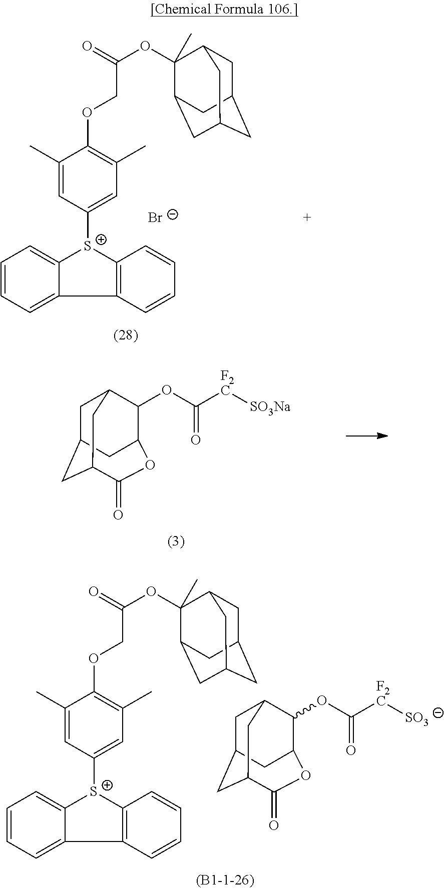

The obtained compound (B1-1-26) was analyzed by ¹H-NMR and ¹⁹F-NMR, and the structure thereof was identified by the following results.

¹H-NMR (DMSO, 400 MHz): δ(ppm)=8.49 (d, 2H, ArH), 8.30 (d, 2H, ArH), 7.93 (t, 2H, ArH), 7.73 (t, 2H, ArH), 7.30 (s, 2H, ArH), 5.02 (s, 1H, CH), 4.52 (s, 2H₂OCH₂), 4.21 (s, 1H, CH), 2.93 (s, 1H, CH), 1.41-2.29 (m, 33H, Ar—CH₃+Adamantane+CH₃+Adamantanelactone).

¹⁹F-NMR (DMSO, 376 MHz): δ(ppm)=−107.3.

From the results of the NMR analyses, it was confirmed that the compound (B1-1-26) had a structure as shown above.

### Example 27

### Synthesis of Compound (B1-1-27)

A compound (29) and a compound (3) were reacted in the same manner as in the synthesis example described in ii) of Example 1, thereby obtaining a compound (B1-1-27).

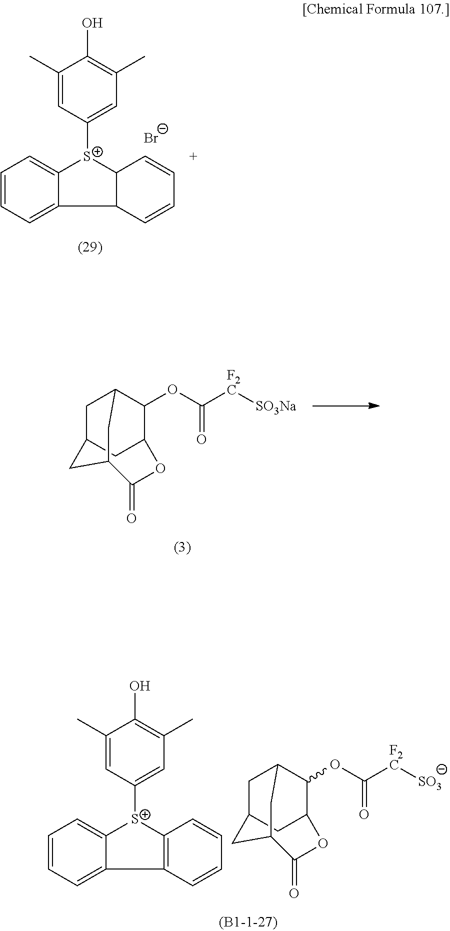

The obtained compound (B1-1-27) was analyzed by ¹H-NMR and ¹⁹F-NMR, and the structure thereof was identified by the following results.

¹H-NMR (DMSO, 400 MHz): δ(ppm)=9.73 (br s, 1H₂OH), 8.47 (d, 2H, ArH), 8.24 (d, 2H, ArH), 7.91 (t, 2H, ArH), 7.71 (t, 2H, ArH), 7.18 (s, 2H, ArH), 5.02 (s, 1H, CH), 4.21 (s, 1H, CH), 2.93 (s, 1H, CH), 1.41-2.29 (m, 16H, ArCH₃+Adamantanelactone).

¹⁹F-NMR (DMSO, 376 MHz): δ(ppm)=−107.3.

From the results of the NMR analyses, it was confirmed that the compound (B1-1-27) had a structure as shown above.

### Example 28

### Synthesis of Compound (B1-1-28)

A compound (30) and a compound (3) were reacted in the same manner as in the synthesis example described in ii) of Example 1, thereby obtaining a compound (B1-1-28).

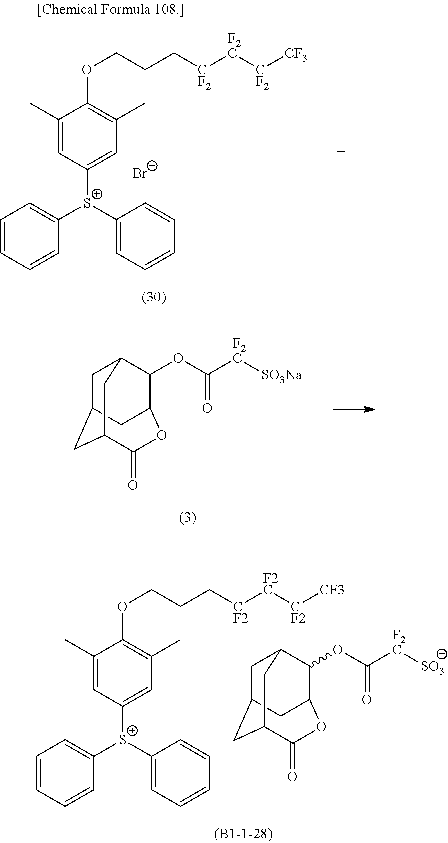

The obtained compound (B1-1-28) was analyzed by ¹H-NMR and ¹⁹F-NMR, and the structure thereof was identified by the following results.

¹H-NMR (DMSO, 400 MHz): δ(ppm)=7.75-7.87 (m, 10H, ArH), 7.62 (s, 2H, ArH), 5.02 (s, 1H, CH), 4.21 (s, 1H, CH), 3.97 (t, 2H, CH₂), 2.93 (s, 1H, CH), 1.41-2.56 (m, 20H, CH₂+CH₃+Adamantanelactone).

¹⁹F-NMR (DMSO, 376 MHz): δ(ppm)=−123.5, −121.8, −111.6, −107.3, −78.3.

From the results of the NMR analyses, it was confirmed that the compound (B1-1-28) had a structure as shown above.

### Example 29

### Synthesis of Compound (B1-1-29)

A compound (31) and a compound (3) were reacted in the same manner as in the synthesis example described in ii) of Example 1, thereby obtaining a compound (B1-1-29).

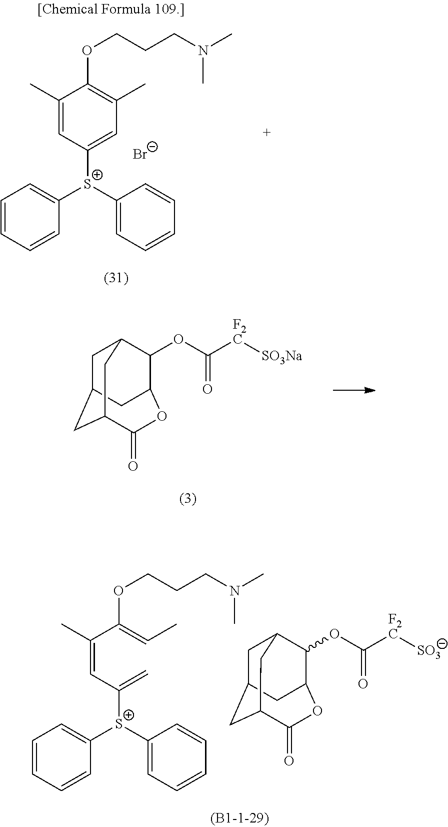

The obtained compound (B1-1-29) was analyzed by ¹H-NMR and ¹⁹F-NMR, and the structure thereof was identified by the following results.

¹H-NMR (DMSO, 400 MHz): δ(ppm)=7.75-7.86 (m, 10H, ArH), 7.60 (s, 2H, ArH), 5.02 (s, 1H, CH), 4.21 (s, 1H, CH), 3.87 (t, 2H, CH₂), 2.93 (s, 1H, CH), 2.40 (m, 2H, CH₂), 1.41-2.35 (m, 24H, CH₂+N—CH₃+CH₂+Adamantanelactone).

¹⁹F-NMR (DMSO, 376 MHz): δ(ppm)=−107.3.

From the results of the NMR analyses, it was confirmed that the compound (B1-1-29) had a structure as shown above.

### Example 30

### Synthesis of Compound (B1-1-30)

A compound (32) and a compound (3) were reacted in the same manner as in the synthesis example described in ii) of Example 1, thereby obtaining a compound (B1-1-30).

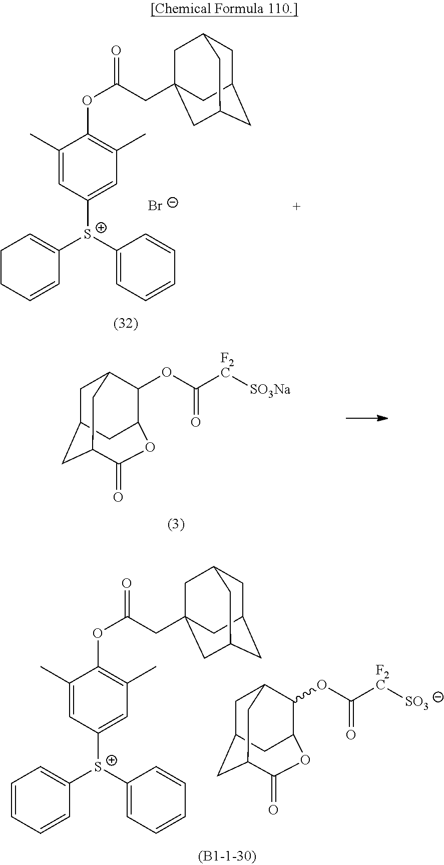

The obtained compound (B1-1-30) was analyzed by ¹H-NMR and ¹⁹F-NMR, and the structure thereof was identified by the following results.

¹H-NMR (DMSO, 400 MHz): δ(ppm)=7.77-7.89 (m, 10H, ArH), 7.71 (s, 2H, ArH), 2.51 (s, 2H, CH₂), 5.02 (s, 1H, CH), 4.21 (s, 1H, CH), 2.93 (s, 1H, CH), 1.41-2.29 (m, 31H, CH₃+Adamantane+Adamantanelactone).

¹⁹F-NMR (DMSO, 376 MHz): δ(ppm)=−107.3.

From the results of the NMR analyses, it was confirmed that the compound (B1-1-30) had a structure as shown above.

### Example 31

### Synthesis of Compound (B1-1-31)

A compound (33) and a compound (3) were reacted in the same manner as in the synthesis example described in ii) of Example 1, thereby obtaining a compound (B1-1-31).

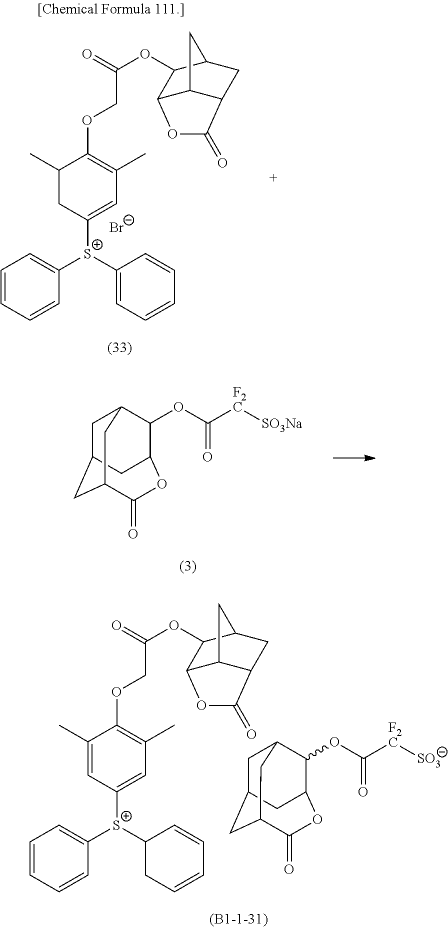

The obtained compound (B1-1-31) was analyzed by ¹H-NMR and ¹⁹F-NMR, and the structure thereof was identified by the following results.

¹H-NMR (DMSO, 400 MHz): δ(ppm)=7.74-7.84 (m, 10H, ArH), 7.61 (s, 2H, ArH), 5.02 (s, 1H, CH), 4.49-4.66 (m, 4H, norbornane+OCH₂), 4.21 (s, 1H, CH), 3.24 (m, 1H, norbornane), 2.93 (s, 1H, CH), 2.44-2.54 (m, 2H, norbornane), 2.37 (s, 6H, ArCH₃), 1.41-2.29 (m, 14H, Norbornane+Adamantanelactone).

¹⁹F-NMR (DMSO, 376 MHz): δ(ppm)=−107.3.

From the results of the NMR analyses, it was confirmed that the compound (B1-1-31) had a structure as shown above.

### Example 32

### Synthesis of Compound (B1-1-32)

A compound (34) and a compound (3) were reacted in the same manner as in the synthesis example described in ii) of Example 1, thereby obtaining a compound (B1-1-32).

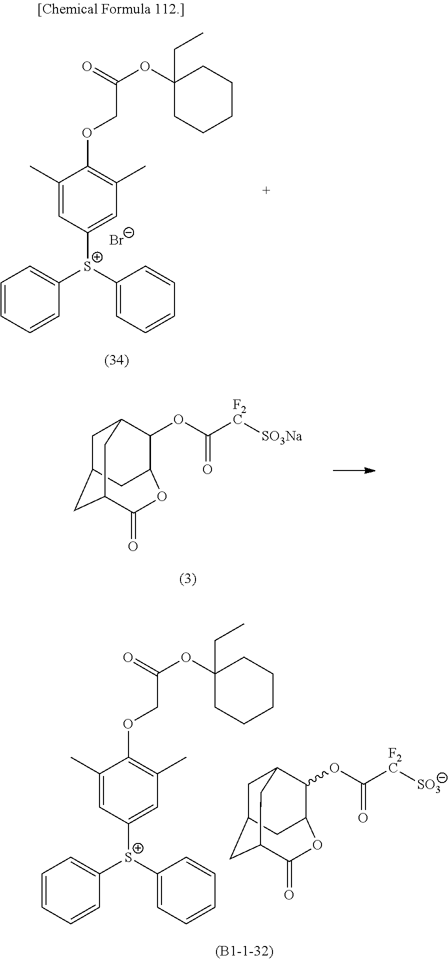

The obtained compound (B1-1-32) was analyzed by ¹H-NMR and ¹⁹F-NMR, and the structure thereof was identified by the following results.

¹H-NMR (DMSO, 400 MHz): δ(ppm)=7.80-7.92 (m, 10H, ArH), 7.67 (s, 2H, ArH), 5.02 (s, 1H, CH), 4.66 (s, 2H, CH₂), 4.21 (s, 1H, CH), 2.93 (s, 1H, CH), 2.37 (s, 6H, ArCH₃), 1.41-2.29 (m, 14H, Cyclohexyl+CH₂+Adamantanelactone), 1.14-1.57 (m, 8H, cyclohexyl), 0.84 (t, 3H, CH₃).

¹⁹F-NMR (DMSO, 376 MHz): δ(ppm)=−107.3.

From the results of the NMR analyses, it was confirmed that the compound (B1-1-32) had a structure as shown above.

### Example 33

### Synthesis of Compound (B1-1-33)

A compound (35) and a compound (3) were reacted in the same manner as in the synthesis example described in ii) of Example 1, thereby obtaining a compound (B1-1-33).

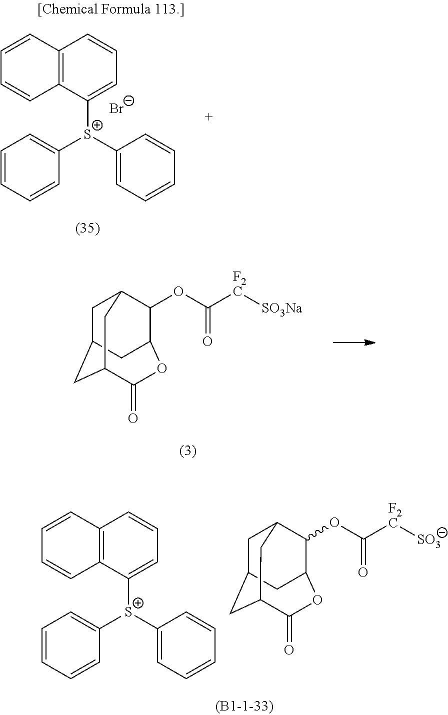

The obtained compound (B1-1-33) was analyzed by ¹H-NMR and ¹⁹F-NMR, and the structure thereof was identified by the following results.

¹H-NMR (DMSO, 400 MHz): δ(ppm)=8.44 (d, 1H, ArH), 8.22 (m, 2H, ArH), 7.73-7.89 (m, 13H, ArH), 7.50 (d, 1H, ArH), 5.02 (s, 1H, CH), 4.21 (s, 1H, CH), 2.93 (s, 1H, CH), 1.41-2.29 (m, 10H, Adamantanelactone).

¹⁹F-NMR (DMSO, 376 MHz): δ(ppm)=−107.3.

From the results of the NMR analyses, it was confirmed that the compound (B1-1-33) had a structure as shown above.

### Example 34

### Synthesis of Compound (B1-1-34)

A compound (36) and a compound (3) were reacted in the same manner as in the synthesis example described in ii) of Example 1, thereby obtaining a compound (B1-1-34).

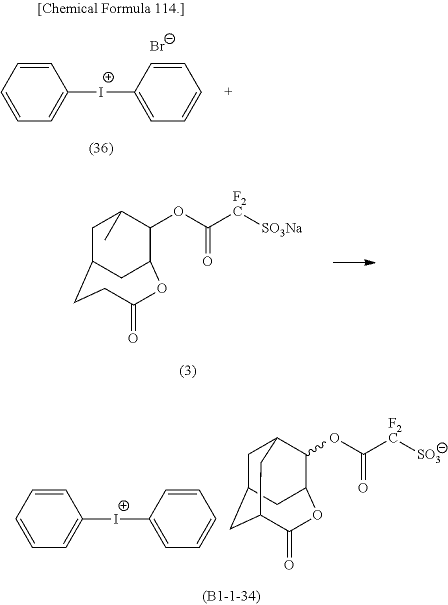

The obtained compound (B1-1-34) was analyzed by ¹H-NMR and ¹⁹F-NMR, and the structure thereof was identified by the following results.

¹H-NMR (DMSO, 400 MHz): δ(ppm)=8.24 (d, 4H, ArH), 7.59 (t, 2H, ArH), 7.47 (t, 4H, ArH), 5.02 (s, 1H, CH), 4.21 (s, 1H, CH), 2.93 (s, 1H, CH), 1.41-2.29 (m, 10H, Adamantanelactone).

¹⁹F-NMR (DMSO, 376 MHz): δ(ppm)=−107.3.

From the results of the NMR analyses, it was confirmed that the compound (B1-1-34) had a structure as shown above.

### Example 35

### Synthesis of Compound (B1-1-35)

A compound (37) and a compound (3) were reacted in the same manner as in the synthesis example described in ii) of Example 1, thereby obtaining a compound (B1-1-35).

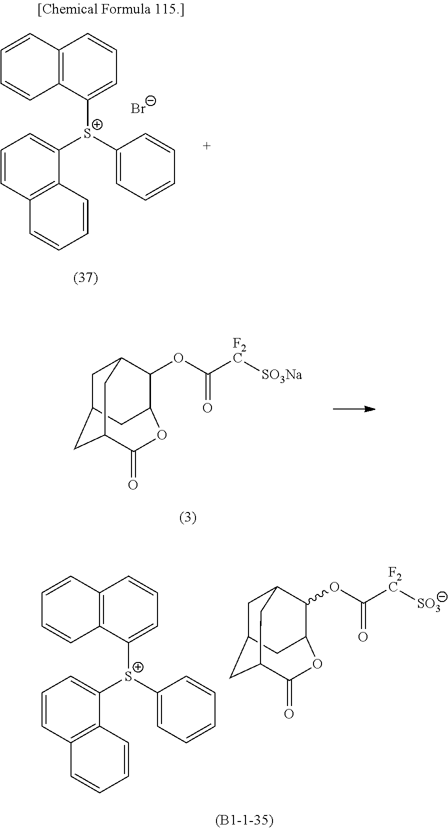

The obtained compound (B1-1-35) was analyzed by ¹H-NMR and ¹⁹F-NMR, and the structure thereof was identified by the following results.

¹H-NMR (DMSO, 400 MHz): δ(ppm)=8.55 (d, 2H, ArH), 8.38 (d, 2H, ArH), 8.32 (d, 2H, ArH), 8.03 (d, 2H, ArH), 7.93-7.97 (m, 1H, ArH), 7.82-7.88 (m, 8H, ArH), 7.55 (d, 2H, ArH), 5.02 (s, 1H, CH), 4.21 (s, 1H, CH), 2.93 (s, 1H, CH), 1.41-2.29 (m, 10H, Adamantanelactone).

¹⁹F-NMR (DMSO, 376 MHz): δ(ppm)=−107.3.

From the results of the NMR analyses, it was confirmed that the compound (B1-1-35) had a structure as shown above.

### Example 36

### Synthesis of Compound (B1-1-36)

A compound (38) and a compound (3) were reacted in the same manner as in the synthesis example described in ii) of Example 1, thereby obtaining a compound (B1-1-36).

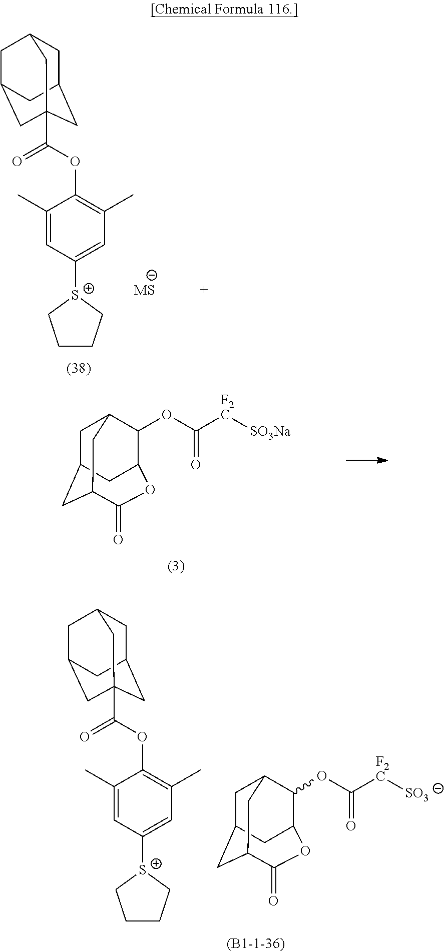

The obtained compound (B1-1-36) was analyzed by ¹H-NMR and ¹⁹F-NMR, and the structure thereof was identified by the following results.

¹H-NMR (DMSO, 400 MHz): δ(ppm)=7.75 (s, 2H, Ar), 5.02 (s, 1H, CH), 4.21 (s, 1H, CH), 3.91-3.96 (m, 2H, CH₂), 3.72-3.79 (m, 2H, CH₂), 2.93 (s, 1H, CH), 1.41-2.41 (m, 35H, CH₂+Ar—CH₃+Adamantane+Adamantanelactone).

¹⁹F-NMR (DMSO, 376 MHz): δ(ppm)=−107.3.

From the results of the NMR analyses, it was confirmed that the compound (B1-1-36) had a structure as shown above.

### Example 37

### Synthesis of Compound (B1-1-37)

A compound (39) and a compound (3) were reacted in the same manner as in the synthesis example described in ii) of Example 1, thereby obtaining a compound (B1-1-37).

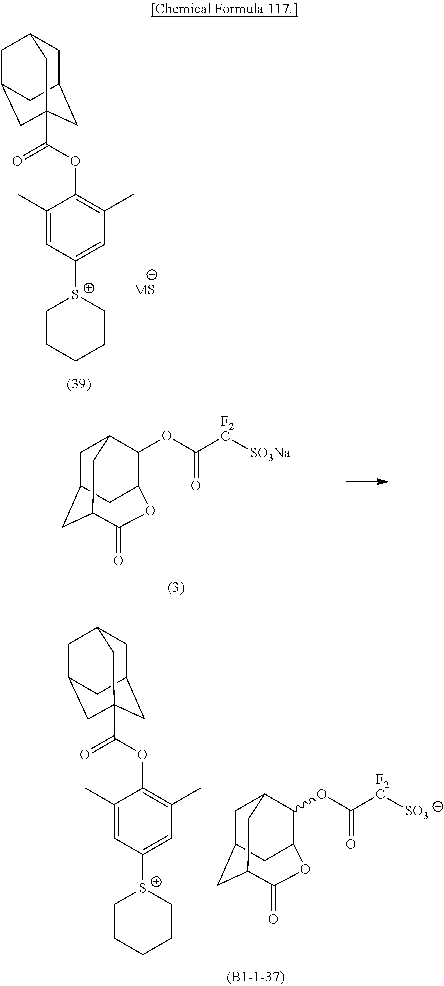

The obtained compound (B1-1-37) was analyzed by ¹H-NMR and ¹⁹F-NMR, and the structure thereof was identified by the following results.

¹H-NMR (DMSO, 400 MHz): δ(ppm)=7.82 (m, 2H, Ar), 5.02 (s, 1H, CH), 4.21 (s, 1H, CH), 3.73-3.91 (m, 4H, CH₂), 2.93 (s, 1H, CH), 1.41-2.29 (m, 37H, Ar—CH₃+CH₂+Adamantane+Adamantanelactone).

¹⁹F-NMR (DMSO, 376 MHz): δ(ppm)=−107.3.

From the results of the NMR analyses, it was confirmed that the compound (B1-1-37) had a structure as shown above.

### Example 38

### Synthesis of Compound (B1-1-38)

A compound (40) and a compound (3) were reacted in the same manner as in the synthesis example described in ii) of Example 1, thereby obtaining a compound (B1-1-38).

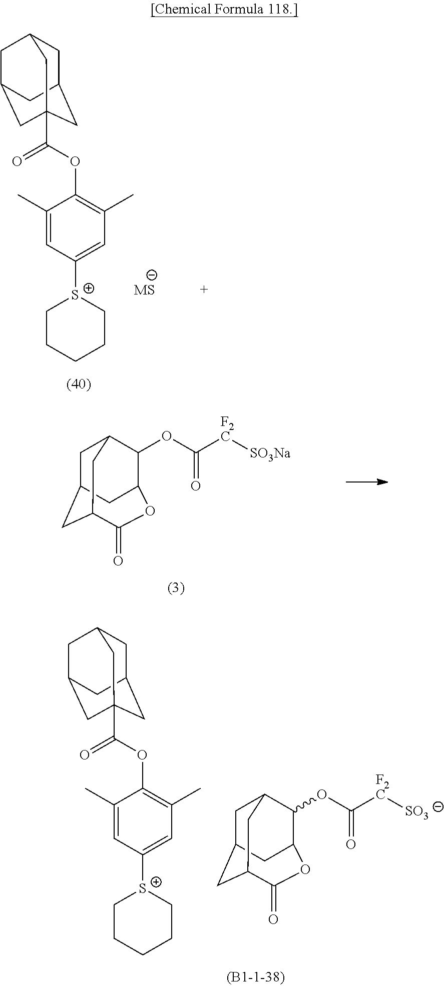

The obtained compound (B1-1-38) was analyzed by ¹H-NMR and ¹⁹F-NMR, and the structure thereof was identified by the following results.

¹H-NMR (DMSO, 400 MHz): δ(ppm)=7.82 (m, 2H, Ar), 5.02 (s, 1H, CH), 4.21 (s, 1H, CH), 3.73-3.91 (m, 4H, CH₂), 2.93 (s, 1H, CH), 1.41-2.29 (m, 37H, Ar—CH₃+CH₂+Adamantane+Adamantanelactone).

¹⁹F-NMR (DMSO, 376 MHz): δ(ppm)=−107.3.

From the results of the NMR analyses, it was confirmed that the compound (B1-1-38) had a structure as shown above.

### Example 39

### Synthesis of Compound (B1-1-39)

A compound (41) and a compound (3) were reacted in the same manner as in the synthesis example described in ii) of Example 1, thereby obtaining a compound (B1-1-39).

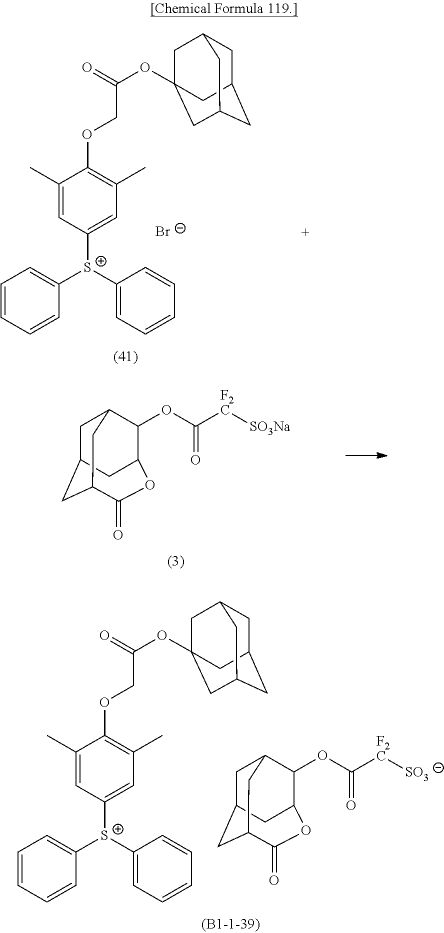

The obtained compound (B1-1-39) was analyzed by ¹H-NMR and ¹⁹F-NMR, and the structure thereof was identified by the following results.

¹H-NMR (DMSO, 400 MHz): δ(ppm)=7.77-7.98 (m, 10H, ArH), 7.64 (s, 2H, ArH), 5.02 (s, 1H, CH), 4.57 (s, 2H, CH₂O), 4.21 (s, 1H, CH), 2.93 (s, 1H, CH), 2.40 (s, 6H, CH₃), 1.41-2.29 (m, 25H, Adamantane+Adamantanelactone).

¹⁹F-NMR (DMSO, 376 MHz): δ(ppm)=−107.3

From the results of the NMR analyses, it was confirmed that the compound (B1-1-39) had a structure as shown above.

### Example 40

### Synthesis of Compound (B1-1-40)

A compound (42) and a compound (3) were reacted in the same manner as in the synthesis example described in ii) of Example 1, thereby obtaining a compound (B1-1-40).

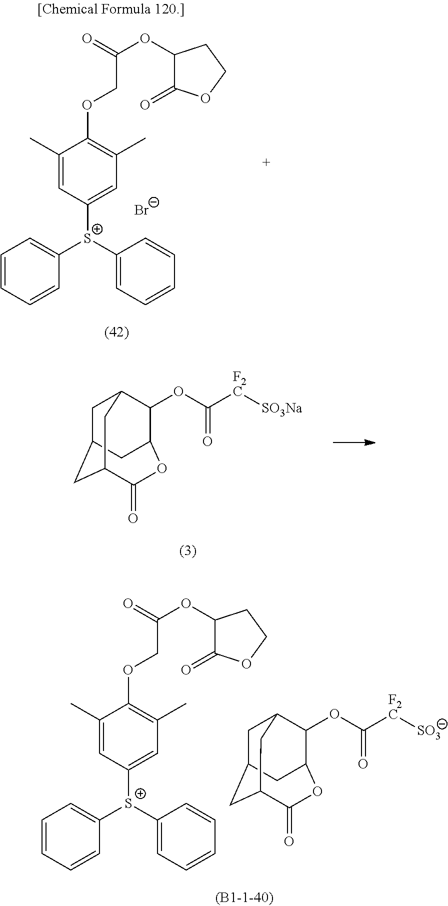

The obtained compound (B1-1-40) was analyzed by ¹H-NMR and ¹⁹F-NMR, and the structure thereof was identified by the following results.

¹H-NMR (DMSO, 400 MHz): δ(ppm)=7.77-7.89 (m, 10H, ArH), 7.64 (s, 2H, ArH), 5.70 (t, 1H₂OCHC═O), 5.02 (s, 1H, CH), 4.82 (s, 2H, ArOCH₂), 4.46-4.30 (m, 2H₂OCOCH₂), 4.21 (s, 1H, CH), 2.93 (s, 1H, CH), 2.71-2.64 (m, 1H₂OCH₂CH₂), 1.41-2.33 (m, 17H, CH₃+OCH₂CH₂+Adamantanelactone).

¹⁹F-NMR (DMSO, 376 MHz): δ(ppm)=−107.3

From the results of the NMR analyses, it was confirmed that the compound (B1-1-40) had a structure as shown above.

### Example 41

### Synthesis of Compound (B1-1-41)

A compound (43) and a compound (3) were reacted in the same manner as in the synthesis example described in ii) of Example 1, thereby obtaining a compound (B1-1-41).

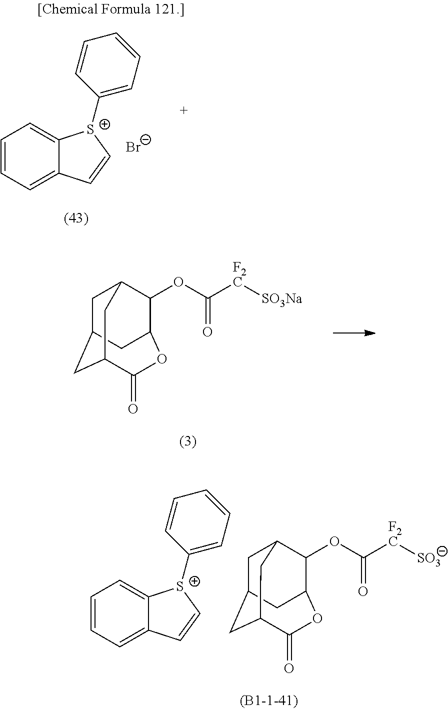

The obtained compound (B1-1-41) was analyzed by ¹H-NMR and ¹⁹F-NMR, and the structure thereof was identified by the following results.

¹H-NMR (DMSO, 400 MHz): δ(ppm)=8.28 (d, 2H, ArH), 8.11 (d, 1H, ArH), 7.86 (t, 1H, ArH), 7.63-7.81 (m, 7H, ArH), 5.02 (s, 1H, CH), 4.21 (s, 1H, CH), 2.93 (s, 1H, CH), 1.41-2.29 (m, 10H, Adamantanelactone).

¹⁹F-NMR (DMSO, 376 MHz): δ(ppm)=−107.3.

From the results of the NMR analyses, it was confirmed that the compound (B1-1-41) had a structure as shown above.

### Example 42

### Synthesis of Compound (B1-1-42)

A compound (44) and a compound (3) were reacted in the same manner as in the synthesis example described in ii) of Example 1, thereby obtaining a compound (B1-1-42).

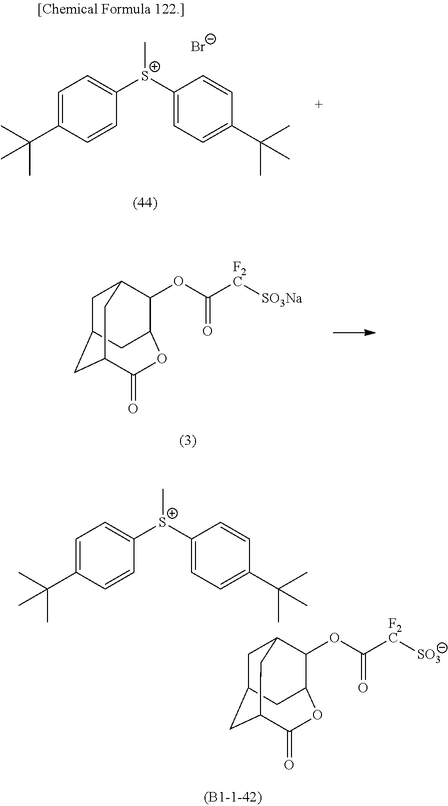

The obtained compound (B1-1-42) was analyzed by ¹H-NMR and ¹⁹F-NMR, and the structure thereof was identified by the following results.

¹H-NMR (DMSO, 400 MHz): δ(ppm)=8.05 (d, 2H, ArH), 7.74 (d, 2H, ArH), 5.02 (s, 1H, CH), 4.21 (s, 1H, CH), 3.85 (s, 3H, S—CH₃), 2.93 (s, 1H, CH), 1.41-2.29 (m, 10H, Adamantanelactone), 1.30 (s, 18H, t-Bu).

¹⁹F-NMR (DMSO, 376 MHz): δ(ppm)=−107.3.

From the results of the NMR analyses, it was confirmed that the compound (B1-1-42) had a structure as shown above.

### Example 43

### Synthesis of Compound (B1-1-43)

A compound (45) and a compound (3) were reacted in the same manner as in the synthesis example described in ii) of Example 1, thereby obtaining a compound (B1-1-43).

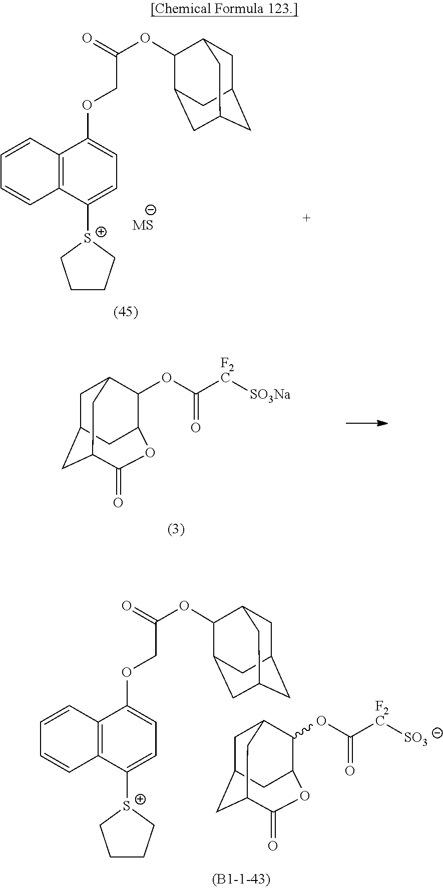

The obtained compound (B1-1-43) was analyzed by ¹H-NMR and ¹⁹F-NMR, and the structure thereof was identified by the following results.

¹H-NMR (DMSO, 400 MHz): δ(ppm)=8.41 (m, 2H, ArH), 8.12 (d, 1H, ArH), 7.73-7.93 (m, 2H, ArH), 7.19 (d, 1H, ArH), 5.23 (s, 2H, CH₂), 5.02 (s, 1H, CH), 4.95 (m, 1H, Adamantane), 4.21 (s, 1H, CH), 4.03 (m, 2H, CH₂S), 3.75 (m, 2H, CH₂S), 2.93 (s, 1H, CH), 1.41-2.43 (m, 28H, SCH₂CH₂+Adamantane+Adamantanelactone).

¹⁹F-NMR (DMSO, 376 MHz): δ(ppm)=−107.3.

From the results of the NMR analyses, it was confirmed that the compound (B1-1-43) had a structure as shown above.

### Example 44

### Synthesis of Compound (B1-1-44)

A compound (46) and a compound (3) were reacted in the same manner as in the synthesis example described in ii) of Example 1, thereby obtaining a compound (B1-1-44).

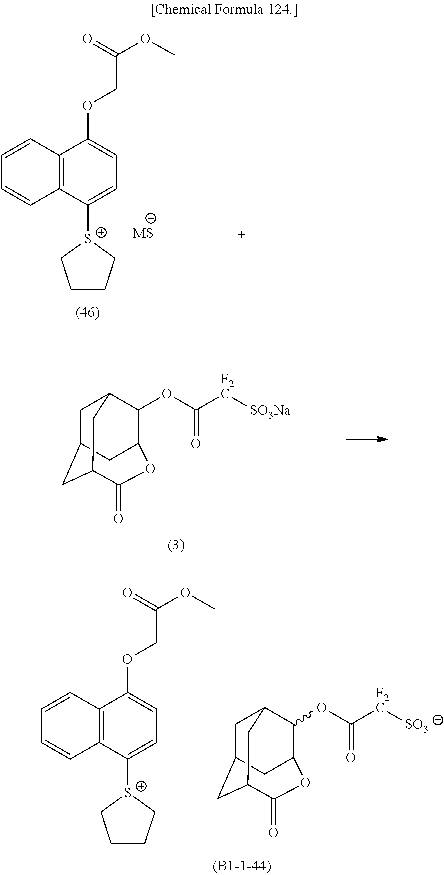

The obtained compound (B1-1-44) was analyzed by ¹H-NMR and ¹⁹F-NMR, and the structure thereof was identified by the following results.

¹H-NMR (DMSO, 400 MHz): δ(ppm)=8.42 (m, 2H, ArH), 8.17 (d, 1H, ArH), 7.78-7.91 (m, 2H, ArH), 7.23 (d, 1H, ArH), 5.26 (s, 2H, CH₂), 5.02 (s, 1H, CH), 4.21 (s, 1H, CH), 3.75-4.19 (m, 7H, SCH₂+CH₃), 1.41-2.60 (m, 14H, SCH₂CH₂+Adamantanelactone).

¹⁹F-NMR (DMSO, 376 MHz): δ(ppm)=−107.3.

From the results of the NMR analyses, it was confirmed that the compound (B1-1-44) had a structure as shown above.

### Example 45

### Synthesis of Compound (B1-1-45)

A compound (47) and a compound (3) were reacted in the same manner as in the synthesis example described in ii) of Example 1, thereby obtaining a compound (B1-1-45).

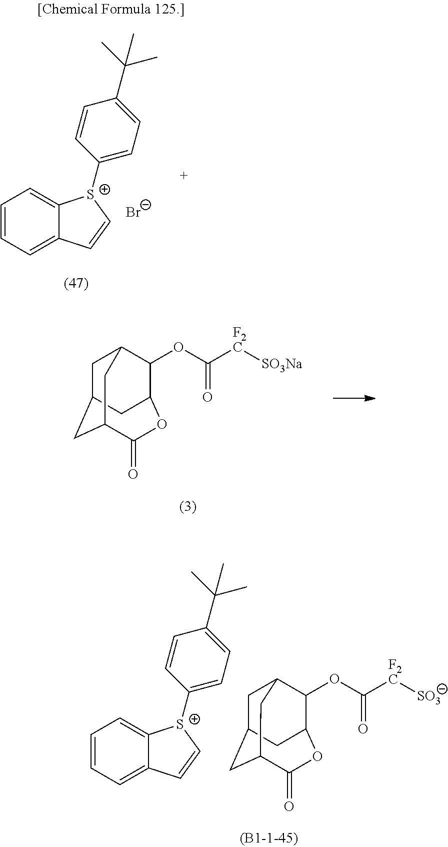

The obtained compound (B1-1-45) was analyzed by ¹H-NMR and ¹⁹F-NMR, and the structure thereof was identified by the following results.

¹H-NMR (DMSO, 400 MHz): δ(ppm)=8.28 (d, 2H, ArH), 8.12 (d, 1H, ArH), 7.88 (t, 1H, ArH), 7.80 (d, 1H, ArH), 7.62-7.74 (m, SH, ArH), 5.02 (s, 1H, CH), 4.21 (s, 1H, CH), 2.93 (s, 1H, CH), 1.41-2.29 (m, 10H, Adamantanelactone), 1.27 (s, 9H, CH₃).

¹⁹F-NMR (DMSO, 376 MHz): δ(ppm)=−107.3.

From the results of the NMR analyses, it was confirmed that the compound (B1-1-45) had a structure as shown above.

### Production of Resist Composition (1)

### Examples 46 to 78, Comparative Examples 1 to 4

The components shown in Table 1 were mixed together and dissolved to obtain positive resist compositions.

TABLE 1

| | Component | Component | Component | Component | | |
|---|---|---|---|---|---|---|
| | (A) | (B) | (D) | (E) | Component (S) | |
| Comp. Ex. 1 | (A)-1 | (B)-1 | (D)-1 | (E)-1 | (S)-1 | (S)-2 |
| | [100] | [8.00] | [1.20] | [1.32] | [10] | [2000] |
| Comp. Ex. 2 | (A)-1 | (B)-2 | (D)-1 | (E)-1 | (S)-1 | (S)-2 |
| | [100] | [8.20] | [1.20] | [1.32] | [10] | [2000] |
| Comp. Ex. 3 | (A)-1 | (B)-3 | (D)-1 | (E)-1 | (S)-1 | (S)-2 |
| | [100] | [8.68] | [1.20] | [1.32] | [10] | [2000] |
| Comp. Ex. 4 | (A)-1 | (B)-4 | (D)-1 | (E)-1 | (S)-1 | (S)-2 |
| | [100] | [8.56] | [1.20] | [1.32] | [10] | [2000] |
| Example 46 | (A)-1 | (B)-5 | (D)-1 | (E)-1 | (S)-1 | (S)-2 |
| | [100] | [8.56] | [1.20] | [1.32] | [10] | [2000] |

The resist compositions of Examples 47 to 78 were produced in the same manner as the resist composition of Example 46, except that the component (B) was changed ((B)-5 was changed to (B)-6 to (B)-37, respectively). Thus, the resist compositions of Examples 47 to 78 had the same formulation (component, amount) as that of the resist composition of Example 46, except for the component (B) (the amount of the component (B) was equimolar to that of (B)-1).

In Table 1 and above, the reference characters indicate the following. Further, the values in brackets [ ] indicate the amount (in terms of parts by weight) of the component added.

(A)-1: a copolymer (A1-11-1) represented by chemical formula shown below. Mw: 7,000, Mw/Mn: 1.80. In the chemical formula, the subscript numerals shown on the bottom right of the parentheses ( ) indicate the percentage (mol %) of the respective structural units.

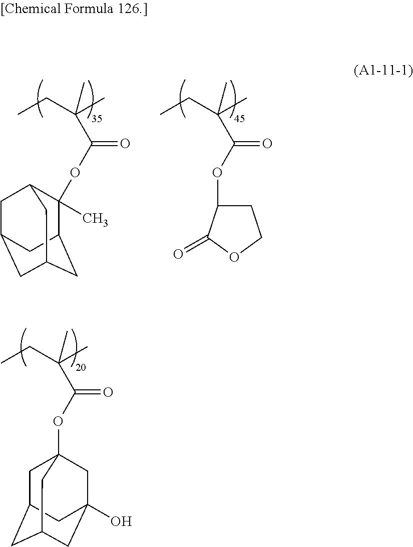

(B)-1: (4-methylphenyl)diphenylsulfonium nonafluoro-n-butanesulfonate

(B)-2: a compound (B2-1) represented by chemical formula shown below

(B)-3: a compound (B2-2) represented by chemical formula shown below

(B)-4: a compound (B2-3) represented by chemical formula shown below

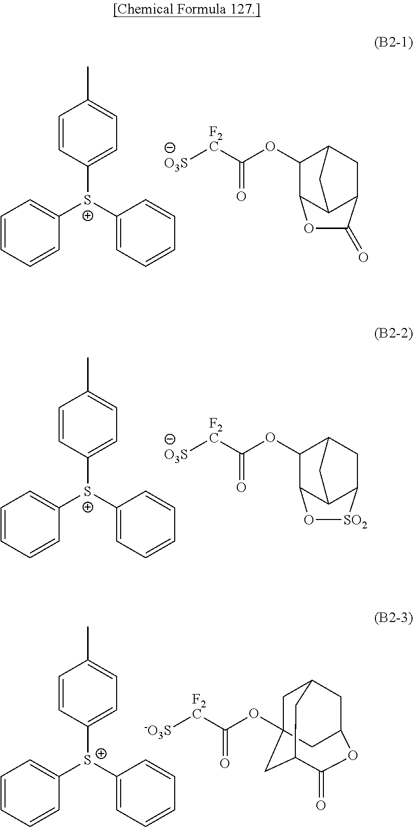

(B)-5: the aforementioned compound (B1-1-1)

(B)-6 to (B)-9: the aforementioned compounds (B1-1-2) to (B1-1-5)

(B)-10 to (B)-25: the aforementioned compounds (B1-1-8) to (B1-1-23)

(B)-26: the aforementioned compound (B1-1-25)

(B)-27 to (B)-31: the aforementioned compounds (B1-1-30) to (B1-1-34)

(B)-32: the aforementioned compound (B1-1-37)

(B)-33 to (B)-35: the aforementioned compounds (B1-1-39) to (B1-1-41)

(B)-36: the aforementioned compound (B1-1-43)

(B)-37: the aforementioned compound (B1-1-45)

(D)-1: tri-n-pentylamine

(E)-1: salicylic acid

(S)-1: γ-butyrolactone

(S)-2: a mixed solvent of PGMEA/PGME=6/4 (weight ratio)

<Evaluation of Lithography Properties and Shape of Resist Pattern (1)>

Using the obtained positive resist compositions, resist patterns were formed in the following manner, and the following evaluations were conducted.

[Formation of Resist Pattern]

An organic anti-reflection film composition (product name: ARC-29A, manufactured by Brewer Science Ltd.) was applied to an 8-inch silicon wafer using a spinner, and the composition was then baked on a hotplate at 205° C. for 60 seconds, thereby forming an organic anti-reflection film having a film thickness of 82 nm.

Then, each positive resist composition was applied to the anti-reflection film using a spinner, and was then prebaked (PAB) on a hotplate at 110° C. for 60 seconds and dried, thereby forming a resist film having a film thickness of 150 nm.

Subsequently, the resist film was selectively irradiated with an ArF excimer laser (193 nm) through a mask, using an ArF exposure apparatus NSR-5302 (manufactured by Nikon Corporation; NA (numerical aperture)=0.60, ⅔ annular illumination).

Thereafter, a post exposure bake (PEB) treatment was conducted at 110° C. for 60 seconds, followed by alkali development for 30 seconds at 23° C. in a 2.38% by weight aqueous solution of tetramethylammonium hydroxide (TMAH) (product name: NMD-3; manufactured by Tokyo Ohka Kogyo Co., Ltd.). Then, the resist film was washed for 30 seconds with pure water, followed by drying by shaking.

As a result, in each of the examples, a space and line resist pattern (hereafter, referred to as “SL pattern (1)”) in which spaces having a space width of 120 nm were provided at equal intervals (pitch: 240 nm) was formed on the resist film.

The optimum exposure dose Eop (mJ/cm²) with which the SL pattern (1) was formed, i.e., sensitivity was determined. The results are shown in Tables 2 and 3.

[Evaluation of Line Width Roughness (LWR)]

SL patterns (1) having a space width of 120 nm and a pitch of 240 nm were formed with the above Eop in the same manner as described above, and the space width at 400 points in the lengthwise direction of the space were measured using a measuring scanning electron microscope (SEM) (product name: S-9220, manufactured by Hitachi, Ltd.; acceleration voltage: 800V). From the results, the value of 3 times the standard deviation s (i.e., 3s) was determined, and the average of the 3s values at 5 points was calculated as a yardstick of LWR. The results are shown in Tables 2 and 3.

The smaller this 3s value is, the lower the level of roughness of the line width, indicating that an SL pattern with a uniform width was obtained.

[Evaluation of Exposure Latitude (EL Margin)]

The exposure dose with which an SL pattern having a dimension of the target dimension (space width: 120 nm) ±5% (i.e., 114 nm to 126 nm) was determined, and the EL margin (unit: %) was determined by the following formula. The results are shown in Tables 2 and 3.

EL margin (%)=(|E1−E2|/Eop)×100

E1: Exposure dose (mJ/cm²) with which an SL pattern having a space width of 114 nm was formed

E2: Exposure dose (mJ/cm²) with which an SL pattern having a line width of 126 nm was formed

The larger the value of the “EL margin”, the smaller the change in the pattern size by the variation of the exposure dose.

[Evaluation of Mask Error Factor (MEF)]

In the same manner as described above, with the above Eop, SL patterns were formed using a mask pattern targeting a space width of 120 nm and a pitch of 260 nm, and a mask pattern targeting a space width of 130 nm and a pitch of 260 nm, and the MEF value was calculated by the following formula. The results are shown in Tables 2 and 3.

MEF=|CD₁₃₀−CD₁₂₀|/|MD₁₃₀−MD₁₂₀|

In the formula, CD₁₃₀ and CD₁₂₀ represent the respective space widths (nm) of the actual SL patterns respectively formed using the mask pattern targeting a space width of 130 nm and the mask pattern targeting a space width of 120 nm. MD₁₃₀ and MD₁₂₀ represent the respective target space widths (nm), meaning MD₁₃₀=130, and MD₁₂₀=120.

A MEF value closer to 1 indicates that a resist pattern faithful to the mask pattern was formed.

[Evaluation of Resist Pattern Shape]

Each of the SL patterns (1) having a space width of 120 nm and a pitch of 240 nm and formed with the above Eop was observed using a scanning electron microscope (SEM), and the cross-sectional shape of the SL pattern (1) was evaluated. The results are shown in Tables 2 and 3.

TABLE 2

| | | | | | | Shape of |
|---|---|---|---|---|---|---|
| | PAB/PEB | Eop | LWR | EL margin | | resist |
| | (° C.) | (mJ/cm²) | (nm) | (%) | MEF | pattern |
| Comp. | 110/110 | 32.8 | 13.2 | 6.32 | 2.53 | Tapered |
| Ex. 1 | | | | | | |
| Comp. | 110/110 | 42.8 | 11.4 | 7.76 | 2.22 | Rounded top |
| Ex. 2 | | | | | | |
| Comp. | 110/110 | 47.8 | 11.6 | 7.36 | 2.10 | Tapered |
| Ex. 3 | | | | | | |
| Comp. | 110/110 | 46.2 | 11.2 | 7.53 | 2.07 | Rectangular |
| Ex. 4 | | | | | | |
| Ex. 46 | 110/110 | 45.4 | 10.7 | 7.95 | 1.97 | Rectangular |
| Ex. 47 | 110/110 | 63.2 | 9.9 | 8.23 | 1.91 | Rectangular |
| Ex. 48 | 110/110 | 49.3 | 9.5 | 8.41 | 1.92 | Rectangular |
| Ex. 49 | 110/110 | 48.9 | 9.3 | 8.42 | 1.93 | Rectangular |
| Ex. 50 | 110/110 | 48.3 | 10.1 | 8.39 | 1.99 | Rectangular |
| Ex. 51 | 110/110 | 48.9 | 9.6 | 8.21 | 1.99 | Rectangular |
| Ex. 52 | 110/110 | 47.3 | 9.3 | 8.51 | 1.93 | Rectangular |
| Ex. 53 | 110/110 | 45.3 | 9.4 | 9.32 | 1.91 | Rectangular |
| Ex. 54 | 110/110 | 44.2 | 9.8 | 8.29 | 1.91 | Rectangular |
| Ex. 55 | 110/110 | 47.3 | 9.9 | 8.89 | 1.92 | Rectangular |
| Ex. 56 | 110/110 | 68.5 | 9.1 | 9.12 | 1.93 | Rectangular |
| Ex. 57 | 110/110 | 78.5 | 10.3 | 10.24 | 1.99 | Rectangular |
| Ex. 58 | 110/110 | 58.9 | 10.2 | 9.37 | 1.92 | Rectangular |
| Ex. 59 | 110/110 | 63.2 | 10.0 | 9.14 | 1.93 | Rectangular |
| Ex. 60 | 110/110 | 59.3 | 9.3 | 8.78 | 1.94 | Rectangular |
| Ex. 61 | 110/110 | 55.6 | 9.5 | 9.98 | 1.92 | Rectangular |
| Ex. 62 | 110/110 | 49.5 | 9.4 | 10.32 | 1.89 | Rectangular |

TABLE 3

| | | Eop | | | | Shape of |
|---|---|---|---|---|---|---|
| | PAB/PEB | (mJ/ | LWR | EL margin | | resist |
| | (° C.) | cm²) | (nm) | (%) | MEF | pattern |
| Ex. 63 | 110/110 | 47.3 | 9.1 | 8.91 | 1.99 | Rectangular |
| Ex. 64 | 110/110 | 45.2 | 9.9 | 8.28 | 1.92 | Rectangular |
| Ex. 65 | 110/110 | 46.3 | 9.8 | 8.51 | 1.94 | Rectangular |
| Ex. 66 | 110/110 | 46.7 | 10.2 | 9.32 | 1.95 | Rectangular |
| Ex. 67 | 110/110 | 35.4 | 11.0 | 8.29 | 1.95 | Rectangular |
| Ex. 68 | 110/110 | 53.4 | 9.3 | 9.63 | 1.95 | Rectangular |
| Ex. 69 | 110/110 | 45.3 | 9.8 | 8.23 | 1.91 | Rectangular |
| Ex. 70 | 110/110 | 45.4 | 9.5 | 8.87 | 1.92 | Rectangular |
| Ex. 71 | 110/110 | 41.3 | 9.1 | 9.90 | 1.93 | Rectangular |
| Ex. 72 | 110/110 | 38.9 | 9.7 | 8.12 | 1.99 | Rectangular |
| Ex. 73 | 110/110 | 49.9 | 10.5 | 8.59 | 1.92 | Rectangular |
| Ex. 74 | 110/110 | 54.1 | 9.1 | 9.83 | 1.99 | Rectangular |
| Ex. 75 | 110/110 | 42.1 | 9.7 | 9.64 | 1.91 | Rectangular |
| Ex. 76 | 110/110 | 47.5 | 9.4 | 9.91 | 1.87 | Rectangular |
| Ex. 77 | 110/110 | 45.3 | 9.9 | 10.73 | 1.89 | Rectangular |
| Ex. 78 | 110/110 | 53.7 | 9.3 | 9.70 | 1.94 | Rectangular |

From the results shown in the tables, it was confirmed that the resist patterns formed using the resist compositions of Examples 46 to 78 exhibited excellent LWR, MEF and EL margin, i.e., excellent lithography properties, and the shape of the patterns was excellent with high rectangularity, as compared to the resist patterns formed using the resist compositions of Comparative Examples 1 to 4.

### Production of Resist Composition (2)

### Example 79, Comparative Example 5

The components shown in Table 4 were mixed together and dissolved to obtain positive resist compositions.

TABLE 4

| | Component | Component | | Component |
|---|---|---|---|---|
| | (A) | (B) | | (S) |
| Comp. Ex. 5 | (A)-2 | (B)-38 | (B)-39 | (S)-2 |
| | [100] | [10.75] | [2.28] | [2000] |
| Ex. 79 | (A)-2 | (B)-5 | (B)-39 | (S)-2 |
| | [100] | [8.16] | [2.28] | [2000] |

In Table 4, the reference characters indicate the following. Further, the values in brackets [ ] indicate the amount (in terms of parts by weight) of the component added.

(A)-2: a copolymer (A1-13-1) represented by chemical formula shown below. Mw: 7,000, Mw/Mn: 1.85. In the chemical formula, the subscript numerals shown on the bottom right of the parentheses ( ) indicate the percentage (mol %) of the respective structural units.

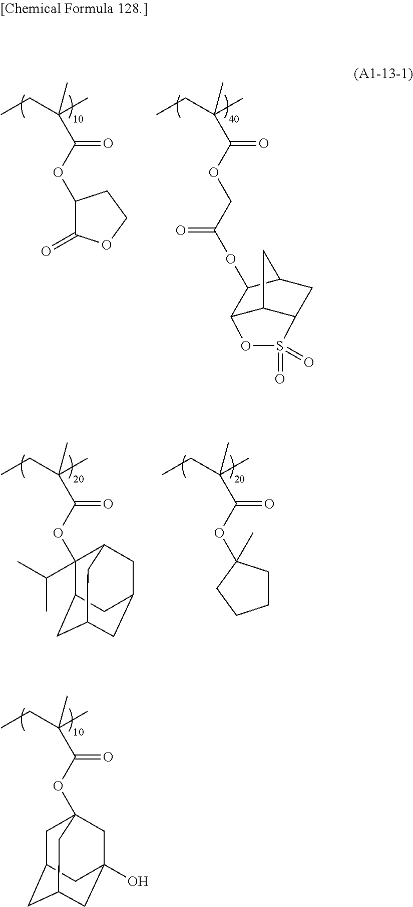

(B)-5: the aforementioned compound (B1-1-1)

(B)-38: a compound (B2-4) represented by chemical formula shown below

(B)-39: a compound (B2-5) represented by chemical formula shown below

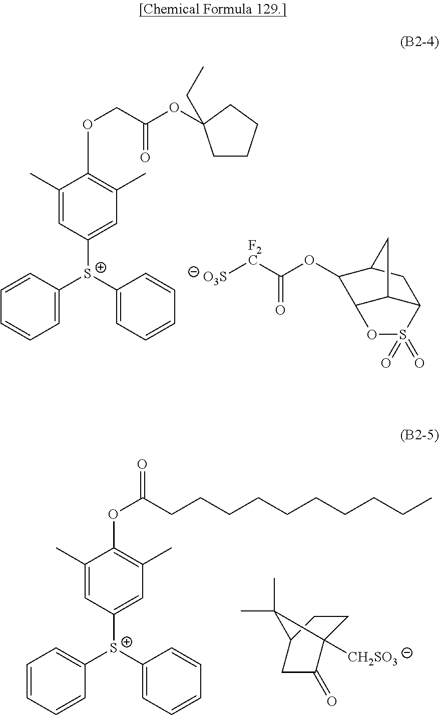

(S)-2: a mixed solvent of PGMEA/PGME=6/4 (weight ratio)

<Evaluation of Lithography Properties and Shape of Resist Pattern (2)>

Using the obtained positive resist compositions, resist patterns were formed in the following manner, and the following evaluations were conducted.

[Formation of Resist Pattern]

An organic anti-reflection film composition (product name: ARC145, manufactured by Brewer Science Ltd.) was applied to an 8-inch silicon wafer using a spinner, and the composition was then baked on a hotplate at 205° C. for 60 seconds, thereby forming an organic anti-reflection film having a film thickness of 40 nm.

Then, another organic anti-reflection film composition (product name: ARC113, manufactured by Brewer Science Ltd.) was applied to the organic anti-reflection film using a spinner, followed by baking on a hotplate at 205° C. for 60 seconds, thereby forming an organic anti-reflection film having a film thickness of 50 nm.

Then, each positive resist composition was applied to the organic anti-reflection film using a spinner, and was then prebaked (PAB) on a hotplate at 90° C. for 60 seconds and dried, thereby forming a resist film having a film thickness of 100 nm.

Subsequently, a coating solution for forming a protection film (product name: TILC-320; manufactured by Tokyo Ohka Kogyo Co., Ltd.) was applied to the resist film using a spinner, and then heated at 90° C. for 60 seconds, thereby forming a top coat with a film thickness of 35 nm.

Thereafter, using an ArF immersion exposure apparatus (product name: NSR-5609B, manufactured by Nikon Corporation, NA (numerical aperture)=1.07, Annular 0.55-0.80), the resist film having a top coat formed thereon was selectively irradiated with an ArF excimer laser (193 nm) through a mask (6% half tone).

Thereafter, a post exposure bake (PEB) treatment was conducted at 85° C. for 60 seconds, followed by alkali development for 40 seconds at 23° C. in a 2.38% by weight aqueous solution of tetramethylammonium hydroxide (TMAH) (product name: NMD-3; manufactured by Tokyo Ohka Kogyo Co., Ltd.). Then, the resist film was washed for 30 seconds with pure water, followed by drying by shaking.

As a result, in each of the examples, a space and line resist pattern (hereafter, referred to as “SL pattern (2)”) in which spaces having a space width of 60 nm were provided at equal intervals (pitch: 120 nm) was formed on the resist film.

The optimum exposure dose Eop (mJ/cm²) with which the SL pattern (2) was formed, i.e., sensitivity was determined. The results are shown in Table 5.

[Evaluation of Exposure Latitude (EL Margin)]

The exposure dose with which an SL pattern having a dimension of the target dimension (space width: 60 nm) ±5% (i.e., 57 nm to 63 nm) was determined, and the EL margin (unit: %) was determined by the following formula. The results are shown in Table 5.

EL margin (%)=(|E1−E2|/Eop)×100

E1: Exposure dose (mJ/cm²) with which an SL pattern having a space width of 57 nm was formed

E2: Exposure dose (mJ/cm²) with which an SL pattern having a space width of 63 nm was formed

[Evaluation of Line Edge Roughness (LER)]

With respect to each of the SL patterns (2) formed with the above Eop, the line width at 100 points were measured using a measuring scanning electron microscope (SEM) (product name: S-9220, manufactured by Hitachi, Ltd.; acceleration voltage: 800V), and from the results, the value of 3 times the standard deviation s (i.e., 3s) was calculated as a yardstick of LER. The results are shown in Table 5.

The smaller this 3s value is, the lower the level of roughness of the line width, indicating that an SL pattern with a uniform width was obtained.

[Evaluation of Mask Error Factor (MEF)]

With the Eop for forming the above SL pattern (2), the mask size was changed from 55 to 65 nm at intervals of 1 nm (with fixed pitch) to form resist patterns. Then, a linear equation line of the pattern size and the mask size was plotted, and the gradient of the linear equation line was determined as the MEF. The results are shown in Table 5.

A MEF value closer to 1 indicates that a resist pattern faithful to the mask pattern was formed.

[Evaluation of Resist Pattern Shape]

Each of the SL patterns (2) having a space width of 60 nm and a pitch of 120 nm and formed with the above Eop was observed using a scanning electron microscope (SEM), and the cross-sectional shape of the SL pattern (2) was evaluated. The results are shown in the tables.

TABLE 5

| | Comp. Ex. 5 | Ex. 79 |
|---|---|---|
| Eop (mJ/cm²) | 37.5 | 30.2 |
| EL margin (%) | 7.73 | 8.28 |
| LER (nm) | 4.83 | 4.68 |
| MEF | 3.25 | 3.24 |
| Shape of resist | T-top | Rectangular |
| pattern | | |

From the results shown in the table, it was confirmed that the resist pattern formed using the resist composition of Example 79 exhibited excellent EL margin, LER and MEF, i.e., excellent lithography properties, and the shape of the patterns was excellent with high rectangularity, as compared to the resist pattern formed using the resist composition of Comparative Example 5.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. A resist composition comprising a base component (A) which exhibits changed solubility in an alkali developing solution under action of acid and an acid-generator component (B) which generates acid upon exposure,
the acid-generator component (B) comprising an acid generator (B1) comprised of a compound represented by general formula (b1-1) shown below: 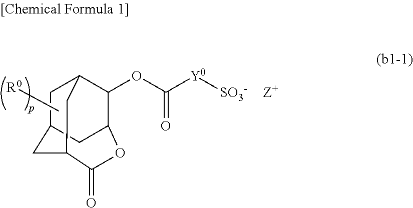
wherein Y⁰ represents an alkylene group of 1 to 4 carbon atoms which may have a substituent or a fluorinated alkylene group which may have a substituent; R⁰ represents an alkyl group, an alkoxy group, a halogen atom, a halogenated alkyl group, a hydroxy group or an oxygen atom (═O); p represents 0 or 1; and Z⁺ represents an organic cation.

2. The resist composition according to claim 1, wherein the amount of the acid generator (B1), relative to 100 parts by weight of the base component (A) is in the range of 0.1 to 50 parts by weight.

3. The resist composition according to claim 1, wherein said base component (A) is a base component which exhibits increased solubility in an alkali developing solution under action of acid.

4. The resist composition according to claim 3, wherein the base component (A) comprises a resin component (A1) comprised of a structural unit (a1) derived from an acrylate ester which may have an atom other than hydrogen or a group bonded to the carbon atom on the α position and containing an acid dissociable, dissolution inhibiting group.

5. A method of forming a resist pattern, comprising: forming a resist film on a substrate using a resist composition of claim 1; conducting exposure of the resist film; and alkali-developing the resist film to form a resist pattern.

6. A compound represented by general formula (b1-1) shown below: 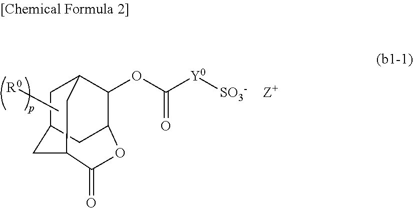
wherein Y⁰ represents an alkylene group of 1 to 4 carbon atoms which may have a substituent or a fluorinated alkylene group which may have a substituent; R⁰ represents an alkyl group, an alkoxy group, a halogen atom, a halogenated alkyl group, a hydroxy group or an oxygen atom (═O); p represents 0 or 1; and Z⁺ represents an organic cation.

7. An acid generator consisting of a compound of claim 6.
